(19) United States
(12) Patent Application Publication
(10) Pub. No.: US 2023/0365589 A1
(43) Pub. Date: Nov. 16, 2023

(54) NOVEL AMINE DERIVATIVES

(71) Applicants: Sumitomo Pharma Co., Ltd., Osaka (JP); CARNA BIOSCIENCES, INC., Hyogo (JP)

(72) Inventors: Shingo TOJO, Osaka-shi, Osaka (JP); Daisuke URABE, Osaka-shi, Osaka (JP); Hitoshi WATANABE, Osaka-shi, Osaka (JP); Wataru KAWAHATA, Kobe-shi, Hyogo (JP); Hideki MORIYAMA, Kobe-shi, Hyogo (JP); Yuko ASAMITSU, Kobe-shi, Hyogo (JP)

(73) Assignees: Sumitomo Pharma Co., Ltd., Osaka (JP); CARNA BIOSCIENCES, INC., Hyogo (JP)

(21) Appl. No.: 18/026,187

(22) PCT Filed: Sep. 17, 2021
(86) PCT No.: PCT/JP2021/034327

§ 371 Date: Mar. 14, 2023

(30) Foreign Application Priority Data
Sep. 18, 2020 (JP) 2020-157398
Jul. 28, 2021 (JP) 2021-122893

(51) Int. Cl. C07D 519/00

(52) U.S. Cl. CPC C07D 519/00

(57) ABSTRACT

The present invention provides an amine derivative having a DYRK-inhibiting activity and represented by formula (1):

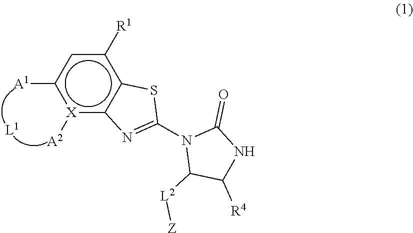

wherein A¹, A², L¹, L², X, Z, R¹ and R⁴ are as defined in the description,
or a pharmaceutically acceptable salt thereof.

### TECHNICAL FIELD

The present invention relates to a medicament, particularly a novel amine derivative having a DYRK inhibitory effect or a pharmaceutically acceptable salt thereof.

### BACKGROUND ART

DYRK (dual-specificity tyrosine-phosphorylation regulated kinase) is one of the bispecific protein kinases that phosphorylate tyrosine, serine, and threonine. DYRK functions as a tyrosine kinase only in the case of autophosphorylation and catalyzes the phosphorylation of serine or threonine residues on exogenous substrates. Five members of the DYRK family are known in humans: DYRK1A, DYRK1B, DYRK2, DYRK3, and DYRK4 (Non Patent Literature 1).

It has been widely reported that DYRK1A is associated with neuropsychiatric diseases. For example, in patients with Alzheimer's disease, the expression of 0-amyloid is significantly consistent with that of DYRK1A (Non Patent Literature 2), and it is speculated that DYRK1A is involved in abnormal phosphorylation of a tau protein (Tau), which is considered to contribute to the onset of Alzheimer's disease (Non Patent Literature 3).

In addition, Parkinson's disease is a neurodegenerative disease caused by the degeneration of dopamine neurons, which are important for motor function, but one of the causes is considered to be mitochondrial dysfunction (Non Patent Literature 4). An enzyme involved in protein degradation called Parkin is known to metabolize abnormal mitochondria and suppress abnormal accumulation, but DYRK1A has been reported to suppress the activity of this parkin protein (Non Patent Literature 5).

The gene for DYRK1A is located in the Down's syndrome critical region, and it has been reported that mice overexpressing DYRK1A exhibit neuropsychiatric dysfunction and appear like Down's syndrome (Non Patent Literature 6). It has also been reported that DYRK1A expression is increased in the brain of patients with Down's syndrome and Down's syndrome-like model mice (Non Patent Literature 7). These reports suggest that DYRK1A is involved in the onset of neurological symptoms in the patients with Down's syndrome (Non Patent Literature 8).

In addition, it has been reported that early-onset Alzheimer's disease occurs frequently in patients with Down's syndrome, thus indicating that DYRK1A is closely related to Alzheimer's disease (Non Patent Literature 8).

Therefore, compounds inhibiting DYRK1A are considered useful for treating neuropsychiatric diseases such as Alzheimer's disease, Down's syndrome, mental retardation, memory impairment, memory loss, and Parkinson's disease.

Recently, it has been reported that DYRK1A is highly expressed in brain tumors such as glioblastoma and regulates the expression of an epidermal growth factor receptor (EGFR) (Non Patent Literature 9). Therefore, compounds inhibiting DYRK1A are considered useful for treating EGFR-dependent cancers by suppressing the proliferation of cancer cells in EGFR-dependent brain tumors and other tumors.

Compounds inhibiting the family enzymes DYRK1B, DYRK2, and DYRK3 are also considered to have various pharmaceutical applications. For example, it has been reported that DYRK1B is highly expressed in quiescent (GO-phase) cancer cells and contributes to resistance to various chemotherapeutic agents (Non Patent Literature 10). It has also been reported that inhibition of DYRK1B promotes withdrawal from the GO phase and enhances sensitivity to chemotherapeutic agents (Non Patent Literature 11). Therefore, compounds inhibiting DYRK1B are considered useful for treating pancreatic cancer, ovarian cancer, osteosarcoma, colorectal cancer, and lung cancer (Non Patent Literatures 11, 12, 13, 14, and 15).

It is suggested that DYRK2 controls p53 to induce apoptosis in response to DNA damages (Non Patent Literature 16). Furthermore, it has been reported that compounds inhibiting DYRK3 are useful for treating sickle cell anemia and chronic kidney disease (Non Patent Literature 17).

In addition to Patent Literature 1 for compounds inhibiting DYRK, Patent Literature 2 has been reported for DYRK1A and DYRK1B inhibitors. However, the alkyne derivative of the present invention is not disclosed therein.

### PRIOR ART DOCUMENT(S)

### Patent Document(s)

[Patent Literature 1] WO2010/10797
[Patent Literature 2] WO2013/26806

### Non-Patent Document(s)

[Non-Patent Literature 1] Becker W. et al., J. Biol. Chem., 1998, 273, 25893-25902
[Non-Patent Literature 2] Kimura R. et al., Hum. Mol. Genet., 2007, 16, 15-23
[Non-Patent Literature 3] Ryoo SR. et al., J. Biol. Chem., 2007, 282, 34850-34857
[Non-Patent Literature 4] Narendra D. et al., J. Cell. Biol., 2008, 183, 795-803
[Non-Patent Literature 5] Im E., J. Neurochem., 2015, 134, 756-768
[Non-Patent Literature 6] Branchi I. et al., J. Neuropathol. Exp. Neurol., 2004, 63, 429-440
[Non-Patent Literature 7] Dowjat WK. et al., Neurosci. Lett., 2007, 413, 77-81
[Non-Patent Literature 8] Wegiel J. et al., FEBS J., 2011, 278, 236-245
[Non-Patent Literature 9] Pozo N. et al., J. Clin. Invest., 2013, 123, 2475-2487.
[Non-Patent Literature 10] Deng X. et al., Cancer Res., 2006, 66, 4149-4158.
[Non-Patent Literature 11] Ewton DZ. et al., Mol. Cancer Ther., 2011, 10, 2104-2114.
[Non-Patent Literature 12] Deng X. et al., Genes Cancer, 2014, 5, 201-211.
[Non-Patent Literature 13] Yang C. et al., Carcinogenesis, 2010, 31, 522-528.
[Non-Patent Literature 14] Jin K. et al., J. Biol. Chem., 2009, 284, 22916-22925.
[Non-Patent Literature 15] Gao J et al., Cancer Cell Int. 2013, 13, 2
[Non-Patent Literature 16] Taira N. et al., Mol. Cell, 2007, 25, 725-738.
[Non-Patent Literature 17] Bogacheva O. et al., J. Biol. Chem., 2008, 283, 36665-36675.

### DISCLOSURE OF INVENTION

### Problem to be Solved by the Invention

An object of the present invention is to provide a medicament, particularly a novel compound having a DYRK inhibitory effect.

### Means for Solving Problem

That is, the present invention is as follows.

[Item 1]

A compound represented by the following formula (1):

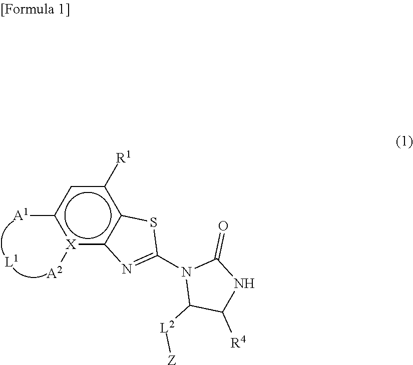

wherein
A¹ represents an oxygen atom or a nitrogen atom (═N—),
A² represents CR^{B}, CR^{C}R^{D}, an oxygen atom, or NR^{A1},
L¹ represents optionally substituted methylene, optionally substituted ethylene, optionally substituted methine, optionally substituted ethanediylidene, ═N—, or NR^{A2},
R^{A1}, R^{A2}, R^{B}, R^{C}, and R^{D} each independently represent a hydrogen atom or optionally substituted C₁₋₆ alkyl,
R¹ represents a hydrogen atom, a halogen atom, or optionally substituted C₁₋₆ alkyl,
X represents a carbon atom or a nitrogen atom,
L² represents optionally substituted C₁₋₄ alkylene,
R^{E} represents optionally substituted C₁₋₆ alkyl,
Z represents —NR²R³ or —OR⁷,
R⁷ represents optionally substituted C₁₋₆ alkyl, or optionally substituted C₁₋₇ alkylene formed together with R⁴, wherein R⁴ and R⁷, together with the carbon atom and the oxygen atom to which they, respectively, are attached, form an optionally substituted 5- to 11-membered saturated heterocycle,
R² represents a hydrogen atom, optionally substituted C₁₋₆ alkyl, C(O)—R^{E}, C₃₋₁₀ cycloalkyl, C₂₋₆ alkynyl, or a cyclic group of a 4- to 11-membered saturated heterocycle,
R³ represents a hydrogen atom, optionally substituted C₁₋₆ alkyl, or C(O)—R^{E}, and
R⁴ represents optionally substituted C₁₋₆ alkyl,
wherein R² and R³, together with the nitrogen atom to which they are attached, may form an optionally substituted 4- to 11-membered saturated heterocycle, or R³ and R⁴, together with the nitrogen atom and the carbon atom to which they, respectively, are attached, may form an optionally substituted 4- to 1l-membered saturated heterocycle, or any carbon atom on the saturated heterocycle constituted by R², R³, and the nitrogen atom, and R⁴ together may form a 4- to 1l-membered saturated heterocycle (as used herein, may also be referred to as “compound (1)” or the “compound represented by formula (1)”),
or a pharmaceutically acceptable salt thereof.

[Item 2]

The compound according to item 1 or a pharmaceutically acceptable salt thereof, wherein Z represents —NR²R³, and R² and R³ each independently represent a hydrogen atom, optionally substituted C₁ alkyl, or C(O)—R^{E}, wherein R² and R³, together with the nitrogen atom to which they are attached, may form an optionally substituted 4- to 8-membered saturated heterocycle.

[Item 3]

The compound according to item 1 or a pharmaceutically acceptable salt thereof, wherein Z represents —NR²R³, and R³ and R⁴, together with the nitrogen atom and the carbon atom to which they, respectively, are attached, form an optionally substituted 4- to 8-membered saturated heterocycle.

[Item 4]

The compound according to any one of items 1 to 3 or a pharmaceutically acceptable salt thereof, wherein R¹ is a hydrogen atom.

[Item 5]

The compound according to any one of items 1 to 4 or a pharmaceutically acceptable salt thereof, wherein X is a carbon atom.

[Item 6]

The compound according to item 5 or a pharmaceutically acceptable salt thereof, wherein A¹ is an oxygen atom, A² is methylene, and L¹ is methylene.

[Item 7]

The compound according to item 1, 2, 4, 5, or 6 or a pharmaceutically acceptable salt thereof, wherein formula (1) is represented by the following formula (1a):

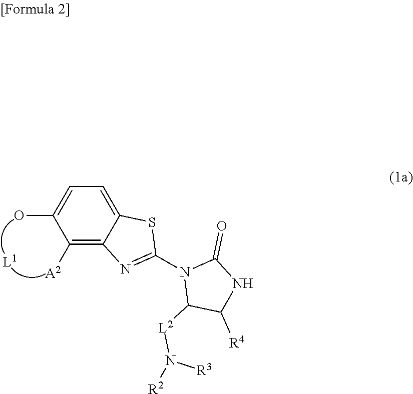

wherein A² represents optionally substituted methylene or an oxygen atom,
L¹ represents optionally substituted methylene or optionally substituted ethylene,
L² represents optionally substituted C₁₋₄ alkylene,
R² and R³ each independently represent a hydrogen atom, optionally substituted C₁₋₆ alkyl, or C(O)—R^{E}, wherein R² and
R³, together with the nitrogen atom to which they are attached, may form an optionally substituted 4- to 8-membered saturated heterocycle,
R^{E} represents optionally substituted C₁₋₆ alkyl, and
R⁴ is optionally substituted C₁₋₆ alkyl.

[Item 8]

The compound according to item 7, wherein R² and R³ each independently are optionally substituted C₁₋₆ alkyl, or a pharmaceutically acceptable salt thereof.

[Item 9]

The compound according to item 7 or a pharmaceutically acceptable salt thereof, wherein R² and R³, together with the nitrogen atom to which they are attached, form an optionally substituted 4- to 8-membered saturated heterocycle.

[Item 10]

The compound according to any one of items 7 to 9 or a pharmaceutically acceptable salt thereof, wherein L² is C₁₋₃ alkylene.

[Item 11]

The compound according to item 1, 3, 4, 5, or 6 or a pharmaceutically acceptable salt thereof, wherein formula (1) is represented by the following formula (1b):

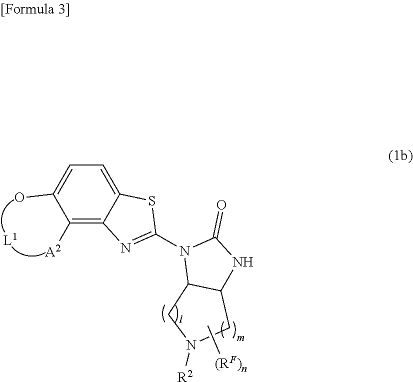

wherein
A² represents optionally substituted methylene or an oxygen atom,
L¹ represents optionally substituted methylene or optionally substituted ethylene,
l and m each independently represent 1, 2, or 3, wherein a sum of 1 and m is 5 or less,
n represents 1, 2, 3, or 4,
R² represents a hydrogen atom, optionally substituted C₁₋₆ alkyl, C₃₋₁₀ cycloalkyl, or C(O)—R^{E},
R^{E} represents optionally substituted C₁₋₆ alkyl,
R^{F} represents a hydrogen atom, a halogen atom, or optionally substituted C₁₋₆ alkyl,
when n is 2, 3, or 4, each R^{E} may be the same or different, and two R^{F} on the same carbon atom, together with the carbon atom to which they are each attached, may form a spiro ring consisting of a 4- to 8-membered saturated heterocycle or a 3- to 8-membered saturated carbocycle, or two R^{F} on different carbon atoms may bond together to form a crosslink.

[Item 12]

The compound according to item 11 or a pharmaceutically acceptable salt thereof, wherein R² is a hydrogen atom, optionally substituted C₁₋₆ alkyl, or C₃₋₁₀ cycloalkyl.

[Item 13]

The compound according to item 11 or a pharmaceutically acceptable salt thereof, wherein R² is optionally substituted C₁₋₆ alkyl.

[Item 14]

The compound according to any one of items 11 to 13 or a pharmaceutically acceptable salt thereof, wherein 1 and m are each independently 1 or 2.

[Item 15]

The compound according to item 1, wherein Z is —OR⁷, and R⁴ and R⁷, together with the carbon atom and the oxygen atom to which they, respectively, are attached, form an optionally substituted 5- to 8-membered saturated heterocycle, or a pharmaceutically acceptable salt thereof.

[Item 16]

The compound according to item 1 or 15 or a pharmaceutically acceptable salt thereof, wherein formula (1) is represented by the following formula (1c):

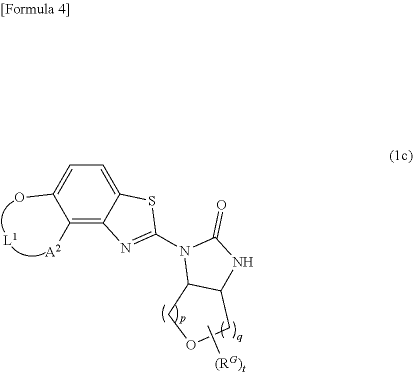

wherein
A² represents optionally substituted methylene or an oxygen atom,
L¹ represents optionally substituted methylene or optionally substituted ethylene,
p and q each independently represent 1, 2, or 3, wherein a sum of p and q is 5 or less,
t represents 1, 2, 3, or 4,
R^{G} represents a hydrogen atom, a halogen atom, optionally substituted C₁₋₆ alkyl, optionally substituted C₁₋₆ alkoxy, or a CN group,
when t is 2, 3, or 4, each R^{G} may be the same or different, and two R^{G} on the same carbon atom, together with the carbon atom to which they are each attached, may form a spiro ring consisting of a 4- to 8-membered saturated heterocycle or a 3- to 8-membered saturated carbocycle, or two R^{G} on different carbon atoms may bond together to form a cross-link.

[Item 17]

The compound according to any one of items 1 to 16 or a pharmaceutically acceptable salt thereof, wherein the compound is selected from the group of the following compounds:
cis-3-(7,8-dihydrofuro[3,2-e][1,3]benzothiazol-2-yl)-5-methyloctahydro-2H-imidazo[4,5-c]pyridin-2-one (Example 7);
cis-3-(7,8-dihydrofuro[3,2-e][1,3]benzothiazol-2-yl)-5-(2-methoxyethyl)octahydro-2H-imidazo[4,5-c]pyridin-2-one (Example 8);
cis-1-(7,8-dihydrofuro[3,2-e][1,3]benzothiazol-2-yl)-5-methyloctahydro-2H-imidazo[4,5-c]pyridin-2-one (Example 9);
cis-3-(7,8-dihydrofuro[3,2-e][1,3]benzothiazol-2-yl)-5-ethyloctahydro-2H-imidazo[4,5-c]pyridin-2-one (Example 11);
cis-1-(7,8-dihydrofuro[3,2-e][1,3]benzothiazol-2-yl)-5-methylhexahydropyrrolo[3,4-d]imidazol-2(1H)-one (Example 14);
cis-3-(7,8-dihydrofuro[3,2-e][1,3]benzothiazol-2-yl)-5-(propan-2-yl)octahydro-2H-imidazo[4,5-c]pyridin-2-one (Example 15);
cis-3-(7,8-dihydrofuro[3,2-e][1,3]benzothiazol-2-yl)-5-methyloctahydroimidazo[4,5-c]azepin-2(1H)-one (Example 17);
(3aS,7aS)-3-(7,8-dihydrofuro[3,2-e][1,3]benzothiazol-2-yl)-5-(oxetan-3-yl)octahydro-2H-imidazo[4,5-c]pyridin-2-one (Example 19);
(3aR,7aR)-1-(2H-[1,3]dioxolo[4,5-e][1,3]benzothiazol-7-yl)hexahydropyrano[3,4-d]imidazol-2(3H)-one (Example 21);
(4S,5R)-1-(7,8-dihydrofuro[3,2-e][1,3]benzothiazol-2-yl)-4-methyl-5-[(morpholin-4-yl)methyl]imidazolidin-2-one (Example 23);
(4R,5R)-1-(7,8-dihydrofuro[3,2-e][1,3]benzothiazol-2-yl)-4-methyl-5-[(morpholin-4-yl)methyl]imidazolidin-2-one (Example 24);
(3aR,7aS)-3-(7,8-dihydrofuro[3,2-e][1,3]benzothiazol-2-yl)-5-methyloctahydro-2H-imidazo[4,5-c]pyridin-2-one (Example 28);
(3aR,7aS)-5-cyclopropyl-3-(7,8-dihydro[1,4]dioxino[2,3-e][1,3]benzothiazol-2-yl)octahydro-2H-imidazo[4,5-c]pyridin-2-one (Example 34);
(3aR,7aS)-5-cyclopropyl-3-(2H-[1,3]dioxolo[4,5-e][1,3]benzothiazol-7-yl)octahydro-2H-imidazo[4,5-c]pyridin-2-one (Example 36);
(3aR,7aR)-1-(7,8-dihydro[1,4]dioxino[2,3-e][1,3]benzothiazol-2-yl)hexahydropyrano[3,4-d]imidazol-2(3H)-one (Example 37);
(3aR,6S,7aR)-1-(7,8-dihydrofuro[3,2-e][1,3]benzothiazol-2-yl)-6-methylhexahydropyrano[3,4-d]imidazol-2(3H)-one (Example 38);
(3aR,6S,7aR)-1-(7,8-dihydro[1,4]dioxino[2,3-e][1,3]benzothiazol-2-yl)-6-methylhexahydropyrano[3,4-d]imidazol-2 (3H)-one (Example 39);
(3aR,6R,7aR)-1-(7,8-dihydrofuro[3,2-e][1,3]benzothiazol-2-yl)-6-(fluoromethyl)hexahydropyrano[3,4-d]imidazol-2(3H)-one(Example 40);
(3aR,6R,7aR)-1-(7,8-dihydro[1,4]dioxino[2,3-e][1,3]benzothiazol-2-yl)-6-(fluoromethyl)hexahydropyrano[3,4-d]imidazol-2 (3H)-one (Example 41);
(3aR,6S,7aR)-1-(7,8-dihydrofuro[3,2-e][1,3]benzothiazol-2-yl)-6-ethylhexahydropyrano[3,4-d]imidazol-2(3H)-one (Example 42);
(3aR,6S,7aR)-1-(7,8-dihydro[1,4]dioxino[2,3-e][1,3]benzothiazol-2-yl)-6-ethylhexahydropyrano[3,4-d]imidazol-2(3H)-one (Example 43);
(4S,5S)-1-(7,8-dihydrofuro[3,2-e][1,3]benzothiazol-2-yl)-5-(methoxymethyl)-4-methylimidazolidin-2-one (Example 44);
(3aR,6aS)-1-(7,8-dihydrofuro[3,2-e][1,3]benzothiazol-2-yl)tetrahydro-1H-furo[3,4-d]imidazol-2(3H)-one (Example 65);
rac-(3aR,7aS)-3-(7,8-dihydrofuro[3,2-e][1,3]benzothiazol-2-yl)-5-(oxetan-3-yl)octahydro-2H-imidazo[4,5-c]pyridin-2-one
(Example 71);
rac-(3aR,7aS)-5-cyclobutyl-3-(7,8-dihydrofuro[3,2-e][1,3]benzothiazol-2-yl)octahydro-2H-imidazo[4,5-c]pyridin-2-one (Example 72);
rac-(3aR,8aS)-3-(7,8-dihydrofuro[3,2-e][1,3]benzothiazol-2-yl)-5-(oxetan-3-yl)octahydroimidazo[4,5-c]azepin-2(1H)-one(Example 73);
rac-(3aR,7aS)-3-(7,8-dihydro[1,4]dioxino[2,3-e][1,3]benzothiazol-2-yl)-5-methyloctahydro-2H-imidazo[4,5-c]pyridin-2-one (Example 76);
rac-(3aR,7aS)-1-(7,8-dihydro[1,4]dioxino[2,3-e][1,3]benzothiazol-2-yl)-5-methyloctahydro-2H-imidazo[4,5-c]pyridin-2-one (Example 77);
rac-(3aR,7aS)-1-(2H-[1,3]dioxolo[4,5-e][1,3]benzothiazol-7-yl)-5-methyloctahydro-2H-imidazo[4,5-c]pyridin-2-one (Example 78);
rac-(3aR,7aS)-3-(2H-[1,3]dioxolo[4,5-e][1,3]benzothiazol-7-yl)-5-methyloctahydro-2H-imidazo[4,5-c]pyridin-2-one (Example 79);
rac-(3aR,7aS)-3-(7,8-dihydro[1,4]dioxino[2,3-e][1,3]benzothiazol-2-yl)-5-(oxetan-3-yl)octahydro-2H-imidazo[4,5-c]pyridin-2-one (Example 80);
rac-(3aR,7aS)-3-(2H-[1,3]dioxolo[4,5-e][1,3]benzothiazol-7-yl)-5-(oxetan-3-yl)octahydro-2H-imidazo[4,5-c]pyridin-2-one(Example 81);
rac-(3aR,8aS)-3-(2H-[1,3]dioxolo[4,5-e][1,3]benzothiazol-7-yl)-5-methyloctahydroimidazo[4,5-c]azepin-2(1H)-one (Example 85);
rac-(3aR,8aS)-3-(2H-[1,3]dioxolo[4,5-e][1,3]benzothiazol-7-yl)-5-(oxetan-3-yl)octahydroimidazo[4,5-c]azepin-2(1H)-one(Example 86);
(3aS,6S,7aR)-1-(7,8-dihydrofuro[3,2-e][1,3]benzothiazol-2-yl)-5,6-dimethyloctahydro-2H-imidazo[4,5-c]pyridin-2-one (Example 93);
rac-(3aR,7aS)-5-cyclopropyl-3-(7,8-dihydrofuro[3,2-e][1,3]benzothiazol-2-yl)octahydro-2H-imidazo[4,5-c]pyridin-2-one (Example 98);
rac-(3aR,7aS)-3-(7,8-dihydrofuro[3,2-e][1,3]benzothiazol-2-yl)-5-(2-propyn-1-yl)octahydro-2H-imidazo[4,5-c]pyridin-2-one(Example 99);
rac-[(3aR,7aS)-3-(7,8-dihydrofuro[3,2-e][1,3]benzothiazol-2-yl)-2-oxooctahydro-5H-imidazo[4,5-c]pyridin-5-yl]acetonitrile(Example 100);
rac-(3aR,6aS)-1-(7,8-dihydrofuro[3,2-e][1,3]benzothiazol-2-yl)-5-(2-propyn-1-yl)hexahydropyrrolo[3,4-d]imidazol-2(1H)-one(Example 102);
rac-[(3aR,6aS)-1-(7,8-dihydrofuro[3,2-e][1,3]benzothiazol-2-yl)-2-oxohexahydropyrrolo[3,4-d]imidazol-5(1H)-yl]acetonitrile(Example 103);
rac-(3aR,8aS)-5-cyclopropyl-3-(7,8-dihydrofuro[3,2-e][1,3]benzothiazol-2-yl)octahydroimidazo[4,5-c]azepin-2(1H)-one(Example 104);
rac-(3aR,8aS)-3-(7,8-dihydrofuro[3,2-e][1,3]benzothiazol-2-yl)-5-(2-propyn-1-yl)octahydroimidazo[4,5-c]azepin-2(1H)-one(Example 105);
rac-[(3aR,8aS)-3-(7,8-dihydrofuro[3,2-e][1,3]benzothiazol-2-yl)-2-oxooctahydroimidazo[4,5-c]azepin-5(1H)-yl]acetonitrile(Example 106);
rac-(3aR,7aS)-3-(7,8-dihydrofuro[3,2-e][1,3]benzothiazol-2-yl)-5-(2,2,2-trifluoroethyl)octahydro-2H-imidazo[4,5-c]pyridin-2-one (Example 107);
rac-(3aR,7aS)-5-(2,2-difluoroethyl)-3-(7,8-dihydrofuro[3,2-e][1,3]benzothiazol-2-yl)octahydro-2H-imidazo[4,5-c]pyridin-2-one (Example 108);
(3aR,7aS)-5-cyclopropyl-3-(7,8-dihydrofuro[3,2-e][1,3]benzothiazol-2-yl)octahydro-2H-imidazo[4,5-c]pyridin-2-one (Example 110);
[(3aR,7aS)-3-(7,8-dihydrofuro[3,2-e][1,3]benzothiazol-2-yl)-2-oxooctahydro-5H-imidazo[4,5-c]pyridin-5-yl]acetonitrile(Example 112);
(3aR,7aS)-3-(7,8-dihydrofuro[3,2-e][1,3]benzothiazol-2-yl)-5-(2-propyn-1-yl)octahydro-2H-imidazo[4,5-c]pyridin-2-one(Example 114);
(4S,5R)-1-(7,8-dihydrofuro[3,2-e][1,3]benzothiazol-2-yl)-5-{[(3S)-3-fluoropyrrolidin-1-yl]methyl}-4-methylimidazolidin-2-one (Example 115);
(4S,5R)-5-[(3,3-difluoroazetidin-1-yl)methyl]-1-(7,8-dihydrofuro[3,2-e][1,3]benzothiazol-2-yl)-4-methylimidazolidin-2-one (Example 116);
(4S,5R)-5-[(3,3-difluoropyrrolidin-1-yl)methyl]-1-(7,8-dihydrofuro[3,2-e][1,3]benzothiazol-2-yl)-4-methylimidazolidin-2-one (Example 117);
(4S,5R)-1-(7,8-dihydrofuro[3,2-e][1,3]benzothiazol-2-yl)-5-[(3-fluoroazetidin-1-yl)methyl]-4-methylimidazolidin-2-one(Example 118);
rac-(3aR,7aR)-3-(7,8-dihydrofuro[3,2-e][1,3]benzothiazol-2-yl)hexahydropyrano[3,4-d]imidazol-2(3H)-one (Example 120);
rac-(3aR,7aR)-1-(7,8-dihydrofuro[3,2-e][1,3]benzothiazol-2-yl)hexahydropyrano[3,4-d]imidazol-2(3H)-one (Example 121);
(3aR,7aR)-1-(7,8-dihydrofuro[3,2-e][1,3]benzothiazol-2-yl)hexahydropyrano[3,4-d]imidazol-2(3H)-one (Example 122);
(3aR,7aR)-1-(7,8-dihydrofuro[3,2-e][1,3]benzothiazol-2-yl)-6,6-dimethylhexahydropyrano[3,4-d]imidazol-2(3H)-one (Example 124);
(3aR,6R,7aR)-1-(7,8-dihydrofuro[3,2-e][1,3]benzothiazol-2-yl)-2-oxooctahydropyrano[3,4-d]imidazole-6-carbonitrile (Example 127);
(3aR,6R,7aR)-1-(7,8-dihydrofuro[3,2-e][1,3]benzothiazol-2-yl)-6-methoxyhexahydropyrano[3,4-d]imidazol-2(3H)-one (Example 129);
(3aR,6R,7aR)-1-(2H-[1,3]dioxolo[4,5-e][1,3]benzothiazol-7-yl)-2-oxooctahydropyrano[3,4-d]imidazole-6-carbonitrile (Example 131);
(3aR,6R, 7aR)-1-(7,8-dihydro[1,4]dioxino[2,3-e][1,3]benzothiazol-2-yl)-2-oxooctahydropyrano[3,4-d]imidazole-6-carbonitrile (Example 133);
(3aR,8aR)-1-(7,8-dihydro[1,4]dioxino[2,3-e][1,3]benzothiazol-2-yl)hexahydro-1H-oxepino[3,4-d]imidazol-2(3H)-one (Example 135);
(3aR,6S,7aR)-1-(2H-[1,3]dioxolo[4,5-e][1,3]benzothiazol-7-yl)-6-ethylhexahydropyrano[3,4-d]imidazol-2(3H)-one (Example 138);
(3aR,6S,7aR)-1-(2H-[1,3]dioxolo[4,5-e][1,3]benzothiazol-7-yl]-6-methylhexahydropyrano[3,4-d]imidazol-2(3H)-one (Example 139);
(3aR,6R,7aR)-1-(2H-[1,3]dioxolo[4,5-e][1,3]benzothiazol-7-yl]-6-(fluoromethyl)hexahydropyrano[3,4-d]imidazol-2(3H)-one(Example 140);
(3aR,6R,7aR)-1-(7,8-dihydrofuro[3,2-e][1,3]benzothiazol-2-yl)-6-(hydroxymethyl)hexahydropyrano[3,4-d]imidazol-2(3H)-one(Example 144);
(3aR,6R, 7aR)-1-(7,8-dihydro[1, 4]dioxino[2,3-e][1,3]benzothiazol-2-yl)-6-(hydroxymethyl)hexahydropyrano[3,4-d]imidazol-2(3H)-one(Example 146);
(3aR,6R,7aR)-1-(2H-[1,3]dioxolo[4,5-e][1,3]benzothiazol-7-yl)-6-(hydroxymethyl)hexahydropyrano[3,4-d]imidazol-2(3H)-one(Example 147);
(3aR,6R,7aR)-1-(7,8-dihydrofuro[3,2-e][1,3]benzothiazol-2-yl)-6-ethynylhexahydropyrano[3,4-d]imidazol-2(3H)-one (Example 149);
(3aR,7aR)-1-(7,8-dihydrofuro[3,2-e][1,3]benzothiazol-2-yl)-6-fluorohexahydropyrano[3,4-d]imidazol-2(3H)-one (Example 151);
(3aR,6S,6aS)-1-(7,8-dihydrofuro[3,2-e][1,3]benzothiazol-2-yl)-6-(hydroxymethyl)tetrahydro-1H-furo[3,4-d]imidazol-2(3H)-one(Example 157);
(3aR,6S,6aS)-1-(7,8-dihydrofuro[3,2-e][1,3]benzothiazol-2-yl)-6-(fluoromethyl)tetrahydro-1H-furo[3,4-d]imidazol-2(3H)-one(Example 159);
(3aR,6R,6aS)-1-(7,8-dihydrofuro[3,2-e][1,3]benzothiazol-2-yl)-6-methyltetrahydro-1H-furo[3,4-d]imidazol-2(3H)-one (Example 161); and
[(3aS,4R,6aR)-3-(7,8-dihydrofuro[3,2-e][1,3]benzothiazol-2-yl)-2-oxohexahydro-1H-furo[3,4-d]imidazol-4-yl]acetonitrile(Example 162).

[Item 18]

A medicament comprising the compound according to any one of items 1 to 17 or a pharmaceutically acceptable salt thereof as an active ingredient.

[Item 19]

A pharmaceutical composition comprising the compound according to any one of items 1 to 17 or a pharmaceutically acceptable salt thereof as an active ingredient.

[Item 20]

A therapeutic agent and/or a prophylactic agent for a disease involving DYRK, comprising the compound according to any one of items 1 to 17 or a pharmaceutically acceptable salt thereof as an active ingredient.

[Item 21]

The therapeutic agent and/or the prophylactic agent according to item 20, wherein the disease involving DYRK is frontotemporal dementia, progressive supranuclear palsy, corticobasal degeneration, Lewy body dementia, vascular dementia, traumatic brain injury, chronic traumatic encephalopathy, stroke, Alzheimer's disease, Parkinson's disease, Down's disease, or depression, and mental retardation, memory impairment, memory loss, learning disability, intellectual disability, cognitive dysfunction, mild cognitive impairment, or dementia symptom associated therewith, or brain tumor, pancreatic cancer, ovarian cancer, osteosarcoma, large intestine cancer, lung cancer, bone resorption disease, osteoporosis, sickle cell anemia, chronic renal disease, or bone resorption disease.

[Item 22]

A method for treating and/or preventing a disease involving DYRK, comprising administration of a therapeutically effective amount of the compound according to any one of items 1 to 17 or a pharmaceutically acceptable salt thereof to a patient in need of treatment.

[Item 23]

Use of the compound according to any one of items 1 to 17 or a pharmaceutically acceptable salt thereof, for producing a therapeutic agent and/or a prophylactic agent for a disease involving DYRK.

[Item 24]

The compound according to any one of items 1 to 17 or a pharmaceutically acceptable salt thereof, for use in treatment and/or prevention of a disease involving DYRK.

[Item 25]

A medicament obtained by combining the medicament according to item 18 and at least one or more agents selected from agents classified into an anticancer agent, an antipsychotic drug, an antidementia drug, an antiepileptic drug, an antidepressant drug, a gastrointestinal drug, a thyroid hormone drug, or an antithyroid drug.

[Item 26]

The medicament according to item 18, for treating frontotemporal dementia, progressive supranuclear palsy, corticobasal degeneration, Levy body dementia, vascular dementia, traumatic brain injury, chronic traumatic encephalopathy, stroke, Alzheimer's disease, Parkinson's disease, Down syndrome, or depression, and complication, mental retardation, memory impairment, memory loss, learning disability, intellectual disability, cognitive dysfunction, mild cognitive impairment, or treating dementia symptom progression or preventing dementia onset associated therewith, or treating brain tumor, pancreatic cancer, ovarian cancer, osteosarcoma, large intestine cancer, lung cancer, bone resorption disease, osteoporosis, sickle cell anemia, chronic renal disease, or bone resorption disease, in combination with at least one or more agents selected from agents classified into an anticancer agent, an antipsychotic drug, an antidementia drug, an antiepileptic drug, an antidepressant drug, a gastrointestinal drug, a thyroid hormone drug, or an antithyroid drug.

### Effect of the Invention

The present inventors have carried out various studies in order to solve the above problems and as a result, have found that the amine derivative represented by the above formula (1) and a pharmaceutically acceptable salt thereof are an excellent group of drugs having an excellent DYRK inhibitory action, and have completed the present invention. The compound provided by the present invention is useful as a pharmaceutical (pharmaceutical composition) for prevention or treatment of a disease known to be associated with a DYRK1A-mediated abnormal cellular response, such as a psychiatric or neurologic disease such as Alzheimer's disease, Parkinson's disease, Down's disease, or depression, and mental retardation, memory impairment, memory loss, learning disability, intellectual disability, cognitive dysfunction, mild cognitive impairment, or a therapeutic drug for dementia symptom progression or a prophylactic drug for dementia onset associated therewith, or further a tumor such as brain tumor. The compound provided by the present invention is, as an inhibitor of DYRK1B, useful as a pharmaceutical (pharmaceutical composition) for prevention or treatment of a tumor such as pancreatic cancer, ovarian cancer, osteosarcoma, large intestine cancer, or lung cancer. Further, the compound provided by the present invention is useful as a pharmaceutical (pharmaceutical composition) for prevention or treatment of bone resorption disease and osteoporosis because DYRK2 controls p53 in response to DNA damage to induce apoptosis. In addition, the compound provided by the present invention is, as an inhibitor of DYRK3, useful as a pharmaceutical (pharmaceutical composition) for prevention or treatment of sickle cell anemia, chronic renal disease, bone resorption disease, and osteoporosis. In addition, the compound provided by the present invention is, as a compound that inhibits DYRK, useful as a reagent for pathological imaging related to the above diseases or a reagent for a basic experiment or for research.

### Best Mode to Carry Out the Invention

The terms used herein will be described below.

“DYRK” stands for Dual-specificity tYrosine-phosphorylation Regulated protein Kinase, and means one or two or more of the DYRK family (DYRK1A, DYRK1B, DYRK2, DYRK3, and DYRK4).

Examples of a “halogen atom” include a fluorine atom, a chlorine atom, a bromine atom, or an iodine atom. The halogen atom is preferably a fluorine atom.

“C₁₋₆ alkyl” means a linear or branched saturated hydrocarbon group having 1 to 6 carbon atoms, and “C₆ alkyl” means a linear or branched saturated hydrocarbon group having 6 carbon atoms. The same also applies to other numbers. The C₁₋₆ alkyl is preferably “C₁₋₄ alkyl” and more preferably “C₁₋₃ alkyl.” Specific examples of the “C₁₋₃ alkyl” include methyl, ethyl, propyl, and 1-methylethyl. Specific examples of the “C₁₋₄ alkyl” include butyl, 1,1-dimethylethyl, 1-methylpropyl, and 2-methylpropyl, in addition to those given as specific examples of the “C₁₋₃ alkyl.” Specific examples of the “C₁₋₆ alkyl” include pentyl, 1,1-dimethylpropyl, 1,2-dimethylpropyl, 1-methylbutyl, 2-methylbutyl, 4-methylpentyl, 3-methylpentyl, 2-methylpentyl, 1-methylpentyl, and hexyl, in addition to those given as specific examples of the “C₁₋₄ alkyl.”

“C₁₋₆ alkoxy” means “C₁₋₆ alkyloxy,” and the “C₁₋₆ alkyl” moiety is defined as the “C₁₋₆ alkyl.” The “C₁₋₆ alkoxy” is preferably “C₁₋₄ alkoxy” and more preferably “C₁₋₃ alkoxy.” Specific examples of the “C₁₋₃ alkoxy” include methoxy, ethoxy, propoxy, and 1-methylethoxy. Specific examples of the “C₁₋₄ alkoxy” include butoxy, 1,1-dimethylethoxy, 1-methylpropoxy, and 2-methylpropoxy, in addition to those given as specific examples of the “C₁₋₃ alkoxy.” Specific examples of the “C₁₋₆ alkoxy” include pentyloxy, 1,1-dimethylpropoxy, 1,2-dimethylpropoxy, 1-methylbutoxy, 2-methylbutoxy, 4-methylpentyloxy, 3-methylpentyloxy, 2-methylpentyloxy, 1-methylpentyloxy, and hexyloxy, in addition to those given as specific examples of the “C₁₋₄ alkoxy.”

“C₁₋₆ alkoxycarbonyl” refers to “carbonyl” substituted with the “C₁₋₆ alkoxy.” The “C₁₋₆ alkoxycarbonyl” is preferably “C₁₋₄ alkoxycarbonyl” and more preferably “C₁₋₃ alkoxycarbonyl.” Specific examples of the “C₁₋₃ alkoxycarbonyl” include methoxycarbonyl, ethoxycarbonyl, propoxycarbonyl, and 1-methylethoxycarbonyl. Specific examples of the “C₁₋₄alkoxycarbonyl” include butoxycarbonyl, 1,1-dimethylethoxycarbonyl, 1-methylpropoxycarbonyl, and 2-methylpropoxycarbonyl, in addition to those given as specific examples of the “C₁₋₃ alkoxycarbonyl.” Specific examples of the “Ca₆ alkoxycarbonyl” include pentyloxycarbonyl, 1,1-dimethylpropoxycarbonyl, 1,2-dimethylpropoxycarbonyl, 1-methylbutoxycarbonyl, 2-methylbutoxycarbonyl, 4-methylpentyloxycarbonyl, 3-methylpentyloxycarbonyl, 2-methylpentyloxycarbonyl, 1-methylpentyloxycarbonyl, and hexyloxycarbonyl, in addition to those given as specific examples of the “C₁₋₄ alkoxycarbonyl.”

“C₁₋₇ alkylene,” “C₁₋₄ alkylene,” or “C₁₋₃ alkylene” means a linear or branched divalent saturated hydrocarbon group having 1 to 7, 1 to 4, or 1 to 3 carbon atoms, respectively. The “C₁₋₇ alkylene” is preferably “C₁₋₄ alkylene,” the “C₁₋₄alkylene” is preferably “C₁₋₃ alkylene,” and the “C₁₋₃ alkylene” is preferably “C₁₋₂ alkylene.” Specific examples of the “C₁₂ alkylene” include methylene and ethylene. Specific examples of the “C₁₋₃ alkylene” include propylene and 1-methylethylene, in addition to those given as specific examples of the “C₁₋₂ alkylene.” Specific examples of the “C₁₋₄ alkylene” include butylene, 1,1-dimethylethylene, 1,2-dimethylethylene, 1-methylpropylene, and 2-methylpropylene, in addition to those given as specific examples of the “C₁₋₃ alkylene.” Specific examples of the “C₁₋₇ alkylene” include pentylene, 1,1-dimethylpropylene, 2,2-dimethylpropylene, 1,2-dimethylpropylene, 1,3-dimethylpropylene, 1-methylbutylene, 2-methylbutylene, hexylene, 1,1-dimethylbutylene, 2,2-dimethylbutylene, 1,2-dimethylbutylene, 1,3-dimethylbutylene, 1,4-dimethylbutylene, 2,3-dimethylbutylene, 1-methylpentylene, 2-methylpentylene, 3-methylpentylene, heptylene, 1,1-dimethylpentylene, 2,2-dimethylpentylene, 3,3-dimethylpentylene, 1,2-dimethylpentylene, 1,3-dimethylpentylene, 1,4-dimethylpentylene, 1,5-dimethylpentylene, 2,3-dimethylpentylene, 2,4-dimethylpentylene, 2,5-dimethylpentylene, 1-methylhexylene, 2-methylhexylene, and 3-methylhexylene, in addition to those given as specific examples of the “C₁₋₄ alkylene.”

“C₂₋₆ alkynyl” means a linear or branched saturated hydrocarbon group having 2 to 6 carbon atoms and having 1 to 3 triple bonds. The “alkynyl” is preferably “C₂₋₄ alkynyl” and more preferably “C₂₋₃ alkynyl.” Specific examples of the “alkynyl” include ethynyl, propargyl, and 2-butynyl.

“C₃₋₁₀ cycloalkyl” means a cyclic saturated hydrocarbon group having 3 to 10 carbon atoms, and also includes one having partially an unsaturated bond and one having a crosslinked structure. The “C₃₋₁₀ cycloalkyl” is preferably “C₃₋₇ cycloalkyl.” Specific examples of the “C₃₋₇ cycloalkyl” include cyclopropyl, cyclobutyl, cyclopentyl, cyclohexyl, and cycloheptyl. Specific examples of the “C₃₋₁₀ cycloalkyl” include cyclooctyl, cyclononyl, cyclodecyl, and adamantyl, in addition to those given as specific examples of the “C₃₋₇ cycloalkyl.”

A “4- to 11-membered saturated heterocycle” means a monocyclic or bicyclic saturated heterocycle containing one or more heteroatoms selected from the group of a nitrogen atom, an oxygen atom, and a sulfur atom, and also includes one having partially an unsaturated bond and one having a crosslinked structure. The “4- to 11-membered saturated heterocycle” is preferably a “4- to 8-membered saturated heterocycle,” more preferably a “5- to 8-membered saturated heterocycle,” and further preferably “5- to 7-membered saturated heterocycle.” Specific examples of the “4- to 8-membered saturated heterocycle” include an azetidine ring, a pyrrolidine ring, a piperidine ring, an azepane ring, an azocane ring, a morpholine ring, a piperazine ring, an oxazocane ring, an azabicycloheptane ring, an azabicyclooctane ring, an oxetane ring, a thietane ring, a tetrahydrofuran ring, a tetrahydrothiophene ring, a tetrahydropyran ring, a thiomorpholine ring, and a 1,4-dioxane ring. Specific examples of the “4- to 11-membered saturated heterocycle” include an azonane ring, an oxazonane ring, an azecane ring, an oxazecane ring, an azacycloundecane ring, an azabicyclononane ring, and an azabicyclodecane ring, in addition to those given as specific examples of the “4- to 8-membered saturated heterocycle.”

A “3- to 8-membered saturated carbocycle” means a monocyclic or bicyclic saturated aliphatic carbocycle, and also includes one having partially an unsaturated bond and one having a crosslinked structure. The “3- to 8-membered saturated carbocycle” is preferably a “4- to 8-membered saturated carbocycle,” more preferably a “5- to 8-membered saturated carbocycle,” and further preferably “5- to 7-membered saturated carbocycle.” Specific examples of the “3- to 8-membered saturated heterocycle” include cyclopropane, cyclobutane, cyclopentane, cyclohexane, cycloheptane, cyclooctane, and bicycloheptane.

In the structural formula represented by formula (1), the tricyclic heterocycle formed by including A¹, A², and L¹ represents a chemically stable heterocycle and also includes one having partially an unsaturated bond. The tricyclic heterocycle preferably has the structure shown below.

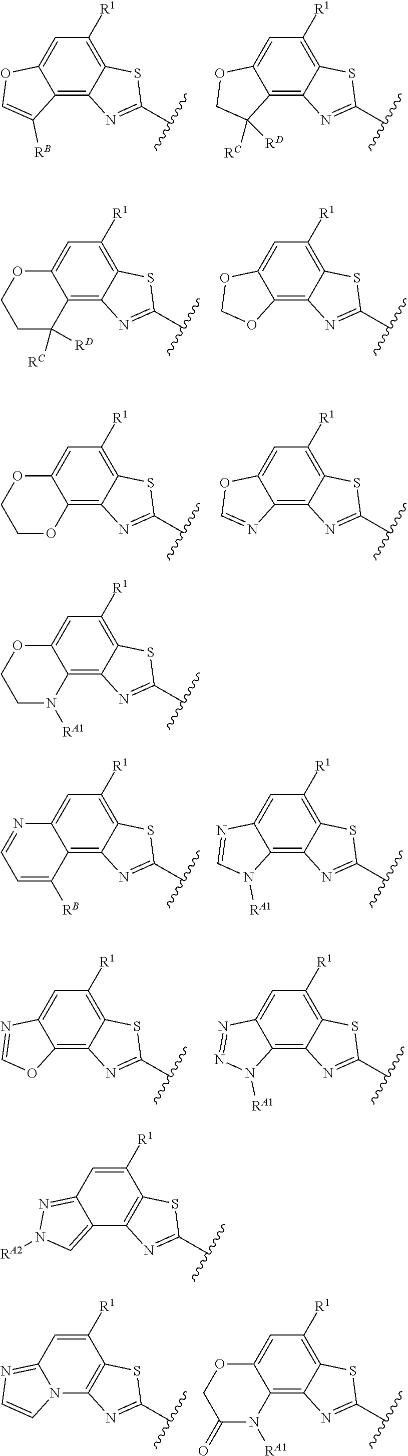

A “4- to 11-membered saturated heterocycle formed by R² and R³ together with the nitrogen atom to which they are attached,” a “4- to 11-membered saturated heterocycle formed by R³ and R⁴ together with the nitrogen atom to which they are attached,” and a “4- to 11-membered saturated heterocycle formed by any carbon atom on a saturated heterocycle constituted by R², R³, and a nitrogen atom, and R⁴ together” mean a monocyclic or bicyclic saturated heterocycle containing one or two or more heteroatoms other than the nitrogen atom as an atom constituting the ring, and also includes one having partially an unsaturated bond and one having a crosslinked structure. The “4- to 11-membered saturated heterocycle formed by R² and R³ together with the nitrogen atom to which they are attached,” the “4- to 11-membered saturated heterocycle formed by R³ and R⁴ together with the nitrogen atom to which they are attached,” and the “4- to 11-membered saturated heterocycle formed by any carbon atom on a saturated heterocycle constituted by R², R³, and a nitrogen atom, and R⁴ together” is each preferably a “4- to 8-membered saturated heterocycle.” Specific examples of the “4- to 8-membered saturated heterocycle” include an azetidine ring, a pyrrolidine ring, a piperidine ring, an azepane ring, an azocane ring, a morpholine ring, a piperazine ring, an oxazocane ring, an azabicycloheptane ring, and an azabicyclooctane ring. Specific examples of the “4- to 1l-membered saturated heterocycle” include an azonane ring, an oxazonane ring, an azecane ring, an oxazecane ring, an azacycloundecane ring, an azabicyclononane ring, and an azabicyclodecane ring, in addition to those given as specific examples of the “4- to 8-membered saturated heterocycle.”

When Z is —OR⁷, a “5- to 11-membered saturated heterocycle formed by R⁴ and R⁷ together with the carbon atom and the oxygen atom to which they, respectively, are attached” means a monocyclic or bicyclic saturated heterocycle containing one or two or more heteroatoms other than the oxygen atom as an atom constituting the ring, and also includes one having partially an unsaturated bond and one having a crosslinked structure. The “5- to 1l-membered saturated heterocycle formed by R⁴ and R⁷ together with the carbon atom and the oxygen atom to which they, respectively, are attached” is preferably a “5- to 8-membered saturated heterocycle.” Specific examples of the “5- to 8-membered saturated heterocycle” include a tetrahydrofuran ring, a tetrahydropyran ring, an oxepane ring, and an oxocane ring. Specific examples of the “5- to 11-membered saturated heterocycle” include an oxonane ring, an oxecane ring, and an oxacycloundecane ring, in addition to those given as specific examples of the “5- to 8-membered saturated heterocycle.”

As used herein, for example, the bond of a substituent drawn in such a way as to cross a pyrrolidine ring as represented by the following formula (W) means substitution with one substituent at any substitutable position on the pyrrolidine ring, and specifically, the compound represented by the following formula (W-1) or (W-2) is included in the compound represented by the following formula (W).

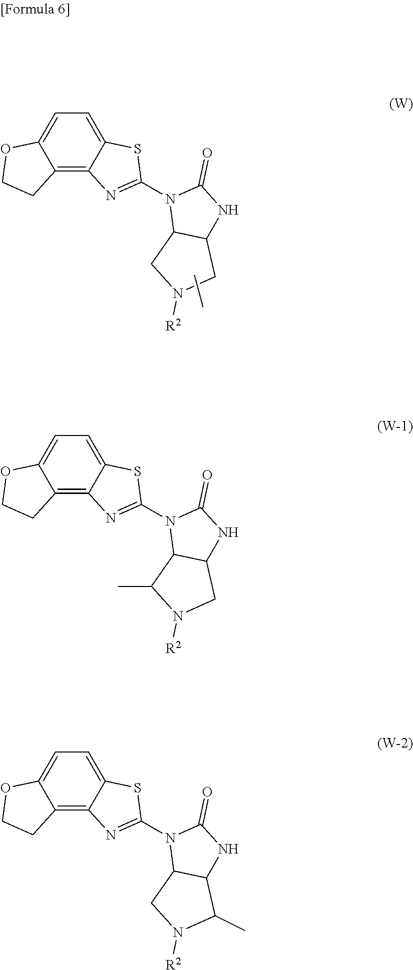

In the compound of the present invention represented by formula (1), the definitions and preferred ranges of A¹, A², L¹, L², R^{A1}, R^{A2}, R^{B}, R^{C}, R^{D}, R^{E}, R^{F}, R¹, R², R³, R⁴, X, l, m, n, Z, R⁷, p, q, t, and R^{G} are as follows, and the technical scope of the present invention is not limited to the scope of compounds listed below.

A¹ is an oxygen atom or a nitrogen atom (═N—) and preferably an oxygen atom.

A² is CR^{B}, CR^{C}R^{D}, an oxygen atom, or NR^{A1}, preferably CR^{C}R^{D} or an oxygen atom, and more preferably methylene or an oxygen atom.

L¹ is optionally substituted methylene, optionally substituted ethylene, optionally substituted methine, optionally substituted ethanediylidene, or ═N—, and preferably methylene.

L² is optionally substituted C₁₋₄ alkylene and preferably methylene, ethylene, or propylene. L² is more preferably methylene or ethylene.

R^{A1}, R^{A2}, R^{B}, R^{C}, and R^{D} are each a hydrogen atom or optionally substituted C₁-6 alkyl and preferably a hydrogen atom.

R^{E} and R^{G} are each optionally substituted C₁₋₆ alkyl and preferably methyl.

R^{F} is a hydrogen atom, a halogen atom, or optionally substituted C₁-6 alkyl and preferably a hydrogen atom or optionally substituted C₁₋₆ alkyl.

R¹ is a hydrogen atom, a halogen atom, or optionally substituted C₁-6 alkyl, preferably a hydrogen atom or a halogen atom, and more preferably a hydrogen atom.

R² is a hydrogen atom, optionally substituted C₁₋₆ alkyl, C(O)—R^{E}, C₃-10 cycloalkyl, C₂₋₆ alkynyl, or a cyclic group of a 4- to 11-membered saturated heterocycle, preferably a hydrogen atom, optionally substituted C₁₋₆ alkyl, or C₃₋₁₀ cycloalkyl, and more preferably a hydrogen atom or optionally substituted C₁₋₆ alkyl.

R³ is a hydrogen atom, optionally substituted C₁₋₆ alkyl, or C(O)—R^{E} and preferably a hydrogen atom or optionally substituted C₁₋₆ alkyl.

R⁴ is optionally substituted C₁₋₆ alkyl, preferably optionally substituted C₁₋₄ alkyl, and more preferably methyl or ethyl.

X is a carbon atom or a nitrogen atom and preferably a carbon atom.

l, m, p, and q are each 1, 2, or 3 and preferably 1 or 2.

n and t are each 1, 2, 3, or 4 and preferably 1 or 2.

Z is —NR²R³ or —OR⁷, and when Z is —NR²R³, preferably R² and R³, together with the nitrogen atom to which they are attached, form an optionally substituted 4- to 8-membered saturated heterocycle, or R³ and R⁴, together with the nitrogen atom and the carbon atom to which they, respectively, are attached, form an optionally substituted 4- to 8-membered saturated heterocycle; when Z is —OR⁷, R⁷ is optionally substituted C₁₋₆ alkyl, or optionally substituted C₁₋₇ alkylene formed together with R⁴, preferably optionally substituted methylene or optionally substituted ethylene, and R⁴ and R⁷, together with the carbon atom and the oxygen atom to which they, respectively, are attached, form an optionally substituted 5- to 8-membered saturated heterocycle.

As used herein, the substituent when the “optionally substituted C₁₋₆ alkyl” is substituted is one or more substituents selected from the group consisting of a halogen atom, hydroxy (wherein the hydroxy is optionally substituted with a protective group for hydroxy), optionally substituted C₃-10 cycloalkyl and optionally substituted C₁₋₆ alkoxy, a C₂₋₆ alkynyl group, nitrile, C₁₋₆ alkoxycarbonyl, formyl, and a 4- to 11-membered saturated heterocycle (wherein the saturated heterocycle is optionally substituted with optionally substituted C₁₋₆ alkyl, a halogen atom, and a protective group for a nitrogen atom), and substitution with such a substituent occurs at any substitutable position. The number of the substituents is preferably 1 to 5, and more preferably 1 to 3. When substitution with two or more substituents occurs, these substituents may be the same or different.

As used herein, the substituent when the “optionally substituted C₃₋₁₀ cycloalkyl” is substituted is one or more substituents selected from the group consisting of a halogen atom, hydroxy, C₁₋₆ alkyl, C₁₋₆ alkoxy, and a C₃₋₁₀ cycloalkyl group, and substitution with such a substituent occurs at any substitutable position. The number of the substituents is preferably 1 to 5, and more preferably 1 to 3. When substitution with two or more substituents occurs, these substituents may be the same or different.

As used herein, the substituent when the “optionally substituted C₁₋₆ alkoxy” is substituted is one or more substituents selected from the group consisting of a halogen atom, hydroxy, C₁₋₆ alkyl, C₁₋₆ alkoxy, and a C₃₋₈ cycloalkyl group, and substitution with such a substituent occurs at any substitutable position. The number of the substituents is preferably 1 to 5, and more preferably 1 to 3. When substitution with two or more substituents occurs, these substituents may be the same or different.

As used herein, the substituent when each of the “optionally substituted C₁₋₇ alkylene,” the “optionally substituted C₁₋₄ alkylene,” and the “optionally substituted C₁₋₃ alkylene” is substituted is one or more substituents selected from the group consisting of a halogen atom, hydroxy, C₁₋₆ alkyl optionally substituted with a halogen atom or hydroxy (wherein the hydroxy is optionally substituted with a protective group for hydroxy), C₁₋₆ alkoxy, a C₃₋₈ cycloalkyl group, nitrile, C₁₋₆ alkoxycarbonyl, formyl, a C₂-6 alkynyl group, an oxo group, and a 4- to 11-membered saturated heterocycle (wherein the saturated heterocycle is optionally substituted with optionally substituted C₁₋₆ alkyl, a halogen atom, and a protective group for a nitrogen atom), and substitution with such a substituent occurs at any substitutable position. The number of the substituents is preferably 1 to 5, and more preferably 1 to 3. When substitution with two or more substituents occurs, these substituents may be the same or different, and two substituents on the same carbon atom, together with the carbon atom to which they are attached, may form a spiro ring consisting of a 4- to 8-membered saturated heterocycle or a 3- to 8-membered saturated carbocycle.

As used herein, the substituent when the “optionally substituted ethylene” is substituted is one or more substituents selected from the group consisting of C₁₋₆ alkyl optionally substituted with a halogen atom or hydroxy (wherein the hydroxy is optionally substituted with a protective group for hydroxy), nitrile, C₁₋₆ alkoxycarbonyl, formyl, a C₂₋₆ alkynyl group, a 4- to 11-membered saturated heterocycle (wherein the saturated heterocycle is optionally substituted with optionally substituted C₁₋₆ alkyl, a halogen atom, and a protective group for a nitrogen atom), and an oxo group, and substitution with such a substituent occurs at any substitutable position. The number of the substituents is preferably 1 to 4. When substitution with two or more substituents occurs, these substituents may be the same or different, and two substituents on the same carbon atom, together with the carbon atom to which they are attached, may form a spiro ring consisting of a 4- to 8-membered saturated heterocycle or a 3- to 8-membered saturated carbocycle.

As used herein, the substituent when each of “optionally substituted methylene,” “optionally substituted methine,” and “optionally substituted ethanediylidene” is substituted is one or more substituents selected from the group consisting of C₁₋₆ alkyl optionally substituted with a halogen atom or hydroxy (wherein the hydroxy is optionally substituted with a protective group for hydroxy), nitrile, C₁₋₆ alkoxycarbonyl, formyl, a C₂₋₆ alkynyl group, an oxo group, and a 4- to 11-membered saturated heterocycle (wherein the saturated heterocycle is optionally substituted with optionally substituted C₁₋₆ alkyl, a halogen atom, and a protective group for a nitrogen atom), and substitution with such a substituent occurs at any substitutable position. The number of the substituents is preferably 1 to 4. When substitution with two or more substituents occurs, these substituents may be the same or different, and two substituents on the same carbon atom, together with the carbon atom to which they are attached, may form a spiro ring consisting of a 4- to 8-membered saturated heterocycle or a 3- to 8-membered saturated carbocycle.

As used herein, the substituent that each of the “optionally substituted 4- to 11-membered saturated heterocycle,” the “optionally substituted 4- to 8-membered saturated heterocycle,” the “optionally substituted 5- to 8-membered saturated heterocycle,” and the “optionally substituted 5- to 11-membered saturated heterocycle” optionally has is one or more substituents selected from the group consisting of a halogen atom, hydroxy, C₁₋₆ alkyl optionally substituted with a halogen atom or hydroxy (wherein the hydroxy is optionally substituted with a protective group for hydroxy), C₁₋₆ alkoxy, C₃₋₈ cycloalkyl, nitrile, C₁₋₆ alkoxycarbonyl, formyl, a C₂₋₆ alkynyl group, an oxo group, and a 4- to 11-membered saturated heterocycle (wherein the saturated heterocycle is optionally substituted with optionally substituted C₁-6 alkyl, a halogen atom, and a protective group for a nitrogen atom), and substitution with such a substituent occurs at any substitutable position. The number of the substituents is preferably 1 to 5, and more preferably 1 to 3. When substitution with two or more substituents occurs, these substituents may be the same or different, and two substituents on the same carbon atom on the ring, together with the carbon atom to which they are attached, may form a spiro ring consisting of a 4- to 8-membered saturated heterocycle or a 3- to 8-membered saturated carbocycle, or two substituents on different carbon atoms on the ring may combine to form a crosslink.

Among the compounds of the present invention represented by formula (1), examples of a preferred compound include the following compounds or pharmaceutically acceptable salts thereof.

A compound wherein A¹ is an oxygen atom, A² is methylene, L¹ is methylene, L² is optionally substituted C₁₋₄ alkylene, X is a carbon atom, R¹ is a hydrogen atom, and R², R³, and R⁴ are each optionally substituted C₁₋₆ alkyl.

A compound wherein A¹ is an oxygen atom, A² is methylene, L¹ is methylene, L² is methylene or ethylene, X is a carbon atom, R¹ is a hydrogen atom, R² and R³ are each optionally substituted C₁₋₆ alkyl, and R⁴ is methyl or ethyl.

A compound wherein A¹ is an oxygen atom, A² is methylene, L¹ is methylene, L² is optionally substituted C₁₋₄ alkylene, X is a carbon atom, R¹ is a hydrogen atom, R² and R³, together with the nitrogen atom to which they are attached, form an optionally substituted 4- to 1l-membered saturated heterocycle, and R⁴ is optionally substituted C₁₋₆ alkyl.

A compound wherein A¹ is an oxygen atom, A² is methylene, L¹ is methylene, L² is methylene or ethylene, X is a carbon atom, R¹ is a hydrogen atom, R² and R³, together with the nitrogen atom to which they are attached, form an optionally substituted 4- to 8-membered saturated heterocycle, and R⁴ is methyl or ethyl.

A compound wherein A¹ is an oxygen atom, A² is methylene, L¹ is methylene, X is a carbon atom, R¹ is a hydrogen atom, R² is a hydrogen atom or optionally substituted C₁₋₆ alkyl, R³ and R⁴, together with the nitrogen atom and the carbon atom to which they, respectively, are attached, form an optionally substituted 4- to 11-membered saturated heterocycle.

A compound wherein A¹ is an oxygen atom, A² is methylene, L¹ is methylene, X is a carbon atom, R¹ is a hydrogen atom, R² is optionally substituted C₁₋₆ alkyl, R³ and
R⁴, together with the nitrogen atom and the carbon atom to which they, respectively, are attached, form an optionally substituted 4- to 8-membered saturated heterocycle.

A compound wherein A¹ and A² are each an oxygen atom, L¹ is methylene or ethylene, X is a carbon atom, R¹ is a hydrogen atom, L² is optionally substituted C₁₋₄ alkylene, and R², R³, and R⁴ are each optionally substituted C₁₋₆ alkyl.

A compound wherein A¹ and A² are each an oxygen atom, L¹ is methylene or ethylene, X is a carbon atom, L² is optionally substituted C₁₋₄ alkylene, R¹ is a hydrogen atom, R² and R³, together with the nitrogen atom to which they are attached, form an optionally substituted 4- to 11-membered saturated heterocycle, and R⁴ is optionally substituted C₁₋₆ alkyl.

A compound wherein A¹ and A² are each an oxygen atom, L¹ is methylene or ethylene, X is a carbon atom, R¹ is a hydrogen atom, R² is a hydrogen atom or optionally substituted C₁₋₆ alkyl, R³ and R⁴, together with the nitrogen atom and the carbon atom to which they, respectively, are attached, form an optionally substituted 4- to 11-membered saturated heterocycle.

A compound wherein A¹ is an oxygen atom, A² is methylene, L¹ is methylene or ethylene, L² is optionally substituted C₁₋₄ alkylene, X is a carbon atom, R¹ is a hydrogen atom, Z is —OR⁷, R⁷ is optionally substituted C₁₋₇ alkylene formed together with R⁴, and R⁴ and R⁷, together with the carbon atom and the oxygen atom to which they, respectively, are attached, form an optionally substituted 5- to 11-membered saturated heterocycle.

A compound wherein A¹ is an oxygen atom, A² is methylene, L¹ is methylene or ethylene, L² is methylene or ethylene, X is a carbon atom, R¹ is a hydrogen atom, Z is —OR⁷, R⁷ is optionally substituted C₁₋₇ alkylene formed together with R⁴, and R⁴ and R⁷, together with the carbon atom and the oxygen atom to which they, respectively, are attached, form an optionally substituted 5- to 11-membered saturated heterocycle.

A compound wherein A¹ and A² are each an oxygen atom, L¹ is methylene or ethylene, L² is optionally substituted C₁₋₄ alkylene, X is a carbon atom, R¹ is a hydrogen atom, Z is —OR⁷, R⁷ is optionally substituted C₁₋₇ alkylene formed together with R⁴, and R⁴ and R⁷, together with the carbon atom and the oxygen atom to which they, respectively, are attached, form an optionally substituted 5- to 11-membered saturated heterocycle.

A compound wherein A¹ and A² are each an oxygen atom, L¹ is methylene or ethylene, L² is methylene or ethylene, X is a carbon atom, R¹ is a hydrogen atom, Z is —OR⁷, R⁷ is optionally substituted C₁₋₇ alkylene formed together with R⁴, and R⁴ and R⁷, together with the carbon atom and the oxygen atom to which they, respectively, are attached, form an optionally substituted 5- to 11-membered saturated heterocycle.

Among the compounds of the present invention represented by formula (1), specific examples of a preferred compound include the following compounds or pharmaceutically acceptable salts thereof.
cis-3-(7,8-Dihydrofuro[3,2-e][1,3]benzothiazol-2-yl)-5-methyloctahydro-2H-imidazo[4,5-c]pyridin-2-one (Example 7);
cis-3-(7,8-dihydrofuro[3,2-e][1,3]benzothiazol-2-yl)-5-(2-methoxyethyl)octahydro-2H-imidazo[4,5-c]pyridin-2-one (Example 8);
cis-1-(7,8-dihydrofuro[3,2-e][1,3]benzothiazol-2-yl)-5-methyloctahydro-2H-imidazo[4,5-c]pyridin-2-one (Example 9);
cis-3-(7,8-dihydrofuro[3,2-e][1,3]benzothiazol-2-yl)-5-ethyloctahydro-2H-imidazo[4,5-c]pyridin-2-one (Example 11);
cis-1-(7,8-dihydrofuro[3,2-e][1,3]benzothiazol-2-yl)-5-methylhexahydropyrrolo[3,4-d]imidazol-2(1H)-one (Example 14);
cis-3-(7,8-dihydrofuro[3,2-e][1,3]benzothiazol-2-yl)-5-(propan-2-yl)octahydro-2H-imidazo[4,5-c]pyridin-2-one (Example 15);
cis-3-(7,8-dihydrofuro[3,2-e][1,3]benzothiazol-2-yl)-5-methyloctahydroimidazo[4,5-c]azepin-2(1H)-one (Example 17);
(3aS,7aS)-3-(7,8-dihydrofuro[3,2-e][1,3]benzothiazol-2-yl)-5-(oxetan-3-yl)octahydro-2H-imidazo[4,5-c]pyridin-2-one (Example 19);
(3aR,7aR)-1-(2H-[1,3]dioxolo[4,5-e][1,3]benzothiazol-7-yl)hexahydropyrano[3,4-d]imidazol-2(3H)-one (Example 21);
(4S,5R)-1-(7,8-dihydrofuro[3,2-e][1,3]benzothiazol-2-yl)-4-methyl-5-[(morpholin-4-yl)methyl]imidazolidin-2-one (Example 23);
(4R,5R)-1-(7,8-dihydrofuro[3,2-e][1,3]benzothiazol-2-yl)-4-methyl-5-[(morpholin-4-yl)methyl]imidazolidin-2-one (Example 24);
(3aR,7aS)-3-(7,8-dihydrofuro[3,2-e][1,3]benzothiazol-2-yl)-5-methyloctahydro-2H-imidazo[4,5-c]pyridin-2-one (Example 28);
(3aR,7aS)-5-cyclopropyl-3-(7,8-dihydro[1,4]dioxino[2,3-e][1,3]benzothiazol-2-yl)octahydro-2H-imidazo[4,5-c]pyridin-2-one (Example 34);
(3aR,7aS)-5-cyclopropyl-3-(2H-[1,3]dioxolo[4,5-e][1,3]benzothiazol-7-yl)octahydro-2H-imidazo[4,5-c]pyridin-2-one (Example 36);
(3aR,7aR)-1-(7,8-dihydro[1,4]dioxino[2,3-e][1,3]benzothiazol-2-yl)hexahydropyrano[3,4-d]imidazol-2(3H)-one (Example 37);
(3aR,6S,7aR)-1-(7,8-dihydrofuro[3,2-e][1,3]benzothiazol-2-yl)-6-methylhexahydropyrano[3,4-d]imidazol-2(3H)-one (Example 38);
(3aR,6S,7aR)-1-(7,8-dihydro[1,4]dioxino[2,3-e][1,3]benzothiazol-2-yl)-6-methylhexahydropyrano[3,4-d]imidazol-2(3H)-one (Example 39);
(3aR,6R,7aR)-1-(7,8-dihydrofuro[3,2-e][1,3]benzothiazol-2-yl)-6-(fluoromethyl)hexahydropyrano[3,4-d]imidazol-2(3H)-one(Example 40);
(3aR,6R,7aR)-1-(7,8-dihydro[1,4]dioxino[2,3-e][1,3]benzothiazol-2-yl)-6-(fluoromethyl)hexahydropyrano[3,4-d]imidazol-2(3H)-one (Example 41);
(3aR,6S,7aR)-1-(7,8-dihydrofuro[3,2-e][1,3]benzothiazol-2-yl)-6-ethylhexahydropyrano[3,4-d]imidazol-2(3H)-one (Example 42);
(3aR,6S,7aR)-1-(7,8-dihydro[1,4]dioxino[2,3-e][1,3]benzothiazol-2-yl)-6-ethylhexahydropyrano[3,4-d]imidazol-2(3H)-one (Example 43);
(4S,5S)-1-(7,8-dihydrofuro[3,2-e][1,3]benzothiazol-2-yl)-5-(methoxymethyl)-4-methylimidazolidin-2-one (Example 44);
(3aR,6aS)-1-(7,8-dihydrofuro[3,2-e][1,3]benzothiazol-2-yl)tetrahydro-1H-furo[3,4-d]imidazol-2(3H)-one (Example 65);
rac-(3aR,7aS)-3-(7,8-dihydrofuro[3,2-e][1,3]benzothiazol-2-yl)-5-(oxetan-3-yl)octahydro-2H-imidazo[4,5-c]pyridin-2-one(Example 71);
rac-(3aR,7aS)-5-cyclobutyl-3-(7,8-dihydrofuro[3,2-e][1,3]benzothiazol-2-yl)octahydro-2H-imidazo[4,5-c]pyridin-2-one (Example 72);
rac-(3aR,8aS)-3-(7,8-dihydrofuro[3,2-e][1,3]benzothiazol-2-yl)-5-(oxetan-3-yl)octahydroimidazo[4,5-c]azepin-2(1H)-one(Example 73);
rac-(3aR,7aS)-3-(7,8-dihydro[1,4]dioxino[2,3-e][1,3]benzothiazol-2-yl)-5-methyloctahydro-2H-imidazo[4,5-c]pyridin-2-one (Example 76);
rac-(3aR,7aS)-1-(7,8-dihydro[1,4]dioxino[2,3-e][1,3]benzothiazol-2-yl)-5-methyloctahydro-2H-imidazo[4,5-c]pyridin-2-one (Example 77);
rac-(3aR,7aS)-1-(2H-[1,3]dioxolo[4,5-e][1,3]benzothiazol-7-yl)-5-methyloctahydro-2H-imidazo[4,5-c]pyridin-2-one (Example 78);
rac-(3aR,7aS)-3-(2H-[1,3]dioxolo[4,5-e][1,3]benzothiazol-7-yl)-5-methyloctahydro-2H-imidazo[4,5-c]pyridin-2-one (Example 79);
rac-(3aR,7aS)-3-(7,8-dihydro[1,4]dioxino[2,3-e][1,3]benzothiazol-2-yl)-5-(oxetan-3-yl)octahydro-2H-imidazo[4,5-c]pyridin-2-one (Example 80);
rac-(3aR,7aS)-3-(2H-[1,3]dioxolo[4,5-e][1,3]benzothiazol-7-yl)-5-(oxetan-3-yl)octahydro-2H-imidazo[4,5-c]pyridin-2-one(Example 81);
rac-(3aR,8aS)-3-(2H-[1,3]dioxolo[4,5-e][1,3]benzothiazol-7-yl)-5-methyloctahydroimidazo[4,5-c]azepin-2(1H)-one (Example 85);
rac-(3aR,8aS)-3-(2H-[1,3]dioxolo[4,5-e][1,3]benzothiazol-7-yl)-5-(oxetan-3-yl)octahydroimidazo[4,5-c]azepin-2(1H)-one(Example 86);
(3aS,6S,7aR)-1-(7,8-dihydrofuro[3,2-e][1,3]benzothiazol-2-yl)-5,6-dimethyloctahydro-2H-imidazo[4,5-c]pyridin-2-one (Example 93);
rac-(3aR,7aS)-5-cyclopropyl-3-(7,8-dihydrofuro[3,2-e][1,3]benzothiazol-2-yl)octahydro-2H-imidazo[4,5-c]pyridin-2-one (Example 98);
rac-(3aR,7aS)-3-(7,8-dihydrofuro[3,2-e][1,3]benzothiazol-2-yl)-5-(2-propyn-1-yl)octahydro-2H-imidazo[4,5-c]pyridin-2-one(Example 99);
rac-[(3aR,7aS)-3-(7,8-dihydrofuro[3,2-e][1,3]benzothiazol-2-yl)-2-oxooctahydro-5H-imidazo[4,5-c]pyridin-5-yl]acetonitrile(Example 100);
rac-(3aR,6aS)-1-(7,8-dihydrofuro[3,2-e][1,3]benzothiazol-2-yl)-5-(2-propyn-1-yl)hexahydropyrrolo[3,4-d]imidazol-2(1H)-one(Example 102);
rac-[(3aR,6aS)-1-(7,8-dihydrofuro[3,2-e][1,3]benzothiazol-2-yl)-2-oxohexahydropyrrolo[3,4-d]imidazol-5(1H)-yl]acetonitrile(Example 103);
rac-(3aR,8aS)-5-cyclopropyl-3-(7,8-dihydrofuro[3,2-e][1,3]benzothiazol-2-yl)octahydroimidazo[4,5-c]azepin-2(1H)-one(Example 104);
rac-(3aR,8aS)-3-(7,8-dihydrofuro[3,2-e][1,3]benzothiazol-2-yl)-5-(2-propyn-1-yl)octahydroimidazo[4,5-c]azepin-2(1H)-one(Example 105);
rac-[(3aR,8aS)-3-(7,8-dihydrofuro[3,2-e][1,3]benzothiazol-2-yl)-2-oxooctahydroimidazo[4,5-c]azepin-5(1H)-yl]acetonitrile(Example 106);
rac-(3aR,7aS)-3-(7,8-dihydrofuro[3,2-e][1,3]benzothiazol-2-yl)-5-(2,2,2-trifluoroethyl)octahydro-2H-imidazo[4,5-c]pyridin-2-one (Example 107);
rac-(3aR,7aS)-5-(2,2-difluoroethyl)-3-(7,8-dihydrofuro[3,2-e][1,3]benzothiazol-2-yl)octahydro-2H-imidazo[4,5-c]pyridin-2-one (Example 108);
(3aR,7aS)-5-cyclopropyl-3-(7,8-dihydrofuro[3,2-e][1,3]benzothiazol-2-yl)octahydro-2H-imidazo[4,5-c]pyridin-2-one (Example 110);
[(3aR,7aS)-3-(7,8-dihydrofuro[3,2-e][1,3]benzothiazol-2-yl)-2-oxooctahydro-5H-imidazo[4,5-c]pyridin-5-yl]acetonitrile(Example 112);
(3aR,7aS)-3-(7,8-dihydrofuro[3,2-e][1,3]benzothiazol-2-yl)-5-(2-propyn-1-yl)octahydro-2H-imidazo[4,5-c]pyridin-2-one(Example 114);
(4S,5R)-1-(7,8-dihydrofuro[3,2-e][1,3]benzothiazol-2-yl)-5-{[(3S)-3-fluoropyrrolidin-1-yl]methyl}-4-methylimidazolidin-2-one (Example 115);
(4S,5R)-5-[(3,3-difluoroazetidin-1-yl)methyl]-1-(7,8-dihydrofuro[3,2-e][1,3]benzothiazol-2-yl)-4-methylimidazolidin-2-one (Example 116);
(4S,5R)-5-[(3,3-difluoropyrrolidin-1-yl)methyl]-1-(7,8-dihydrofuro[3,2-e][1,3]benzothiazol-2-yl)-4-methylimidazolidin-2-one (Example 117);
(4S,5R)-1-(7,8-dihydrofuro[3,2-e][1,3]benzothiazol-2-yl)-5-[(3-fluoroazetidin-1-yl)methyl]-4-methylimidazolidin-2-one(Example 118);
rac-(3aR,7aR)-3-(7,8-dihydrofuro[3,2-e][1,3]benzothiazol-2-yl)hexahydropyrano[3,4-d]imidazol-2(3H)-one (Example 120);
rac-(3aR,7aR)-1-(7,8-dihydrofuro[3,2-e][1,3]benzothiazol-2-yl)hexahydropyrano[3,4-d]imidazol-2(3H)-one (Example 121);
(3aR,7aR)-1-(7,8-dihydrofuro[3,2-e][1,3]benzothiazol-2-yl)hexahydropyrano[3,4-d]imidazol-2(3H)-one (Example 122);
(3aR,7aR)-1-(7,8-dihydrofuro[3,2-e][1,3]benzothiazol-2-yl)-6,6-dimethylhexahydropyrano[3,4-d]imidazol-2(3H)-one (Example 124);
(3aR,6R,7aR)-1-(7,8-dihydrofuro[3,2-e][1,3]benzothiazol-2-yl)-2-oxooctahydropyrano[3,4-d]imidazole-6-carbonitrile (Example 127);
(3aR,6R,7aR)-1-(7,8-dihydrofuro[3,2-e][1,3]benzothiazol-2-yl)-6-methoxyhexahydropyrano[3,4-d]imidazol-2(3H)-one (Example 129);
(3aR,6R,7aR)-1-(2H-[1,3]dioxolo[4,5-e][1,3]benzothiazol-7-yl)-2-oxooctahydropyrano[3,4-d]imidazole-6-carbonitrile (Example 131);
(3aR,6R,7aR)-1-(7,8-dihydro[1,4]dioxino[2,3-e][1,3]benzothiazol-2-yl)-2-oxooctahydropyrano[3,4-d]imidazole-6-carbonitrile (Example 133);
(3aR,8aR)-1-(7,8-dihydro[1,4]dioxino[2,3-e][1,3]benzothiazol-2-yl)hexahydro-1H-oxepino[3,4-d]imidazol-2(3H)-one (Example 135);
(3aR,6S,7aR)-1-(2H-[1,3]dioxolo[4,5-e][1,3]benzothiazol-7-yl)-6-ethylhexahydropyrano[3,4-d]imidazol-2(3H)-one (Example 138);
(3aR,6S,7aR)-1-(2H-[1,3]dioxolo[4,5-e][1,3]benzothiazol-7-yl]-6-methylhexahydropyrano[3,4-d]imidazol-2(3H)-one (Example 139);
(3aR,6R,7aR)-1-(2H-[1,3]dioxolo[4,5-e][1,3]benzothiazol-7-yl]-6-(fluoromethyl)hexahydropyrano[3,4-d]imidazol-2(3H)-one(Example 140);
(3aR,6R,7aR)-1-(7,8-dihydrofuro[3,2-e][1,3]benzothiazol-2-yl)-6-(hydroxymethyl)hexahydropyrano[3,4-d]imidazol-2(3H)-one(Example 144);
(3aR,6R, 7aR)-1-(7,8-dihydro[1,4]dioxino[2,3-e][1,3]benzothiazol-2-yl)-6-(hydroxymethyl)hexahydropyrano[3,4-d]imidazol-2(3H)-one(Example 146);
(3aR,6R,7aR)-1-(2H-[1,3]dioxolo[4,5-e][1,3]benzothiazol-7-yl)-6-(hydroxymethyl)hexahydropyrano[3,4-d]imidazol-2(3H)-one(Example 147);
(3aR,6R,7aR)-1-(7,8-dihydrofuro[3,2-e][1,3]benzothiazol-2-yl)-6-ethynylhexahydropyrano[3,4-d]imidazol-2(3H)-one (Example 149);
(3aR,7aR)-1-(7,8-dihydrofuro[3,2-e][1,3]benzothiazol-2-yl)-6-fluorohexahydropyrano[3,4-d]imidazol-2(3H)-one (Example 151);
(3aR,6S,6aS)-1-(7,8-dihydrofuro[3,2-e][1,3]benzothiazol-2-yl)-6-(hydroxymethyl)tetrahydro-1H-furo[3,4-d]imidazol-2(3H)-one(Example 157);
(3aR,6S,6aS)-1-(7,8-dihydrofuro[3,2-e][1,3]benzothiazol-2-yl)-6-(fluoromethyl)tetrahydro-1H-furo[3,4-d]imidazol-2(3H)-one(Example 159);
(3aR,6R,6aS)-1-(7,8-dihydrofuro[3,2-e][1,3]benzothiazol-2-yl)-6-methyltetrahydro-1H-furo[3,4-d]imidazol-2(3H)-one (Example 161); and
[(3aS,4R,6aR)-3-(7,8-dihydrofuro[3,2-e][1,3]benzothiazol-2-yl)-2-oxohexahydro-1H-furo[3,4-d]imidazol-4-yl]acetonitrile(Example 162).

Among the compounds of the present invention represented by formula (1), specific examples of a more preferable compound include the following compounds or pharmaceutically acceptable salts thereof.
(3aR,7aR)-1-(2H-[1,3]Dioxolo[4,5-e][1,3]benzothiazol-7-yl)hexahydropyrano[3,4-d]imidazol-2(3H)-one (Example 21);
(4S,5R)-1-(7,8-dihydrofuro[3,2-e][1,3]benzothiazol-2-yl)-4-methyl-5-[(morpholin-4-yl)methyl]imidazolidin-2-one (Example 23);
(3aR,7aS)-5-cyclopropyl-3-(7,8-dihydro[1,4]dioxino[2,3-e][1,3]benzothiazol-2-yl)octahydro-2H-imidazo[4,5-c]pyridin-2-one (Example 34);
(3aR,7aS)-5-cyclopropyl-3-(2H-[1,3]dioxolo[4,5-e][1,3]benzothiazol-7-yl)octahydro-2H-imidazo[4,5-c]pyridin-2-one (Example 36);
(3aR,7aR)-1-(7,8-dihydro[1,4]dioxino[2,3-e][1,3]benzothiazol-2-yl)hexahydropyrano[3,4-d]imidazol-2(3H)-one (Example 37);
(3aR,6S,7aR)-1-(7,8-dihydrofuro[3,2-e][1,3]benzothiazol-2-yl)-6-methylhexahydropyrano[3,4-d]imidazol-2(3H)-one (Example 38);
(3aR,6S,7aR)-1-(7,8-dihydro[1,4]dioxino[2,3-e][1,3]benzothiazol-2-yl)-6-methylhexahydropyrano[3,4-d]imidazol-2 (3H)-one (Example 39);
(3aR,6R,7aR)-1-(7,8-dihydrofuro[3,2-e][1,3]benzothiazol-2-yl)-6-(fluoromethyl)hexahydropyrano[3,4-d]imidazol-2(3H)-one(Example 40);
(3aR,6R,7aR)-1-(7,8-dihydro[1,4]dioxino[2,3-e][1,3]benzothiazol-2-yl)-6-(fluoromethyl)hexahydropyrano[3,4-d]imidazol-2(3H)-one (Example 41);
(3aR,6S,7aR)-1-(7,8-dihydrofuro[3,2-e][1,3]benzothiazol-2-yl)-6-ethylhexahydropyrano[3,4-d]imidazol-2(3H)-one (Example 42);
(3aR,6S, 7aR)-1-(7,8-dihydro[1,4]dioxino[2,3-e][1,3]benzothiazol-2-yl)-6-ethylhexahydropyrano[3,4-d]imidazol-2(3H)-one (Example 43);
(4S,5S)-1-(7,8-dihydrofuro[3,2-e][1,3]benzothiazol-2-yl)-5-(methoxymethyl)-4-methylimidazolidin-2-one (Example 44);
rac-(3aR,8aS)-3-(7,8-dihydrofuro[3,2-e][1,3]benzothiazol-2-yl)-5-(oxetan-3-yl)octahydroimidazo[4,5-c]azepin-2(1H)-one(Example 73);
rac-(3aR,7aS)-5-cyclopropyl-3-(7,8-dihydrofuro[3,2-e][1,3]benzothiazol-2-yl)octahydro-2H-imidazo[4,5-c]pyridin-2-one (Example 98);
rac-(3aR,8aS)-5-cyclopropyl-3-(7,8-dihydrofuro[3,2-e][1,3]benzothiazol-2-yl)octahydroimidazo[4,5-c]azepin-2(1H)-one(Example 104);
(3aR,7aS)-5-cyclopropyl-3-(7,8-dihydrofuro[3,2-e][1,3]benzothiazol-2-yl)octahydro-2H-imidazo[4,5-c]pyridin-2-one (Example 110);
[(3aR,7aS)-3-(7,8-dihydrofuro[3,2-e][1,3]benzothiazol-2-yl)-2-oxooctahydro-5H-imidazo[4,5-c]pyridin-5-yl]acetonitrile(Example 112);
(3aR,7aS)-3-(7,8-dihydrofuro[3,2-e][1,3]benzothiazol-2-yl)-5-(2-propyn-1-yl)octahydro-2H-imidazo[4,5-c]pyridin-2-one(Example 114);
(3aR,7aR)-1-(7,8-dihydrofuro[3,2-e][1,3]benzothiazol-2-yl)hexahydropyrano[3,4-d]imidazol-2(3H)-one (Example 122);
(3aR,7aR)-1-(7,8-dihydrofuro[3,2-e][1,3]benzothiazol-2-yl)-6,6-dimethylhexahydropyrano[3,4-d]imidazol-2(3H)-one (Example 124);
(3aR,6R,7aR)-1-(7,8-dihydrofuro[3,2-e][1,3]benzothiazol-2-yl)-2-oxooctahydropyrano[3,4-d]imidazole-6-carbonitrile (Example 127);
(3aR,6R,7aR)-1-(2H-[1,3]dioxolo[4,5-e][1,3]benzothiazol-7-yl)-2-oxooctahydropyrano[3,4-d]imidazole-6-carbonitrile (Example 131);
(3aR,6R, 7aR)-1-(7,8-dihydro[1,4]dioxino[2,3-e][1,3]benzothiazol-2-yl)-2-oxooctahydropyrano[3,4-d]imidazole-6-carbonitrile (Example 133);
(3aR,6S,7aR)-1-(2H-[1,3]dioxolo[4,5-e][1,3]benzothiazol-7-yl)-6-ethylhexahydropyrano[3,4-d]imidazol-2(3H)-one (Example 138);
(3aR,6S,7aR)-1-(2H-[1,3]dioxolo[4,5-e][1,3]benzothiazol-7-yl]-6-methylhexahydropyrano[3,4-d]imidazol-2(3H)-one (Example 139);
(3aR,6R,7aR)-1-(2H-[1,3]dioxolo[4,5-e][1,3]benzothiazol-7-yl]-6-(fluoromethyl)hexahydropyrano[3,4-d]imidazol-2(3H)-one(Example 140);
(3aR,6R,7aR)-1-(7,8-dihydrofuro[3,2-e][1,3]benzothiazol-2-yl)-6-ethynylhexahydropyrano[3,4-d]imidazol-2(3H)-one (Example 149); and
(3aR,7aR)-1-(7,8-dihydrofuro[3,2-e][1,3]benzothiazol-2-yl)-6-fluorohexahydropyrano[3,4-d]imidazol-2(3H)-one (Example 151).

Hereinafter, the method for producing the compound represented by formula (1) in the present invention will be described with reference to examples, but the present invention is not limited thereto.

The compound of the present invention is synthesized by a production method shown below and a method combining a known compound and a known synthesis method.

Each of the compounds in a reaction scheme also includes a salt thereof, and examples of the salt include the same as a salt of compound (1). These reactions are merely examples, and the compound of the present invention can also be appropriately produced by other methods based on the knowledge of a person who is familiar organic synthesis.

In each of the production methods described below, even if the use of a protective group is not specifically specified, when a functional group that requires protection is present, the target product may be obtained by, if necessary, protecting the functional group and deprotecting the same after completion of the reaction or after carrying out a series of reactions.

As the protective group, a usual protective group described in, for example, reference (T. W. Greene and P. G. M. Wuts, “Protective Groups in Organic Synthesis,” 3rd Ed., John Wiley and Sons, inc., New York (1999)) can be used, and more specifically, examples of a protective group for an amino group include tert-butoxycarbonyl, benzyloxycarbonyl, dimethylformamide, p-toluenesulfonyl, o-nitrobenzenesulfonyl, and tetrahydropyranyl, examples of a protective group for a hydroxy group include trialkylsilyl, acetyl, benzyl, tetrahydropyranyl, and methoxymethyl, examples of a protective group for an aldehyde group include dialkyl acetal, and cyclic alkyl acetal, and examples of a protective group for a carboxyl group include a tert-butyl ester, an ortho ester, and an acid amide.

The compounds wherein these functional groups are protected are also encompassed by the compound of the above formula (1).

The introduction and elimination of a protective group can be carried out by a method commonly used in organic synthetic chemistry (for example, a method described in T. W. Greene and P. G. M. Wuts, “Protective Groups in Organic Synthesis,” 3rd Ed., John Wiley and Sons, inc., New York (1999)) or a method similar thereto.

Production Method 1

The compound represented by formula (1-9) is produced, for example, by the method shown below.

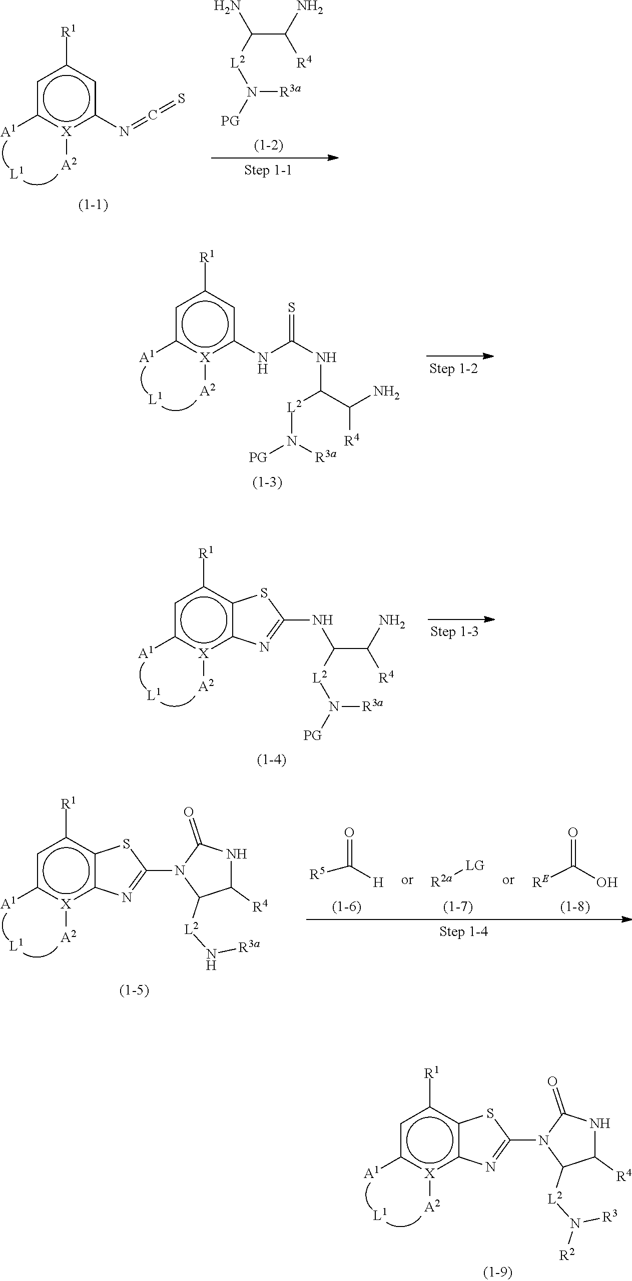

wherein R¹, R², R³, R⁴, A¹, A², L¹, L², R^{E}, and X are defined as described in item 1 above; R^{2a} and R^{3a} each represent optionally substituted Cia alkyl; R⁵ represents optionally substituted C₁₋₅ alkyl; LG represents a leaving group (for example, an iodine atom, a bromine atom, a chlorine atom, or —O-substituted sulfonyl group (for example, a methanesulfonyl group or a p-toluenesulfonyl group)); and PG represents a protective group (for example, a tert-butoxycarbonyl group, a benzyloxycarbonyl group, or a benzyl group).

Step 1-1: Production Step of Compound (1-3)

Compound (1-3) can be obtained by reacting compound (1-1) with compound (1-2) by a method similar to a known synthesis method (for example, Chemical & Pharmaceutical Bulletin, 1406, (2007), or Advanced Synthesis & Catalysis, 1643, (2005)). As compound (1-1), a compound produced by a known synthesis method (for example, Bioorganic & Medicinal Chemistry Letters, 28, (2007), or J. Org. Chem. 2613, (1986)) or a synthesis method similar thereto can be used. As compound (1-2), a commercially available product or a compound produced by a known synthesis method (for example, Tetrahedron Letters, 946, (2015), or Synlett, 426, (1999)) or a synthesis method similar thereto can be used.

Step 1-2: Production Step of Compound (1-4)

Compound (1-4) is produced by cyclizing compound (1-3) by a method similar to a known synthesis method (for example, Journal of Organic Chemistry, 8693, (2003) or WO2013043001).

Step 1-3: Production Step of Compound (1-5)

Compound (1-5) is produced by cyclizing compound (1-4) by a method similar to a known synthesis method (for example, Organic Letters, 5136, (2015), or Bioorganic & Medicinal Chemistry, 822, (2008)).

Step 1-4: Production Step of Compound (1-9)

Compound (1-9) can be obtained by reacting compound (1-5) with compound (1-6) in an inert solvent in the presence of a borohydride compound and, if necessary, an acid. As compound (1-6), a commercially available product or a compound produced by a known synthesis method (for example, US200619965, or Journal of Medicinal Chemistry, 3680, (2003)) or a synthesis method similar thereto can be used.

Specific examples of the inert solvent include an ether-based solvent such as tetrahydrofuran, tetrahydropyran, 1,4-dioxane, or 1,2-dimethoxyethane; a halogenated hydrocarbon such as chloroform, dichloromethane, or 1,2-dichloroethane; a protic polar solvent such as methanol, ethanol, 1-propanol, 2-propanol, or water; and mixed solvents thereof. The inert solvent is preferably tetrahydrofuran, dichloromethane, chloroform, or methanol.

Specific examples of the acid include a carboxylic acid such as formic acid, propionic acid, acetic acid, or trifluoroacetic acid; and a mineral acid such as hydrochloric acid.

Specific examples of the borohydride compound include sodium triacetoxyborohydride, sodium cyanoborohydride, and sodium borohydride. The borohydride compound is preferably sodium triacetoxyborohydride or sodium cyanoborohydride.

The reaction temperature is not particularly limited, and is usually selected from the range from 0° C. to the boiling point of the solvent used. The reaction temperature is preferably 0° C. to 20° C. The reaction time is usually 30 minutes to 72 hours.

Compound (1-9) is also produced by using compound (1-7) and a base and reacting the same with compound (1-5) in an inert solvent, in the presence of a halogenating agent, if necessary. As compound (1-7), a commercially available product or a compound produced by a known synthesis method (for example, US2013/1503254 or EP1679308, (2006)) or a synthesis method similar thereto can be used.

Specific examples of the inert solvent include an ether-based solvent such as tetrahydrofuran, tetrahydropyran, 1,4-dioxane, or 1,2-dimethoxyethane; an aromatic hydrocarbon such as toluene, xylene, or pyridine; an aprotic polar solvent such as acetonitrile, propionitrile, N,N-dimethylformamide, N,N-dimethylacetamide, N-methyl-2-pyrrolidinone, or dimethyl sulfoxide; a halogenated hydrocarbon such as chloroform, dichloromethane, or 1,2-dichloroethane, and mixed solvents thereof. The inert solvent is preferably acetonitrile, N,N-dimethylacetamide, or dimethyl sulfoxide.

Specific examples of the base include an organic base such as triethylamine, diisopropylethylamine, or pyridine; and an inorganic base such as potassium carbonate, sodium carbonate, cesium carbonate, potassium hydroxide, or sodium hydroxide. The base is preferably diisopropylethylamine, potassium carbonate, or cesium carbonate.

Specific examples of the halogenating agent include an inorganic halide such as potassium iodide or sodium iodide; and an organic halide such as tetrabutylammonium iodide.

The reaction temperature is not particularly limited, and is usually selected from the range from 0° C. to the boiling point of the solvent used. The reaction temperature is preferably 0° C. to 100° C. The reaction time is usually 30 minutes to 72 hours.

Compound (1-9) is also produced by using a carboxylic acid (1-8) and a condensing agent or an acid anhydride corresponding to the carboxylic acid (1-8) and reacting the same with compound (1-5) in an inert solvent, in the presence of a base and an additive, if necessary. As the carboxylic acid (1-8) or the acid anhydride corresponding thereto, a commercially available product or a compound produced by a known synthesis method (for example, Journal of Organic Chemistry 2564, (1999), or Organic Letters 4739, (2004)) or a synthesis method similar thereto can be used.

Specific examples of the inert solvent include an ether-based solvent such as tetrahydrofuran, tetrahydropyran, 1,4-dioxane, or 1,2-dimethoxyethane; an aromatic hydrocarbon such as toluene, xylene, or pyridine; an aprotic polar solvent such as acetonitrile, propionitrile, N,N-dimethylformamide, N,N-dimethylacetamide, N-methyl-2-pyrrolidinone, or dimethyl sulfoxide; a halogenated hydrocarbon such as chloroform, dichloromethane, or 1,2-dichloroethane, and mixed solvents thereof. The inert solvent is preferably N,N-dimethylacetamide, N-methyl-2-pyrrolidinone, chloroform, or dichloromethane.

Specific examples of the condensing agent include dicyclohexylcarbodiimide, diisopropylcarbodiimide, 1-ethyl-3-(3-dimethylaminopropyl)-carbodiimide, benzotriazol-1-yl-tris(dimethylamino)phosphonium hexafluorophosphate, diphenylphosphonyldiamide, N,N-carbonyldimidazole, 0-(benzotriazol-1-yl)-N,N,N′,N′-tetramethyluronium hexafluorophosphate, and O-(7-azabenzotriazol-1-yl)-N,N,N′,N′-tetramethyluronium hexafluorophosphate.

Specific examples of the additive include N-hydroxysuccinimide, 1-hydroxybenzotriazole, and 3-hydroxy-4-oxo-3,4-dihydro-1,2,3-benzotriazine, and the reaction can be carried out by adding such an additive.

Specific examples of the base include an organic base such as triethylamine, diisopropylethylamine, or pyridine.

The reaction temperature is not particularly limited, and is usually selected from the range from about −20° C. to the boiling point of the solvent used. The reaction temperature is preferably 0° C. to 20° C. The reaction time is usually 10 minutes to 48 hours.

Production Method 2

The compound represented by formula (2-7) is produced, for example, by the method shown below.

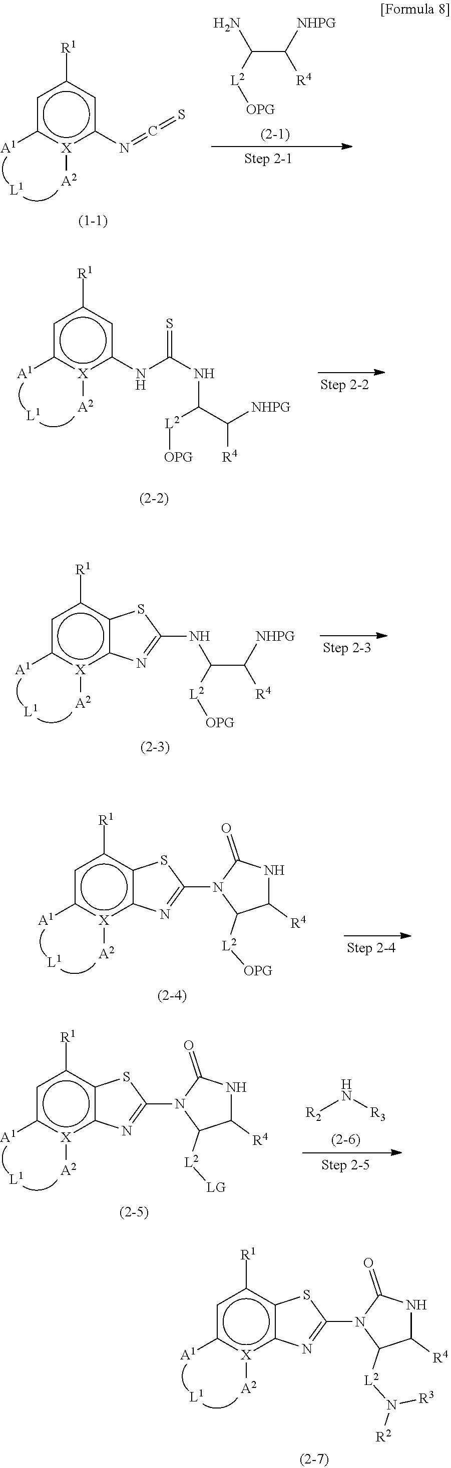

wherein R¹, R², R³, R⁴, A¹, A², L¹, L², and X are defined as described in item 1 above; LG represents a leaving group (for example, an iodine atom, a bromine atom, a chlorine atom, or an —O-substituted sulfonyl group (for example, a methanesulfonyl group or a p-toluenesulfonyl group); and PG represents a protective group (for example, a tert-butoxycarbonyl group, a benzyloxycarbonyl group, or a benzyl group).

Step 2-1: Production Step of Compound (2-2)

Compound (2-2) is produced according to the method described in step 1-1 by using compound (1-1) and compound (2-1). As compound (2-1), a compound produced by a known synthesis method (for example, Chemical Communications 7693, (2015) or WO2015061572) or a synthesis method similar thereto can be used.

Step 2-2: Production Step of Compound (2-3)

Compound (2-3) is produced according to the method described in step 1-2 by using compound (2-2).

Step 2-3: Production Step of Compound (2-4)

Compound (2-4) is produced by using compound (2-3) and according to the method described in step 1-3 after deprotection of the protective group.

Step 2-4: Production Step of Compound (2-5)

When LG is a substituted sulfonyl group, compound (2-5) can be obtained by deprotecting the protective group and then reacting compound (2-4) with a sulfonyl chloride in an inert solvent in the presence of a base. When LG is a halogen, compound (2-5) can be obtained by deprotecting the protective group and then reacting compound (2-4) with a halogenating agent in an inert solvent.

Specific examples of the inert solvent include an ether-based solvent such as tetrahydrofuran, tetrahydropyran, 1,4-dioxane, or 1,2-dimethoxyethane; an aromatic hydrocarbon such as toluene or xylene; a halogenated hydrocarbon such as chloroform, dichloromethane, or 1,2-dichloroethane; an aprotic polar solvent such as acetonitrile, N,N-dimethylformamide, N,N-dimethylacetamide, or N-methyl-2-pyrrolidinone; and mixed solvents thereof. The inert solvent is preferably tetrahydrofuran, chloroform, or dichloromethane.

Specific examples of the base include an organic base such as triethylamine, diisopropylethylamine, pyridine, 2,4,6-trimethylpyridine, or 4-dimethylaminopyridine. The base is preferably triethylamine or diisopropylamine.

Specific examples of the sulfonyl chloride include methanesulfonyl chloride, monochloromethanesulfonyl chloride, benzenesulfonyl chloride, p-toluenesulfonyl chloride, o-nitrobenzenesulfonyl chloride, and p-nitrobenzenesulfonyl chloride. The sulfonyl chloride is preferably methanesulfonyl chloride.

Specific examples of the halogenating agent include thionyl chloride, oxalyl dichloride, and phosphorus tribromide. The halogenating agent is preferably thionyl chloride or phosphorus tribromide.

The reaction temperature is not particularly limited, and is usually selected from the range from −20° C. to the boiling point of the solvent used. The reaction temperature is preferably 0° C. to 60° C. The reaction time is usually 5 minutes to 24 hours.

Step 2-5: Production Step of Compound (2-7)

Compound (2-7) can be obtained by reacting compound (2-5) with (2-6) in an inert solvent in the presence of a base and, if necessary, a halide. As compound (2-6), a commercially available product or a compound produced by a known synthesis method (for example, WO20073965 or US2010216812) or a synthesis method similar thereto can be used.

Specific examples of the inert solvent include an ether-based solvent such as tetrahydrofuran, tetrahydropyran, 1,4-dioxane, or 1,2-dimethoxyethane; a halogenated hydrocarbon such as chloroform, dichloromethane, or 1,2-dichloroethane; an aprotic polar solvent such as acetonitrile, N,N-dimethylformamide, N,N-dimethylacetamide, N-methyl-2-pyrrolidinone, or dimethyl sulfoxide; and mixed solvents thereof. The inert solvent is preferably acetonitrile, tetrahydrofuran, dichloromethane, or N,N-dimethylformamide.

Specific examples of the base include an organic base such as triethylamine, diisopropylethylamine, pyridine, 2,4,6-trimethylpyridine, or 4-dimethylaminopyridine; and an inorganic base such as potassium carbonate, sodium carbonate, or cesium carbonate.

Specific examples of the halide include an organic halide such as tetrabutylammonium iodide or tetrabutylammonium bromide; and an inorganic halide such as potassium iodide, potassium bromide, sodium iodide, or sodium bromide.

The reaction temperature is not particularly limited, and is usually selected from the range from −20° C. to the boiling point of the solvent used or compound (2-6). The reaction temperature is preferably 0° C. to 120° C. The reaction time is usually 30 minutes to 72 hours.

Production Method 3

The compound represented by formula (1-4) is also produced, for example, by the method shown below.

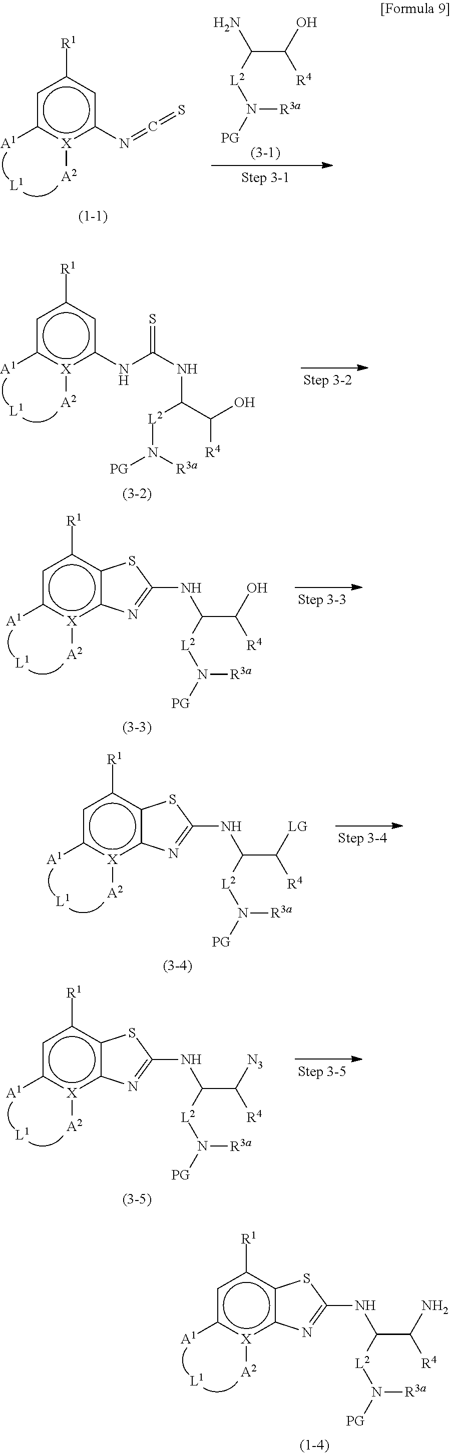

wherein R¹, R², R³, R⁴, A¹, A², L¹, L², and X are defined as described in item 1 above; R^{3a} each represent optionally substituted C₁-6 alkyl; LG represents a leaving group (for example, an iodine atom, a bromine atom, a chlorine atom, or a —O-substituted sulfonyl group (for example, a methanesulfonyl group or a p-toluenesulfonyl group)); and PG represents a protective group (for example, a tert-butoxycarbonyl group, a benzyloxycarbonyl group, or a benzyl group).

Step 3-1: Production Step of Compound (3-2)

Compound (3-2) is produced according to the method described in step 1-1 by using compound (1-1) and compound (3-1). As compound (3-1), a commercially available product or a compound produced by a known synthesis method (for example, WO2008136457 or WO2014015905) or a synthesis method similar thereto can be used.

Step 3-2: Production Step of Compound (3-3)

Compound (3-3) is produced according to the method described in step 1-2 by using compound (3-2).

Step 3-3: Production Step of Compound (3-4)

Compound (3-4) is produced according to the method described in step 2-4 by using compound (3-3).

Step 3-4: Production Step of Compound (3-5)

Compound (3-5) is produced by synthesizing the same by a method similar to a known synthesis method (for example, Journal of Medicinal Chemistry 3918, (1995) or WO2015177326) by using compound (3-4).

Step 3-5: Production Step of Compound (1-4)

Compound (1-4) is also produced by synthesizing the same by a method similar to a known synthesis method (for example, Bioorganic & Medicinal Chemistry Letters 5227 (2007) or WO2016044641) by using compound (3-5).

Production Method 4

The compound represented by formula (4-5) is produced, for example, by the method shown below.

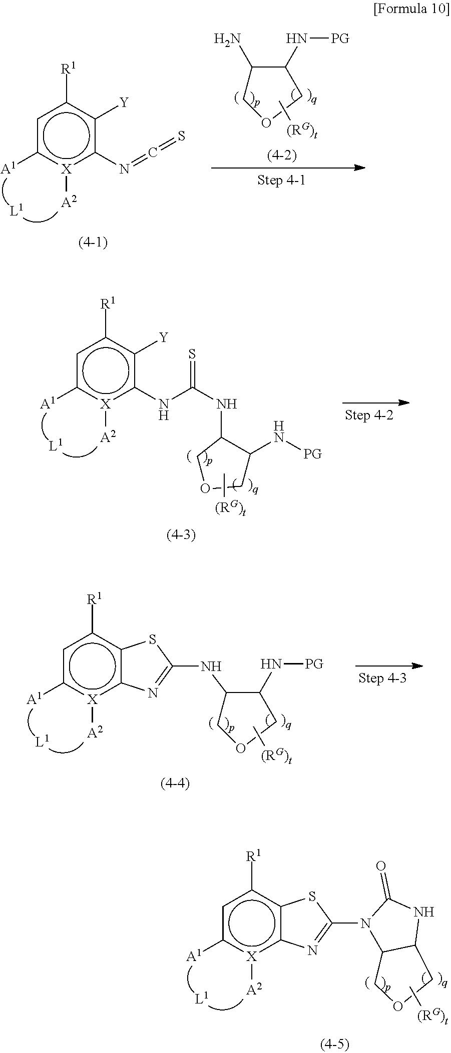

wherein R¹, A¹, A², L¹, and X are defined as described in item 1 above, and R^{G}, p, q, and t are defined as described in item 16 above; Y Represents a halogen atom (for example, an iodine atom, a bromine atom, or a chlorine atom); and PG represents a protective group (for example, a tert-butoxycarbonyl group or a benzyloxycarbonyl group).

Step 4-1: Production Step of Compound (4-3)

Compound (4-3) is produced according to the method described in step 1-1 by using compound (4-1) and compound (4-2). As compound (4-1), a compound produced by a known synthesis method (for example, Bioorganic & Medicinal Chemistry Letters, 28, (2007) or Journal of Organic Chemistry 2613, (1986)) or a synthesis method similar thereto can be used. As compound (4-2), a commercially available product or a compound produced by a known synthesis method (for example, WO2012061418 or WO2010097248) or a synthesis method similar thereto can be used.

Step 4-2: Production Step of Compound (4-4)

Compound (4-4) is produced by using compound (4-3) and cyclizing the same by a method similar to a known synthesis method (for example, Chemical Communications 446, (2004) or Journal of Organic Chemistry 8719, (2009)).

Step 4-3: Production Step of Compound (4-5)

Compound (4-5) is produced by using compound (4-4) and according to the method described in step 1-3 after deprotection of the protective group.

Production Method 5

The compound represented by formula (5-4) is produced, for example, by the method shown below.

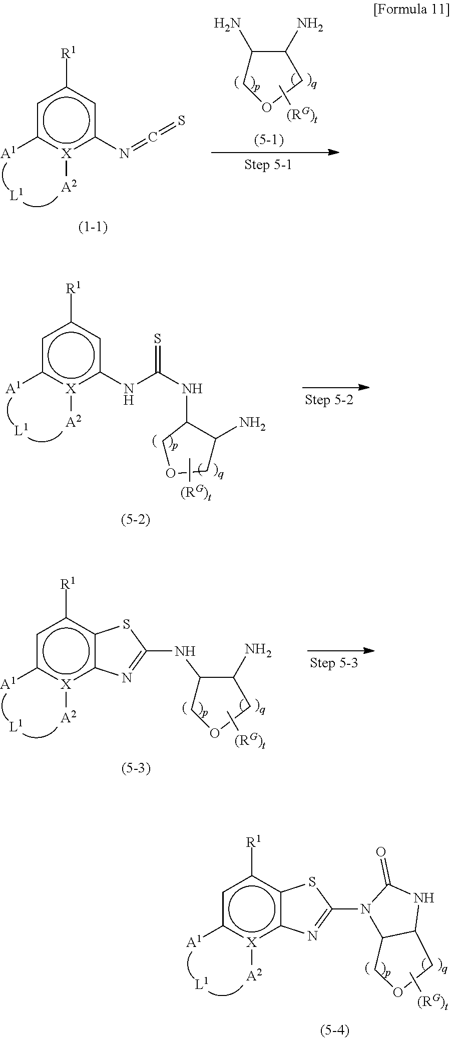

wherein R¹, A¹, A², L¹, and X are defined as described in item 1 above, and R^{G}, p, q, and t are defined as described in item 16 above.

Step 5-1: Production Step of Compound (5-2)

Compound (5-2) is produced according to the method described in step 1-1 by using compound (1-1) and compound (5-1). As compound (5-1), a commercially available product or a compound produced by a known synthesis method (for example, Journal of the American Chemical Society, 12521, (1996) or Green Chemistry 451, (2005)) or a synthesis method similar thereto can be used.

Step 5-2: Production Step of Compound (5-3)

Compound (5-3) is produced according to the method described in step 1-2 by using compound (5-2).

Step 5-3: Production Step of Compound (5-4)

Compound (5-4) is produced according to the method described in step 1-3 by using compound (5-3).

By carrying out the above production methods in an appropriate combination, the compound of the present invention having a desired functional group at a desired position can be obtained. Isolation and purification of intermediates and products in the above production methods can be carried out by appropriately combining methods used in ordinary organic synthesis, such as filtration, extraction, washing, drying, concentration, crystallization, and various chromatography. In addition, such an intermediate can also be subjected to the next reaction without any particular purification.

Examples of the “pharmaceutically acceptable salt” include an acid addition salt and a base addition salt. Examples of the acid addition salt include an inorganic acid salt such as a hydrochloride, a hydrobromide, a sulfate, a hydroiodide, a nitrate, or a phosphate, or an organic acid salt such as a citrate, an oxalate, a phthalate, a fumarate, a maleate, a succinate, a malate, an acetate, a formate, a propionate, a benzoate, a trifluoroacetate, a methanesulfonate, a benzenesulfonate, a para-toluenesulfonate, or a camphorsulfonate. In addition, examples of the base addition salt include an inorganic base salt such as a sodium salt, a potassium salt, a calcium salt, a magnesium salt, a barium salt, or an aluminum salt, or an organic base salt such as trimethylamine, triethylamine, pyridine, picoline, 2,6-lutidine, ethanolamine, diethanolamine, triethanolamine, tromethamine[tris(hydroxymethyl)methylamine], tert-butylamine, cyclohexylamine, dicyclohexylamine, or N,N-dibenzylethylamine. Further, examples of the “pharmaceutically acceptable salt” also include an amino acid salt with a basic amino acid or an acidic amino acid, such as arginine, lysine, ornithine, aspartic acid, or glutamic acid.

Suitable salts of a raw material compound and an intermediate and a salt acceptable as a raw material for a pharmaceutical are conventional nontoxic salts, and examples thereof include an acid addition salt such as an organic acid salt (for example, an acetate, a trifluoroacetate, a maleate, a fumarate, a citrate, a tartrate, a methanesulfonate, a benzenesulfonate, a formate, or a p-toluenesulfonate) and an inorganic acid salt (for example, a hydrochloride, a hydrobromide, a hydroiodide, a sulfate, a nitrate, or a phosphate), a salt with an amino acid (for example, arginine, aspartic acid, or glutamic acid), a metal salt such as an alkali metal salt (for example, a sodium salt or a potassium salt) and an alkaline earth metal salt (for example, a calcium salt or a magnesium salt), an ammonium salt, or an organic base salt (for example, a trimethylamine salt, a triethylamine salt, a pyridine salt, a picoline salt, a dicyclohexylamine salt, or an N,N′-dibenzylethylenediamine salt), and such a nontoxic salt can be appropriately selected by those skilled in the art.

Some of the raw material compounds or intermediates in the above production methods can exist in the form of a salt such as a hydrochloride depending on the reaction conditions and the like, and can be used as they are or in free form. When a raw material compound or an intermediate is obtained in the form of a salt and the raw material compound or intermediate is to be used or obtained in free form, this salt can be converted to a free form by dissolving or suspending the salt in a suitable solvent and neutralizing the same with, for example, a base such as a sodium hydrogen carbonate aqueous solution.

For some compounds (1) or pharmaceutically acceptable salts thereof, an isomer such as a tautomer such as a keto-enol form, a regioisomer, a geometric isomer, or an optical isomer can exist, and all possible isomers, including these, and mixtures of the isomers at any ratio are also encompassed by the present invention.

In addition, the optical isomer can be separated by carrying out a known separation step such as a method using an optically active column or a fractional crystallization method in an appropriate step of the above production methods. In addition, an optically active substance can also be used as a starting material.

As used herein, a compound with stereochemistry (S, R) notation in its chemical structural formula means an optically active form, and when stereochemistry is not particularly indicated, the compound means a racemic form. Regarding the cyclic urea moiety of the compound of the present invention, a cis form or a trans form exists, and in particular, when stereochemistry (S, R) is not indicated, the moiety means a racemic form.

When a salt of compound (1) is to be obtained, if the salt of compound (1) can be obtained, the salt may be purified as it is, and if compound (1) is obtained in free form, the salt thereof may be formed by dissolving or suspending compound (1) in a suitable solvent and adding an acid or a base. In addition, compound (1) or a pharmaceutically acceptable salt thereof may exist in the form of a solvate with water or any of various solvents, and such a solvate is also encompassed by the present invention.

As used herein, the “hydrogen atom” includes 1H and²H (D), and a deuterium conversion form obtained by converting any one or two or more 1H in the compound represented by formula (1) into ²H (D) is also encompassed by the compound represented by formula (1).

The compound of the present invention can be administered, directly or by being formulated into an appropriate dosage form, by oral administration or parenteral administration. Examples of the dosage form include, but are not limited to, a tablet, a capsule, a powder, a granule, a liquid, a suspension, an injection, a patch, and a cataplasm. The formulation is produced by a known method by using a pharmaceutically acceptable additive. As an additive, an excipient, a disintegrant, a binding agent, a plasticizer, a lubricant, a coating agent, a solubilizing agent, a dissolution aid, a thickening agent, a dispersing agent, a stabilizing agent, a sweetening agent, a flavoring agent, or the like can be used depending on the purpose. Specifically, examples thereof include lactose, mannitol, crystalline cellulose, low substituted hydroxypropylcellulose, corn starch, partially pregelatinized starch, carmellose calcium, croscarmellose sodium, hydroxypropylcellulose, hydroxypropylmethylcellulose, polyvinyl alcohol, magnesium stearate, sodium stearyl fumarate, polyethylene glycol, propylene glycol, titanium oxide, and talc.

The administration route of the compound of the present invention may be oral administration, parenteral administration, or rectal administration, and the daily dosage thereof varies depending on the type of the compound, the administration method, the symptom/age of the patient, and the like. For example, in the case of oral administration, usually about 0.01 to 1000 mg, further preferably about 0.1 to 500 mg, per kg of human or mammal body weight can be administered in one to several divided doses. In the case of parenteral administration such as intravenous injection, usually, for example, about 0.01 to 300 mg, further preferably about 1 to 100 mg, per kg of human or mammal body weight can be administered.

In addition, compound (1) of the present invention or a pharmaceutically acceptable salt thereof can be used, as a DYRK inhibitor, as a reagent for pathological imagery related to the above diseases or a reagent for a basic experiment or for research.

### EXAMPLES

Hereinafter, the present invention will be described more specifically with reference to Examples, Reference Examples, and Test Examples, but the present invention is not limited thereto at all. The compound names shown in the following Examples and Reference Examples do not necessarily follow the IUPAC nomenclature.

The following abbreviations may be used herein.
(Boc)₂O: di-tert-butyl dicarbonate
Cbz: benzyloxycarbonyl
DIAD: diisopropyl azodicarboxylate
Boc: tert-butoxycarbonyl
Bn: benzyl
TBDMS: tert-butyldimethylsilyl
Ac: acetyl
Ms: methanesulfonyl
DMSO: dimethyl sulfoxide
Ts: p-toluenesulfonyl
Tf: trifluoromethanesulfonyl
Rt: retention time

Physical property data of each compound of the Examples and the Reference Examples were measured under the following conditions.
Nuclear magnetic resonance spectrum (¹H-NMR):
Device used: JEOL JNM-AL400; Brucker AVANCE 400 Spectrometer
Liquid chromatography/mass spectrometry (LC/MS):

(1) Method A and Method B
Detection device: ACQUITY® SQ deteceter (Waters Corporation)
HPLC: ACQUITY UPLC®
SYSTEM Column: Waters ACQUITY UPLC® BEH C18 (1.7 um, 2.1 mm×30 mm)
Flow rate: 0.8 mL/min; Detection UV: 220 nm and 254 nm;
Temperature: 40° C.

TABLE 1

| Method | Solvent | Gradient condition |
|---|---|---|
| Method A | Solvent A: | 0.0-1.3 min Linear |
| | 0.05% formic acid/water | gradient from B 2% to 96% |
| | Solvent B: | |
| | CH₃CN | |
| Method B | Solvent A: | 0.0-1.3 min Linear |
| | 0.05% formic acid/water | gradient from B 10% to 95% |
| | Solvent B: | |
| | CH₃CN | |

(2) Method C
Detection device: ACQUITY® QDa deteceter (Waters Corporation)
HPLC: ACQUITY UPLC®
SYSTEM Column: Waters ACQUITY UPLC® BEH C₁₈ (1.7 um, 2.1 mm×30 mm)
Flow rate: 0.6 mL/min; Detection UV: 20 to 400 nm;
Temperature: 40° C.

TABLE 2

| Method | Solvent | Gradient condition |
|---|---|---|
| Method C | Solvent A: | 0.5-2.5 min Linear |
| | 0.1% formic acid/water | gradient from B 3% to 95% |
| | Solvent B: | |
| | 0.1% formic acid/CH₃CN | |

(3) Method D
Detection device: ACQUITY® SQ deteceter 2 (Waters Corporation)
HPLC: ACQUITY UPLC®
SYSTEM Column: Waters ACQUITY UPLC® BEH C18 (1.7 um, 2.1 mm×50 mm)
Flow rate: 0.6 mL/min; Detection UV: 210 nm to 400 nm;
Temperature: 35° C.

TABLE 3

| Method | Solvent | Gradient condition |
|---|---|---|
| Method D | Solvent A: | 0.3-2.7 min Linear |
| | 0.07% formic acid/water | gradient from B 3% to 98% |
| | Solvent B: | |
| | 0.07% formic acid/CH₃CN | |

(4) Method E
Detection device: LCMS-2010A (Shimadzu Corporation)
HPLC: Prominence®
SYSTEM Column: Cadenza CD-C18 (3.0 um, 2.0 mm×50 mm)
Flow rate: 0.5 mL/min; Detection UV: 215 and 254 nm;
Temperature: 40° C.

TABLE 4

| Method | Solvent | Gradient condition |
|---|---|---|
| Method E | Solvent A: | 0.5-2.5 min Linear |
| | 0.1% formic acid/water | gradient from B 5% to 95% |
| | Solvent B: | |
| | 0.1% formic acid/CH₃CN | |

The compound names in the Reference Examples and the Examples were named by using ACD/Name (ACD/Labs 12.0, Advanced Chemistry Development Inc.).

### Reference Example 1 and Reference Example 2

### Reference Example 1: tert-butyl cis-3-amino-4-{[(2,3-dihydro-1-benzofuran-4-yl)carbamothioyl]amino}piperidine-1-carboxylate

### Reference Example 2: tert-butyl cis-4-amino-3-{[(2,3-dihydro-1-benzofuran-4-yl)carbamothioyl]amino}piperidine-1-carboxylate

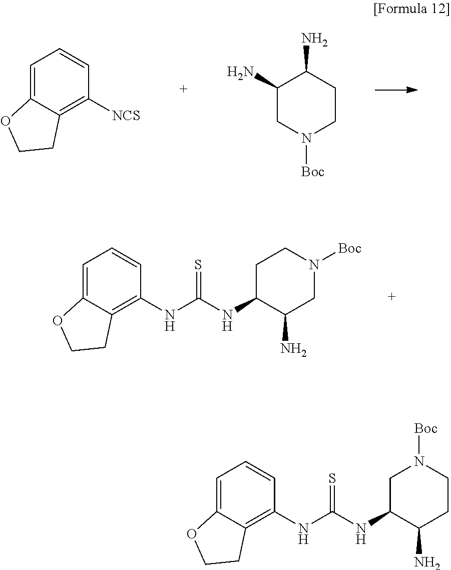

Isothiocyanato-2,3-dihydrobenzofuran (886 mg) was added to a chloroform solution (20 mL) of cis-1-Boc-3,4-diaminopiperidine (1.08 g) and N,N-diisopropylethylamine (647 mg) under ice cooling, and the resulting mixture was stirred under ice cooling for 2 hours. The reaction mixture was directly purified by silica gel column chromatography (chloroform/methanol) to obtain the title compounds (Reference Example 1: 1.04 g, Reference Example 2: 680 mg).

Reference Example 1: LC-MS [M+H]⁺/Rt (min): 393.2/0.562 (Method B)

Reference Example 2: LC-MS [M+H]⁺/Rt (min): 393.2/0.559 (Method B)

### Reference Examples 3 to 7

The compounds of Reference Examples 3 to 7 were obtained by using the corresponding raw material compounds according to the method described in Reference Example 1 and Reference Example 2.

TABLE 5

| Reference | | |
|---|---|---|
| Example | Chemical structure | Physical property data |
| 3 | 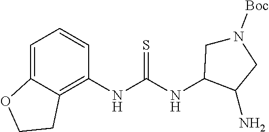 [SMILES: CC(C)(C)OC(=O)N1CC(N)C(NC(=S)Nc2cccc3c2CCO3)C1] | LC-MS [M + H]⁺/Rt (min): 378.2/0.630 (Method A). |
| 4 | 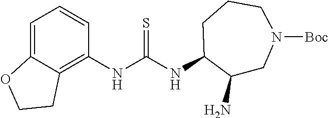 [SMILES: CC(C)(C)OC(=O)N1CCC[C@H](NC(=S)Nc2cccc3c2CCO3)[C@H](N)C1] | LC-MS [M + H]⁺/Rt (min): 407.2/0.698 (Method B). |
| 5 | 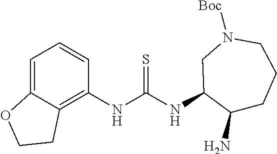 [SMILES: CC(C)(C)OC(=O)N1CCC[C@@H](N)[C@@H](NC(=S)Nc2cccc3c2CCO3)C1] | LC-MS [M + H]⁺/Rt (min): 407.2/0.682 (Method B). |
| 6 | 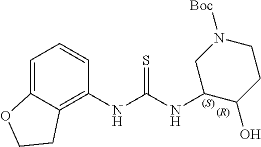 | ¹H-NMR (CDCl₃) δ: 7.58 (1H, brs), 7.16 (1H, dd, J = 7.9, 8.0 Hz), 6.73 (1H, d, J = 8.0 Hz), 6.73 (1H, d, J = 7.9 Hz), 6.45 (1H, brs), 4.69-4.56 (3H, m), 4.17-4.05 (2H, m), 3.60- 3.46 (4H, m), 3.19 (2H, t, J = 8.5 Hz), 1.86-1.77 (1H, m), 1.53-1.46 (1H, m), 2.40 (9H, s). |
| 7 | 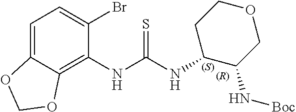 | ¹H-NMR (CDCl₃) δ: 7.58 (1H, brs), 7.16 (1H, dd, J = 7.9, 8.0 Hz), 6.73 (1H, d, J = 8.0 Hz), 6.73 (1H, d, J = 7.9 Hz), 6.45 (1H, brs), 4.69-4.56 (3H, m), 4.17-4.05 (2H, m), 3.60-3.46 (4H, m), 3.19 (2H, t, J = 8.5 Hz), 1.86-1.77 (1H, m), 1.53-1.46 (1H, m), 2.40 (9H, s). |

### Reference Example 8

### tert-Butyl cis-4-amino-3-[(7,8-dihydrofuro[3,2-e][1,3]benzothiazol-2-yl)amino]piperidine-1-carboxylate

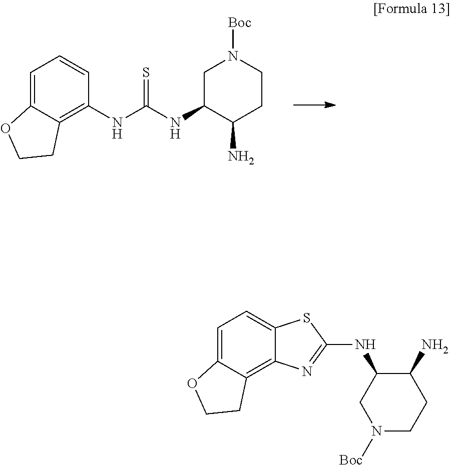 [SMILES: CC(C)(C)OC(=O)N1CC[C@@H](N)[C@@H](NC(=S)Nc2cccc3c2CCO3)C1 ; CC(C)(C)OC(=O)N1CC[C@H](N)[C@H](Nc2nc3c4c(ccc3s2)OCC4)C1]

Sodium hydrogen carbonate (580 mg) and benzyltrimethylammonium tribromide (2.69 g) were added to a chloroform solution (70 mL) of the compound of Reference Example 2 (2.71 g), and the resulting mixture was stirred for 1 hour. Saturated aqueous sodium bicarbonate was added to the reaction mixture, and the resulting mixture was extracted with chloroform. The organic layer was dried over sodium sulfate, filtered, and then concentrated under reduced pressure to obtain the title compound (2.64 g).

LC-MS [M+H]⁺/Rt (min): 391.2/0.600 (Method B)

### Reference Examples 9 to 13

The compounds of Reference Examples 9 to 13 were obtained by using the corresponding raw material compounds according to the method described in Reference Example 8.

TABLE 6

| Reference | | |
|---|---|---|
| Example | Chemical structure | Physical property data |
| 9 | 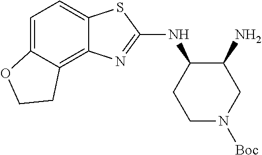 [SMILES: CC(C)(C)OC(=O)N1CC[C@@H](Nc2nc3c4c(ccc3s2)OCC4)[C@@H](N)C1] | LC-MS [M + H]⁺/Rt (min): 391.2/0.568 (Method B). |
| 10 | 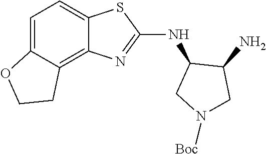 [SMILES: CC(C)(C)OC(=O)N1C[C@H](N)[C@H](Nc2nc3c4c(ccc3s2)OCC4)C1] | LC-MS [M + H]⁺/Rt (min): 377.2/0.689 (Method A). |
| 11 | 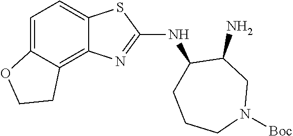 [SMILES: CC(C)(C)OC(=O)N1CCC[C@@H](Nc2nc3c4c(ccc3s2)OCC4)[C@@H](N)C1] | LC-MS [M + H]⁺/Rt (min): 405.2/0.700 (Method B). |
| 12 | 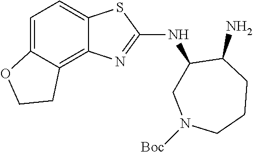 [SMILES: CC(C)(C)OC(=O)N1CCC[C@H](N)[C@H](Nc2nc3c4c(ccc3s2)OCC4)C1] | LC-MS [M + H]⁺/Rt (min): 405.2/0.676 (Method B). |
| 13 | 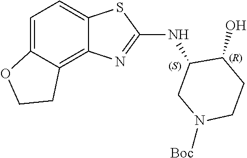 | LC-MS [M + H]⁺/Rt (min) : 392.5/1.792 (Method D). |

### Reference Example 14

### tert-Butyl cis-3-(7,8-dihydrobenzofuro[3,2-e][1,3]benzothiazol-2-yl)-2-oxooctahydro-5H-imidazo[4,5-c]pyridine-5-carboxylate

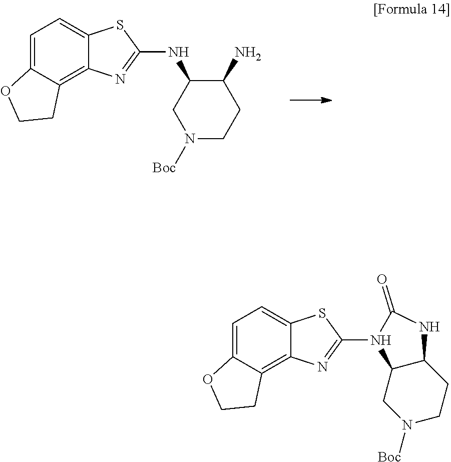 [SMILES: CC(C)(C)OC(=O)N1CC[C@H](N)[C@H](Nc2nc3c4c(ccc3s2)OCC4)C1 ; CC(C)(C)OC(=O)N1CC[C@@H]2NC(=O)N(c3nc4c5c(ccc4s3)OCC5)[C@@H]2C1]

Triethylamine (0.162 mL) and di(N-succinimidyl) carbonate (99 mg) were added to a chloroform solution (4 mL) of the compound of Reference Example 8 (151 mg), and the resulting mixture was stirred for 0.5 hours. Saturated aqueous sodium bicarbonate was added to the reaction mixture, and the resulting mixture was extracted with chloroform. The organic layer was dried over sodium sulfate, filtered, and then concentrated under reduced pressure to obtain the title compound (114 mg).

LC-MS [M+H]⁺/Rt (min): 417.2/0.803 (Method B)

### Reference Examples 15 to 19

The compounds of Reference Examples 15 to 19 were obtained by using the corresponding raw material compounds according to the method described in Reference Example 14.

TABLE 7

| Reference | | |
|---|---|---|
| Example | Chemical structure | Physical property data |
| 15 | 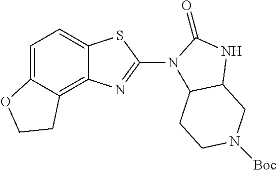 [SMILES: CC(C)(C)OC(=O)N1CCC2C(C1)NC(=O)N2c1nc2c3c(ccc2s1)OCC3] | LC-MS [M + H]⁺/Rt (min): 417.3/0.898 (Method B) |
| 16 | 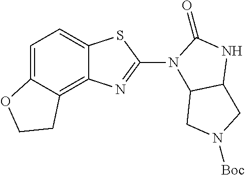 [SMILES: CC(C)(C)OC(=O)N1CC2NC(=O)N(c3nc4c5c(ccc4s3)OCC5)C2C1] | LC-MS [M + H]⁺/Rt (min): 403.2/0.907 (Method A). |
| 17 | 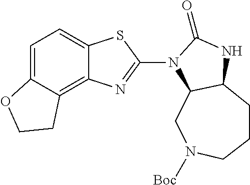 [SMILES: CC(C)(C)OC(=O)N1CCC[C@@H]2NC(=O)N(c3nc4c5c(ccc4s3)OCC5)[C@@H]2C1] | LC-MS [M + H]⁺/Rt (min): 431.3/0.906 (Method B) |
| 18 | 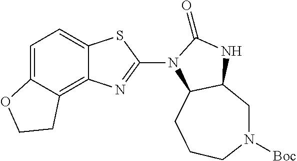 [SMILES: CC(C)(C)OC(=O)N1CCC[C@@H]2[C@H](C1)NC(=O)N2c1nc2c3c(ccc2s1)OCC3] | LC-MS [M + H]⁺/Rt (min): 431.3/1.000 (Method B) |
| 19 | 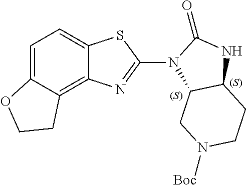 | LC-MS [M + H]⁺/Rt (min): 417.4/2.180 (Method D) |

### Reference Example 20

### (2S,3R)-3-(Benzylamino)-4-{[tert-butyl (dimethyl)silyl]oxy}butan-2-ol

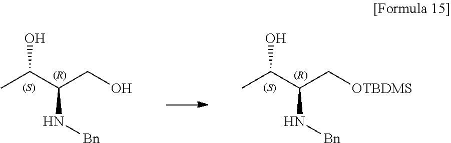

Triethylamine (2.4 mL), N,N-dimethylaminopyridine (1.3 mL), and TBDMS chloride were added to a chloroform solution (65 mL) of (2R,3S)-2-(benzylamino)butane-1,3-diol (4.46 g) under ice cooling, and the resulting mixture was stirred under ice cooling for 4 hours. Saturated aqueous sodium bicarbonate was added to the reaction mixture, and the resulting mixture was extracted with chloroform. The organic layer was dried over sodium sulfate, filtered, and then concentrated under reduced pressure. The residue was purified by silica gel column chromatography (chloroform/methanol) to obtain the title compound (5.42 g).

LC-MS [M+H]⁺/Rt (min): 310.3/0.872 (Method A)

### Reference Example 21

The compound of Reference Example 21 was obtained by using the corresponding raw material compounds according to the method described in Reference Example 20.

TABLE 8

| Reference | | |
|---|---|---|
| Example | Chemical structure | Physical property data |
| 21 | 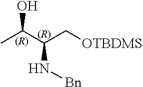 | LC-MS [M + H]⁺/Rt (min): 310.2/0.820 (Method B) |

### Reference Example 22

### Benzyl benzyl[(2R,3S)-1-{[tert-butyl(dimethyl)silyl]oxy}-3-hydroxybutan-2-yl]carbamate

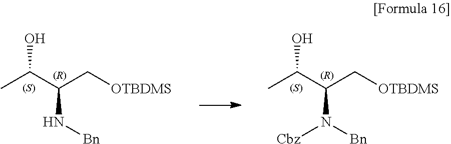

Sodium carbonate (5.57 g) and benzyl chloroformate (2.96 mL) were added to a mixed solution of the compound of Reference Example 20 (5.42 g) in tetrahydrofuran (45 mL) and water (30 mL), and the resulting mixture was stirred at room temperature for 14 hours. Water was added to the reaction mixture, and the resulting mixture was extracted with ethyl acetate. The organic layer was dried over sodium sulfate, filtered, and then concentrated under reduced pressure. The residue was purified by silica gel column chromatography (hexane/ethyl acetate) to obtain the title compound (6.61 g).

LC-MS [M+H]⁺/Rt (min): 444.4/1.389 (Method A)

### Reference Example 23

The compound of Reference Example 23 was obtained by using the corresponding raw material compounds according to the method described in Reference Example 22.

TABLE 9

| Reference | | |
|---|---|---|
| Example | Chemical structure | Physical property data |
| 23 | 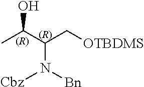 | LC-MS [M + H]⁺/Rt (min): 444.3/1.367 (Method B). |

### Reference Example 24

### Benzyl benzyl[(2S,3R)-1-{[tert-butyl(dimethyl)silyl]oxy}-3-(1,3-dioxo-1,3-dihydro-2H-isoindol-2-yl)butan-2-yl]carbamate

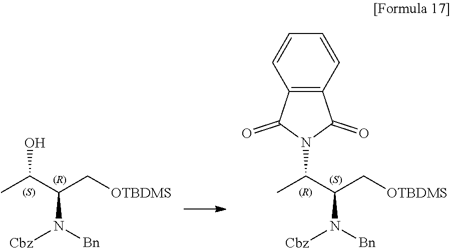

DIAD (3.81 mL) was added dropwise to a toluene solution (60 mL) of the compound of Reference Example 22 (6.61 g) and phthalimide (2.74 g) and triphenylphosphine (4.89 g) under ice cooling, and then the resulting mixture was stirred at room temperature for 7 hours. The reaction mixture was concentrated under reduced pressure, and the residue was purified by silica gel column chromatography (hexane/ethyl acetate) to obtain the title compound (7.03 g).

LC-MS [M+H]⁺/Rt (min): 573.4/1.488 (Method A)

### Reference Example 25

The compound of Reference Example 25 was obtained by using the corresponding raw material compounds according to the method described in Reference Example 24.

TABLE 10

| Reference | | |
|---|---|---|
| Example | Chemical structure | Physical property data |
| 25 | 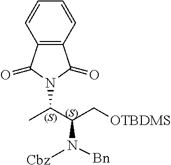 | LC-MS [M + H]⁺/Rt (min): 573.3/1.465 (Method B). |

### Reference Example 26

### 2-[(2R,3S)-3-amino-4-{[tert-butyl(dimethyl)silyl]oxy}butan-2-yl]-1H-isoindole-1,3(2H)-dione

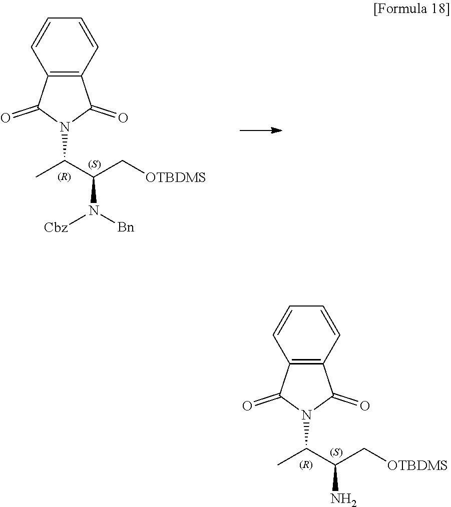

Palladium hydroxide carbon (1.63 g) was added to an ethanol solution (150 mL) of the compound of Reference Example 24 (7.03 g), and the resulting mixture was stirred at room temperature for 35 hours in a hydrogen atmosphere. The reaction mixture was filtered through cerite and then concentrated under reduced pressure. The residue was purified by amino silica gel column chromatography (hexane/ethyl acetate, chloroform/methanol) to obtain the title compound (816 mg).

LC-MS [M+H]⁺/Rt (min): 349.3/0.906 (Method A).

### Reference Example 27

The compound of Reference Example 27 was obtained by using the corresponding raw material compounds according to the method described in Reference Example 26.

TABLE 11

| Reference | | |
|---|---|---|
| Example | Chemical structure | Physical property data |
| 27 | 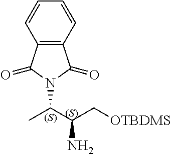 | LC-MS [M + H]⁺/Rt (min): 349.1/0.797 (Method B). |

### Reference Example 28

### N-[(2S,3S)-1-{[tert-butyl(dimethyl)silyl]oxy}-3-(1,3-dioxo-1,3-dihydro-2H-isoindol-2-yl)butan-2-yl]-N′-(2,3-dihydro-1-benzofuran-7-yl)thiourea

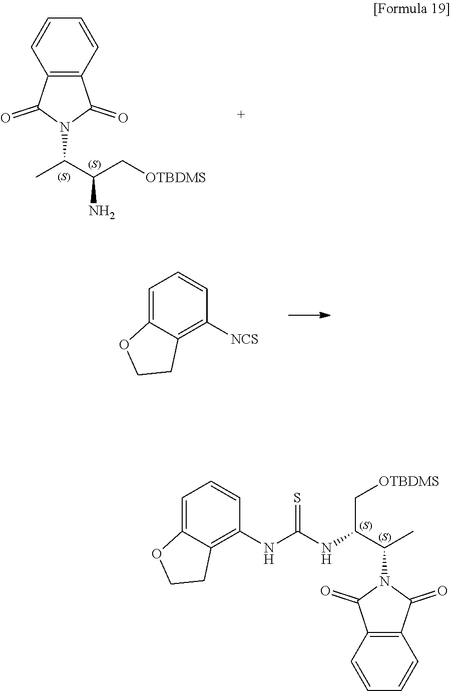

Diisopropylethylamine (0.65 mL) and 4-isothiocyanato-2,3-dihydrobenzofuran (226 mg) were added to a chloroform solution (5 mL) of the compound of Reference Example 27 (444 mg) under ice cooling, and the resulting mixture was stirred at normal temperature for 3 hours. 4-Isothiocyanato-2,3-dihydrobenzofuran (22.6 mg) was added, and the resulting mixture was stirred overnight. Saturated aqueous sodium bicarbonate and water were added to the reaction mixture, and the resulting mixture was extracted with chloroform. The organic layer was dried over sodium sulfate, filtered, and then concentrated under reduced pressure. The residue was purified by silica gel column chromatography (hexane/ethyl acetate) to obtain the title compound (660 mg).

LC-MS [M+H]⁺/Rt (min): 526.3/1.251 (Method B)

### Reference Example 29

The compound of Reference Example 29 was obtained by using the corresponding raw material compounds according to the method described in Reference Example 28.

TABLE 12

| Reference | | |
|---|---|---|
| Example | Chemical structure | Physical property data |
| 29 | 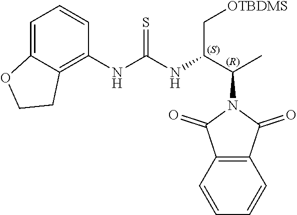 | LC-MS [M + H]⁺/Rt (min): 526.4/1.327 (Method A). |

### Reference Example 30

### 2-{(2S,3S)-4-{[tert-Butyl(dimethyl)silyl]oxy}-3-[(6,7-dihydrofuro[2,3-e][1,3]benzothiazol-2-yl)amino]butan-2-yl}-1H-isoindole-1,3(2H)-dione

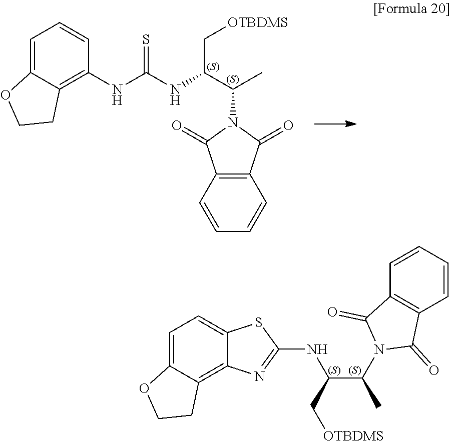

Sodium hydrogen carbonate (105 mg) was added to a chloroform solution (7 mL) of the compound of Reference Example 28 (660 mg) at normal temperature. Benzyltrimethylammonium tribromide (441 mg) was added under ice cooling, and the resulting mixture was stirred under ice cooling for 2 hours. Saturated aqueous sodium bicarbonate and water were added to the reaction mixture, and the resulting mixture was extracted with chloroform. The organic layer was dried over sodium sulfate, filtered, and then concentrated under reduced pressure. Imidazole (129 mg) and TBDMS chloride (189 mg) were added to a chloroform/tetrahydrofuran solution (4 mL/8 mL) of the residue, and the resulting mixture was stirred for 4 hours. Imidazole (200 mg) and TBDMS chloride (190 mg) were added, and the resulting mixture was stirred for 1 hour. Water was added to the reaction mixture, and the resulting mixture was extracted with ethyl acetate. The organic layer was dried over sodium sulfate, filtered, and then concentrated under reduced pressure. The residue was purified by silica gel column chromatography (hexane/ethyl acetate) to obtain the title compound (596 mg).

LC-MS [M+H]⁺/Rt (min): 524.4/1.353 (Method A).

### Reference Example 31

The compound of Reference Example 31 was obtained by using the corresponding raw material compounds according to the method described in Reference Example 30.

TABLE 13

| Reference | | |
|---|---|---|
| Example | Chemical structure | Physical property data |
| 31 | 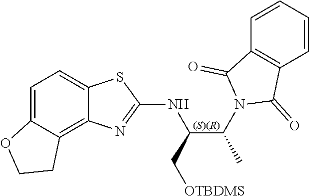 [SMILES: C[C@H]([C@@H](CO[Si](C)(C)C(C)(C)C)Nc1nc2c3c(ccc2s1)OCC3)N1C(=O)c2ccccc2C1=O] | LC-MS [M + H]⁺/Rt (min): 524.4/1.407 (Method A). |

### Reference Example 32

### (2S,3S)-1-{[tert-Butyl(dimethyl)silyl]oxy}-N²-(6,7-dihydrofuro[2,3-e][1,3]benzothiazol-2-yl)butane-2,3-diamine

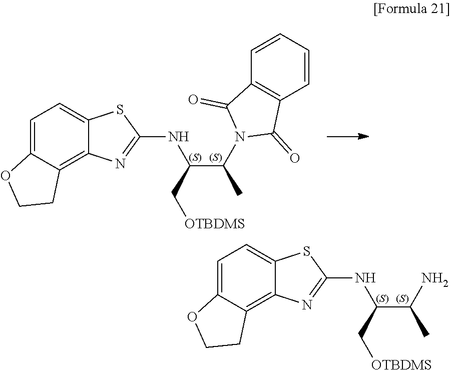

Hydrazine monohydrate (0.554 mL) was added to a methanol/tetrahydrofuran solution (10 mL/10 mL) of the compound of Reference Example 30 (596 mg), and the resulting mixture was stirred at 70° C. for 4 hours. After cooling to normal temperature, ethyl acetate (20 mL) was added to the reaction mixture, and the resulting mixture was stirred for 20 minutes. The reaction mixture was filtered through cerite and then concentrated under reduced pressure to obtain the title compound (490 mg).

LC-MS [M+H]⁺/Rt (min): 394.3/0.948 (Method A).

### Reference Example 33

The compound of Reference Example 33 was obtained by using the corresponding raw material compounds according to the method described in Reference Example 32.

TABLE 14

| Reference | | |
|---|---|---|
| Example | Chemical structure | Physical property data |
| 33 | 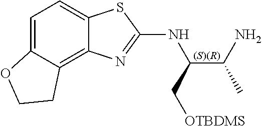 [SMILES: C[C@@H](N)[C@@H](CO[Si](C)(C)C(C)(C)C)Nc1nc2c3c(ccc2s1)OCC3] | LC-MS [M + H]⁺/Rt (min): 394.3/1.017 (Method A). |

### Reference Example 34

### (4S,5S)-5-({[tert-Butyl(dimethyl)silyl]oxy}methyl)-1-(6,7-dihydrofuro[2,3-e][1,3]benzothiazol-2-yl)-4-methylimidazolidin-2-one

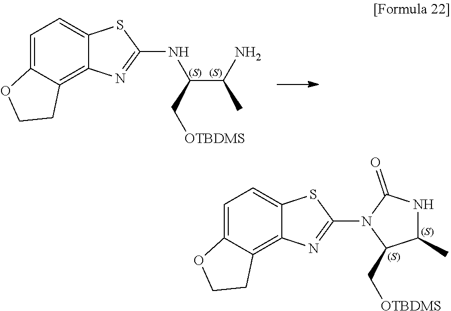

Diisopropylethylamine (0.582 mL) and di(N-succinimidyl) carbonate (307 mg) were added to a chloroform solution (10 mL) of the compound of Reference Example 32 (490 mg), and the resulting mixture was stirred for 2 hours. Saturated aqueous sodium bicarbonate was added to the reaction mixture, and the resulting mixture was extracted with chloroform. The organic layer was dried over sodium sulfate, filtered, and then concentrated under reduced pressure. The residue was purified by silica gel column chromatography (chloroform/methanol) to obtain the title compound (295 mg).

LC-MS [M+H]⁺/Rt (min): 420.3/1.241 (Method A).

### Reference Example 35

The compound of Reference Example 35 was obtained by using the corresponding raw material compounds according to the method described in Reference Example 34.

TABLE 15

| Reference | | |
|---|---|---|
| Example | Chemical structure | Physical property data |
| 35 | 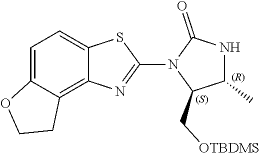 | LC-MS [M + H]⁺/Rt (min): 420.4/1.279 (Method A). |

### Reference Example 36

### (4S,5S)-1-(6,7-dihydrofuro[2,3-e][1,3]benzothiazol-2-yl)-5-(hydroxymethyl)-4-methylimidazolidin-2-one

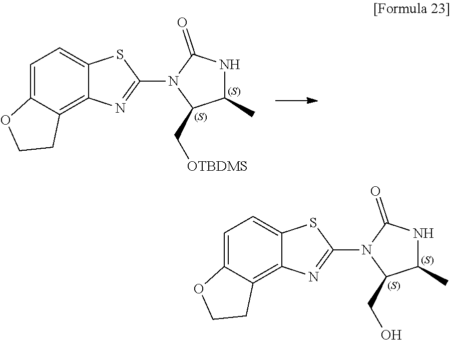

A 2 M hydrogen chloride ethanol solution (1 mL) was added to a methanol suspension (5 mL) of the compound of Reference Example 34 (295 mg), and the resulting mixture was stirred for 1 hour. A 2 M hydrogen chloride ethanol solution (4 mL) was added, and the resulting mixture was stirred for 5 hours. A 2 M hydrogen chloride ethanol solution (4 mL) was further added, and the resulting mixture was stirred overnight. The reaction mixture was concentrated under reduced pressure, saturated aqueous sodium bicarbonate was added, and the resulting mixture was extracted with chloroform/ethanol (3/1). The organic layer was dried over sodium sulfate, filtered, and then concentrated under reduced pressure. The residue was purified by silica gel column chromatography (chloroform/methanol) to obtain the title compound (235 mg).

LC-MS [M+H]⁺/Rt (min): 306.1/0.600 (Method B).

### Reference Example 37

The compound of Reference Example 37 was obtained by using the corresponding raw material compounds according to the method described in Reference Example 36.

TABLE 16

| Reference | | |
|---|---|---|
| Example | Chemical structure | Physical property data |
| 37 | 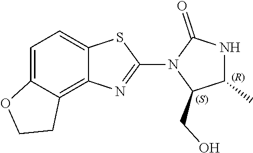 | LC-MS [M + H]⁺/Rt (min): 306.10/0.700 (Method A). |

### Reference Example 38

### [(4S,5S)-3-(6,7-Dihydrofuro[2,3-e][1,3]benzothiazol-2-yl)-5-methyl-2-oxoimidazolidin-4-yl]methyl methanesulfonate

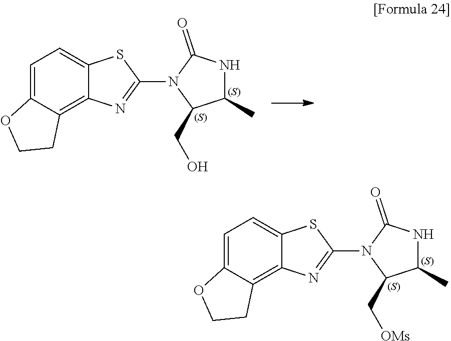

Triethylamine (0.063 mL) and methanesulfonyl chloride (0.017 mL) were added to a tetrahydrofuran solution (4 mL) of the compound of Reference Example 36 (46 mg) under ice cooling, and the resulting mixture was stirred at room temperature for 40 minutes. Triethylamine (0.063 mL) and methanesulfonyl chloride (0.023 mL) were added to the reaction mixture under ice cooling, and the resulting mixture was stirred at room temperature for 3 hours. Saturated aqueous sodium bicarbonate and water were added to the reaction mixture, and the resulting mixture was extracted with chloroform. The organic layer was dried over sodium sulfate, filtered, and then concentrated under reduced pressure to obtain the title compound (122 mg). The next reaction was allowed to proceed without further purification.

LC-MS [M+H]⁺/Rt (min): 306.1/0.600 (Method B).

### Reference Examples 39 to 41

The compounds of Reference Examples 39 to 41 were obtained by using the corresponding raw material compounds according to the method described in Reference Example 38.

TABLE 17

| Reference | | |
|---|---|---|
| Example | Chemical structure | Physical property data |
| 39 | 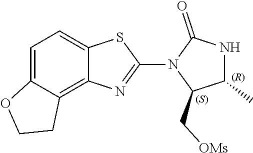 | LC-MS [M + H]⁺/Rt (min): 384.1/0.785 (Method A). |
| 40 | 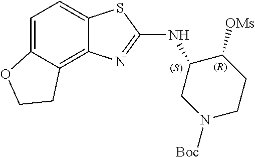 | LC-MS [M + H]⁺/Rt (min): 470.25/2.437 (Method D). |
| 41 | 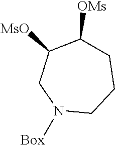 | LC-MS [M + H]⁺/Rt (min): 388.1/0.782 (Method B). |

### Reference Example 42

### tert-Butyl cis-3,4-diazidoazepane-1-carboxylate

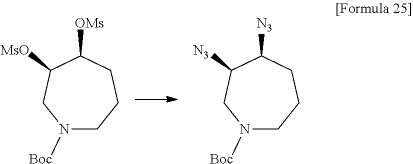 [SMILES: CC(C)(C)OC(=O)N1CCC[C@H](OS(C)(=O)=O)[C@H](OS(C)(=O)=O)C1 ; CC(C)(C)OC(=O)N1CCC[C@H](N=[N+]=[N-])[C@H](N=[N+]=[N-])C1]

Sodium azide (1.41 g) was added to a dimethylformamide solution (5 mL) of the compound of Reference Example 41 (1.25 g), and the resulting mixture was stirred at 65° C. for 4 hours and at 70° C. for 8 hours. Dimethyl sulfoxide (3 mL) was added, and the resulting mixture was stirred at 90° C. for 24 hours. Water was added to the reaction mixture, and the resulting mixture was extracted with ethyl acetate. The organic layer was washed with saturated brine, then dried over sodium sulfate, filtered, and then concentrated under reduced pressure. The residue was purified by silica gel column chromatography (hexane/ethyl acetate) to obtain the title compound (639 mg).

LC-MS [M+H]⁺/Rt (min): 282.2/1.045 (Method B).

### Reference Example 43

The compound of Reference Example 43 was obtained by using the corresponding raw material compounds according to the method described in Reference Example 42.

TABLE 18

| Reference | | |
|---|---|---|
| Example | Chemical structure | Physical property data |
| 43 | 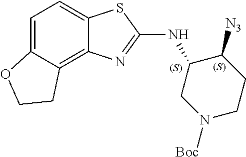 | LC-MS [M + H]⁺/Rt (min): 417.47/2.232 (Method D). |

### Reference Example 44

### tert-Butyl cis-3,4-diaminoazepane-1-carboxylate

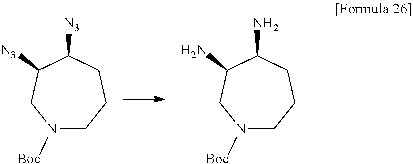 [SMILES: CC(C)(C)OC(=O)N1CCC[C@H](N=[N+]=[N-])[C@H](N=[N+]=[N-])C1 ; CC(C)(C)OC(=O)N1CCC[C@H](N)[C@H](N)C1]

Palladium hydroxide carbon (130 mg) was added to an ethanol solution (15 mL) of the compound of Reference Example 42 (639 mg), and the resulting mixture was stirred for 8 hours in a hydrogen atmosphere. The reaction mixture was filtered through cerite and then concentrated under reduced pressure. The residue was purified by amino silica gel column chromatography (chloroform/methanol) to obtain the title compound (380 mg).

LC-MS [M+H]⁺/Rt (min): 230.2/0.276 (Method B).

### Reference Example 45

The compound of Reference Example 45 was obtained by using the corresponding raw material compounds according to the method described in Reference Example 44.

TABLE 19

| Reference | | |
|---|---|---|
| Example | Chemical structure | Physical property data |
| 45 | 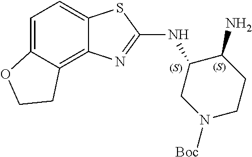 | LC-MS [M + H]⁺/Rt (min): 391.45/1.586 (Method D). |

### Reference Example 46

### 5-Bromo-4-isothiocyanato-2H-1,3-benzodioxole

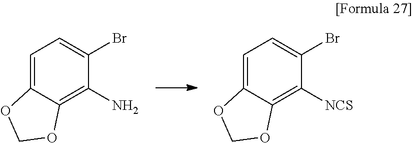

Thiophosgene (17.8 mL) was added to a solution of 5-bromo-1,3-benzodioxol-4-amine (20 g) in water (40 mL) under ice cooling, and the resulting mixture was stirred at room temperature for 4 hours. Water was added to the reaction mixture, and the resulting mixture was extracted with ethyl acetate. The organic layer was dried over sodium sulfate, filtered, and then concentrated under reduced pressure. The residue was purified by silica gel column chromatography (petroleum ether) to obtain the title compound (10 g).

¹H-NMR (DMSO-D₆) δ: 7.18 (1H, d, J=8.4 Hz), 6.91 (1H, d, J=8.4 Hz), 6.22 (2H, s).

### Reference Example 47

### tert-Butyl {(3R,4R)-4-[(2H-[1,3]dioxolo[4,5-e][1,3]benzothiazol-7-yl)amino]oxan-3-yl}carbamate

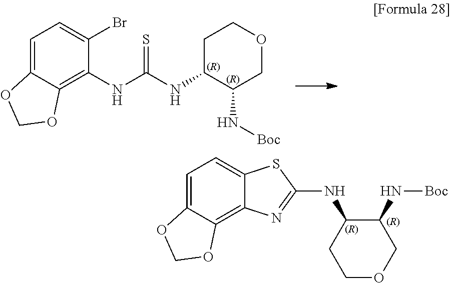

1,10-Phenanthroline (84 mg), cesium carbonate (1.51 g), and copper(I) iodide (22 mg) were added to an acetonitrile solution (23 mL) of the compound of Reference Example 7 (1.1 g), and the resulting mixture was stirred at 95° C. for 4 hours. The reaction mixture was filtered through cerite and then concentrated under reduced pressure. The residue was purified by silica gel column chromatography (hexane/ethyl acetate) to obtain the title compound (759 mg).

1H-NMR (CDCl₃) δ: 7.00 (1H, d, J=8.2 Hz), 6.68 (1H, d, J=8.2 Hz), 6.16 (1H, s), 6.07-5.99 (2H, m), 5.27 (1H, s), 4.21 (1H, d, J=8.2 Hz), 4.05 (1H, s), 3.97 (1H, dd, J=4.5, 11.9 Hz), 3.90-3.83 (1H, m), 3.69-3.61 (1H, m), 3.61-3.51 (1H, m), 2.38-2.25 (1H, m), 1.73 ((1H, dtd, J=4.9, 12.0, 13.5 Hz), 1.47 (9H, s).

### Reference Example 48

### 5-Isothiocyanato-2,3-dihydro-1,4-benzodioxin

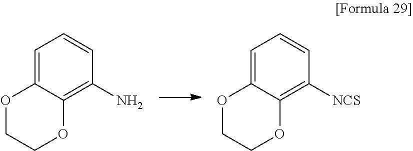

5-Amino-1,4-benzodioxane (2.75 g) was added to a chloroform solution (20 mL) of 1,1′-thiocarbonyldi-2(1H)-pyridone (4.43 g) at room temperature, and the resulting mixture was stirred at room temperature overnight. Water was added to the reaction mixture, and the resulting mixture was extracted with chloroform. The organic layer was dried over sodium sulfate, filtered, and then concentrated under reduced pressure. The residue was purified by silica gel column chromatography (hexane/ethyl acetate) to obtain the title compound (3.38 g).

1H-NMR (CDCl₃) δ: 6.80-6.67 (3H, m), 4.37-4.33 (2H, m), 4.28-4.24 (2H, m).

### Reference Examples 49 to 57

The compounds of Reference Examples 49 to 57 were obtained by using the corresponding raw material compounds according to the method described in Reference Example 1 and Reference Example 2.

TABLE 20

| Reference | | |
|---|---|---|
| Example | Chemical structure | Physical property data |
| 49 | 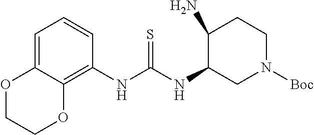 [SMILES: CC(C)(C)OC(=O)N1CC[C@H](N)[C@H](NC(=S)Nc2cccc3c2OCCO3)C1] | LC-MS [M + H]⁺/Rt (min): 409.2/0.594 (Method B). |
| 50 | 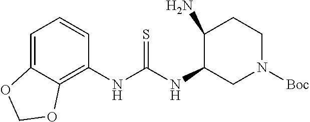 [SMILES: CC(C)(C)OC(=O)N1CC[C@H](N)[C@H](NC(=S)Nc2cccc3c2OCO3)C1] | LC-MS [M + H]⁺/Rt (min): 395.2/0.675 (Method A). |
| 51 | 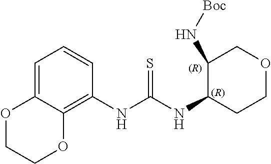 | ¹H-NMR (CDCl₃): 7.42 (1H, s), 6.87-6.70 (3H, m), 5.15-5.07 (1H, m), 4.68-4.56 (1H, m), 4.39-4.18 (4H, m), 3.98-3.89 (2H, m), 3.85- 3.76 (1H, m), 3.62 (1H, dd, J = 1.6, 11.9 Hz), 3.57-3.50 (1H, m), 2.29 (1H, s), 1.60 (2H, s), 1.35 (9H, s). |
| 52 | 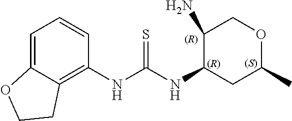 | LC-MS [M + H]⁺/Rt (min): 308.1/1.624 (Method C). |
| 53 | 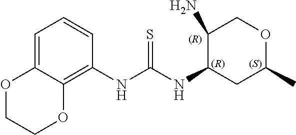 | LC-MS [M + H]⁺/Rt (min): 324.1/1.623 (Method C). |
| 54 | 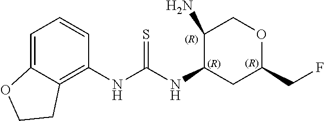 | LC-MS [M + H]⁺/Rt (min): 326.1/1.490 (Method C). |
| 55 | 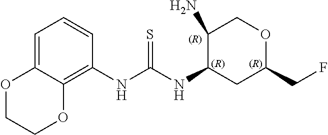 | LC-MS [M + H]⁺/Rt (min): 342.1/1.474 (Method C). |
| 56 | 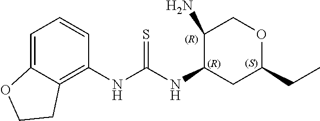 | LC-MS [M + H]⁺/Rt (min): 322.2/1.757 (Method C) . |
| 57 | 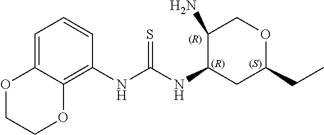 | LC-MS [M + H]⁺/Rt (min): 338.2/1.707 (Method C). |

The compounds of Reference Examples 58 to 66 were obtained by using the corresponding raw material compounds according to the method described in Reference Example 8.

TABLE 21

| Ref. | | |
|---|---|---|
| Example | Chemical structure | Physical property data |
| 58 | 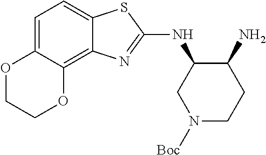 [SMILES: CC(C)(C)OC(=O)N1CC[C@H](N)[C@H](Nc2nc3c4c(ccc3s2)OCCO4)C1] | LC-MS [M + H]⁺/Rt (min): 407.2/0.609 (Method B). |
| 59 | 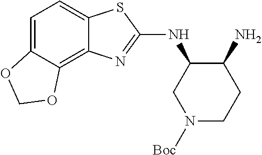 [SMILES: CC(C)(C)OC(=O)N1CC[C@H](N)[C@H](Nc2nc3c4c(ccc3s2)OCO4)C1] | ¹H-NMR (CDCl₃) δ: 6.99 (1H, d, J = 5.8 Hz), 6.67 (1H, d, J = 7.9 Hz), 6.01 (2H, s), 5.64 (1H, d, J = 6.1 Hz), 4.01 (2H, bs), 3.79 (1H, bs). 3.27 (1H, d, J = 12.8 Hz), 3.22-3.18 (1H, m), 3.09 (1H, t, J = 10.1 Hz), 1.77-1.71 (1H, m), 1.56-1.49 (1H, m), 1.35 (9H, s). |
| 60 | 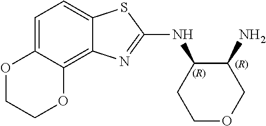 | LC-MS [M + H]⁺/Rt (min): 308.1/2.024 (Method C). |
| 61 | 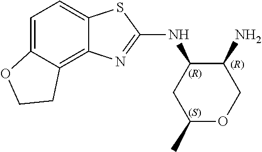 | LC-MS [M + H]⁺/Rt (min): 306.1/1.657 (Method C). |
| 62 | 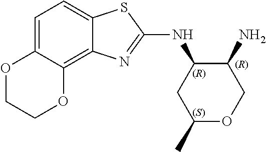 | LC-MS [M + H]⁺/Rt (min): 322.2/1.657 (Method C). |
| 63 | 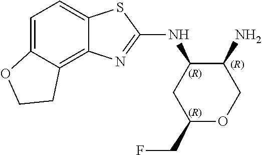 | LC-MS [M + H]⁺/Rt (min): 324.1/1.574 (Method C). |
| 64 | 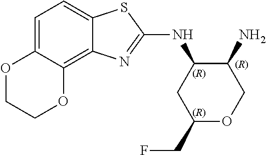 | LC-MS [M + H]⁺/Rt (min): 340.1/1.657 (Method C). |
| 65 | 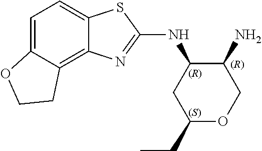 | LC-MS [M + H]⁺/Rt (min): 320.2/1.757 (Method C) |
| 66 | 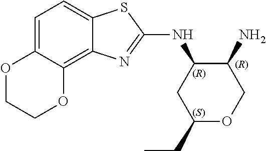 | LC-MS [M + H]⁺/Rt (min): 336.2/1.740 (Method C) |

### Reference Examples 67 and 68

The compounds of Reference Examples 67 and 68 were obtained by using the corresponding raw material compounds according to the method described in Reference Example 14.

TABLE 22

| Reference | | |
|---|---|---|
| Example | Chemical structure | Physical property data |
| 67 | 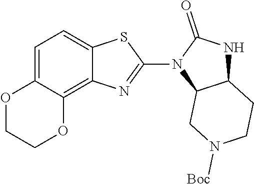 [SMILES: CC(C)(C)OC(=O)N1CC[C@@H]2NC(=O)N(c3nc4c5c(ccc4s3)OCCO5)[C@@H]2C1] | LC-MS [M + H]⁺/Rt (min): 433.2/0.764 (Method B) |
| 68 | 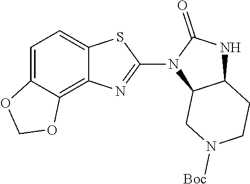 [SMILES: CC(C)(C)OC(=O)N1CC[C@@H]2NC(=O)N(c3nc4c5c(ccc4s3)OCO5)[C@@H]2C1] | LC-MS [M + H]⁺/Rt (min): 419.2/0.819 (Method B) |

### Reference Example 69

### (3R,4S,6R)-6-cyanooxane-3,4-diyl diacetate

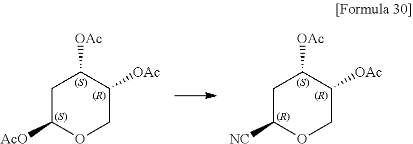

Trimethylsilyl cyanide (1.93 mL) and a boron trifluoride diethyl ether complex (4.56 mL) were added to a dichloromethane solution (40 mL) of (2S,4S,5R)-tetrahydro-2H-pyran-2,4,5-triyltriacetate (3.12 g) under ice cooling, and the resulting mixture was stirred under ice cooling for 1 hour. Saturated aqueous sodium bicarbonate was added to the reaction mixture, and the resulting mixture was extracted with chloroform. The organic layer was dried over sodium sulfate, filtered, and then concentrated under reduced pressure to obtain the title compound (2.94 g).

1H-NMR (CDCl₃) δ: 5.38-5.28 (1H, m), 5.21-5.13 (1H, m), 4.84 (1H, t, J=4.6 Hz), 4.01 (1H, dd, J=2.5, 12.9 Hz), 3.93 (1H, dd, J=4.5, 12.9 Hz), 2.42-2.29 (1H, m), 2.14-2.08 (4H, m), 2.07 (3H, s).

### Reference Example 70

### Methyl 2,6-anhydro-3-deoxy-D-ribo-hexonate

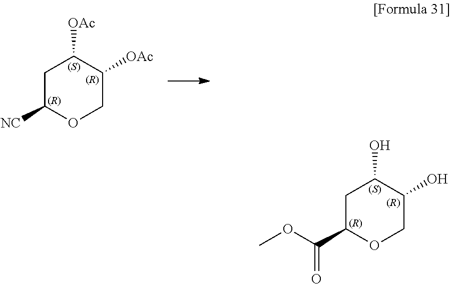

Reference Example 69 was added to a 5% hydrogen chloride methanol solution (30 mL) at room temperature, and the resulting mixture was stirred under reflux overnight. The reaction mixture was cooled to 0° C., and then the resulting insoluble matter was filtered. The filtrate was concentrated under reduced pressure, and then the residue was purified by silica gel column chromatography (chloroform/methanol) to obtain the title compound (2.82 g).

1H-NMR (CDCl₃) δ: 4.39 (1H, ddd, J=0.7, 3.0, 10.8 Hz), 4.19-4.10 (1H, m), 3.94-3.87 (1H, m), 3.86-3.79 (1H, m), 3.77 (3H, s), 3.66 (1H, dd, J=9.4, 10.8 Hz), 2.41 (1H, dd, J=1.1, 2.9 Hz), 2.28-2.18 (2H, m), 1.97-1.86 (1H, m).

### Reference Example 71

### Methyl 2,6-anhydro-4,5-O-benzylidene-3-deoxy-D-ribo-hexonate

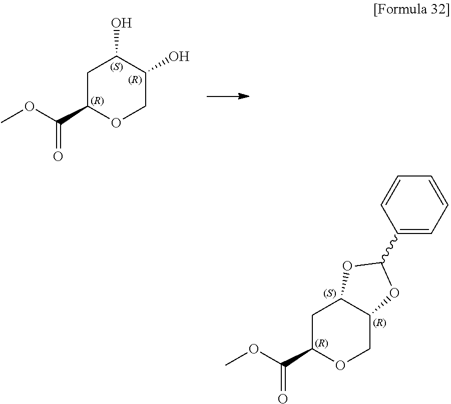

Benzaldehyde dimethyl acetal (5.55 mL) and para-toluenesulfonic acid hydrate (0.424 g) were added to a dimethylformamide solution (30 mL) of Reference Example 70 (2.67 g) at room temperature, and the resulting mixture was stirred at 60 to 70° C. overnight. The reaction mixture was cooled to room temperature, then water was added, and the resulting mixture was extracted with hexane/ethyl acetate (1:1). The organic layer was dried over sodium sulfate, filtered, and then concentrated under reduced pressure. The residue was purified by silica gel column chromatography (hexane/ethyl acetate) to obtain the title compound (4.3 g) as a mixture at about 3:2. 1H-NMR (CDCl₃) δ: 7.60-7.31 (5H, m), 6.35 (0.4H, s), 5.79 (0.6H, s), 4.58-3.98 (4H, m), 3.83-3.72 (3H, m), 3.72-3.47 (1H, m), 2.60-2.40 (1H, m), 2.15-1.96 (1H, m).

### Reference Example 72

### 2,6-Anhydro-4,5-O-benzylidene-3-deoxy-D-ribo-hexitol

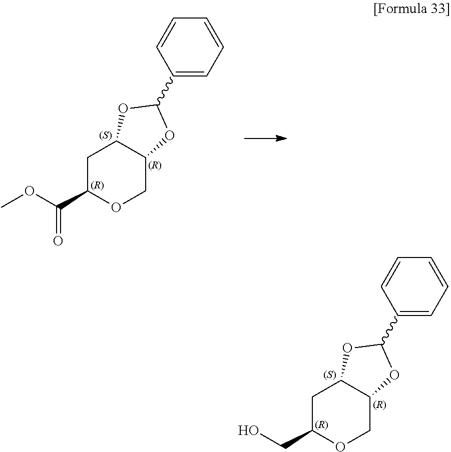

Sodium borohydride (2.25 g) was added to a methanol solution (40 mL) of Reference Example 71 (3.93 g) under ice cooling, and the resulting mixture was stirred under ice cooling for 1 hour. A saturated ammonium chloride aqueous solution was added to the reaction mixture, and the resulting mixture was extracted with ethyl acetate. The organic layer was dried over sodium sulfate, filtered, and then concentrated under reduced pressure. The residue was purified by silica gel column chromatography (hexane/ethyl acetate) to obtain the title compound (3.08 g) as a mixture at about 3:2.

¹H-NMR (CDCl₃) δ: 7.59-7.31 (5H, m), 6.34 (0.4H, s), 5.83 (0.6H, s), 4.54-4.20 (2H, m), 4.11-3.92 (2H, m), 3.84-3.62 (2H, m), 2.24-1.73 (3H, m).

### Reference Example 73

### 2,6-Anhydro-4,5-O-benzylidene-1,3-dideoxy-1-iodo-D-ribo-hexitol

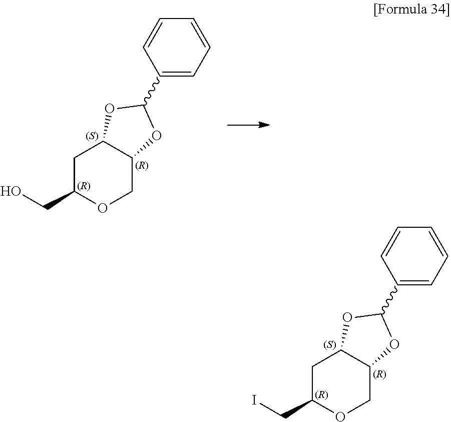

Triphenylphosphine (13.8 g), imidazole (8.96 g), and iodine (13.4) were added to a tetrahydrofuran solution (210 mL) of Reference Example 72 (10.4 g) under ice cooling, and the resulting mixture was stirred under ice cooling for 30 minutes and at room temperature for 3.5 hours. Ice water was added to the reaction mixture, and the resulting mixture was extracted with ethyl acetate. The organic layer was washed with a 5% sodium thiosulfate aqueous solution, then dried over sodium sulfate, filtered, and then concentrated under reduced pressure. The residue was purified by silica gel column chromatography (hexane/ethyl acetate) to obtain the title compound (14.0 g) as a mixture at about 3:2.

1H-NMR (CDCl₃) δ: 7.61-7.31 (5H, m), 6.34 (0.4H, s), 5.75 (0.6H, s), 4.55-4.23 (2H, m), 4.14-3.94 (1H, m), 3.73-3.47 (2H, m), 3.34-3.10 (2H, m), 2.41-2.30 (1H, m), 1.94-1.67 (1H, m).

### Reference Example 74

### 2,6-Anhydro-4,5-O-benzylidene-1,3-dideoxy-1-fluoro-D-ribo-hexitol

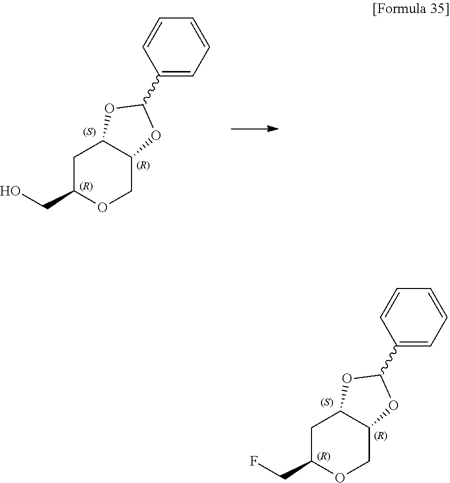

Diisopropylethylamine (3.44 mL) and diethylaminosulfur trifluoride (1.04 mL) were added to a dichloromethane solution (33 mL) of Reference Example 72 (10.4 g) under ice cooling, and the resulting mixture was stirred under reflux for 19 hours. Diisopropylethylamine (1.72 mL) and diethylaminosulfur trifluoride (0.52 mL) were further added at room temperature, and the resulting mixture was stirred under reflux for 2 hours. Saturated aqueous sodium bicarbonate was added to the reaction mixture under ice cooling, and the resulting mixture was extracted with chloroform. The organic layer was dried over sodium sulfate, filtered, and then concentrated under reduced pressure. The residue was purified by silica gel column chromatography (hexane/ethyl acetate) to obtain the title compound (1.25 g) as a mixture at about 3:2.

¹H-NMR (CDCl₃) δ: 7.57-7.32 (5H, m), 6.38 (0.4H, s), 5.90 (0.6H, s), 4.57-4.26 (4H, m), 4.15-3.85 (2H, m), 3.63-3.49 (1H, m), 2.22-1.84 (2H, m).

### Reference Example 75

### 2,6-Anhydro-4,5-O-benzylidene-3-deoxy-D-ribo-hexose

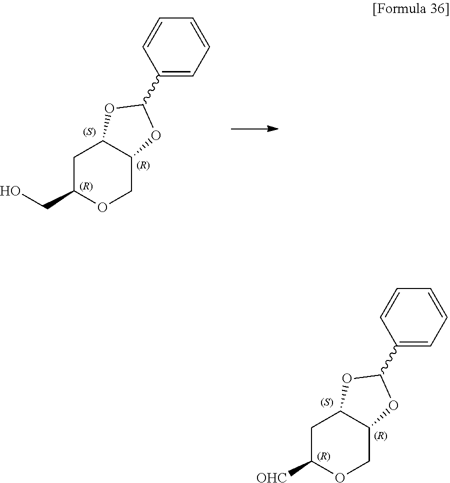

The Dess-Martin reagent (4.31 g) was added to a dichloromethane solution (85 mL) of Reference Example 72 (2.00 g) under ice cooling, and the resulting mixture was stirred at room temperature overnight. Saturated aqueous sodium bicarbonate and a saturated sodium thiosulfate aqueous solution were added to the reaction mixture under ice cooling, and the resulting mixture was extracted with chloroform. The organic layer was washed with saturated brine, then dried over sodium sulfate, filtered, and then concentrated under reduced pressure. The residue was purified by silica gel column chromatography (hexane/ethyl acetate) to obtain the title compound (0.83 g).

1H-NMR (CDCl₃) δ: 9.70 (1H, s), 7.58-7.45 (2H, m), 7.43-7.37 (1H, m), 5.92 (1H, s), 4.57-4.43 (1H, s), 4.35-4.14 (2H, m), 4.01 (1H, dd, J=5.3, 12.1 Hz), 3.64 (1H, dd, J=7.2, 12.1 Hz), 2.44 (1H, ddd, J=2.6, 3.8, 15.1 Hz), 1.92 (1H, ddd, J=3.8, 11.9, 15.1 Hz).

### Reference Example 76

### 3,7-Anhydro-5,6-O-benzylidene-1,2,4-trideoxy-D-ribo-hept-1-ynitol

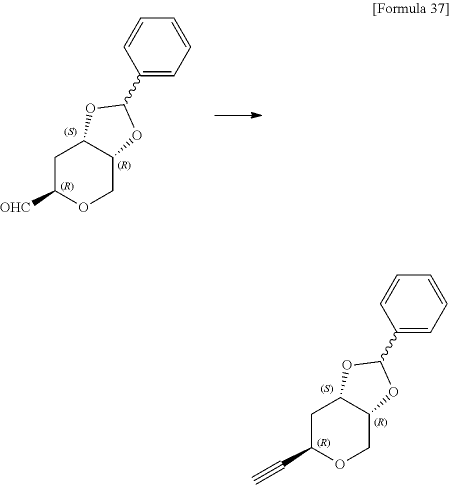

Potassium carbonate (0.979 mg) and dimethyl (1-diazo-2-oxopropyl)phosphonate (0.638 mL) were added to a methanol solution (35 mL) of Reference Example 75 (0.83 g) at room temperature, and the resulting mixture was stirred at room temperature overnight. Water was added to the reaction mixture, and the resulting mixture was extracted with ethyl acetate. The organic layer was washed with water and saturated brine, then dried over sodium sulfate, filtered, and then concentrated under reduced pressure. The residue was purified by silica gel column chromatography (hexane/ethyl acetate) to obtain the title compound (0.415 mg).

1H-NMR (CDCl₃) δ: 7.57-7.46 (2H, m), 7.45-7.34 (3H, m), 5.91 (1H, s), 4.56-4.45 (2H, m), 4.27-4.23 (1H, m), 4.10 (1H, dd, J=4.9, 12.5 Hz), 3.71 (1H, dd, J=6.1, 12.5 Hz), 2.50 (1H, d, J=2.2 Hz), 2.38-2.32 (1H, m), 2.20-2.13 (1H, m).

### Reference Example 77

### 2,6-Anhydro-4,5-O-benzylidene-1,3-dideoxy-D-ribo-hexitol

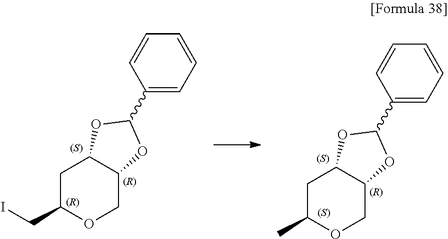

Sodium borohydride (7.64 g) was added to a dimethyl sulfoxide solution (200 mL) of Reference Example 73 (10.4 g) under ice cooling, and the resulting mixture was stirred at room temperature overnight. The reaction mixture was poured into ice water and then extracted with hexane/ethyl acetate (1:1). The organic layer was washed with water, then dried over sodium sulfate, filtered, and then concentrated under reduced pressure. The residue was purified by silica gel column chromatography (hexane/ethyl acetate) to obtain the title compound (7.34 g) as a mixture at about 3:2.

1H-NMR (CDCl₃) δ: 7.60-7.32 (5H, m), 6.37 (0.4H, s), 5.78 (0.6H, s), 4.09-3.91 (1H, m), 3.82-3.64 (1H, m), 3.56-3.32 (1H, m), 2.32-2.11 (1H, m), 1.85-1.62 (1H, m), 1.24-1.14 (3H, m).

### Reference Example 78

### 2,6-Anhydro-1,3-dideoxy-D-ribo-hexitol

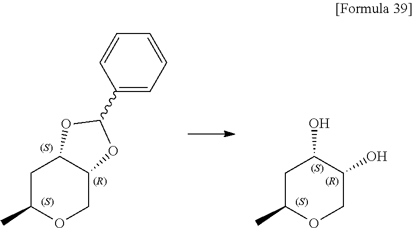

Para-toluenesulfonic acid hydrate (0.951 g) was added to a methanol solution (160 mL) of Reference Example 77 (7.34 g) at room temperature, and the resulting mixture was stirred at room temperature overnight. Diisopropylethylamine (1.16 mL) was added to the reaction mixture at room temperature, and the resulting mixture was stirred at room temperature for 15 minutes, and then concentrated under reduced pressure. The residue was purified by silica gel column chromatography (chloroform/methanol) to obtain the title compound (3.83 g).

1H-NMR (CDCl₃) δ: 4.18-4.02 (1H, m), 3.91-3.66 (3H, m), 3.64-3.44 (1H, m), 2.18 (1H, d, J=3.1 Hz), 2.04-1.94 (1H, m), 1.90 (1H, ddd, J=2.2, 3.8, 14.3 Hz), 1.54 (1H, dddd, J=1.2, 2.7, 11.1, 14.1 Hz), 1.16 (3H, d, J=6.3 Hz).

### Reference Example 79

The compound of Reference Example 79 was obtained by using the corresponding raw material compounds according to the method described in Reference Example 78.

TABLE 23

| Reference | | |
|---|---|---|
| Example | Chemical structure | Physical property data |
| 79 | 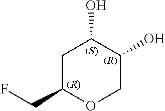 | ¹H-NMR (CDCl₃) δ: 4.53-4.26 (2H, m), 4.22-4.17 (1H, m), 4.03- 3.90 (1H, m), 3.85-3.75 (2H, m), 3.66-3.56 (1H, m), 2.27-2.22 (1H, m), 1.98-1.91 (1H, m), 1.90- 1.82 (1H, m), 1.77-1.68 (1H, m). |

### Reference Example 80

### 3,7-Anhydro-1,2,4-trideoxy-D-ribo-heptitol

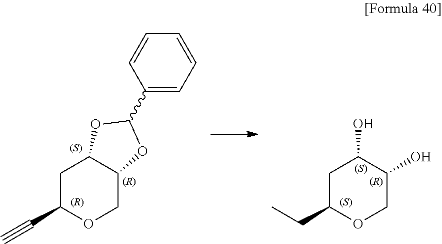

10% Palladium carbon (95 mg) was added to a methanol solution (18 mL) of Reference Example 76 (410 mg) at room temperature, and the resulting mixture was stirred overnight in a room temperature hydrogen atmosphere. The reaction mixture was filtered through cerite, and then the filtrate was concentrated under reduced pressure. The residue was purified by silica gel column chromatography (hexane/ethyl acetate) to obtain the title compound (240 mg).

1H-NMR (CDCl₃) δ: 4.16-4.07 (1H, m), 3.81-3.69 (2H, m), 3.62-3.49 (2H, m), 2.33-2.27 (1H, m), 2.16 (1H, d, J=6.3 Hz), 1.91 (1H, ddd, J=2.2, 3.8, 14.3 Hz), 1.59-1.34 (3H, m), 0.93 (3H, t, J=7.5 Hz).

### Reference Examples 81 to 83

The compounds of Reference Examples 81 to 83 were obtained by using the corresponding raw material compounds according to the method described in Reference Example 38.

TABLE 24

| Reference | | |
|---|---|---|
| Example | Chemical structure | Physical property data |
| 81 | 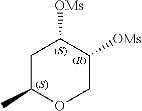 | ¹H-NMR (CDCl₃) δ: 5.24- 5.11 (1H, m), 4.75 (1H, ddd, J = 3.0, 5.5, 10.7 Hz), 3.96-3.84 (2H, m), 3.80 (1H, dd, J = 10.7, 10.9 Hz), 3.13 (3H, s), 3.10 (3H, s), 2.21 (1H, ddd, J = 2.1, 4.0, 14.9 |
| | | Hz), 1.74 (1H, ddd, J = 2.3, |
| | | 11.2, 14.9 Hz), 1.20 (3H, |
| | | d, J = 6.2 Hz). |
| 82 | 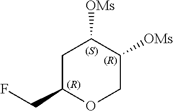 | ¹H-NMR (CDCl₃) δ: 5.31- 5.23 (1H, m), 4.83-4.72 (1H, m), 4.59-4.27 (2H, m), 4.07-3.92 (2H, m), 3.84 (1H, t, J = 10.9 Hz), 3.15 (3H, s), 3.11 (3H, s), 2.27-2.16 (1H, m), 2.04- |
| | | 1.96 (1H, m). |
| 83 | 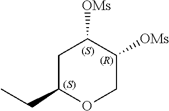 | ¹H-NMR (CDCl₃) δ: 5.23- 5.17 (1H, m), 4.78-4.70 (1H, m), 3.98-3.91 (1H , m), 3.78 (dd, J = 10.9, 10.9 Hz), 3.68-3.59 (1H, m), 3.13 (3H, s), 3.10 (3H, s), 2.24-2.17 (1H, m), |
| | | 1.79-1.68 (1H, m), 1.57- |
| | | 1.41 (2H, m), 0.94 (3H, |
| | | t, J = 7.5 Hz). |

### Reference Examples 84 to 86

The compounds of Reference Examples 84 to 86 were obtained by using the corresponding raw material compounds according to the method described in Reference Example 42.

TABLE 25

| Reference | | |
|---|---|---|
| Example | Chemical structure | Physical property data |
| 84 | 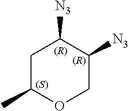 | ¹H-NMR (CDCl₃) δ: 4.12 (1H, dd, J = 2.0, 12.7 Hz), 3.67 (1H, dd, J = 1.4, 2.8 Hz), 3.63-3.43 (3H, m), 1.89-1.76 (3H, d, J = 6.2 Hz). |
| 85 | 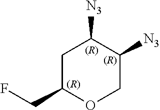 | ¹H-NMR (CDCl₃) δ: 4.55-4.32 (2H, m), 4.19 (1H, dd, J = 2.0, 12.8 Hz), 3.76-3.64 (3H, m), 3.61 (1H, dd, J = 1.5, 12.8 Hz), 1.97-1.81 (2H, m). |
| 86 | 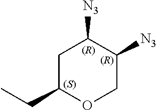 | ¹H-NMR (CDCl₃) δ: 4.13 (1H, dd, J = 2.0, 12.7 Hz), 3.70-3.64 (1H, m), 3.63-3.57 (1H, m), 3.55 (1H, dd, J = 1.5, 12.7 Hz), 3.31-3.22 (1H, m), 1.89-1.48 (4H, m), 0.96 (3H, t, J = 7.5 Hz). |

### Reference Examples 87 to 89

The compounds of Reference Examples 87 to 89 were obtained by using the corresponding raw material compounds according to the method described in Reference Example 44.

TABLE 26

| Reference | | |
|---|---|---|
| Example | Chemical structure | Physical property data |
| 87 | 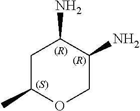 | ¹H-NMR (CDCl3) δ: 3.87 (1H, dd, J = 2.0, 11.7 Hz), 3.54 (1H, dd, J = 1.7, 11.7 Hz), 3.41 (1H, dqd, J = 2.2, 6.2, 11.1 Hz), 2.90 (1H, ddd, J = 3.6, 4.5, 12.0 Hz), 2.76-2.64 (1H, m), 1.57-1.52 (1H, m), 1.47-1.32 (4H, m), |
| | | 1.31-1.21 (1H, m), 1.20 (3H, |
| | | d, J = 6.2 Hz). |
| 88 | 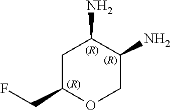 | LC-MS [M + H]⁺/Rt (min): 149.1/0.224 (Method C) |
| 89 | 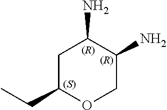 | LC-MS [M + H]⁺/Rt (min): 145.1/0.224 (Method C). |

### Reference Example 90

### tert-Butyl 3-buten-1-yl(1-(hydroxymethyl)cyclopropyl)carbamate

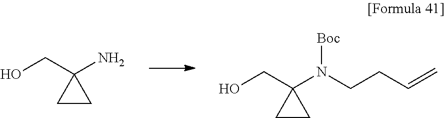 [SMILES: NC1(CO)CC1 ; C=CCCN(C(=O)OC(C)(C)C)C1(CO)CC1]

Potassium carbonate (4.56 g), sodium iodide (4.95 g), and 4-bromo-1-butene (3.02 mL) were added to an acetonitrile solution (50 mL) of (1-aminocyclopropyl)methanol at room temperature, and the resulting mixture was stirred at 85° C. for 12.5 hours. The reaction mixture was cooled to room temperature, and then filtered and concentrated. Tetrahydrofuran (50 mL), triethylamine (8.36 mL), and (Boc)₂O (7.20 g) were added to the residue, and the resulting mixture was stirred at room temperature for 1.5 hours. Saturated aqueous sodium bicarbonate was added to the reaction mixture, and the resulting mixture was extracted with ethyl acetate. The organic layer was dried over sodium sulfate, filtered, and then concentrated under reduced pressure. The residue was purified by silica gel chromatography (hexane/ethyl acetate) to obtain the title compound (3.52 g).

LC-MS [M+H]⁺/Rt (min): 242.5/0.936 (Method B)

### Reference Example 91

### tert-Butyl 3-buten-1-yl(1-formylcyclopropyl)carbamate

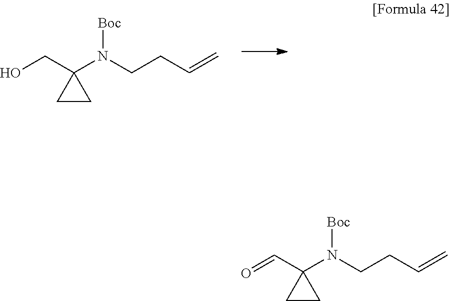 [SMILES: C=CCCN(C(=O)OC(C)(C)C)C1(CO)CC1 ; C=CCCN(C(=O)OC(C)(C)C)C1(C=O)CC1]

A chloroform suspension (20 mL) of the Dess-Martin reagent (2.80 g) was added to a chloroform solution (30 mL) of the compound of Reference Example 90 (1.52 g) at room temperature, and the resulting mixture was stirred at room temperature for 1 hour. The Dess-Martin reagent (900 mg) was added to the reaction mixture at room temperature, and the resulting mixture was stirred for 40 minutes. Sodium hydrogen carbonate (2.80 g) and diethyl ether were added to the reaction mixture, and the resulting mixture was stirred at room temperature, then filtered through cerite, and concentrated under reduced pressure. The residue was purified by column chromatography (hexane/ethyl acetate) to obtain the title compound (1.35 g).

LC-MS [M−56+H]⁺/Rt (min): 184.3/1.029 (Method B)

### Reference Example 92

### tert-Butyl 3-buten-1-yl(1-vinylcyclopropyl)carbamate

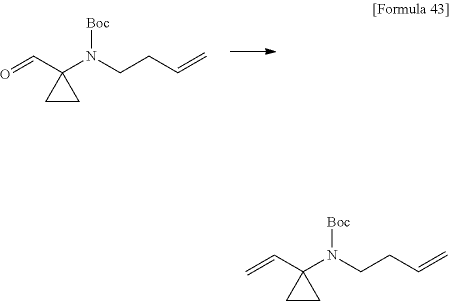 [SMILES: C=CCCN(C(=O)OC(C)(C)C)C1(C=O)CC1 ; C=CCCN(C(=O)OC(C)(C)C)C1(C=C)CC1]

Potassium tert-butoxide (1.27 g) was added to a tetrahydrofuran suspension (19 mL) of methyltriphenylphosphonium bromide (4.03 g) at normal temperature, and the resulting mixture was stirred for 1 hour. A tetrahydrofuran solution (9 mL) of the compound of Reference Example 91 (1.35 g) was added to the reaction mixture at room temperature, and the resulting mixture was stirred for 1 hour. Water was added to the reaction mixture, and the resulting mixture was extracted with ethyl acetate. The organic layer was dried over sodium sulfate, then filtered, and concentrated under reduced pressure. The residue was purified by column chromatography (hexane/ethyl acetate) to obtain the title compound (1.23 g).

LC-MS [M−56+H]⁺/Rt (min): 182.3/1.282 (Method B)

### Reference Example 93

### tert-Butyl 2-propen-1-yl[1-(2-propen-1-yl)cyclopropyl]carbamate

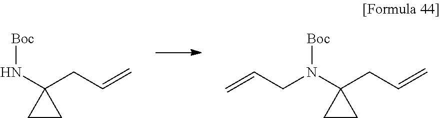 [SMILES: C=CCC1(NC(=O)OC(C)(C)C)CC1 ; C=CCN(C(=O)OC(C)(C)C)C1(CC=C)CC1]

Sodium hydride (60% in oil; 150 mg) and allyl bromide (0.318 mL) were added to a dimethylformamide solution (10 mL) of tert-butyl (1-allylcyclopropyl)carbamate (2.90 mL) at 0° C., and the resulting mixture was stirred at room temperature for 4 hours. Water was added to the reaction mixture, and the resulting mixture was extracted with ethyl acetate. The organic layer was dried over sodium sulfate, then filtered, and concentrated under reduced pressure. The residue was purified by silica gel column chromatography (hexane/ethyl acetate) to obtain the title compound (0.68 g).

1H-NMR (DMSO-d₆) δ: 5.82-5.72 (2H, m), 5.21-4.80 (4H, m), 3.79 (2H, brs), 2.29 (2H, brs), 1.42 (9H, s), 0.85 (2H, brs), 0.64 (2H, brs).

### Reference Example 94

The compound of Reference Example 94 was obtained by using the corresponding raw material compounds according to the method described in Reference Example 93.

TABLE 27

| Reference | | |
|---|---|---|
| Example | Chemical structure | Physical property data |
| 94 | 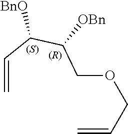 | ¹H-NMR (CDCl₃) δ: 7.39-7.20 (10H, m), 5.96-5.81 (2H, m), 5.38-5.11 (4H, m), 4.71 (2H, s), 4.63 (1H, d, J = 11.9 Hz), 4.40 (1H, d, J = 11.9 Hz), 4.04-3.92 (3H, m), 3.72 (1H, dt, J = 4.5, 5.9 Hz), 3.66-3.52 (2H, m). |

### Reference Example 95

### tert-Butyl 4-azaspiro[2.5]-7-octene-4-carboxylate

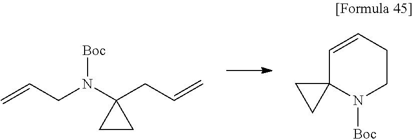 [SMILES: C=CCN(C(=O)OC(C)(C)C)C1(CC=C)CC1 ; CC(C)(C)OC(=O)N1CCC=CC12CC2]

The Grubbs second generation catalyst (220 mg) was added to a solution of the compound of Reference Example 93 (1.23 g) in toluene (52 mL) at room temperature, and the resulting mixture was stirred at 50° C. for 7.5 hours. The reaction mixture was concentrated under reduced pressure, and then the residue was purified by column chromatography (hexane/ethyl acetate) to obtain the title compound (1.04 g).

LC-MS [M−56+H]⁺/Rt (min): 154.1/1.151 (Method B)

### Reference Examples 96 and 97

The compounds of Reference Examples 96 and 97 were obtained by using the corresponding raw material compounds according to the method described in Reference Example 95.

TABLE 28

| Reference | | |
|---|---|---|
| Example | Chemical structure | Physical property data |
| 96 | 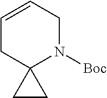 [SMILES: CC(C)(C)OC(=O)N1CC=CCC12CC2] | ¹H-NMR (DMSO-d₆) δ: 5.80-5.68 (2H, m), 3.93 (2H, brs), 2.03 (2H, brs), 1.43 (9H, s), 0.88-0.85 (2H, m), 0.61-0.58 (2H, m). |
| 97 | 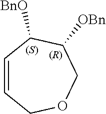 | ¹H-NMR (CDCl₃) δ: 7.39-7.26 (10H, m), 5.84-5.69 (2H, m), 4.81-4.52 (5H, m), 4.31-4.15 (2H, m), 4.07- 4.00 (1H, m), 3.98-3.90 (1H, m), 3.78 (1H, dd, J = 3.8, 12.6 Hz). |

### Reference Example 100

### tert-Butyl (2S)-2-methyl-4-[(trifluoromethanesulfonyl)oxy]-3,6-dihydropyridine-1(2H)-carboxylate

### Reference Example 101

### tert-Butyl (6S)-6-methyl-4-[(trifluoromethanesulfonyl)oxy]-3,6-dihydropyridine-1(2H)-carboxylate

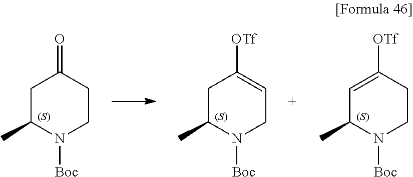

A tetrahydrofuran solution (7 mL) of tert-butyl (S)-2-methyl-4-oxopiperidine-1-carboxylate (1.20 g) was added to a tetrahydrofuran solution (7 mL) of lithium diisopropylamide (1.1 M tetrahydrofuran solution, 6.6 mL) at 0° C., and the resulting mixture was stirred at 0° C. for 10 minutes. A tetrahydrofuran solution (14 mL) of N-phenylbis(trifluoromethanesulfonimide) (2.60 g) was added dropwise at 0° C. over 5 minutes, and the resulting mixture was stirred at room temperature for 2 hours. A saturated ammonium chloride aqueous solution was added to the reaction mixture, and the resulting mixture was extracted with ethyl acetate. The organic layer was dried over sodium sulfate, then filtered, and concentrated under reduced pressure. The residue was purified by silica gel column chromatography (hexane/ethyl acetate) to obtain the title compound (0.70 g) as a mixture with N-phenylbis(trifluoromethanesulfonimide).

1H-NMR (DMSO-d₆) δ: 5.73-5.69 (1H, m), 4.64-3.60 (2H, m), 2.97-2.51 (1H, m), 2.23-2.02 (1H, m), 1.46 (s, 4.5H), 1.45 (s, 4.5H), 1.21 (1.5H, d, J=6.8 Hz), 1.16 (1.5H, d, J=6.8 Hz).

### Reference Examples 102 and 103

The compounds of Reference Examples 102 and 103 were obtained by using the corresponding raw material compounds according to the method described in Reference Examples 100 and 101.

TABLE 29

| Reference | Chemical | |
|---|---|---|
| Example | structure | Physical property data |
| 102 | 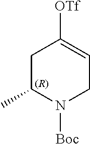 | Obtained as a mixture. ¹H-NMR (DMSO-d₆) δ: 5.74-5.69 (1H, m), 4.64-3.59 (2H, m), 2.97-2.51 (1H, m), 2.21-2.02 (1H, m), 1.45 (s, 4.5H), 1.45 (s, 4.5H), 1.21 (1.5H, d, J = 6.8 Hz), 1.15 (1.5H, d, J = 6.8 Hz). |
| 103 | 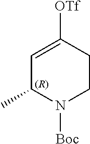 | |

### Reference Example 104

### tert-Butyl (2S)-2-methyl-3,6-dihydropyridine-1(2H)-carboxylate

### Reference Example 105

### tert-Butyl (6S)-6-methyl-3,6-dihydropyridine-1(2H)-carboxylate

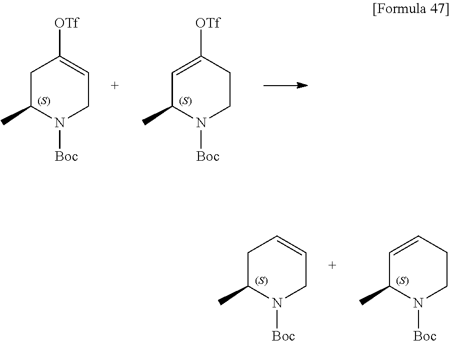

Triphenylphosphine (52.0 mg) and palladium acetate (22.0 mg) were added to a tetrahydrofuran solution (2 mL) of a mixture (345 mg) of Reference Example 100, Reference Example 101, and N-phenylbis(trifluoromethanesulfonimide) at room temperature, and the resulting mixture was stirred at room temperature for 5 minutes. A tetrahydrofuran solution (4 mL) of formic acid (51.0 mg) and diisopropylethylamine (116 mg) was added dropwise at room temperature over 3 minutes, and then the resulting mixture was stirred under reflux for 1 hour. Water was added to the reaction mixture, and the resulting mixture was extracted with ethyl acetate. The organic layer was dried over sodium sulfate, then filtered, and concentrated under reduced pressure. The residue was purified by silica gel column chromatography (hexane/ethyl acetate) to obtain the title compounds (45.0 mg) as a mixture.

¹H-NMR (DMSO-d₆) δ: 5.78-5.55 (2H, m), 4.46-3.47 (2H, m), 2.80-1.78 (2H, m), 1.45 (s, 4.5H), 1.44 (s, 4.5H), 1.14 (1.5H, d, J=6.8 Hz), 1.08 (1.5H, d, J=6.8 Hz).

### Reference Examples 106 and 107

The compounds of Reference Examples 106 and 107 were obtained by using the corresponding raw material compounds according to the method described in Reference Examples 104 an d 105.

TABLE 30

| Reference | Chemical | |
|---|---|---|
| Example | structure | Physical property data |
| 106 | 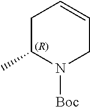 | Obtained as a mixture. ¹H-NMR (DMSO-d₆) δ: 5.78-5.55 (2H, m), 4.66-3.47 (2H, m), 2.80-1.79 (2H, m), 1.44 (s, 4.5H), 1.44 (s, 4.5H), 1.14 (1.5H, d, J = 6.8 Hz), 1.08 (1.5H d, J = 6.8 Hz). |
| 107 | 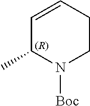 | |

### Reference Example 108

The compound of Reference Example 108 was obtained by using the corresponding raw material compounds according to the method described in Reference Example 20.

TABLE 31

| Reference | | |
|---|---|---|
| Example | Chemical structure | Physical property data |
| 108 | 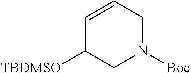 [SMILES: CC(C)(C)OC(=O)N1CC=CC(O[Si](C)(C)C(C)(C)C)C1] | LC-MS (min): 314.2/1.423 (Method B) |

### Reference Example 109

### tert-Butyl rac-(3R,4R,5R)-3-{[tert-butyl(dimethyl)silyl]oxy}-4,5-dihydroxypiperidine-1-carboxylate

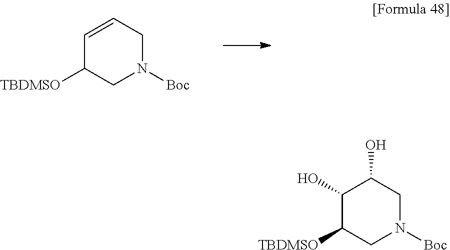 [SMILES: CC(C)(C)OC(=O)N1CC=CC(O[Si](C)(C)C(C)(C)C)C1 ; CC(C)(C)OC(=O)N1C[C@@H](O)[C@@H](O)[C@H](O[Si](C)(C)C(C)(C)C)C1]

4-Methylmorpholine-4-oxide monohydrate (2.68 g) and osmium tetroxide (immobilized catalyst I) (2.06 g) were added to a water/acetone/acetonitrile solution (15 mL/15 mL/15 mL) of the compound of Reference Example 108 (4.76 g) at room temperature, and the resulting mixture was stirred at room temperature for 16 hours. 4-Methylmorpholine 4-oxide monohydrate (3.48 g) and osmium tetroxide (immobilized catalyst I) (6.18 g) were added at room temperature, and the resulting mixture was stirred at room temperature for 25 hours. The reaction mixture was filtered and concentrated under reduced pressure. The residue was purified by column chromatography (hexane/ethyl acetate) to obtain the title compound (4.80 g).

LC-MS [M+H]⁺/Rt (min): 348.2/1.066 (Method B)

### Reference Example 110

### 1,6-Anhydro-2,3-dideoxy-D-erythro-2-hexenitol

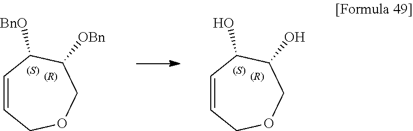

A catalytic amount of 10% palladium carbon was added to a methanol solution (50 mL) of the compound of Reference Example 97 (2.36 g) at room temperature, and the resulting mixture was stirred at room temperature overnight in a normal pressure hydrogen atmosphere. The reaction mixture was filtered and concentrated under reduced pressure to obtain the title compound (1.00 g).

¹H-NMR (CDCl₃) δ: 3.87-3.81 (1H, m), 3.79-3.71 (4H, m), 3.63-3.56 (1H, m), 1.95-1.81 (2H, m), 1.81-1.71 (1H, m), 1.72-1.56 (1H, m).

### Reference Examples 111 to 118

The compounds of Reference Examples 111 to 118 were obtained by using the corresponding raw material compounds according to the method described in Reference Example 38.

TABLE 32

| Reference | | |
|---|---|---|
| Example | Chemical structure | Physical property data |
| 111 | 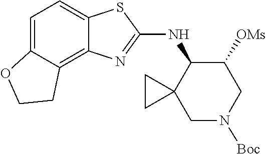 [SMILES: CC(C)(C)OC(=O)N1C[C@@H](OS(C)(=O)=O)[C@H](Nc2nc3c4c(ccc3s2)OCC4)C2(CC2)C1] | LC-MS [M + H]⁺/Rt (min): 496.3/1.035 (Method B) |
| 112 | 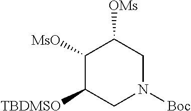 [SMILES: CC(C)(C)OC(=O)N1C[C@@H](O[Si](C)(C)C(C)(C)C)[C@H](OS(C)(=O)=O)[C@H](OS(C)(=O)=O)C1] | LC-MS [M + H]⁺/Rt (min): 504.3/1.224 (Method B) |
| 113 | 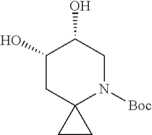 [SMILES: CC(C)(C)OC(=O)N1C[C@@H](O)[C@@H](O)CC12CC2] | ¹H-NMR (DMSO-d₆) δ: 4.00-3.83 (3H, m), 3.14-3.11 (1H, m), 2.32-2.08 (2H, m), 1.49-1.39 (1H, m), 1.44 (s, 9H), 1.28-1.00 (2H, m), 0.84-0.78 (1H, m), 0.66 (brs, 1H), 0.48-0.43 (1H, m). |
| 114 | 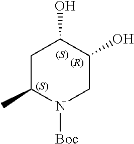 | Obtained as a mixture. LC-MS [M + H]⁺/Rt (min): 232.1/0.496, 232.1/0.496, 232.1/0.496 (Method B) |
| 115 | 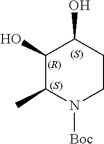 | |
| 116 | 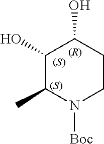 | |
| 117 | 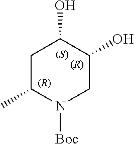 | Obtained as a mixture. LC-MS [M + H]⁺/Rt (min): 232.2/0.502, 232.2/0.502 (Method B) |
| 118 | 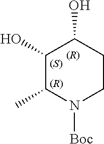 | |

### Reference Example 119

### (2R,4S,5R)-2-Cyano-5-[(4-methylbenzene-1-sulfonyl)oxy]oxane-4-yl 4-methylbenzene-1-sulfonate

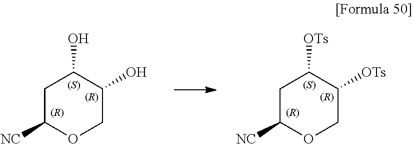

p-Toluenesulfonyl chloride (3.53 g) was added to a pyridine solution (15 mL) of (2R,4S,5R)-4,5-dihydroxytetrahydro-2H-pyran-2-carbonitrile (1.06 g) at 0° C., and the resulting mixture was stirred at 50° C. overnight. The reaction mixture was cooled to room temperature, then ice water was added, and the resulting mixture was extracted with ethyl acetate. The organic layer was washed with 1 N hydrochloric acid and saturated aqueous sodium bicarbonate, then filtered, and concentrated under reduced pressure. Diethyl ether was added to the residue, the resulting mixture was stirred, and then the resulting solid was collected by filtration to obtain the title compound (2.55 g).

1H-NMR (CDCl₃) δ: 7.74 (2H, d, J=6.4 Hz), 7.71 (2H, d, J=6.4 Hz), 7.39-7.32 (4H, m), 4.78-4.68 (2H, m), 4.51 (1H, dt, J=2.9, 5.6 Hz), 4.01 (1H, dd, J=5.6, 12.9 Hz), 3.80 (1H, dd, J=2.7, 12.9 Hz), 2.49-2.42 (1H, m), 2.47 (6H, s), 2.01-1.87 (1H, m).

### Reference Example 120

The compound of Reference Example 120 was obtained by using the corresponding raw material compounds according to the method described in Reference Example 119.

TABLE 33

| Reference | | |
|---|---|---|
| Example | Chemical structure | Physical property data |
| 120 | 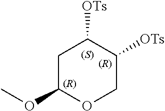 | ¹H-NMR (CDCl₃) δ: 7.80 (2H, d, J = 8.3 Hz), 7.67 (2H, d, J = 8.3 Hz), 7.39-7.28 (4H, m), 4.85-4.67 (2H, m), 4.61 (1H, td, J = 1.8, 3.3 Hz), 3.88 (1H, dd, J = 3.0, 13.1 Hz), 3.72 (1H, dd, J = 1.4, 13.1 Hz), 3.28 (3H, s), 2.45 (6H, s), 2.14 (1H, ddd, J = 3.4, 11.6, 12.8 Hz), 1.86-1.70 (1H, m). |

### Reference Examples 121 to 132

The compounds of Reference Examples 121 to 132 were obtained by using the corresponding raw material compounds according to the method described in Reference Example 38.

TABLE 34-1

| Reference | | |
|---|---|---|
| Example | Chemical structure | Physical property data |
| 121 | 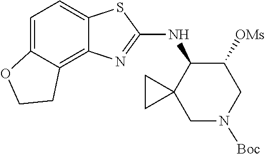 [SMILES: CC(C)(C)OC(=O)N1C[C@@H](OS(C)(=O)=O)[C@H](Nc2nc3c4c(ccc3s2)OCC4)C2(CC2)C1] | LC-MS [M + H]⁺/Rt (min): 496.3/1.035 (Method B) |
| 122 | 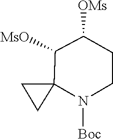 | LC-MS [M + H]⁺/Rt (min): 347.2/0.945 (Method B) |
| 123 | 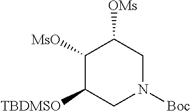 [SMILES: CC(C)(C)OC(=O)N1C[C@@H](O[Si](C)(C)C(C)(C)C)[C@H](OS(C)(=O)=O)[C@H](OS(C)(=O)=O)C1] | LC-MS [M + H]⁺/Rt (min): 504.3/1.224 (Method B) |
| 124 | 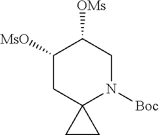 [SMILES: CC(C)(C)OC(=O)N1C[C@@H](OS(C)(=O)=O)[C@@H](OS(C)(=O)=O)CC12CC2] | ¹H-NMR (CDCl₃) δ: 4.97 (2H, brs), 3.12-3.08 (8H, m), 1.47-1.15 (2H, m), 1.46 (s, 9H), 1.28-1.00 (2H, m), 0.93 (2H, brs), 0.57 (brs, 2H). |
| 125 | 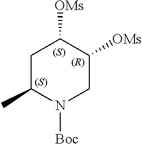 | Obtained as a mixture. LC-MS [M + H]⁺/Rt (min): 388.1/0.775, 388.1/0.775, 388.1/0.775 (Method B) |
| 126 | 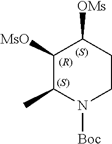 | |
| 127 | 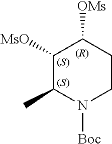 | |
| 128 | 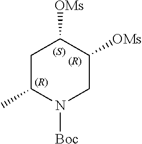 | Obtained as a mixture. LC-MS [M + H]⁺/Rt (min): 388.2/0.773, 388.2/0.773 (Method B) |
| 129 | 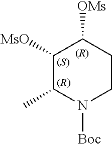 | |
| 130 | 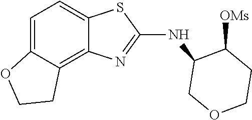 [SMILES: CS(=O)(=O)O[C@H]1CCOC[C@H]1Nc1nc2c3c(ccc2s1)OCC3] | LC-MS [M + H]⁺/Rt (min): 371.2/2.851 (Method C) |

TABLE 34-2

| | | |
|---|---|---|
| 131 | 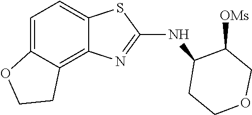 [SMILES: CS(=O)(=O)O[C@H]1COCC[C@H]1Nc1nc2c3c(ccc2s1)OCC3] | LC-MS [M + H]⁺/Rt (min): 371.2/2.825 (Method C) |
| 132 | 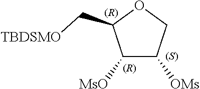 | ¹H-NMR (CDCl₃) δ: 5.25-5.19 (1H, m), 5.19-5.14 (1H, m), 4.24-4.19 (1H, m), 4.19-4.16 (1H, m), 4.06-4.00 (1H, m), 3.83 (2H, dd, J = 2.5, 11.7 Hz), 3.77 (1H, dd, J = 2.4, 11.7 Hz), 3.14 (3H, s), 3.13 (3H, s), 0.91 (9H, s), |
| | | 0.09 (3H, s), 0.08 (3H, s). |

### Reference Examples 133 to 146

The compounds of Reference Examples 133 to 146 were obtained by using the corresponding raw material compounds according to the method described in Reference Example 42.

TABLE 35-1

| Reference | | |
|---|---|---|
| Example | Chemical structure | Physical property data |
| 133 | 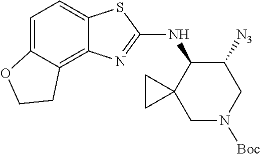 [SMILES: CC(C)(C)OC(=O)N1C[C@@H](N=[N+]=[N-])[C@H](Nc2nc3c4c(ccc3s2)OCC4)C2(CC2)C1] | LC-MS [M + H]⁺/Rt (min): 443.3/1.091 (Method B) |
| 134 | 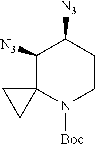 [SMILES: CC(C)(C)OC(=O)N1CC[C@H](N=[N+]=[N-])[C@H](N=[N+]=[N-])C12CC2] | LC-MS [M + H]⁺/Rt (min): 294.2/1.030 (Method B) |
| 135 | 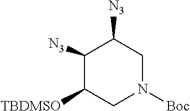 [SMILES: CC(C)(C)OC(=O)N1C[C@H](N=[N+]=[N-])[C@H](N=[N+]=[N-])[C@H](O[Si](C)(C)C(C)(C)C)C1] | LC-MS [M + H]⁺/Rt (min): 398.4/1.327 (Method B) |
| 136 | 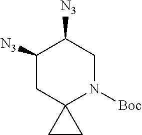 [SMILES: CC(C)(C)OC(=O)N1C[C@H](N=[N+]=[N-])[C@H](N=[N+]=[N-])CC12CC2] | LC-MS [M + H]⁺/Rt (min): 294.2/1.031 (Method B) |
| 137 | 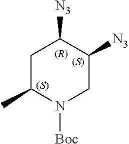 | Obtained as a mixture. LC-MS [M + H]⁺/Rt (min): 282.2/1.053, 282.2/1.053 (Method B) |
| 138 | 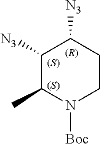 | |
| 139 | 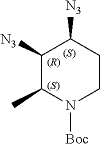 | LC-MS [M + H]⁺/Rt (min): 282.2/1.053 (Method B) |
| 140 | 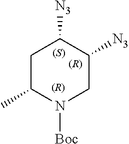 | Obtained as a mixture. LC-MS [M + H]⁺/Rt (min): 282.2/1.065, 282.2/1.065 (Method B) |
| 141 | 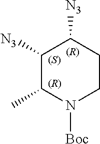 | |
| 142 | 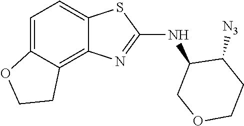 [SMILES: [N-]=[N+]=N[C@@H]1CCOC[C@H]1Nc1nc2c3c(ccc2s1)OCC3] | LC-MS [M + H]⁺/Rt (min): 318.1/3.218 (Method C) |

TABLE 35-2

| | | |
|---|---|---|
| 143 | 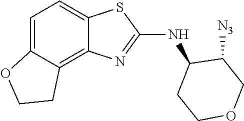 [SMILES: [N-]=[N+]=N[C@@H]1COCC[C@H]1Nc1nc2c3c(ccc2s1)OCC3] | LC-MS [M + H]⁺/Rt (min): 318.1/3.192 (Method C) |
| 144 | 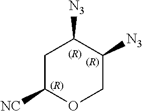 | ¹H-NMR (CDCl₃) δ: 4.45 (1H, dd, J = 4.1, 7.5 Hz), 4.16-4.12 (1H, m), 3.89- 3.83 (1H, m), 3.76-3.66 (2H, m), 2.34 (1H, ddd, J = 7.5, 8.4, 13.8 Hz), 2.19 (1H, dtd, J = 0.7, 4.1, 13.8 Hz). |
| 145 | 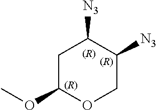 | ¹H-NMR (CDCl₃) δ: 4.53-4.41 (1H, m), 4.14-4.02 (1H, m), 3.79 (1H, ddd, J = 3.4, 5.2, 8.1 Hz), 3.65 (1H, td, J = 3.2, 6.0 Hz), 3.63-3.52 (1H, m), 3.45 (3H, s), 2.10-1.98 (2H, m). |
| 146 | 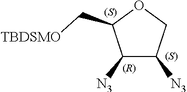 | ¹H-NMR (CDCl₃) δ: 4.26-4.12 (2H, m), 4.10-3.99 (2H, m), 3.89-3.72 (3H, m), 0.90 (9H, s), 0.10 (3H, s), 0.08 (3H, s). |

### Reference Example 147

### tert-Butyl rac-(3R,4R,5S)-3,4-diazido-5-hydroxypiperidine-1-carboxylate

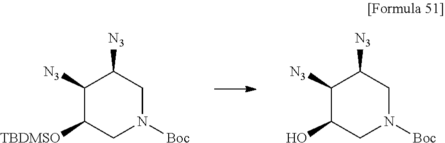 [SMILES: CC(C)(C)OC(=O)N1C[C@H](N=[N+]=[N-])[C@H](N=[N+]=[N-])[C@H](O[Si](C)(C)C(C)(C)C)C1 ; CC(C)(C)OC(=O)N1C[C@@H](O)[C@@H](N=[N+]=[N-])[C@@H](N=[N+]=[N-])C1]

Tetrabutylammonium fluoride (1 M tetrahydrofuran solution, 3.01 mL) was added to a tetrahydrofuran solution (10 mL) of the compound of Reference Example 135 (798 mg) at room temperature, and the resulting mixture was stirred for 1 hour. Methanol was added to the reaction mixture, and the resulting mixture was concentrated under reduced pressure. The residue was purified by column chromatography (hexane/ethyl acetate) to obtain the title compound (542 mg).

LC-MS [M+H]⁺/Rt (min): 284.2/0.829 (Method B)

### Reference Example 148

### tert-Butyl rac-(3R,4R,5R)-3,4-diazido-5-fluoropiperidine-1-carboxylate

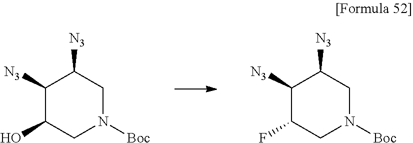 [SMILES: CC(C)(C)OC(=O)N1C[C@@H](O)[C@@H](N=[N+]=[N-])[C@@H](N=[N+]=[N-])C1 ; CC(C)(C)OC(=O)N1C[C@H](N=[N+]=[N-])[C@H](N=[N+]=[N-])[C@@H](F)C1]

Bis(2-methoxyethyl)aminosulfur-trifluoride (0.548 mL) was added to a chloroform solution (10 mL) of the compound of Reference Example 147 (442 mg) at room temperature, and the resulting mixture was stirred at room temperature overnight. The reaction mixture was directly purified by column chromatography (hexane/ethyl acetate) to obtain the title compound (343 mg).

LC-MS [M+H]⁺/Rt (min): 286.1/0.944 (Method B)

### Reference Example 149

### tert-Butyl 7-oxa-3-azaspiro[bicyclo[4.1.0]heptane-2,1′-cyclopropane]-3-carboxylate

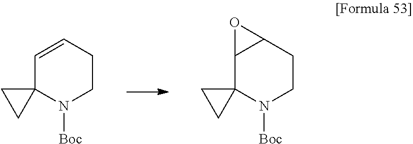 [SMILES: CC(C)(C)OC(=O)N1CCC=CC12CC2 ; CC(C)(C)OC(=O)N1CCC2OC2C12CC2]

m-Chloroperbenzoic acid (592 mg) was added to a chloroform solution (10 mL) of the compound of Reference Example 95 (418 mg) at 0° C., and the resulting mixture was stirred at 0° C. for 2 hours and at room temperature for 3 hours. Saturated aqueous sodium bicarbonate was added to the reaction mixture, and the resulting mixture was extracted with chloroform. The organic layer was washed with a mixed solution of saturated aqueous sodium bicarbonate and a saturated sodium thiosulfate aqueous solution, dried over sodium sulfate, then filtered, and concentrated under reduced pressure. The residue was purified by column chromatography (hexane/ethyl acetate) to obtain the title compound (333 mg).

LC-MS [M+H]⁺/Rt (min): 226.1/0.979 (Method B)

### Reference Example 150

### tert-Butyl rac-(7R,8R)-8-azido-7-hydroxy-4-azaspiro[2.5]octane-4-carboxylate

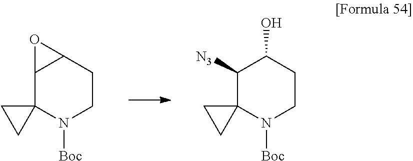 [SMILES: CC(C)(C)OC(=O)N1CCC2OC2C12CC2 ; CC(C)(C)OC(=O)N1CC[C@@H](O)[C@H](N=[N+]=[N-])C12CC2]

Sodium azide (115 mg) and ammonium chloride (95 mg) were added to a methanol/water solution (6 mL/4 mL) of the compound of Reference Example 149 (333 mg) at room temperature, and the resulting mixture was stirred at 65° C. for 24 hours and at 100° C. for 11 hours. Saturated aqueous sodium bicarbonate was added to the reaction mixture, and the resulting mixture was extracted with ethyl acetate. The organic layer was dried over sodium sulfate, then filtered, and concentrated under reduced pressure. The residue was purified by column chromatography (hexane/ethyl acetate) to obtain the title compound (100 mg).

LC-MS [M+H]⁺/Rt (min): 269.3/0.892 (Method A)

### Reference Example 151

### tert-Butyl rac-(6R,7S)-6,7-diamino-4-azaspiro[2.5]octane-4-carboxylate

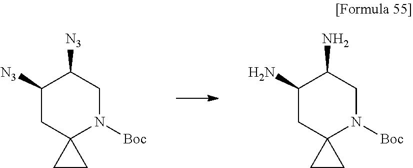 [SMILES: CC(C)(C)OC(=O)N1C[C@H](N=[N+]=[N-])[C@H](N=[N+]=[N-])CC12CC2 ; CC(C)(C)OC(=O)N1C[C@H](N)[C@H](N)CC12CC2]

Triphenylphosphine (1.41 g) was added to a tetrahydrofuran-water solution (10:1) (18 mL) of the compound of Reference Example 136 (530 mg) at room temperature, and the resulting mixture was stirred at 60° C. for 4 hours. The reaction mixture was concentrated under reduced pressure, and then the residue was purified by silica gel column chromatography (ethyl acetate/methanol) to obtain the title compound (380 mg).

LC-MS [M+H]⁺/Rt (min): 242.2/0.241 (Method B)

### Reference Examples 152 to 166

The compounds of Reference Examples 152 to 166 were obtained by using the corresponding raw material compounds according to the method described in Reference Example 44 or Reference Example 151.

TABLE 36-1

| Reference | | |
|---|---|---|
| Example | Chemical structure | Physical property data |
| 152 | 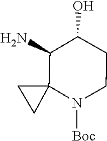 | LC-MS [M + H]⁺/Rt (min): 243.2/0.392 (Method B) |
| 153 | 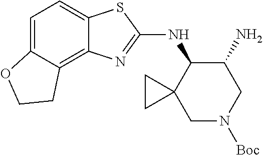 [SMILES: CC(C)(C)OC(=O)N1C[C@@H](N)[C@H](Nc2nc3c4c(ccc3s2)OCC4)C2(CC2)C1] | LC-MS [M + H]⁺/Rt (min): 417.3/0.766 (Method B) |
| 154 | 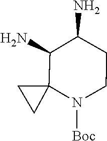 | LC-MS [M + H]⁺/Rt (min): 242.2/0.329 (Method B) |
| 155 | 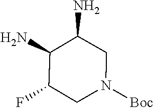 | LC-MS [M + H]⁺/Rt (min): 234.1/0.194 (Method B) |
| 156 | 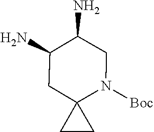 [SMILES: CC(C)(C)OC(=O)N1C[C@H](N)[C@H](N)CC12CC2] | LC-MS [M + H]⁺/Rt (min): 242.2/0.251 (Method B) |
| 157 | 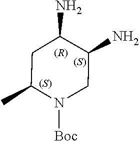 | Obtained as a mixture. LC-MS [M + H]⁺/Rt (min): 230.2/0.287, 230.2/0.287 (Method B) |
| 158 | 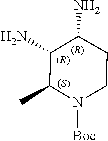 | |
| 159 | 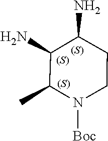 | LC-MS [M + H]⁺/Rt (min): 230.2/0.221 (Method B) |
| 160 | 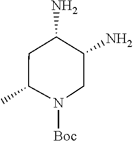 | Obtained as a mixture. LC-MS [M + H]⁺/Rt (min): 230.2/0.198, 230.2/0.198 (Method B) |
| 161 | 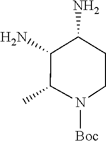 | |

TABLE 36-2

| | | |
|---|---|---|
| 162 | 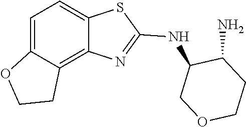 [SMILES: N[C@@H]1CCOC[C@H]1Nc1nc2c3c(ccc2s1)OCC3] | LC-MS [M + H]⁺/Rt (min): 292.2/2.089 (Method C) |
| 163 | 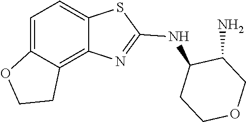 [SMILES: N[C@@H]1COCC[C@H]1Nc1nc2c3c(ccc2s1)OCC3] | LC-MS [M + H]⁺/Rt (min): 292.2/2.050 (Method C) |
| 164 | 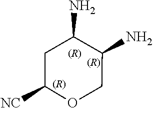 | ¹H-NMR (CDCl₃) δ: 4.29-4.19 (1H, m), 3.93 (1H, dd, J = 3.7, 11.9 Hz), 3.56 (1H, dd, J = 2.3, 11.9 Hz), 3.04- 2.92 (1H, m), 2.83 (1H, dt, J = 2.4, 3.8 Hz), 2.02-1.82 (2H, m), 1.69-1.38 (4H, m). |
| 165 | 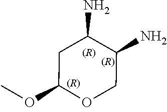 | LC-MS [M + H]⁺/Rt (min): 147.1/0.331 (Method C) |
| 166 | 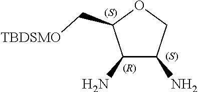 | LC-MS [M + H]⁺/Rt (min): 247.2/1.690 (Method C) |

### Reference Example 167

### Di-tert-butyl (3R,4R)-2,3,4,7-tetrahydrooxepin-3,4-diylbis rac-carbamate

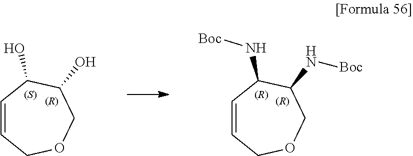

Triethylamine (6.33 mL) and methanesulfonyl chloride (1.77 mL) were added to a dichloromethane solution (8 mL) of the compound of Reference Example 110 (1.00 g) under ice cooling, and the resulting mixture was stirred under ice cooling for 2 hours. Water was added to the reaction mixture, and the resulting mixture was extracted with dichloromethane. The organic layer was washed with saturated aqueous sodium bicarbonate and saturated brine, then dried over sodium hydrogen sulfate, filtered, and then concentrated under reduced pressure. Sodium azide (1.94 g) was added to a dimethylformamide solution (12 mL) of the resulting crude product at room temperature, and the resulting mixture was stirred at 65° C. for 18 hours. Water was added to the reaction mixture, and the resulting mixture was extracted with ethyl acetate. The organic layer was washed with water and saturated brine, then dried over sodium hydrogen sulfate, filtered, and then concentrated under reduced pressure. 10% Palladium carbon (0.10 g) was added to an ethanol solution (15 mL) of the resulting crude product at room temperature, and the resulting mixture was stirred at room temperature for 3 days in a 35 bar hydrogen atmosphere. The reaction mixture was filtered and then concentrated under reduced pressure. Triethylamine (1.59 mL) and (Boc)₂O (2.64 mL) were added to a methanol solution (28 mL) of the resulting crude product at room temperature, and the resulting mixture was stirred at room temperature overnight. The reaction mixture was concentrated under reduced pressure and then purified by silica gel chromatography (hexane/ethyl acetate) to obtain the title compound (314 mg).

¹H-NMR (CDCl₃) δ: 5.26 (2H, s), 4.08-3.90 (1H, m), 3.89-3.71 (2H, m), 3.72-3.50 (3H, m), 1.92-1.62 (4H, m), 1.46 (9H, s), 1.45 (9H, s).

### Reference Examples 168 to 206

The compounds of Reference Examples 168 to 206 were obtained by using the corresponding raw material compounds according to the method described in Reference Example 1 and Reference Example 2 or Reference Example 24.

TABLE 37-1

| Reference | | |
|---|---|---|
| Example | Chemical structure | Physical property data |
| 168 | 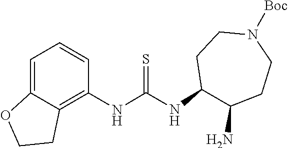 [SMILES: CC(C)(C)OC(=O)N1CC[C@@H](N)[C@@H](NC(=S)Nc2cccc3c2CCO3)CC1] | LC-MS [M + H]⁺/Rt (min): 407.5/0.655 (Method B) |
| 169 | 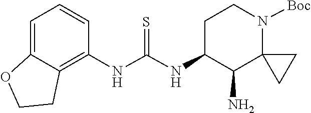 [SMILES: CC(C)(C)OC(=O)N1CC[C@H](NC(=S)Nc2cccc3c2CCO3)[C@H](N)C12CC2] | LC-MS [M + H]⁺/Rt (min): 419.3/0.711 (Method B) |
| 170 | 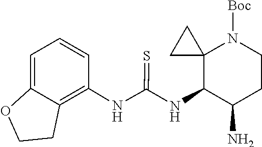 [SMILES: CC(C)(C)OC(=O)N1CC[C@@H](N)[C@@H](NC(=S)Nc2cccc3c2CCO3)C12CC2] | LC-MS [M + H]⁺/Rt (min): 419.3/0.693 (Method B) |
| 171 | 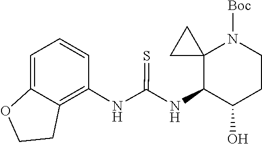 [SMILES: CC(C)(C)OC(=O)N1CC[C@H](O)[C@@H](NC(=S)Nc2cccc3c2CCO3)C12CC2] | LC-MS [M + H]⁺/Rt (min): 420.3/0.838 (Method B) |
| 172 | 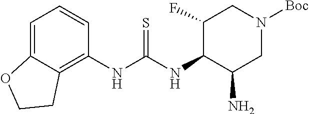 [SMILES: CC(C)(C)OC(=O)N1C[C@@H](N)[C@@H](NC(=S)Nc2cccc3c2CCO3)[C@H](F)C1] | LC-MS [M + H]⁺/Rt (min): 411.2/0.586 (Method B) |
| 173 | 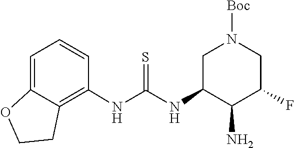 [SMILES: CC(C)(C)OC(=O)N1C[C@H](NC(=S)Nc2cccc3c2CCO3)[C@H](N)[C@@H](F)C1] | LC-MS [M + H]⁺/Rt (min): 411.3/0.600 (Method B) |
| 174 | 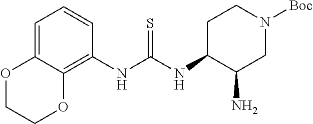 [SMILES: CC(C)(C)OC(=O)N1CC[C@H](NC(=S)Nc2cccc3c2OCCO3)[C@H](N)C1] | LC-MS [M + H]⁺/Rt (min): 409.2/0.576 (Method B) |
| 175 | 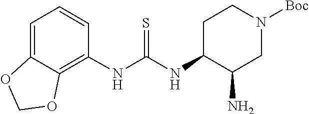 [SMILES: CC(C)(C)OC(=O)N1CC[C@H](NC(=S)Nc2cccc3c2OCO3)[C@H](N)C1] | LC-MS [M + H]⁺/Rt (min): 395.2/0.659 (Method A) |
| 176 | 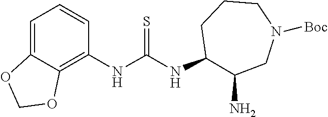 [SMILES: CC(C)(C)OC(=O)N1CCC[C@H](NC(=S)Nc2cccc3c2OCO3)[C@H](N)C1] | LC-MS [M + H]⁺/Rt (min): 409.4/0.786 (Method A) |
| 177 | 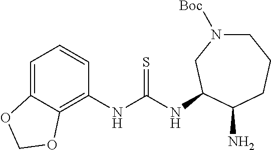 [SMILES: CC(C)(C)OC(=O)N1CCC[C@@H](N)[C@@H](NC(=S)Nc2cccc3c2OCO3)C1] | LC-MS [M + H]⁺/Rt (min): 409.4/0.769 (Method A). |

TABLE 37-2

| | | |
|---|---|---|
| 178 | 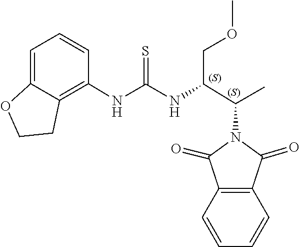 | LC-MS [M + H]⁺/Rt (min): 426.2/0.846 (Method B) |
| 179 | 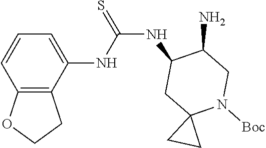 [SMILES: CC(C)(C)OC(=O)N1C[C@H](N)[C@H](NC(=S)Nc2cccc3c2CCO3)CC12CC2] | LC-MS [M + H]⁺/Rt (min): 419.2/0.682 (Method B) |
| 180 | 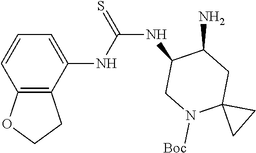 [SMILES: CC(C)(C)OC(=O)N1C[C@@H](NC(=S)Nc2cccc3c2CCO3)[C@@H](N)CC12CC2] | LC-MS [M + H]⁺/Rt (min): 419.2/0.682 (Method B) |
| 181 | 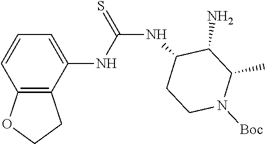 [SMILES: C[C@H]1[C@@H](N)[C@@H](NC(=S)Nc2cccc3c2CCO3)CCN1C(=O)OC(C)(C)C] | LC-MS [M + H]⁺/Rt (min): 407.2/0.591 (Method B) |
| 182 | 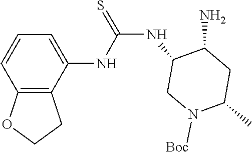 [SMILES: C[C@H]1C[C@@H](N)[C@@H](NC(=S)Nc2cccc3c2CCO3)CN1C(=O)OC(C)(C)C] | LC-MS [M + H]⁺/Rt (min): 407.3/0.675 (Method B) |
| 183 | 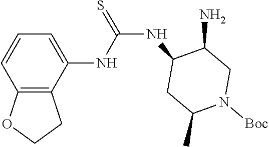 [SMILES: C[C@H]1C[C@@H](NC(=S)Nc2cccc3c2CCO3)[C@@H](N)CN1C(=O)OC(C)(C)C] | Obtained as a mixture. LC-MS [M + H]⁺/Rt (min): 407.3/0.641, 407.3/0.678 (Method B) |
| 184 | 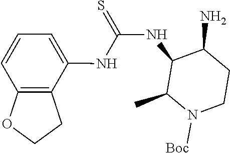 [SMILES: C[C@H]1[C@@H](NC(=S)Nc2cccc3c2CCO3)[C@@H](N)CCN1C(=O)OC(C)(C)C] | |
| 185 | 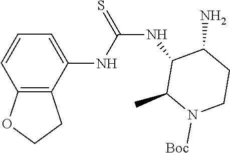 [SMILES: C[C@H]1[C@H](NC(=S)Nc2cccc3c2CCO3)[C@H](N)CCN1C(=O)OC(C)(C)C] | LC-MS [M + H]⁺/Rt (min): 407.2/0.627 (Method B) |
| 186 | 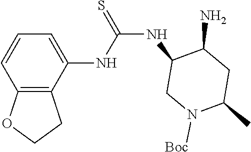 [SMILES: C[C@@H]1C[C@H](N)[C@H](NC(=S)Nc2cccc3c2CCO3)CN1C(=O)OC(C)(C)C] | LC-MS [M + H]⁺/Rt (min): 407.3/0.624 (Method B) |
| 187 | 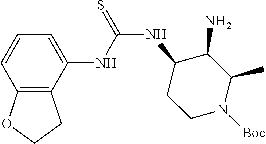 [SMILES: C[C@@H]1[C@H](N)[C@H](NC(=S)Nc2cccc3c2CCO3)CCN1C(=O)OC(C)(C)C] | LC-MS [M + H]⁺/Rt (min): 407.3/0.558 (Method B) |

TABLE 37-3

| | | |
|---|---|---|
| 188 | 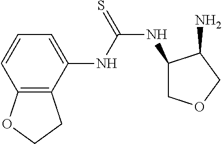 [SMILES: N[C@H]1COC[C@H]1NC(=S)Nc1cccc2c1CCO2] | LC-MS [M + H]⁺/Rt (min): 280.1/1.653 (Method C) |
| 189 | 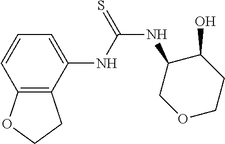 [SMILES: O[C@H]1CCOC[C@H]1NC(=S)Nc1cccc2c1CCO2] | LC-MS [M + H]⁺/Rt (min): 295.2/2.502 (Method C) |
| 190 | 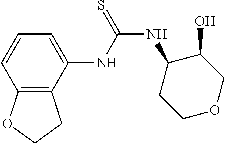 [SMILES: O[C@H]1COCC[C@H]1NC(=S)Nc1cccc2c1CCO2] | LC-MS [M + H]⁺/Rt (min): 295.2/2.436 (Method C) |
| 191 | 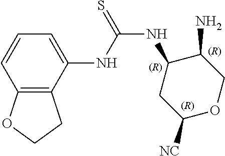 | Obtained as a mixture. LC-MS [M + H]⁺/Rt (min): 319.1/1.507, 319.1/1.540 (Method C) |
| 192 | 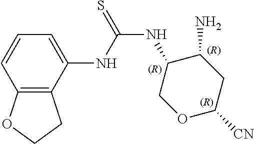 | |
| 193 | 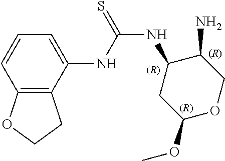 | Obtained as a mixture. LC-MS [M + H]⁺/Rt (min): 324.1/1.907, 324.1/1.857 (Method C) |
| 194 | 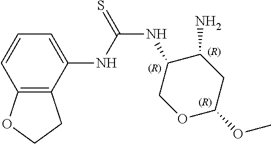 | |
| 195 | 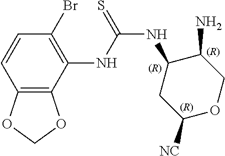 | LC-MS [M + 2H]⁺/Rt (min): 401.0/1.623 (Method C) |
| 196 | 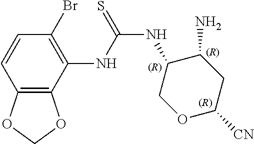 | LC-MS [M + 2H]⁺/Rt (min): 401.0/1.623 (Method C) |
| 197 | 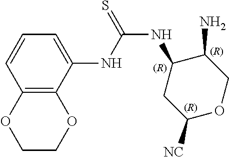 | LC-MS [M + H]⁺/Rt (min): 335.0/1.573 (Method C) |

TABLE 37-4

| | | |
|---|---|---|
| 198 | 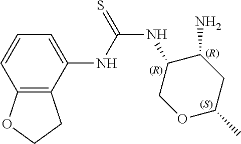 | LC-MS [M + H]⁺/Rt (min): 308.1/1.590 (Method C) |
| 199 | 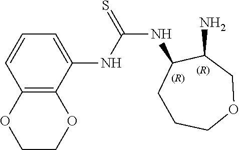 | ¹H-NMR (DMSO-d₆) δ: 9.17 (1H, s), 7.09 (1H, dd, J = 1.6, 8.0 Hz), 6.76 (1H, t, J = 8.0 Hz), 6.69 (1H, dd, J = 1.6, 8.0 Hz), 4.36- 4.13 (6H, m), 3.76-3.60 (2H, m), 3.60-3.48 (1H, m), 3.37-3.27 (1H, m), 2.95-2.85 (1H, m), 1.84-1.49 (5H, m). |
| 200 | 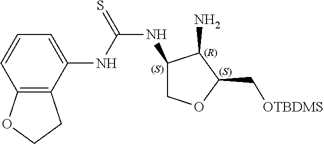 | LC-MS [M + H]⁺/Rt (min): 424.3/2.090 (Method C) |
| 201 | 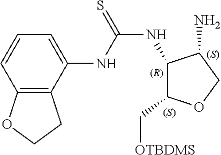 | LC-MS [M + H]⁺/Rt (min): 424.3/2.090 (Method C) |
| 202 | 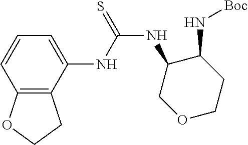 [SMILES: CC(C)(C)OC(=O)N[C@H]1CCOC[C@H]1NC(=S)Nc1cccc2c1CCO2] | LC-MS [M + H]⁺/Rt (min): 394.1/3.063 (Method C) |
| 203 | 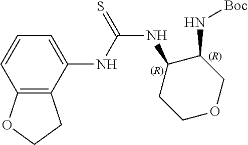 | ¹H-NMR (CDCl₃) δ: 7.47 (1H, s), 7.12 (1H, t, J = 8.0 Hz), 6.73-6.65 (2H, m), 5.09 (1H, d, J = 8.6 Hz), 4.72-4.52 (3H, m), 3.99-3.87 (2H, m), 3.87-3.76 (1H, m), 3.66- 3.47 (2H, m), 3.30-3.06 (2H, m), 2.32-2.23 (1H, m), 1.64-1.50 (1H, m), 1.31 (9H, s). |
| 204 | 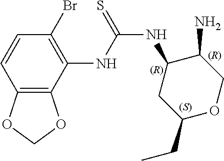 | LC-MS [M + 2H]⁺/Rt (min): 404.0/1.790 (Method C) |
| 205 | 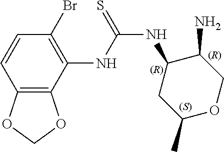 | LC-MS [M + 2H]⁺/Rt (min): 390.1/1.673 (method C) |
| 206 | 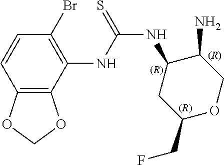 | LC-MS [M + 2H]⁺/Rt (min): 408.1/1.673 (method C) |

### Reference Example 207

### tert-Butyl rac-{(3R,4S)-4-[(7,8-dihydrofuro[3,2-e]1,3]benzothiazol-2-yl)amino]oxolan-3-yl}carbamate

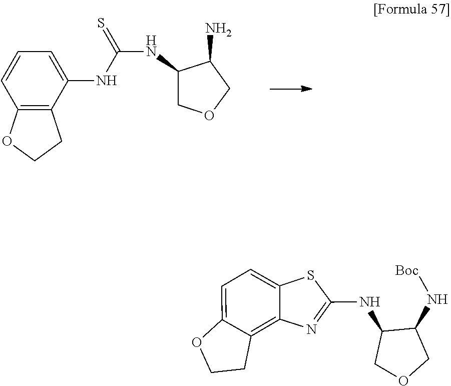 [SMILES: N[C@H]1COC[C@H]1NC(=S)Nc1cccc2c1CCO2 ; CC(C)(C)OC(=O)N[C@H]1COC[C@H]1Nc1nc2c3c(ccc2s1)OCC3]

Benzyltrimethylammonium tribromide (1.30 g) was added to a chloroform suspension (30 mL) of the compound of Reference Example 188 (947 mg) at 0° C., and the resulting mixture was stirred at 0° C. for 15 minutes. Triethylamine (0.945 mL) and (Boc)₂O (814 mg) were added at 0° C., and the resulting mixture was stirred at room temperature for 3 hours. Saturated aqueous sodium bicarbonate was added to the reaction mixture, and the resulting mixture was extracted with chloroform. The organic layer was dried over sodium sulfate, filtered, and then concentrated under reduced pressure. The residue was purified by silica gel chromatography (hexane/ethyl acetate) to obtain the title compound (688 mg).

LC-MS [M+H]⁺/Rt (min): 378.1/3.197 (Method C)

### Reference Examples 208 to 248

The compounds of Reference Examples 208 to 248 were obtained by using the corresponding raw material compounds according to the methods described in Reference Example 8, Reference Example 26, and Reference Example 47.

TABLE 38-1

| Reference | | |
|---|---|---|
| Example | Chemical structure | Physical property data |
| 208 | 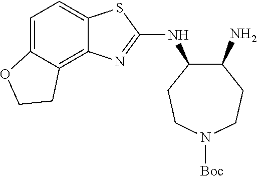 [SMILES: CC(C)(C)OC(=O)N1CC[C@H](N)[C@H](Nc2nc3c4c(ccc3s2)OCC4)CC1] | LC-MS [M + H]⁺/Rt (min): 405.3/0.770 (Method B) |
| 209 | 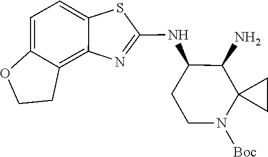 [SMILES: CC(C)(C)OC(=O)N1CC[C@@H](Nc2nc3c4c(ccc3s2)OCC4)[C@@H](N)C12CC2] | LC-MS [M + H]⁺/Rt (min): 417.3/0.673 (Method B) |
| 210 | 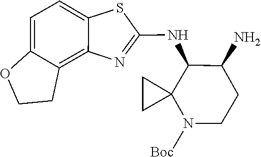 [SMILES: CC(C)(C)OC(=O)N1CC[C@H](N)[C@H](Nc2nc3c4c(ccc3s2)OCC4)C12CC2] | LC-MS [M + H]⁺/Rt (min): 417.3/0.717 (Method B) |
| 211 | 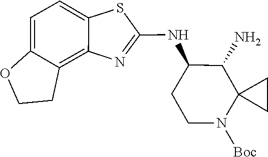 [SMILES: CC(C)(C)OC(=O)N1CC[C@@H](Nc2nc3c4c(ccc3s2)OCC4)[C@H](N)C12CC2] | LC-MS [M + H]⁺/Rt (min): 417.5/0.694 (Method B) |
| 212 | 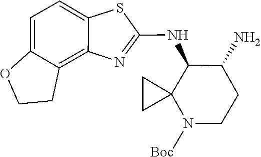 [SMILES: CC(C)(C)OC(=O)N1CC[C@@H](N)[C@H](Nc2nc3c4c(ccc3s2)OCC4)C12CC2] | LC-MS [M + H]⁺/Rt (min): 417.3/0.766 (Method B) |
| 213 | 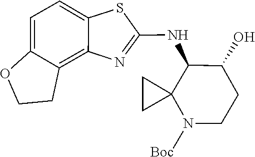 [SMILES: CC(C)(C)OC(=O)N1CC[C@@H](O)[C@H](Nc2nc3c4c(ccc3s2)OCC4)C12CC2] | LC-MS [M + H]⁺/Rt (min): 418.2/0.879 (Method B) |
| 214 | 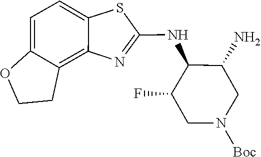 [SMILES: CC(C)(C)OC(=O)N1C[C@@H](N)[C@H](Nc2nc3c4c(ccc3s2)OCC4)[C@@H](F)C1] | LC-MS [M + H]⁺/Rt (min): 409.2/0.571 (Method B) |
| 215 | 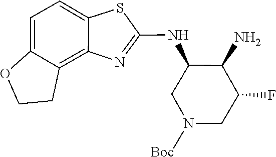 [SMILES: CC(C)(C)OC(=O)N1C[C@@H](F)[C@H](N)[C@H](Nc2nc3c4c(ccc3s2)OCC4)C1] | LC-MS [M + H]⁺/Rt (min): 409.2/0.600 (Method B) |
| 216 | 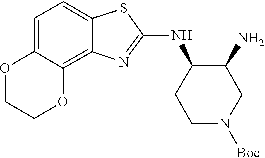 [SMILES: CC(C)(C)OC(=O)N1CC[C@@H](Nc2nc3c4c(ccc3s2)OCCO4)[C@@H](N)C1] | LC-MS [M + H]⁺/Rt (min): 407.2/0.547 (Method B). |

TABLE 38-2

| | | |
|---|---|---|
| 217 | 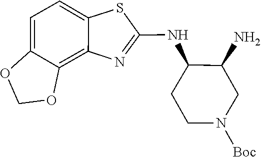 [SMILES: CC(C)(C)OC(=O)N1CC[C@@H](Nc2nc3c4c(ccc3s2)OCO4)[C@@H](N)C1] | ¹H-NMR (CDCl₃) δ: 6.97 (1H, d, J = 7.9 Hz), 6.64 (1H, d, J = 7.9 Hz), 6.02-6.00 (3H, m), 4.03-4.00 (3H, m), 3.13 (1H, brs), 3.06-3.02 (1H, m), 2.87 (1H, t, J = 12.4 Hz), 2.02-1.98 (1H, m), 1.64-1.54 (3H, m), 1.45 (9H, s). |
| 218 | 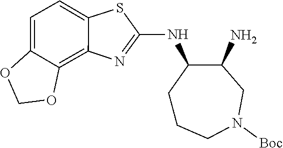 [SMILES: CC(C)(C)OC(=O)N1CCC[C@@H](Nc2nc3c4c(ccc3s2)OCO4)[C@@H](N)C1] | LC-MS [M + H]⁺/Rt (min): 407.3/0.723 (Method A) |
| 219 | 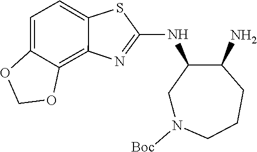 [SMILES: CC(C)(C)OC(=O)N1CCC[C@H](N)[C@H](Nc2nc3c4c(ccc3s2)OCO4)C1] | LC-MS [M + H]⁺/Rt (min): 407.3/0.678 (Method A) |
| 220 | 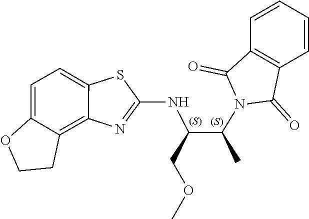 | LC-MS [M + H]⁺/Rt (min): 424.2/0.907 (Method B) |
| 221 | 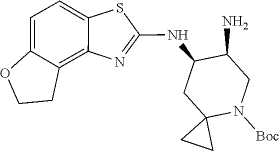 [SMILES: CC(C)(C)OC(=O)N1C[C@H](N)[C@H](Nc2nc3c4c(ccc3s2)OCC4)CC12CC2] | LC-MS [M + H]⁺/Rt (min): 417.3/0.643 (Method B) |
| 222 | 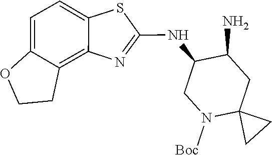 [SMILES: CC(C)(C)OC(=O)N1C[C@@H](Nc2nc3c4c(ccc3s2)OCC4)[C@@H](N)CC12CC2] | LC-MS [M + H]⁺/Rt (min): 417.3/0.663 (Method B) |
| 223 | 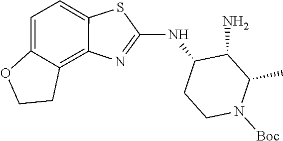 [SMILES: C[C@H]1[C@@H](N)[C@@H](Nc2nc3c4c(ccc3s2)OCC4)CCN1C(=O)OC(C)(C)C] | LC-MS [M + H]⁺/Rt (min): 405.3/0.600 (Method B) |
| 224 | 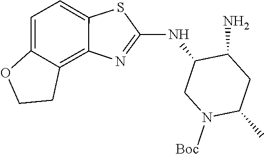 [SMILES: C[C@H]1C[C@@H](N)[C@@H](Nc2nc3c4c(ccc3s2)OCC4)CN1C(=O)OC(C)(C)C] | LC-MS [M + H]⁺/Rt (min): 405.2/0.663 (Method B) |
| 225 | 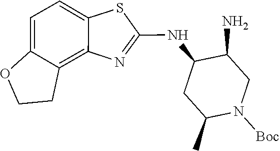 [SMILES: C[C@H]1C[C@@H](Nc2nc3c4c(ccc3s2)OCC4)[C@@H](N)CN1C(=O)OC(C)(C)C] | Obtained as a mixture. LC-MS [M + H]⁺/Rt (min): 405.3/0.662, 405.3/0.689 (Method B) |
| 226 | 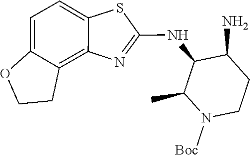 [SMILES: C[C@H]1[C@@H](Nc2nc3c4c(ccc3s2)OCC4)[C@@H](N)CCN1C(=O)OC(C)(C)C] | |

TABLE 38-3

| | | |
|---|---|---|
| 227 | 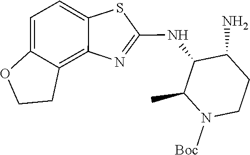 [SMILES: C[C@H]1[C@H](Nc2nc3c4c(ccc3s2)OCC4)[C@H](N)CCN1C(=O)OC(C)(C)C] | LC-MS [M + H]⁺/Rt (min): 405.3/0.642 (Method B) |
| 228 | 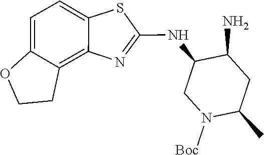 [SMILES: C[C@@H]1C[C@H](N)[C@H](Nc2nc3c4c(ccc3s2)OCC4)CN1C(=O)OC(C)(C)C] | LC-MS [M + H]⁺/Rt (min): 405.3/0.653 (Method B) |
| 229 | 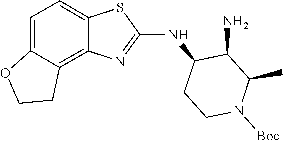 [SMILES: C[C@@H]1[C@H](N)[C@H](Nc2nc3c4c(ccc3s2)OCC4)CCN1C(=O)OC(C)(C)C] | LC-MS [M + H]⁺/Rt (min): 405.3/0.623 (Method B) |
| 230 | 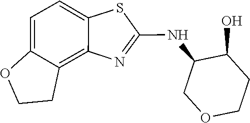 [SMILES: O[C@H]1CCOC[C@H]1Nc1nc2c3c(ccc2s1)OCC3] | LC-MS [M + H]⁺/Rt (min): 293.2/2.575 (Method C) |
| 231 | 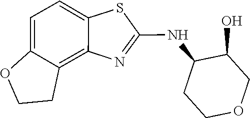 [SMILES: O[C@H]1COCC[C@H]1Nc1nc2c3c(ccc2s1)OCC3] | LC-MS [M + H]⁺/Rt (min): 293.2/2.649 (Method C) |
| 232 | 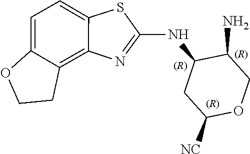 | LC-MS [M + H]⁺/Rt (min): 317.1/1.624 (Method C) |
| 233 | 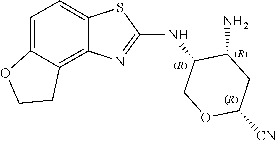 | LC-MS [M + H]⁺/Rt (min): 317.2/1.673 (Method C) |
| 234 | 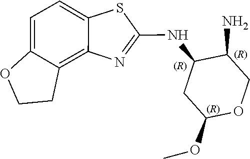 | LC-MS [M + H]⁺/Rt (min): 322.1/2.179 (Method C) |
| 235 | 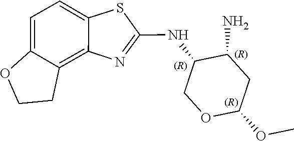 | LC-MS [M + H]⁺/Rt (min): 322.2/2.169 (Method C) |
| 236 | 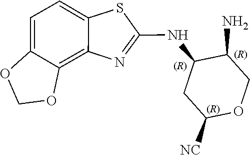 | LC-MS [M + H]⁺/Rt (min): 319.1/1.607 (Method C) |
| 237 | 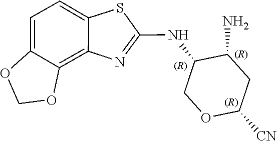 | LC-MS [M + H]⁺/Rt (min): 319.1/1.673 (Method C) |

TABLE 38-4

| | | |
|---|---|---|
| 238 | 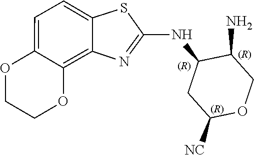 | LC-MS [M + H]⁺/Rt (min): 333.1/1.640 (Method C) |
| 239 | 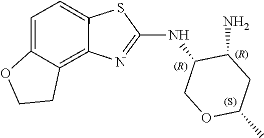 | LC-MS [M + H]⁺/Rt (min): 306.1/1.624 (Method C) |
| 240 | 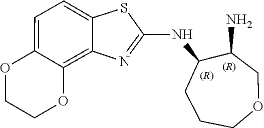 | LC-MS [M + H]⁺/Rt (min): 322.1/1.656 (Method C) |
| 241 | 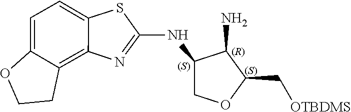 | LC-MS [M + H]⁺/Rt (min): 422.3/2.290 (Method C) |
| 242 | 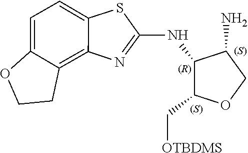 | LC-MS [M + H]⁺/Rt (min): 422.3/2.107 (Method C) |
| 243 | 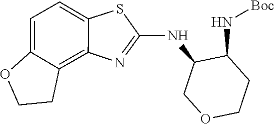 [SMILES: CC(C)(C)OC(=O)N[C@H]1CCOC[C@H]1Nc1nc2c3c(ccc2s1)OCC3] | LC-MS [M + H]⁺/Rt (min): 392.3/3.259 (Method C) |
| 244 | 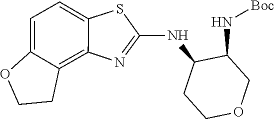 [SMILES: CC(C)(C)OC(=O)N[C@H]1COCC[C@H]1Nc1nc2c3c(ccc2s1)OCC3] | ¹H-NMR (CDCl₃) δ: 7.28 (1H, dd, J = 0.9, 8.4 Hz), 6.62 (1H, d, J = 8.4 Hz), 6.00 (1H, brs), 5.24 (1H, brs), 4.64 (2H, t, J = 8.7 Hz), 4.08-3.96 (3H, m), 3.89-3.84 (1H, m), 3.69-3.52 (2H, m), 3.46-3.33 (2H, m), 2.28-2.23 (1H, m), 1.80-1.65 (1H, m), 1.47 (9H, s). |
| 245 | 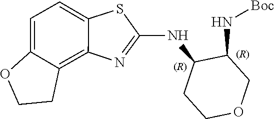 | ¹H-NMR (CDCl₃) δ: 7.28 (1H, dd, J = 0.8, 8.3 Hz), 6.62 (1H, d, J = 8.3 Hz), 6.00 (1H, brs), 5.24 (1H, brs), 4.64 (2H, t, J = 8.7 Hz), 4.11-3.94 (3H, m), 3.88-3.86 (1H, m), 3.70-3.52 (2H, m), 3.45-3.35 (2H, m), 2.28-2.23 (1H, m), 1.80-1.63 (1H, m), 1.47 (9H, s). |
| 246 | 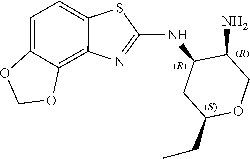 | LC-MS [M + H]⁺/Rt (min): 322.2/1.740 (Method C) |

TABLE 38-5

| | | |
|---|---|---|
| 247 | 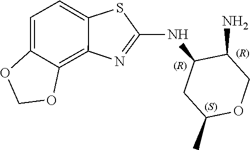 | LC-MS [M + H]⁺/Rt (min): 308.1/1.690 (Method C) |
| 248 | 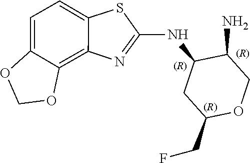 | LC-MS [M + H]⁺/Rt (min): 326.1/1.673 (Method C) |

### Reference Examples 249 to 268

The compounds of Reference Examples 249 to 268 were obtained by using the corresponding raw material compounds according to the method described in Reference Example 14.

TABLE 39-1

| Reference | | |
|---|---|---|
| Example | Chemical structure | Physical property data |
| 249 | 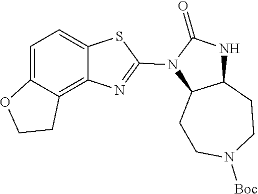 [SMILES: CC(C)(C)OC(=O)N1CC[C@@H]2NC(=O)N(c3nc4c5c(ccc4s3)OCC5)[C@@H]2CC1] | LC-MS [M + H]⁺/Rt (min): 431.2/1.097 (Method B) |
| 250 | 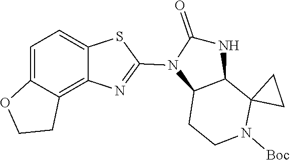 [SMILES: CC(C)(C)OC(=O)N1CC[C@@H]2[C@@H](NC(=O)N2c2nc3c4c(ccc3s2)OCC4)C12CC2] | LC-MS [M + H]⁺/Rt (min): 443.2/0.975 (Method B) |
| 251 | 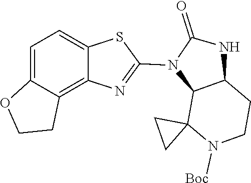 [SMILES: CC(C)(C)OC(=O)N1CC[C@@H]2NC(=O)N(c3nc4c5c(ccc4s3)OCC5)[C@@H]2C12CC2] | LC-MS [M + H]⁺/Rt (min): 443.2/0.904 (Method B) |
| 252 | 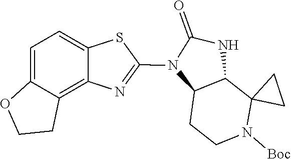 [SMILES: CC(C)(C)OC(=O)N1CC[C@@H]2[C@H](NC(=O)N2c2nc3c4c(ccc3s2)OCC4)C12CC2] | LC-MS [M + H]⁺/Rt (min): 443.3/1.089 (Method B) |
| 253 | 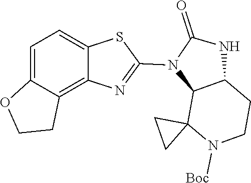 [SMILES: CC(C)(C)OC(=O)N1CC[C@H]2NC(=O)N(c3nc4c5c(ccc4s3)OCC5)[C@@H]2C12CC2] | LC-MS [M + H]⁺/Rt (min): 443.3/1.047 (Method B) |
| 254 | 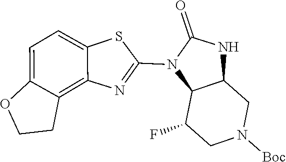 [SMILES: CC(C)(C)OC(=O)N1C[C@@H]2NC(=O)N(c3nc4c5c(ccc4s3)OCC5)[C@@H]2[C@@H](F)C1] | LC-MS [M + H]⁺/Rt (min): 435.2/0.912 (Method B) |
| 255 | 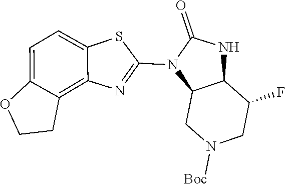 [SMILES: CC(C)(C)OC(=O)N1C[C@@H](F)[C@@H]2NC(=O)N(c3nc4c5c(ccc4s3)OCC5)[C@@H]2C1] | LC-MS [M + H]⁺/Rt (min): 435.2/0.899 (Method B) |
| 256 | 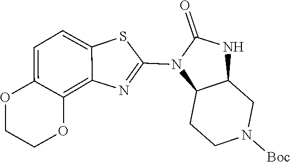 [SMILES: CC(C)(C)OC(=O)N1CC[C@@H]2[C@H](C1)NC(=O)N2c1nc2c3c(ccc2s1)OCCO3] | LC-MS [M + H]⁺/Rt (min): 433.2/0.854 (Method B) |
| 257 | 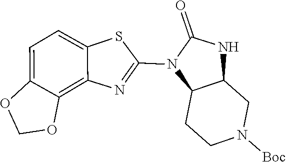 [SMILES: CC(C)(C)OC(=O)N1CC[C@@H]2[C@H](C1)NC(=O)N2c1nc2c3c(ccc2s1)OCO3] | LC-MS [M + H]⁺/Rt (min): 419.2/0.917 (Method B) |

TABLE 39-2

| | | |
|---|---|---|
| 258 | 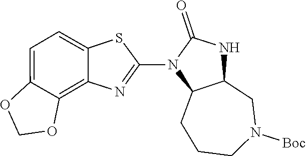 [SMILES: CC(C)(C)OC(=O)N1CCC[C@@H]2[C@H](C1)NC(=O)N2c1nc2c3c(ccc2s1)OCO3] | LC-MS [M + H]⁺/Rt (min): 433.3/1.000 (Method A |
| 259 | 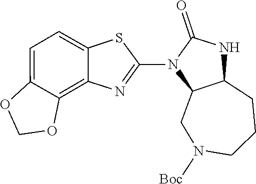 [SMILES: CC(C)(C)OC(=O)N1CCC[C@@H]2NC(=O)N(c3nc4c5c(ccc4s3)OCO5)[C@@H]2C1] | LC-MS [M + H]⁺/Rt (min): 433.3/0.915 (Method A) |
| 260 | 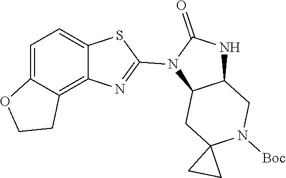 [SMILES: CC(C)(C)OC(=O)N1C[C@@H]2NC(=O)N(c3nc4c5c(ccc4s3)OCC5)[C@@H]2CC12CC2] | LC-MS [M + H]⁺/Rt (min): 443.3/0.976 (Method B) |
| 261 | 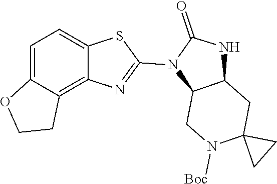 [SMILES: CC(C)(C)OC(=O)N1C[C@@H]2[C@H](CC13CC3)NC(=O)N2c1nc2c3c(ccc2s1)OCC3] | LC-MS [M + H]⁺/Rt (min): 443.3/0.999 (Method B) |
| 262 | 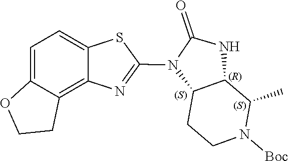 | LC-MS [M + H]⁺/Rt (min): 431.2/0.982 (Method B) |
| 263 | 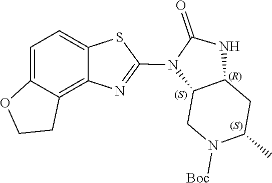 | LC-MS [M + H]⁺/Rt (min): 431.2/0.999 (Method B) |
| 264 | 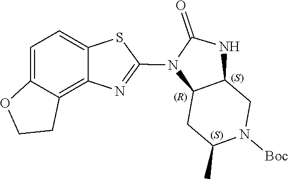 | LC-MS [M + H]⁺/Rt (min): 431.3/0.997 (Method B) |
| 265 | 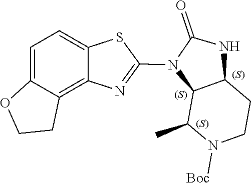 | LC-MS [M + H]⁺/Rt (min): 431.3/0.963 (Method B) |
| 266 | 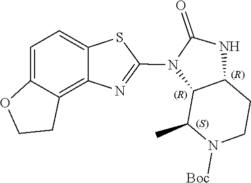 | LC-MS [M + H]⁺/Rt (min): 431.2/1.011 (Method B) |

TABLE 39-3

| | | |
|---|---|---|
| 267 | 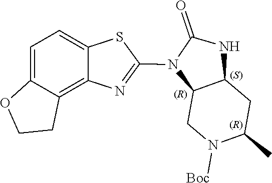 | LC-MS [M + H]⁺/Rt (min): 431.2/0.997 (Method B) |
| 268 | 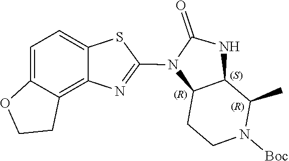 | LC-MS [M + H]⁺/Rt (min): 431.2/0.981 (Method B) |

### Reference Example 269

The compound of Reference Example 269 was obtained by using the corresponding raw material compounds according to the method described in Reference Example 28.

TABLE 40

| Reference | | |
|---|---|---|
| Example | Chemical structure | Physical property data |
| 269 | 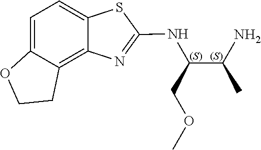 | LC-MS [M + H]⁺/Rt (min): 294.2/0.419 (Method B) |

### Reference Example 270

### tert-Butyl rac-(3aR,6aS)-3-(7,8-dihydrofuro[3,2-e][1,3]benzothiazol-2-yl)-2-oxohexahydro-1H-furo[3,4-d]imidazole-1-carboxylate

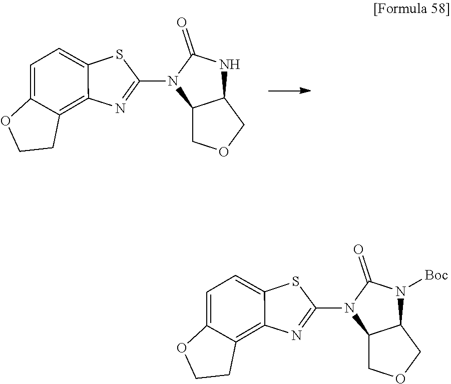 [SMILES: O=C1N[C@H]2COC[C@H]2N1c1nc2c3c(ccc2s1)OCC3 ; CC(C)(C)OC(=O)N1C(=O)N(c2nc3c4c(ccc3s2)OCC4)[C@@H]2COC[C@@H]21]

Sodium hydride (60% in oil, 130 mg) and (Boc)₂O (0.33 mL) were added to a tetrahydrofuran solution (10 mL) of the compound of Example 119 (380 mg) at 0° C., and the resulting mixture was stirred at room temperature for 16 hours. Water was added to the reaction mixture, and the resulting mixture was extracted with ethyl acetate. The organic layer was dried over sodium sulfate, filtered, and then concentrated under reduced pressure. The residue was purified by silica gel chromatography (petroleum ether/ethyl acetate) to obtain the title compound (465 mg).

LC-MS [M+H]⁺/Rt (min): 404.6/2.568 (Method D)

### Reference Example 271

### tert-Butyl (3aS,6aR)-3-(7,8-dihydrofuro[3,2-e][1,3]benzothiazol-2-yl)-2-oxohexahydro-1H-furo[3,4-d]imidazole-1-carboxylate

### Reference Example 272

### tert-Butyl (3aR,6aS)-3-(7,8-dihydrofuro[3,2-e][1,3]benzothiazol-2-yl)-2-oxohexahydro-1H-furo[3,4-d]imidazole-1-carboxylate

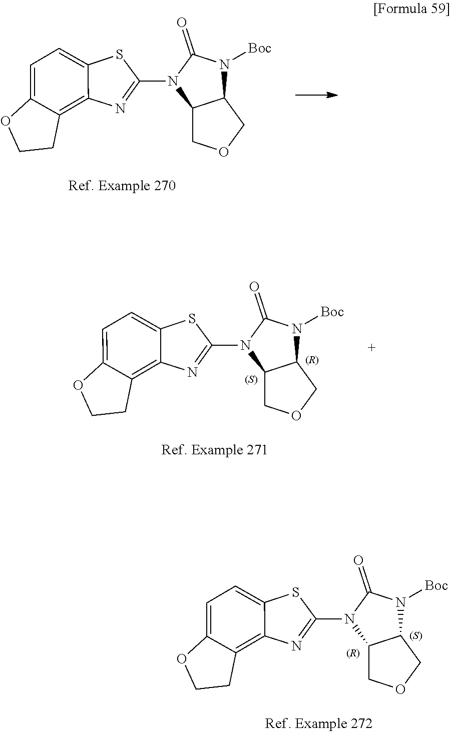

The compound of Reference Example 270 (465 mg) was optically fractionated under the following conditions of supercritical fluid chromatography to obtain the title compounds (Reference Example 271: 160 mg-first peak: 2.01 min, Reference Example 272: 160 mg-second peak: 4.03 min).

Column: CHIRALCEL® OJ-H; Solvents: liquid A: carbon dioxide, liquid B: methanol; Mobile phase condition: A/B (%)=55/45; Flow rate: 90 mL/min (during analysis: 3 mL/min); Detection UV: 214 nm; Column temperature: 30° C.

TABLE 41

| Reference | |
|---|---|
| Example | Physical property data |
| 271 | LC-MS [M + H]⁺/Rt (min): 404.5/2.542 (Method D) |
| 272 | LC-MS [M + H]⁺/Rt (min): 404.5/2.542 (Method D) |

### Reference Example 273

### tert-Butyl {(3R,4R)-4-[(7,8-dihydrofuro[3,2-e][1,3]benzothiazol-2-yl)amino)-6,6-dimethyloxan-3-yl}carbamate

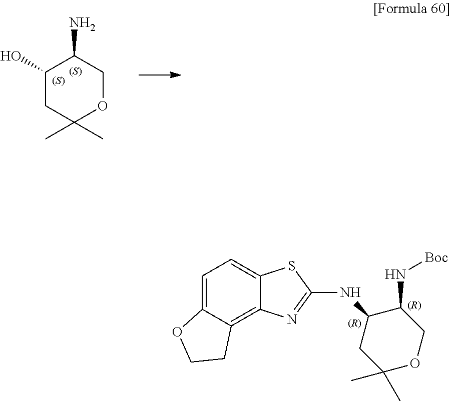

Triethylamine (0.065 mL) and (Boc)₂O (0.107 mL) were added to a dichloromethane solution (4 mL) of (4S,5S)-5-amino-2,2-dimethyltetrahydro-2H-pyran-4-ol) (56.0 mg) at room temperature, and the resulting mixture was stirred at room temperature overnight. The reaction mixture was concentrated under reduced pressure and then purified by silica gel chromatography (hexane/ethyl acetate) to obtain a Boc form (70.0 mg). Triethylamine (0.048 mL) and methanesulfonyl chloride (0.024 mL) were added to a dichloromethane solution (6 mL) of the obtained Boc form (70.0 mg) under ice cooling, and the resulting mixture was stirred at room temperature for 2 hours. Water was added to the reaction mixture, and the resulting mixture was extracted with ethyl acetate. The organic layer was dried over sodium sulfate, then filtered, and concentrated under reduced pressure to obtain a crude product (106 mg). Sodium azide (63.9 mg) and sodium acetate (53.8 mg) were added to a dimethylformamide solution (5 mL) of the obtained crude product (106 mg) at room temperature, and the resulting mixture was stirred at 80° C. for 3 days. Water was added to the reaction mixture, and the resulting mixture was extracted with ethyl acetate. The organic layer was washed with saturated brine, then dried over sodium sulfate, filtered, and concentrated under reduced pressure to obtain a crude product. A catalytic amount of 10% palladium carbon was added to a methanol solution (10 mL) of the obtained crude product at room temperature, and the resulting mixture was stirred at room temperature overnight in a normal pressure hydrogen atmosphere. The reaction mixture was filtered through cerite and then concentrated under reduced pressure to obtain a crude product (84.0 mg). 4-Isothiocyanato-2,3-dihydrobenzofuran (67.0 mg) was added to a tetrahydrofuran solution (3 mL) of the obtained crude product (84.0 mg) at room temperature, and the resulting mixture was stirred at room temperature overnight. The reaction mixture was concentrated under reduced pressure and then purified by silica gel chromatography (hexane/ethyl acetate) to obtain the title compound (18.0 mg).

LC-MS [M+H]⁺/Rt (min): 420.2/2.273 (Method C)

### Reference Example 274

The compound of Reference Example 274 was obtained by using the corresponding raw material compounds according to the method described in Reference Example 273.

TABLE 42

| Reference | | |
|---|---|---|
| Example | Chemical structure | Physical property data |
| 274 | 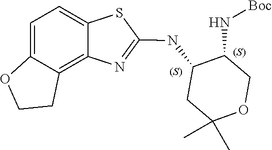 | LC-MS [M + H]⁺/Rt (min): 420.2/2.273 (Method C) |

### Reference Example 275

### 1,4-Anhydro-5-O-[tert-butyl(dimethyl)silyl]-2,3-bis-O-(trifluoromethanesulfonyl)-D-ribitol

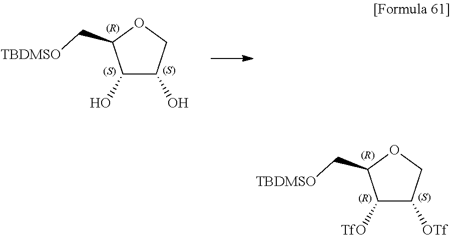

Pyridine (7.81 mL) and trifluoromethanesulfonic anhydride (8.16 mL) were added to a dichloromethane solution (322 mL) of (2R,3S,4S)-2-[{(tert-butyldimethylsilyl)oxy}methyl]tetrahydrofuran-3,4-diol (4.00 g) under ice cooling, and the resulting mixture was stirred under ice cooling for 1 hour. Ice water was added to the reaction mixture, and the resulting mixture was extracted with chloroform. The organic layer was dried over sodium hydrogen sulfate, filtered, and then concentrated under reduced pressure. The residue was purified by silica gel chromatography (hexane/ethyl acetate) to obtain the title compound (7.04 g).

1H-NMR (CDCl₃) δ: 5.47-5.36 (2H, s), 4.34-4.25 (2H, m), 4.19-4.00 (1H, m), 3.87-3.70 (2H, m), 0.90 (9H, s), 0.09 (3H, s), 0.08 (3H, s).

### Reference Example 276

### 2,5-Anhydro-1-O-[tert-butyl(dimethyl)silyl]-D-arabinitol

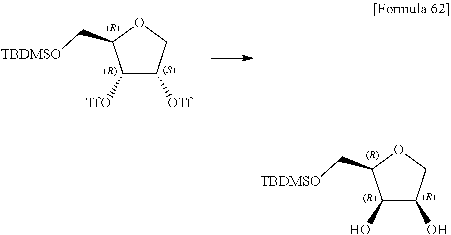

18-Crown-6 (7.82 g) and cesium acetate (7.91 g) were added to a dimethylformamide solution (92 mL) of the compound of Reference Example 275 (7.04 g) at room temperature, and the resulting mixture was stirred at 40° C. for 13 hours. Cesium acetate (7.91 g) was added, and the resulting mixture was further stirred at 40° C. for 3 hours. The reaction mixture was concentrated under reduced pressure, then ethyl acetate and water were added to the residue, and the resulting mixture was extracted with ethyl acetate. The organic layer was washed with saturated brine, then dried over sodium hydrogen sulfate, filtered, and concentrated under reduced pressure. The residue was purified by silica gel chromatography (hexane/ethyl acetate) to obtain a monoacetate form (2.78 g) as a mixture. The obtained monoacetate form (2.78 g) was dissolved in a 7 N ammonia methanol solution (70 mL), and the resulting solution was stirred at room temperature for 10 hours. The reaction mixture was concentrated under reduced pressure, and then the residue was purified by silica gel chromatography (hexane/ethyl acetate) to obtain the title compound (2.15 g).

1H-NMR (DMSO-d₆) δ: 4.80 (1H, d, J=6.1 Hz), 4.58 (1H, d, J=4.6 Hz), 4.17-4.07 (1H, m), 3.97-3.90 (1H, m), 3.82-3.74 (2H, m), 3.30 (1H, dd, J=6.7, 8.2 Hz), 3.66-3.57 (1H, m), 3.45 (1H, dd, J=6.8, 8.2 Hz), 0.86 (9H, s), 0.03 (3H, s), 0.03 (3H, s).

### Reference Example 277

The compound of Reference Example 277 was obtained by using the corresponding raw material compounds according to the method described in Reference Example 38.

TABLE 43

| Reference | | |
|---|---|---|
| Example | Chemical structure | Physical property data |
| 277 | 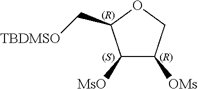 | ¹H-NMR (CDCl₃) δ: 5.32-5.22 (2H, m), 4.19-4.06 (2H, m), 3.97 (1H, dd, J = 6.8, 9.5 Hz), 3.90-3.76 (2H, m), 3.15 (6H, s), 0.89 (9H, s), 0.09 (3H, s), 0.08 (3H, s). |

### Reference Example 278

The compound of Reference Example 278 was obtained by using the corresponding raw material compounds according to the method described in Reference Example 42.

TABLE 44

| Reference | | |
|---|---|---|
| Example | Chemical structure | Physical property data |
| 278 | 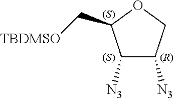 | ¹H-NMR (CDCl₃) δ: 4.19-4.04 (3H, m), 3.94-3.89 (1H, m), 3.85-3.71 (3H, m), 0.90 (9H, s), 0.09 (3H, s), 0.08 (3H, s). |

### Reference Example 279

The compound of Reference Example 279 was obtained by using the corresponding raw material compounds according to the method described in Reference Example 44.

TABLE 45

| Reference | | |
|---|---|---|
| Example | Chemical structure | Physical property data |
| 279 | 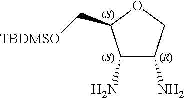 | LC-MS [M + H]⁺/Rt (min): 247.2/1.723 (Method C) |

### Reference Examples 280 and 281

The compounds of Reference Examples 280 and 281 were obtained by using the corresponding raw material compounds according to the method described in Reference Example 1 and Reference Example 2.

TABLE 46

| Reference | | |
|---|---|---|
| Example | Chemical structure | Physical property data |
| 280 | 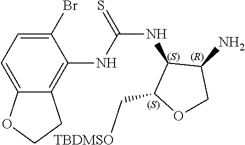 | LC-MS [M + 2H]⁺/Rt (min): 504.2/1.907 (Method C) |
| 281 | 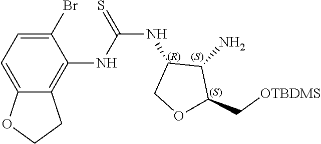 | LC-MS [M + 2H]⁺/Rt (min): 504.2/1.973 (Method C) |

### Reference Examples 282 and 283

The compounds of Reference Examples 282 and 283 were obtained by using the corresponding raw material compounds according to the method described in Reference Example 47.

TABLE 47

| Reference | | |
|---|---|---|
| Example | Chemical structure | Physical property data |
| 282 | 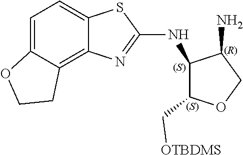 | LC-MS [M + H]⁺/Rt (min): 422.2/2.223 (Method C) |
| 283 | 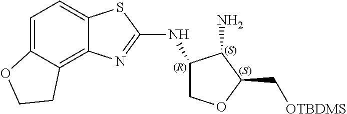 | LC-MS [M + H]⁺/Rt (min): 422.2/2.223 (Method C) |

### Reference Examples 284 and 285

The compounds of Reference Examples 284 and 285 were obtained by using the corresponding raw material compounds according to the method described in Reference Example 38.

TABLE 48

| Reference | | |
|---|---|---|
| Example | Chemical structure | Physical property data |
| 284 | 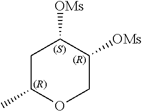 | ¹H-NMR (CDCl₃) δ: 4.96-4.91 (1H, m), 4.91-4.83 (1H, m), 4.24 (1H, dd, J = 2.3, 13.6 Hz), 3.62 (1H, dd, J = 1.0, 13.6 Hz), 3.59-3.51 (1H, m), 3.14 (3H, s), 3.12 (3H, s), 2.07-1.86 (2H, m), 1.29 (3H, d, J = 6.2 Hz). |
| 285 | 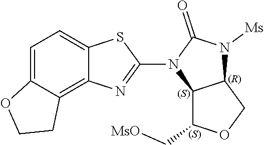 | LC-MS [M + H]⁺/Rt (min): 490.1/2.290 (Method C) |

### Reference Example 286

The compound of Reference Example 286 was obtained by using the corresponding raw material compounds according to the method described in Reference Example 42.

TABLE 49

| Refer- | | |
|---|---|---|
| ence | | |
| Example | Chemical structure | Physical property data |
| 286 | 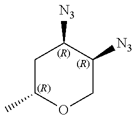 | ¹H-NMR (CDCl₃) δ: 4.08 (1H, dd, J = 2.8, 4.1 Hz), 3.84 (1H, ddd, J = 1.4, 4.6, 10.9 Hz), 3.77-3.65 (2H, m), 3.60 (1H, ddd, J = 3.2, 4.6, 10.7 Hz), 1.91 (1H, ddd, J = 2.1, 3.6, 14.3 Hz), 1.65-1.55 (1H, m), 1.17 (3H, d, J = 6.2 Hz). |

### Reference Example 287

The compound of Reference Example 287 was obtained by using the corresponding raw material compounds according to the method described in Reference Example 44.

TABLE 50

| Refer- | | |
|---|---|---|
| ence | | |
| Example | Chemical structure | Physical property data |
| 287 | 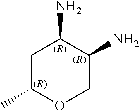 | ¹H-NMR (CDCl₃) δ: 3.84-3.72 (1H, m), 3.64-3.55 (1H, m), 3.45 (1H, dd, J = 10.5, 11.2 Hz), 3.25-3.22 (1H, m), 2.92 (1H, ddd, J = 3.7, 4.8, 10.5 Hz), 1.73-1.56 (6H, m), 1.16 (3H, d, J = 6.3 Hz). |

### Reference Example 288

The compound of Reference Example 288 was obtained by using the corresponding raw material compounds according to the method described in Reference Example 1 and Reference Example 2.

TABLE 51

| Reference | | |
|---|---|---|
| Example | Chemical structure | Physical property data |
| 288 | 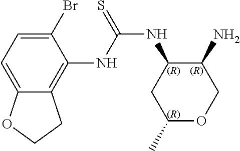 | LC-MS [M + 2H]⁺/Rt (min): 388.1/1.740 (Method C) |

### Reference Example 289

The compound of Reference Example 289 was obtained by using the corresponding raw material compounds according to the method described in Reference Example 47.

TABLE 52

| Reference | | |
|---|---|---|
| Example | Chemical structure | Physical property data |
| 289 | 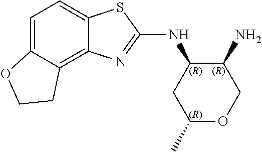 | LC-MS [M + H]⁺/Rt (min): 306.1/1.673 (Method C) |

### Reference Example 290

The compound of Reference Example 290 was obtained by using the corresponding raw material compounds according to the method described in Reference Example 77.

TABLE 53

| Reference | | |
|---|---|---|
| Example | Chemical structure | Physical property data |
| 290 | 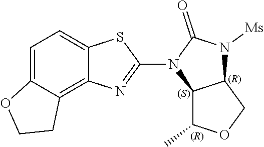 | LC-MS [M + H]⁺/Rt (min): 396.1/2.340 (Method C) |

### Example 1

### cis-3-(7,8-Dihydrofuro[3,2-e][1,3]benzothiazol-2-yl)octahydro-2H-imidazo[4,5-c]pyridin-2-one

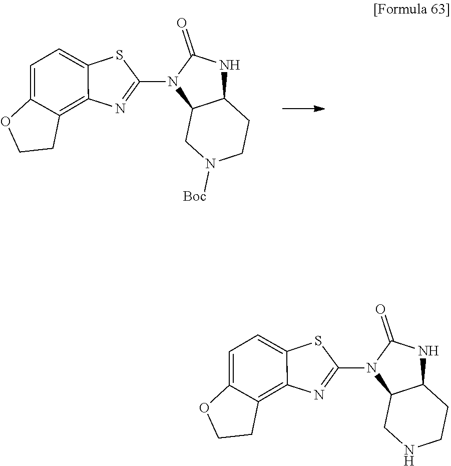 [SMILES: CC(C)(C)OC(=O)N1CC[C@@H]2NC(=O)N(c3nc4c5c(ccc4s3)OCC5)[C@@H]2C1 ; O=C1N[C@H]2CCNC[C@H]2N1c1nc2c3c(ccc2s1)OCC3]

Trifluoroacetic acid (3 mL) was added to the compound of Reference Example 14 (114 mg), and the resulting mixture was stirred for 11 minutes. The reaction mixture was concentrated under reduced pressure, and the residue was purified by amino silica gel column chromatography (chloroform/methanol) to obtain the title compound (62 mg).

LC-MS [M+H]⁺/Rt (min): 317.1/0.399 (Method B); 1H-NMR (DMSO-d₆) δ: 7.73 (1H, s), 7.57 (1H, d, J=8.5 Hz), 6.72 (1H, d, J=8.5 Hz), 4.64-4.56 (2H, m), 4.52-4.44 (1H, m), 4.04-3.99 (1H, m), 3.53-3.46 (1H, m), 3.39-3.33 (2H, m), 2.77-2.69 (1H, m), 2.62-2.51 (2H, m), 2.30-2.19 (1H, m), 1.81-1.69 (2H, m).

### Examples 2 to 6

The compounds of Examples 2 to 6 were obtained by using the corresponding raw material compounds according to the method described in Example 1.

TABLE 54

| Example | Chemical structure | Physical property data |
|---|---|---|
| 2 | 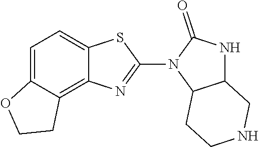 [SMILES: O=C1NC2CNCCC2N1c1nc2c3c(ccc2s1)OCC3] | LC-MS [M + H]⁺/Rt (min): 317.1/0.432 (Method B); ¹H-NMR (DMSO-d₆) δ: 7.73 (1H, s), 7.57 (1H, d, J = 7.9 Hz), 6.72 (1H, d, J = 7.9 Hz), 4.66-4.55 (3H, m), 4.11-4.05 (1H, m), 3.74-3.70 (1H, m), 3.35 (2H, t, J = 8.9 Hz), 2.98-2.92 (1H, m), 2.84-2.77 (2H, m), 2.50-2.41 (1H, m), 2.33-2.25 (1H, m), 1.58-1.47 (1H, m). |
| 3 | 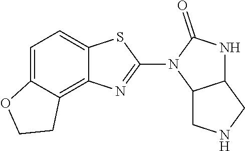 [SMILES: O=C1NC2CNCC2N1c1nc2c3c(ccc2s1)OCC3] | LC-MS [M + H]⁺/Rt (min): 303.1/0.533 (Method A); ¹H-NMR (400 MHz, DMSO-d₆) δ: 8.01 (1H, s), 7.57 (1H, d, J = 8.5 Hz), 6.72 (1H, d, J = 8.5 Hz), 5.36 (1H, brs), 4.94 (1H, dd, J = 4.9, 7.9 Hz), 4.60 (2H, t, J = 9.2 Hz), 4.28 (1H, dd, J = 5.2, 8.2 Hz), 3.42- 3.31 (5H, m), 2.99-2.93 (2H, m), 2.81 (1H, dd, J = 5.2, 12.5 Hz). |
| 4 | 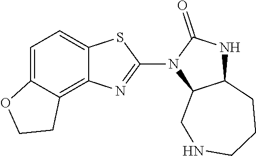 [SMILES: O=C1N[C@H]2CCCNC[C@H]2N1c1nc2c3c(ccc2s1)OCC3] | LC-MS [M + H]⁺/Rt (min): 331.2/0.454 (Method B); ¹H-NMR (CDCl₃, 50° C.) δ: 7.44 (1H, d, J = 8.6 Hz), 6.75 (1H, d, J = 8.6 Hz), 4.87- 4.77 (2H, m), 4.68-4.60 (2H, m), 4.29-4.21 (1H, m), 3.55-3.39 (4H, m), 2.91-2.84 (2H, m), 2.02-1.83 (3H, m), 1.62-1.51 (2H, m). |
| 5 | 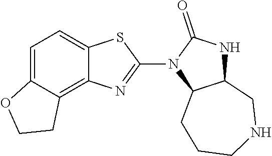 [SMILES: O=C1N[C@H]2CNCCC[C@H]2N1c1nc2c3c(ccc2s1)OCC3] | LC-MS [M + H]⁺/Rt (min): 331.2/0.523 (Method B); ¹H-NMR (CDCl₃, 50° C.) δ: 7.45 (1H, d, J = 8.6 Hz), 6.75 (1H, d, J = 8.6 Hz), 4.94- 4.88 (1H, m), 4.83 (1H, s), 4.70-4.60 (2H, m), 4.18-4.10 (1H, m), 3.45 (2H, t, J = 8.9 Hz), 3.15-3.08 (1H, m), 2.97-2.81 (3H, m), 2.42-2.27 (2H, m), 1.76-1.53 (3H, m). |
| 6 | 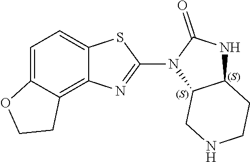 | LC-MS [M + H]⁺/Rt (min): 317.2/1.275 (Method D); ¹H-NMR (DMSO-d₆) δ: 7.94 (1H, s), 7.60 (1H, d, J = 8.3 Hz), 6.76 (1H, d, J = 8.3 Hz), 4.61 (2H, dt, J = 2.4, 8.7 Hz), 4.25 (1H, dd, J = 3.5, 11.8 Hz), 4.12 (1H, brs), 3.67-3.57 (1H, m), 3.40-3.34 (2H, m), 3.17 (1H, d, J = 2.8 Hz), 3.01 (1H, dd, J = 2.4, 13.4 Hz), 2.80-2.71 (1H, m), 2.62-2.53 (1H, m), 1.94-1.83 (1H, m), 1.59-1.49 (1H, m). |

### Example 7

### cis-3-(7,8-Dihydrofuro[3,2-e][1,3]benzothiazol-2-yl)-5-methyloctahydro-2H-imidazo[4,5-c]pyridin-2-one

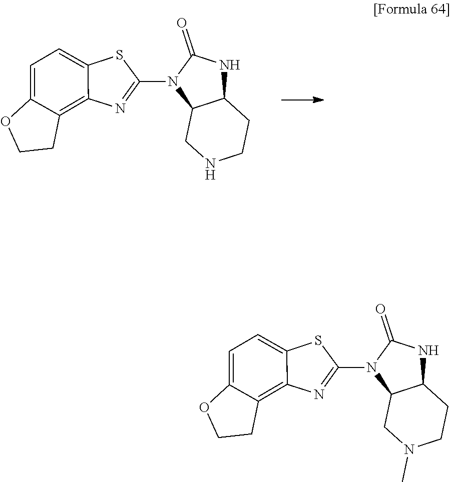 [SMILES: O=C1N[C@H]2CCNC[C@H]2N1c1nc2c3c(ccc2s1)OCC3 ; CN1CC[C@@H]2NC(=O)N(c3nc4c5c(ccc4s3)OCC5)[C@@H]2C1]

A 37% formaldehyde solution (0.02 mL) and methanol (1 mL) were added to a methanol (2 mL) solution of the compound of Example 1 (21.8 mg), and the resulting mixture was stirred for 10 minutes. Sodium triacetoxyborohydride (50 mg) was added, and the resulting mixture was stirred for 20 minutes. Saturated aqueous sodium bicarbonate was added to the reaction mixture, and the resulting mixture was extracted with chloroform. The organic layer was dried over sodium sulfate, filtered, and then concentrated under reduced pressure. The residue was purified by silica gel column chromatography (chloroform/methanol). Methanol was added to the resulting solid, the resulting mixture was stirred, and the solid was collected by filtration and then dried to obtain the title compound (12 mg).

LC-MS [M+H]⁺/Rt (min): 331.1/0.412 (Method B); 1H-NMR (DMSO-d₆) δ: 7.73 (1H, s), 7.58 (1H, d, J=7.9 Hz), 6.73 (1H, d, J=7.9 Hz), 4.69-4.56 (3H, m), 4.00-3.94 (1H, m), 3.43-3.27 (3H, m), 2.56-2.51 (1H, m), 2.16 (3H, s), 2.12-2.04 (1H, m), 1.96-1.75 (3H, m).

### Example 8

### cis-3-(7,8-Dihydrofuro[3,2-e][1,3]benzothiazol-2-yl)-5-(2-methoxyethyl)octahydro-2H-imidazo[4,5-c]pyridin-2-one

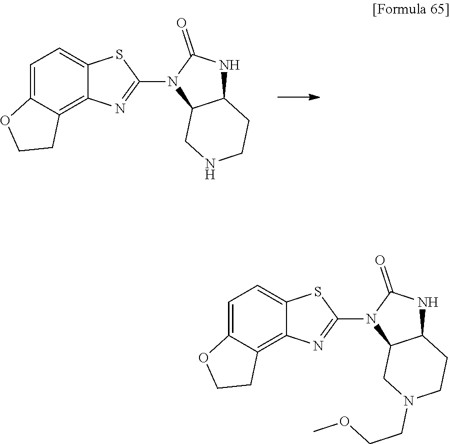 [SMILES: O=C1N[C@H]2CCNC[C@H]2N1c1nc2c3c(ccc2s1)OCC3 ; COCCN1CC[C@@H]2NC(=O)N(c3nc4c5c(ccc4s3)OCC5)[C@@H]2C1]

2-Chloroethylmethyl ether (0.036 mL), potassium iodide (66 mg), and potassium carbonate (111 mg) were added to a suspension of the compound of Example 1 (32 mg) in acetonitrile (1 mL), and the resulting mixture was stirred at 80° C. for 15 hours. Saturated aqueous sodium bicarbonate was added to the reaction mixture, and the resulting mixture was extracted with chloroform. The organic layer was dried over sodium sulfate, filtered, and then concentrated under reduced pressure. The residue was purified by reverse phase column chromatography (0.035% trifluoroacetic acid in acetonitrile/0.05% trifluoroacetic acid in water) and amino silica gel column chromatography (chloroform/methanol). Ethyl acetate was added to the resulting solid, the resulting mixture was stirred, and the solid was collected by filtration and then dried to obtain the title compound (19 mg).

LC-MS [M+H]⁺/Rt (min): 375.1/0.463 (Method B); 1H-NMR (CDCl₃) δ: 7.45 (1H, d, J=8.2 Hz), 6.76 (1H, d, J=8.2 Hz), 4.92-4.85 (1H, m), 4.77 (1H, s), 4.70-4.61 (2H, m), 4.09-4.05 (1H, m), 3.69-3.63 (1H, m), 3.51-3.38 (4H, m), 3.34 (3H, s), 2.82-2.76 (1H, m), 2.65-2.54 (2H, m), 2.34-2.26 (1H, m), 2.20-2.02 (2H, m), 1.91-1.87 (1H, m).

### Examples 9 to 19

The compounds of Examples 9 to 19 were obtained by using the corresponding raw material compounds according to the method described in Example 7 or Example 8.

TABLE 55-1

| Example | Chemical structure | Physical property data |
|---|---|---|
| 9 | 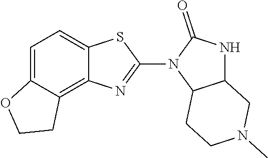 [SMILES: CN1CCC2C(C1)NC(=O)N2c1nc2c3c(ccc2s1)OCC3] | LC-MS [M + H]⁺/Rt (min): 331.1/0.432 (Method B); ¹H-NMR (DMSO-d₆) δ: 7.84 (1H, s), 7.57 (1H, d, J = 8.2 Hz), 6.72 (1H, d, J = 8.2 Hz), 4.64-4.49 (3H, m), 3.90-3.87 (1H, m), 3.35 (2H, t, J = 8.9 Hz), 2.79-2.73 (1H, m), 2.59-2.52 (1H, m), 2.41-2.24 (2H, m), 2.17 (3H, s), 2.03-1.95 (1H, m), 1.84-1.73 (1H, m). |
| 10 | 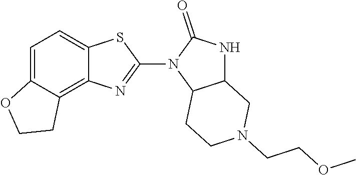 [SMILES: COCCN1CCC2C(C1)NC(=O)N2c1nc2c3c(ccc2s1)OCC3] | LC-MS [M + H]⁺/Rt (min): 375.1/0.444 (Method B); ¹H-NMR (DMSO-d₆) δ: 7.83 (1H, s), 7.57 (1H, d, J = 8.2 Hz), 6.72 (1H, d, J = 8.2 Hz), 4.64-4.51 (3H, m), 3.89-3.86 (1H, m), 3.42 (2H, t, J = 5.8 Hz), 3.35 (2H, t, J = 8.9 Hz), 3.23 (3H, s), 2.87-2.82 (1H, m), 2.69-2.63 (1H, m), 2.55- 2.49 (2H, m), 2.47-2.42 (1H, m), 2.37-2.32 (1H, m), 2.18-2.11 (1H, m), 1.87-1.77 (1H, m). |
| 11 | 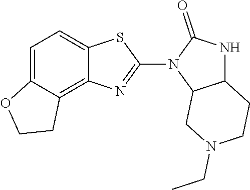 [SMILES: CCN1CCC2NC(=O)N(c3nc4c5c(ccc4s3)OCC5)C2C1] | LC-MS [M + H]⁺/Rt (min): 345.2/0.449 (Method B); ¹H-NMR (CDCl₃) δ: 7.47 (1H, d, J = 8.5 Hz), 6.78 (1H, d, J = 8.5 Hz), 4.92-4.82 (2H, m), 4.73- 4.64 (2H, m), 4.13-4.07 (1H, m), 3.66-3.60 (1H, m), 3.49-3.42 (2H, m), 2.79-2.71 (1H, m), 2.51-2.42 (2H, m), 2.31-2.23 (1H, m), 2.20- 2.01 (2H, m), 2.00-1.89 (1H, m), 1.08 (3H, t, J = 7.1 Hz). |
| 12 | 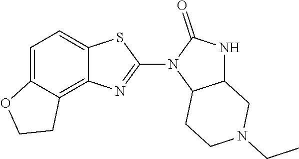 [SMILES: CCN1CCC2C(C1)NC(=O)N2c1nc2c3c(ccc2s1)OCC3] | LC-MS [M + H]⁺/Rt (min): 345.1/0.448 (Method B); ¹H-NMR (CDCl₃) δ: 7.47 (1H, d, J = 8.5 Hz), 6.77 (1H, d, J = 8.5 Hz), 5.36 (1H, s), 4.78-4.63 (3H, m), 4.04-3.98 (1H, m), 3.50- 3.42 (2H, m), 3.02-2.95 (1H, m), 2.81-2.74 (1H, m), 2.65-2.40 (4H, m), 2.18-1.99 (2H, m), 1.11 (3H, t, J = 7.1 Hz). |

TABLE 55-2

| | | |
|---|---|---|
| 13 | 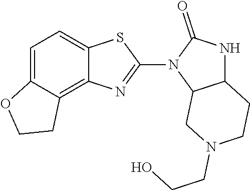 [SMILES: O=C1NC2CCN(CCO)CC2N1c1nc2c3c(ccc2s1)OCC3] | LC-MS [M + H⁺/Rt (min): 361.2/0.428 (Method B); ¹H-NMR (CDCl₃) δ: 7.47 (1H, d, J = 8.5 Hz), 6.79 (1H, d, J = 8.5 Hz), 4.87-4.82 (2H, m), 4.73-4.64 (2H, m), 4.13-4.09 (1H, m), 3.67-3.59 (3H, m), 3.53-3.45 (2H, m), 2.78-2.74 (1H, m), 2.65-2.54 (3H, m), 2.47-2.35 (2H, m), 2.12-2.03 (1H, m), 1.98-1.93 (1H, m). |
| 14 | 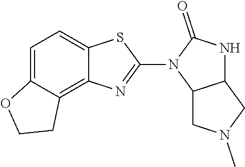 [SMILES: CN1CC2NC(=O)N(c3nc4c5c(ccc4s3)OCC5)C2C1] | LC-MS [M + H]⁺/Rt (min): 317.10/0.532 (Method A); ¹H-NMR (400 MHz, DMSO-d₆) δ: 8.03 (1H, s), 7.57 (1H, d, J = 8.5 Hz), 6.72 (1H, d, J = 8.5 Hz), 4.92 (1H, dd, J = 5.2, 8.2 Hz), 4.59 (2H, t, J = 8.5 Hz), 4.24 (1H, dd, J = 5.2, 8.2 Hz), 3.35 (2H, t, J = 8.5 Hz), 3.29-3.23 (1H, m), 2.82 (1H, d, J = 9.8 Hz), 2.33-2.27 (1H, m), 2.24 (3H, s), 2.18 (1H, dd, J = 4.9, 9.8 Hz). |
| 15 | 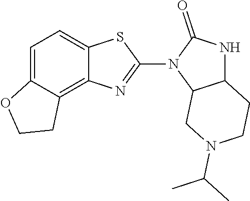 [SMILES: CC(C)N1CCC2NC(=O)N(c3nc4c5c(ccc4s3)OCC5)C2C1] | LC-MS [M + H]⁺/Rt (min): 359.1/0.491 (Method B); ¹H-NMR (CDCl₃) δ: 7.47 (1H, d, J = 8.2 Hz), 6.77 (1H, d, J = 8.2 Hz), 4.96 (1H, s), 4.88-4.80 (1H, m), 4.73-4.64 (2H, m), 4.12-4.08 (1H, m), 3.59-3.53 (1H, m), 3.48-3.42 (2H, m), 2.88-2.79 (1H, m), 2.69-2.65 (1H, m), 2.50-2.42 (1H, m), 2.39-2.32 (1H, m), 2.08-1.91 (2H, m), 1.04-0.98 (6H, m). |
| 16 | 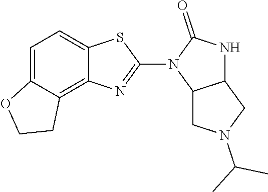 [SMILES: CC(C)N1CC2NC(=O)N(c3nc4c5c(ccc4s3)OCC5)C2C1] | LC-MS [M + H]⁺/Rt (min): 345.2/0.593 (Method A); ¹H-NMR (400 MHz, CDCl₃) δ: 7.40 (1H, d, J = 8.2 Hz), 6.71 (1H, d, J = 8.2 Hz), 5.14 (1H, s), 5.02-4.96 (1H, m), 4.61 (1H, t, J = 8.7 Hz), 4.28-4.22 (1H, m), 3.41 (2H, t, J = 8.7), 3.28 (1H, d, J = 10.1 Hz), 2.88 (1H, d, J = 10.1 Hz), 2.63 (1H, dd, J = 5.7, 10.1 Hz), 2.50-2.37 (2H, m), 1.01 (3H, d, J = 6.4 Hz), 0.99 (3H, d, J = 6.4 Hz). |

TABLE 55-3

| | | |
|---|---|---|
| 17 | 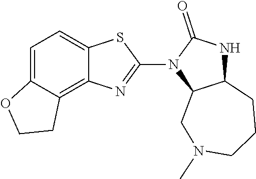 [SMILES: CN1CCC[C@@H]2NC(=O)N(c3nc4c5c(ccc4s3)OCC5)[C@@H]2C1] | LC-MS [M + H]⁺/Rt (min): 345.2/0.484 (Method B); ¹H-NMR (CDCl₃) δ: 7.45 (1H, d, J = 8.5 Hz), 6.75 (1H, d, J = 8.5 Hz), 5.02 (1H, s), 4.94-4.86 (1H, m), 4.71-4.60 (2H, m), 4.25-4.17 (1H, m), 3.51-3.28 (3H, m), 2.97-2.87 (1H, m), 2.79-2.71 (1H, m), 2.42-2.36 (1H, m), 2.39 (3H, s), 2.06-1.99 (1H, m), 1.92-1.76 (2H, m), 1.66-1.56 (1H, m). |
| 18 | 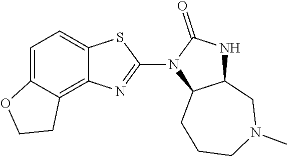 [SMILES: CN1CCC[C@@H]2[C@H](C1)NC(=O)N2c1nc2c3c(ccc2s1)OCC3] | LC-MS [M + H]⁺/Rt (min): 345.2/0.525 (Method B); ¹H-NMR (CDCl₃) δ: 7.45 (1H, d, J = 8.5 Hz), 6.75 (1H, d, J = 8.5 Hz), 5.05-4.99 (1H, m), 4.88-4.80 (1H, m), 4.69-4.59 (2H, m), 4.25-4.16 (1H, m), 3.50-3.40 (2H, m), 2.78-2.64 (3H, m), 2.58-2.48 (1H, m), 2.46-2.38 (1H, m), 2.41 (3H, s), 2.09-1.96 (1H, m), 1.82-1.76 (1H, m), 1.70-1.63 (1H, m). |
| 19 | 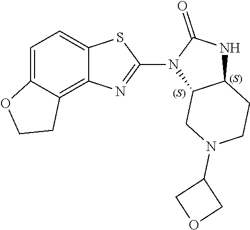 | LC-MS [M + H]⁺/Rt (min): 373.11/1.404 (Method D); ¹H-NMR (DMSO-d₆) δ: 8.01 (1H, s), 7.61 (1H, d, J = 8.3 Hz), 6.76 (1H, d, J = 8.3 Hz), 4.67-4.56 (4H, m), 4.55-4.45 (2H, m), 4.28-4.20 (1H, m), 3.82-3.68 (2H, m), 3.43-3.35 (3H, m), 2.85 (1H, d, J = 10.8 Hz), 2.25 (1H, t, J = 10.0 Hz), 2.16-2.08 (1H, m), 2.00-1.93 (1H, m), 1.75-1.62 (1H, m). |

### Example 20

### cis-5-Acetyl-3-(7,8-dihydrofuro[3,2-e][1,3]benzothiazol-2-yl) octahydro-2H-imidazo[4,5-c]pyridin-2-one

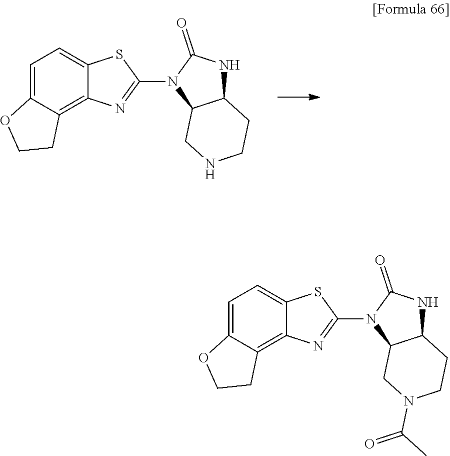 [SMILES: O=C1N[C@H]2CCNC[C@H]2N1c1nc2c3c(ccc2s1)OCC3 ; CC(=O)N1CC[C@@H]2NC(=O)N(c3nc4c5c(ccc4s3)OCC5)[C@@H]2C1]

Triethylamine (0.028 mL) and acetic anhydride (0.011 mL) were added to a chloroform solution (2 mL) of the compound of Example 1 (32 mg), and the resulting mixture was stirred overnight. Saturated aqueous sodium bicarbonate was added to the reaction mixture, and the resulting mixture was extracted with chloroform/ethanol (3/1). The organic layer was dried over sodium sulfate, filtered, and then concentrated under reduced pressure. The residue was purified by reverse phase column chromatography (0.035% trifluoroacetic acid in acetonitrile/0.05% trifluoroacetic acid in water), amino silica gel column chromatography (chloroform/methanol), and silica gel column chromatography (chloroform/methanol) to obtain the title compound (27 mg).

LC-MS [M+H]⁺/Rt (min): 359.1/0.566 (Method B); 1H-NMR (DMSO-d₆, 100° C.) δ: 7.59 (1H, br s), 7.54 (1H, d, J=7.9 Hz), 6.72 (1H, d, J=7.9 Hz), 4.83-4.78 (1H, m), 4.62 (2H, t, J=8.8 Hz), 4.30-4.17 (2H, m), 3.85-3.78 (1H, m), 3.52-3.41 (2H, m), 3.39 (2H, t, J=8.8 Hz), 2.13-2.02 (1H, m), 1.94-1.68 (4H, m).

### Example 21

### (3aR,7aR)-1-(2H-[1,3]dioxolo[4,5-e][1,3]benzothiazol-7-yl)hexahydropyrano[3,4-d]imidazol-2(3H)-one

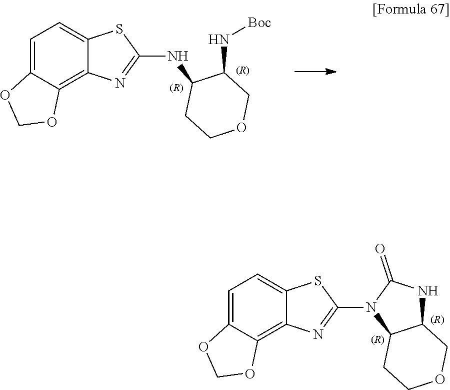

A 4 M hydrogen chloride/ethyl acetate solution (5 mL) was added to an ethyl acetate solution (5 mL) of the compound of Reference Example 47 (759 mg), and the resulting mixture was stirred at room temperature overnight. Hexane was added to the reaction mixture, the resulting solid was collected by filtration and washed with hexane, and then the solid was dried under reduced pressure. Triethylamine (1.41 mL) and di(N-succinimidyl) carbonate (519 mg) were added to a dimethylformamide solution (20 mL) of the resulting solid (668 mg), and the resulting mixture was stirred at room temperature for 1 hour. Water was added to the reaction mixture, and the resulting mixture was extracted with ethyl acetate. The organic layer was dried over sodium sulfate, filtered, and then concentrated under reduced pressure to obtain the title compound (364 mg).

LC-MS [M+H]⁺/Rt (min): 320.0/1.990 (Method C); ¹H-NMR (DMSO-d₆) δ: 7.96 (1H, s), 7.37 (1H, d, J=8.2 Hz), 6.93 (1H, d, J=8.2 Hz), 6.10 (2H, dd, J=1.1, 5.0 Hz), 4.78 (1H, ddd, J=6.1, 7.3, 8.6 Hz), 3.90-3.72 (3H, m), 3.67 (1H, dd, J=2.7, 12.8 Hz), 3.44 (1H, ddd, J=3.0, 10.2, 11.5), 2.46-2.28 (1H, m), 1.85-1.68 (1H, m).

### Example 22

### (4R,5R)-1-(7,8-dihydrofuro[3,2-e][1,3]benzothiazol-2-yl)-5-[(dimethylamino)methyl]-4-methylimidazolidin-2-one

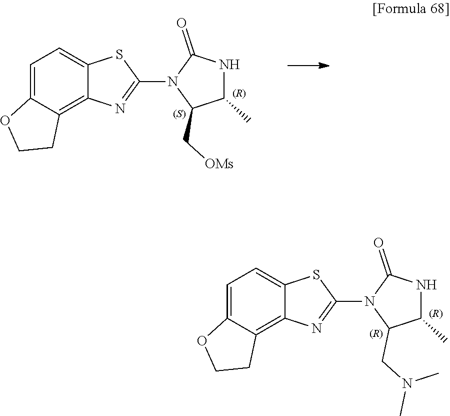

Dimethylamine (2.0 M tetrahydrofuran solution) (0.31 mL), potassium iodide (26.2 mg), and potassium carbonate (55.6 mg) were added to an acetonitrile solution (3 mL) of the compound of Reference Example 39 (59.4 mg), and the resulting mixture was stirred at 60° C. for 15 hours. The reaction mixture was cooled to room temperature, then saturated aqueous sodium bicarbonate was added, and the resulting mixture was extracted with chloroform-methanol (10:1). The organic layer was dried over sodium sulfate, filtered, and then concentrated under reduced pressure. The residue was purified by silica gel column chromatography (chloroform/methanol) and amino silica gel column chromatography (chloroform/methanol) to obtain the title compound (22.6 mg).

LC-MS [M+H]⁺/Rt (min): 333.2/0.569 (Method A); ¹H-NMR (400 MHz, CDCl₃) δ: 7.45 (1H, d, J=7.9 Hz), 6.76 (1H, d, J=7.9 Hz), 4.93 (1H, s), 4.66 (2H, t, J=8.9 Hz), 4.40-4.34 (1H, m), 3.90-3.84 (1H, m), 3.44 (2H, dt, J=2.0, 8.9 Hz), 2.87 (1H, dd, J=2.4, 11.9 Hz), 2.34 (1H, dd, J=10.1, 11.9 Hz), 2.34 (6H, s), 1.35 (3H, d, J=7.3 Hz).

### Examples 23 to 26

The compounds of Examples 23 to 26 were obtained by using the corresponding raw material compounds according to the method described in Example 22.

TABLE 56

| Example | Chemical structure | Physical property data |
|---|---|---|
| 23 | 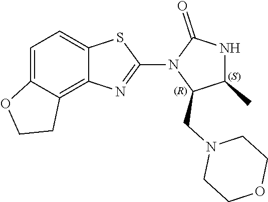 | LC-MS [M + H]⁺/Rt (min): 375.2/0.446 (Method B); ¹H-NMR (CDCl₃) δ: 7.47 (1H, d, J = 8.2 Hz), 6.78 (1H, d, J = 8.2 Hz), 4.93 (1H, s), 4.79-4.73 (1H, m), 4.68 (2H, t, J = 8.9 Hz), 4.29-4.22 (1H, m), 3.73-3.64 (4H, m), 3.50- 3.36 (2H, m), 2.84-2.72 (4H, m), 2.53-2.48 (2H, m), 1.45 (3H, d, J = 6.7 Hz). |
| 24 | 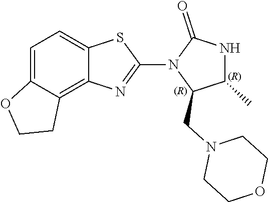 | LC-MS [M + H]⁺/Rt (min): 375.2/0.573 (Method A); ¹H-NMR (400 MHz, CDCl₃) δ: 7.45 (1H, d, J = 8.7 Hz), 6.76 (1H, d, J = 8.7 Hz), 4.93 (1H, s), 4.67 (2H, t, J = 8.7 Hz), 4.42-4.37 (1H, m), 3.91-3.85 (1H, m), 3.75-3.63 (4H, m), 3.43 (2H, t, J = 8.7 Hz), 3.05 (1H, dd, J = 3.2, 12.3 Hz), 2.73-2.66 (2H, m), 2.55 (1H, dd, J = 9.6, 12.3 Hz), 2.51-2.44 (2H, m), 1.35 (3H, d, J = 6.4 Hz). |
| 25 | 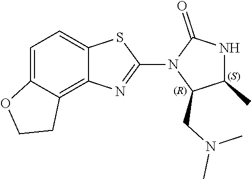 | LC-MS [M + H]⁺/Rt (min): 333.1/0.462 (Method B); ¹H-NMR (CDCl₃) δ: 7.47 (1H, d, J = 8.5 Hz), 6.77 (1H, d, J = 8.5 Hz), 4.88 (1H, s), 4.75-4.65 (3H, m), 4.28-4.21 (1H, m), 3.51- 3.37 (2H, m), 2.79 (1H, dd, J = 12.8, 9.8 Hz), 2.63 (1H, dd, J = 12.8, 1.8 Hz), 2.37 (6H, s), 1.45 (3H, d, J = 6.7 Hz). |
| 26 | 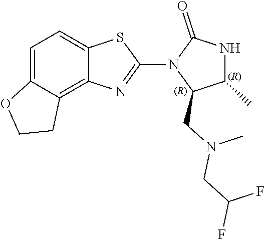 | LC-MS [M + H]⁺/Rt (min): 333.1/0.462 (Method B); ¹H-NMR (CDCl₃) δ: 6.95 (1H, d, J = 7.9 Hz), 6.43 (1H, d, J = 7.9 Hz), 5.79 (1H, tt, J = 4.3, 56.2 (J_{H-F}) Hz), 4.69 (1H, d, J = 9.2 Hz), 4.64-4.56 (3H, m), 4.15-4.08 (1H, m), 4.05 (t, J = 8.5 Hz), 3.79 (t, J = 8.5 Hz), 3.64-3.45 (2H, m), 3.36-3.21 (2H, m), 2.85 (3H, s), 1.36 (3H, d, J = 6.7 Hz). |

### Example 27

### (3aS,7aR)-3-(7,8-Dihydrofuro[3,2-e][1,3]benzothiazol-2-yl)-5-methyloctahydro-2H-imidazo[4,5-c]pyridin-2-one

### Example 28

### (3aR,7aS)-3-(7,8-Dihydrofuro[3,2-e][1,3]benzothiazol-2-yl)-5-methyloctahydro-2H-imidazo[4,5-c]pyridin-2-one

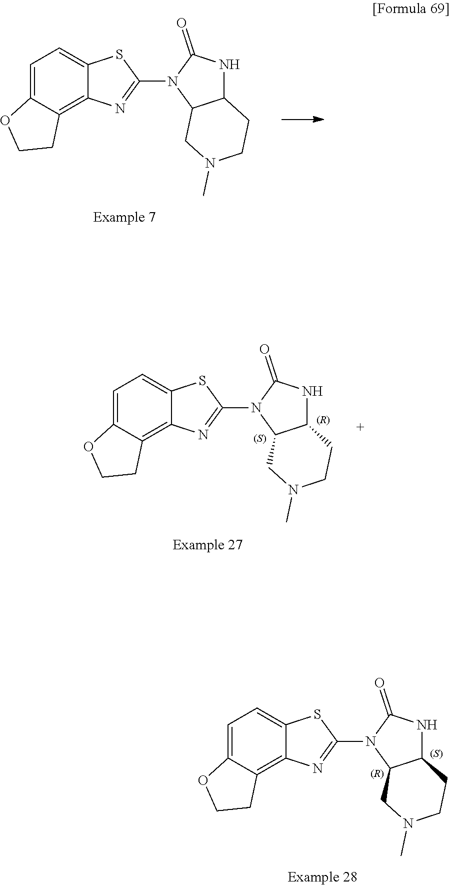

The compound of Example 7 (30.0 mg) was optically fractionated under the following conditions to obtain the title compounds (Example 27: 12.2 mg-first peak: 15.3 m, Example 28: 8.1 mg-second peak: 16.4 min).

Column: CHIRALPAK® IG; Solvents: liquid A: chloroform, liquid B: 2-propanol; Mobile phase condition: A/B=9/1; Flow rate: 5 mL/min; Detection UV: 254 nm; Column temperature: 40° C.

TABLE 57

| Example | Physical property data |
|---|---|
| 27 | LC-MS [M + H]⁺/Rt (min): 331.1/0.434 (Method B); ¹H-NMR (CDCl₃) δ: 7.45 |
| | (1H, d, J = 8.5 Hz), 6.76 (1H, d, J = 8.5 Hz), 4.89-4.82 (1H, m) , 4.74 |
| | (1H, s), 4.69-4.63 (2H, m), 4.08-4.03 (1H, m), 3.58-3.51 (1H, m), 3.44 |
| | (2H, t, J = 8.9 Hz), 2.66-2.60 (1H, m), 2.29 (3H, s), 2.27-2.19 (1H, |
| | m), 2.12-2.02 (2H, m), 1.94-1.88 (1H, m). |
| 28 | LC-MS [M + H]⁺/Rt (min): 331.1/0.430 (Method B); ¹H-NMR (CDCl₃) δ: 7.45 |
| | (1H, d, J = 8.5 Hz), 6.76 (1H, d, J = 8.5 Hz), 4.90-4.82 (1H, m), 4.76 |
| | (1H, s), 4.69-4.63 (2H, m), 4.09-4.03 (1H, m), 3.58-3.52 (1H, m), 3.44 |
| | (2H, t, J = 8.5 Hz), 2.67-2.60 (1H, m), 2.30 (3H, s), 2.29-2.20 (1H, |
| | m), 2.11-2.04 (2H, m), 1.94-1.88 (1H, m). |

### Examples 29 and 30

The compounds of Examples 29 and 30 were obtained by using the corresponding raw material compounds according to the method described in Example 1.

TABLE 58

| Example | Chemical structure | Physical property data |
|---|---|---|
| 29 | 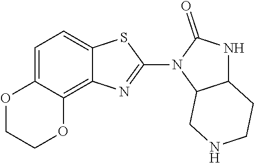 [SMILES: O=C1NC2CCNCC2N1c1nc2c3c(ccc2s1)OCCO3] | LC-MS [M + H]⁺/Rt (min): 333.1/0.430 (Method B); ¹H-NMR (DMSO-d₆) δ: 7.71 (1H, s), 7.26 (1H, d, J = 8.6 Hz), 6.78 (1H, d, J = 8.6 Hz), 4.48-4.40 (1H, m), 4.38-4.24 (4H, m), 4.03-3.98 (1H, m), 3.49-3.42 (1H, m), 2.76-2.69 (1H, m), 2.62-2.48 (2H, m), 2.36-2.15 (1H, m), 1.83-1.66 (2H, m). |
| 30 | 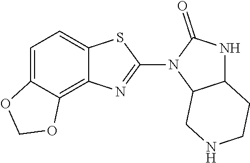 [SMILES: O=C1NC2CCNCC2N1c1nc2c3c(ccc2s1)OCO3] | LC-MS [M + H]⁺/Rt (min): 319.1/0.419 (Method B); ¹H-NMR (DMSO-d₆) δ: 7.79 (1H, s), 7.34 (1H, d, J = 7.9 Hz), 6.90 (1H, d, J = 7.9 Hz), 6.10-6.07 (2H, m), 4.50-4.43 (1H, m), 4.04-4.01 (1H, m), 3.46-3.40 (1H, m), 2.75-2.72 (1H, m), 2.66-2.53 (2H, m), 2.43-2.21 (1H, m), 1.82-1.67 (2H, m). |

### Example 31

### cis-5-Cyclopropyl-3-(7,8-dihydro[1,4]dioxino[2,3-e][1,3]benzothiazol-2-yl)octahydro-2H-imidazo[4,5-c]pyridin-2-one

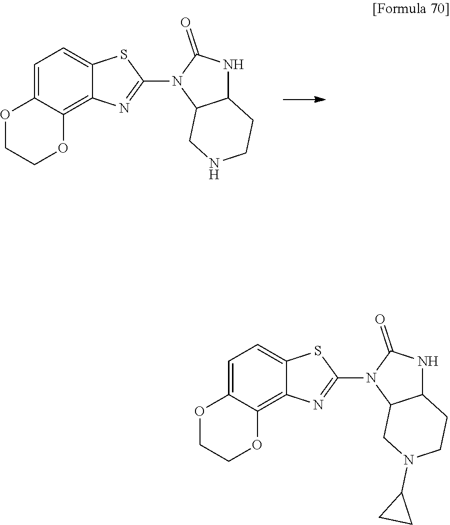 [SMILES: O=C1NC2CCNCC2N1c1nc2c3c(ccc2s1)OCCO3 ; O=C1NC2CCN(C3CC3)CC2N1c1nc2c3c(ccc2s1)OCCO3]

(1-Ethoxycyclopropoxy)trimethylsilane (0.121 mL), acetic acid (0.172 mL), and sodium cyanoborohydride (94 mg) were added to a methanol/tetrahydrofuran solution (1.5 mL/1.2 mL) of the compound of Example 29 (100 mg), and the resulting mixture was stirred at 60° C. for 3 hours. The reaction mixture was cooled to room temperature, then saturated aqueous sodium bicarbonate was added, and the resulting mixture was extracted with chloroform/ethanol (3/1). The organic layer was dried over sodium sulfate, filtered, and then concentrated under reduced pressure. The residue was purified by silica gel column chromatography (chloroform/methanol) to obtain the title compound (77 mg).

LC-MS [M+H]⁺/Rt (min): 373.3/0.585 (Method A); 1H-NMR (DMSO-d₆) δ: 7.72 (1H, s), 7.27 (1H, d, J=8.5 Hz), 6.79 (1H, d, J=8.5 Hz), 4.61-4.53 (1H, m), 4.41-4.25 (4H, m), 3.98-3.92 (1H, m), 3.49-3.41 (1H, m), 2.75-2.67 (1H, m), 2.45-2.36 (1H, m), 2.35-2.27 (1H, m), 1.86-1.75 (2H, m), 1.68-1.63 (1H, m), 0.43-0.37 (2H, m), 0.33-0.23 (2H, m).

### Example 32

The compound of Example 32 was obtained by using the corresponding raw material compounds according to the method described in Example 31.

TABLE 59

| Example | Chemical structure | Physical property data |
|---|---|---|
| 32 | 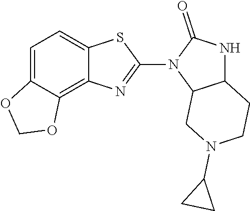 [SMILES: O=C1NC2CCN(C3CC3)CC2N1c1nc2c3c(ccc2s1)OCO3] | LC-MS [M + H]⁺/Rt (min): 359.2/0.613 (Method A); ¹H-NMR (DMSO-d₆) δ: 7.80 (1H, s), 7.35 (1H, d, J = 8.2 Hz), 6.91 (1H, d, J = 8.2 Hz), 6.12-6.08 (2H, m), 4.62-4.53 (1H, m), 4.00-3.94 (1H, m), 3.51-3.42 (1H, m), 2.76-2.67 (1H, m), 2.46-2.36 (1H, m), 2.35-2.27 (1H, m), 1.87-1.74 (2H, m), 1.69-1.64 (1H, m), 0.43-0.38 (2H, m), 0.33-0.23 (2H, m). |

### Example 33

### (3aS,7aR)-5-Cyclopropyl-3-(7,8-dihydro[1,4]dioxino[2,3-e][1,3]benzothiazol-2-yl)octahydro-2H-imidazo[4,5-c]pyridin-2-one

### Example 34

### (3aR,7aS)-5-Cyclopropyl-3-(7,8-dihydro[1,4]dioxino[2,3-e][1,3]benzothiazol-2-yl)octahydro-2H-imidazo[4,5-c]pyridin-2-one

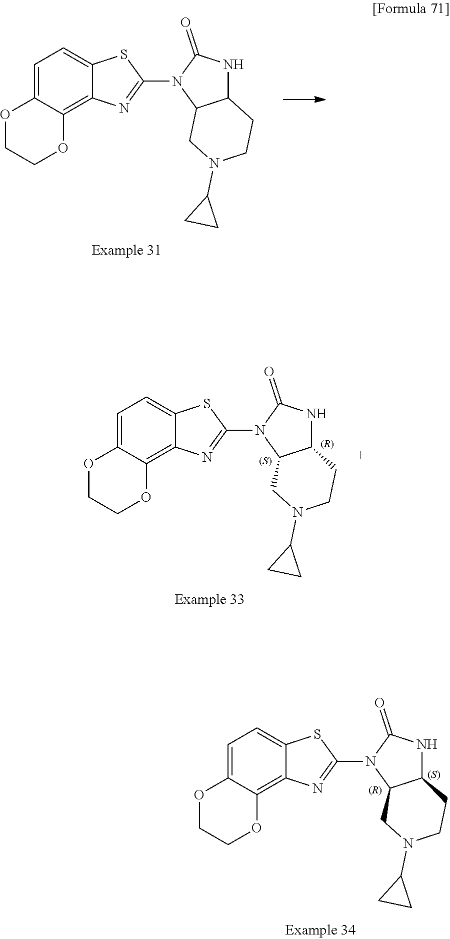

The compound of Example 31 (104 mg) was optically fractionated under the following conditions to obtain the title compounds (Example 33: 37 mg-first peak: 13.0 min, Example 34: 36 mg-second peak: 14.9 min).

Column: CHIRALPAK® IG; Solvents: liquid A: chloroform, liquid B: methanol, liquid C: diethylamine; Mobile phase condition: A/B/C=99/1/0.001; Flow rate: 0.5 mL/min; Detection UV: 280 nm; Column temperature: 25° C.

TABLE 60

| Example | Physical property data |
|---|---|
| 33 | LC-MS [M + H]⁺/Rt (min): 373.3/0.587 (Method A); ¹H-NMR (DMSO-d₆) δ: |
| | 7.72 (1H, s), 7.27 (1H, d, J = 8.5 Hz), 6.79 (1H, d, J = 8.5 Hz), |
| | 4.60-4.52 (1H, m), 4.41-4.28 (4H, m), 3.97-3.93 (1H, m), 3.47-3.41 |
| | (1H, m), 2.75-2.67 (1H, m), 2.45-2.37 (1H, m), 2.33-2.28 (1H, m), |
| | 1.86-1.75 (2H, m), 1.68-1.63 (1H, m), 0.43-0.33 (2H, m), 0.32-0.22 |
| | (2H, m). |
| 34 | LC-MS [M + H]⁺/Rt (min): 373.2/0.585 (Method A); ¹H-NMR (DMSO-d₆) δ: |
| | 7.73 (1H, s), 7.28 (1H, d, J = 8.8 Hz), 6.79 (1H, d, J = 8.8 Hz), |
| | 4.60-4.53 (1H, m) , 4.41-4.25 (4H, m), 3.97-3.93 (1H, m), 3.48-3.41 |
| | (1H, m), 2.72-2.60 (1H, m), 2.45-2.36 (1H, m), 2.33-2.28 (1H, m), |
| | 1.86-1.75 (2H, m), 1.68-1.63 (1H, m), 0.45-0.36 (2H, m), 0.31-0.22 |
| | (2H, m). |

### Example 35

### (3aS,7aR)-5-Cyclopropyl-3-(2H-[1,3]dioxolo[4,5-e][1,3]benzothiazol-7-yl)octahydro-2H-imidazo[4,5-c]pyridin-2-one

### Example 36

### (3aR,7aS)-5-Cyclopropyl-3-(2H-[1,3]dioxolo[4,5-e][1,3]benzothiazol-7-yl)octahydro-2H-imidazo[4,5-c]pyridin-2-on e

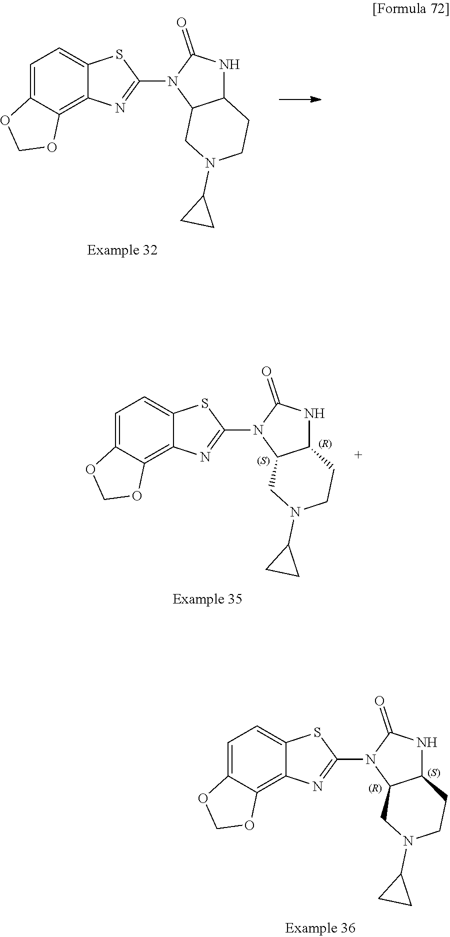

The compound of Example 32 (85.0 mg) was optically fractionated under the following conditions to obtain the title compounds (Example 35: 37.1 mg-first peak: 10.7 min, Example 36: 36.4 mg-second peak: 12.3 min).

Column: CHIRALPAK® IG; Solvents: liquid A: chloroform, liquid B: methanol, liquid C: diethylamine; Mobile phase condition: A/B/C=99/1/0.002; Flow rate: 5 mL/min; Detection UV: 280 nm; Column temperature: 40° C.

TABLE 61

| Example O | Physical property data |
|---|---|
| 35 | LC-MS [M + H]⁺/Rt (min): 359.2/0.657 (Method A); 1H-NMR (DMSO-d₆) δ: |
| | 7.80 (1H, s), 7.35 (1H, d, J = 7.9 Hz), 6.91 (1H, d, J = 7.9 Hz), |
| | 6.12-6.09 (2H, m), 4.61-4.53 (1H, m), 3.98-3.95 (1H, m), 3.50-3.43 |
| | (1H, m), 2.75-2.68 (1H, m), 2.45-2.37 (1H, m), 2.35-2.27 (1H, m) , |
| | 1.86-1.75 (2H, m), 1.69-1.64 (1H, m), 0.45-0.37 (2H, m), 0.33-0.23 |
| | (2H, m). |
| 36 | LC-MS [M+H]⁺/Rt (min): 359.3/0.660 (Method A); 1H-NMR (DMSO-d₆) δ: |
| | 7.81 (1H, s), 7.35 (1H, d, J = 8.3 Hz), 6.91 (1H, d, J = 8.3 Hz), 6.10 |
| | (2H, d, J = 6.1 Hz), 4.61-4.53 (1H, m), 3.99-3.94 (1H, m), 3.50-3.42 |
| | (1H, m), 2.75-2.67 (1H, m), 2.44-2.36 (1H, m), 2.34-2.27 (1H, m), |
| | 1.86-1.75 (2H, m), 1.69-1.64 (1H, m), 0.44-0.37 (2H, m), 0.33-0.23 |
| | (2H, m). |

### Examples 37 to 43

The compounds of Examples 37 to 43 were obtained by using the corresponding raw material compounds according to the method described in Reference Example 14.

TABLE 62-1

| Example | Chemical structure | Physical property data |
|---|---|---|
| 37 | 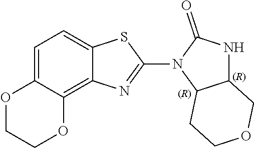 | LC-MS [M + H]⁺/Rt (min): 334.1/1.939 (Method C); ¹H-NMR (DMSO-d₆) δ: 7.88 (1H, s), 7.29 (1H, d, J = 8.6 Hz), 6.80 (1H, d, J = 8.6 Hz), 4.79- 4.73 (1H, m), 4.42-4.25 (4H, m), 3.87-3.71 (3H, m), 3.67 (1H, dd, J = 3.0, 13.0 Hz), 3.44 (1H, ddd, J = 3.0, 10.2, 11.5 Hz), 2.41-2.29 (1H, m), 1.87-1.72 (1H, m). |
| 38 | 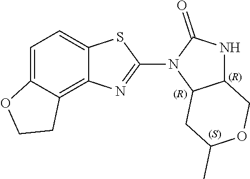 | LC-MS [M + H]⁺/Rt (min): 332.1/2.090 (Method C); ¹H-NMR (DMSO-d₆) δ: 7.89 (1H, s), 7.59 (1H, d, J = 8.4 Hz), 6.74 (1H, d, J = 8.4 Hz), 4.85- 4.79 (1H, m), 4.67-4.49 (2H, m), 3.95 (1H, d, J = 13.1 Hz), 3.77 (1H, dd, J = 2.4, 7.1 Hz), 3.67 (1H, dd, J = 2.7, 13.2 Hz), 3.53-3.41 (1H, m), 3.41-3.31 (2H, m), 2.49-2.38 (1H, m), 1.19-1.13 (1H, m), 1.10 (3H, d, J = 6.1 Hz). |
| 39 | 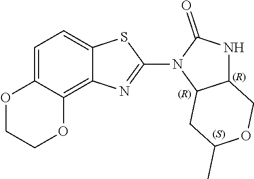 | LC-MS [M + H]⁺/Rt (min): 348.1/2.040 (Method C); ¹H-NMR (DMSO-d₆) δ: 7.85 (1H, s), 7.29 (1H, d, J = 8.6 Hz), 6.80 (1H, d, J = 8.6 Hz), 4.81- 4.75 (1H, m), 4.43-4.20 (4H, m), 3.95 (1H, d, J = 13.1 Hz), 3.77 (1H, dd, J = 2.4, 7.3 Hz), 3.67 (1H, dd, J = 2.7, 13.1 Hz), 3.46 (1H, dqd, J = 1.8, 6.1, 12.3 Hz), 2.44 (1H, ddd, J = 2.0, 6.7, 13.3 Hz), 1.22-1.11 (1H, m), 1.10 (3H, d, J = 6.1 Hz). |
| 40 | 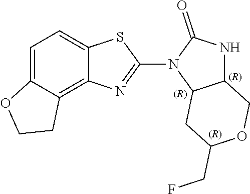 | LC-MS [M + H]⁺/Rt (min): 350.1/2.073 (Method C); ¹H-NMR (DMSO-d₆) δ: 7.93 (1H, s), 7.60 (1H, d, J = 8.4 Hz), 6.75 (1H, d, J = 8.4 Hz), 4.96- 4.85 (1H, m), 4.68-4.56 (2H, m), 4.51-4.23 (2H, m), 4.01 (1H, d, J = 13.0 Hz), 3.87-3.80 (1H, m), 3.78-3.66 (2H, m), 3.41-3.35 (2H, m), 2.48-2.41 (1H, m), 1.37-1.24 (1H, m). |

TABLE 62-2

| | | |
|---|---|---|
| 41 | 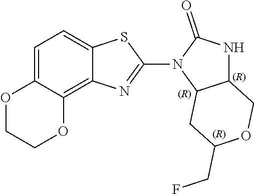 | LC-MS [M + H]⁺/Rt (min): 366.1/2.023 (Method C); ¹H-NMR (DMSO-d₆) δ: 7.91 (1H, brs), 7.30 (1H, d, J = 8.6 Hz), 6.81 (1H, d, J = 8.6 Hz), 4.90-4.84 (1H, m), 4.53-4.22 (6H, m), 4.05-3.97 (1H, m), 3.87-3.80 (1H, m), 3.79-3.63 (2H, m), 2.46-2.38 (1H, m), 1.36- 1.21 (1H, m). |
| 42 | 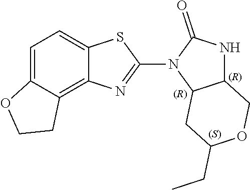 | LC-MS [M + H]⁺/Rt (min): 346.1/2.257 (Method C); ¹H-NMR (DMSO-d₆) δ: 7.87 (1H, s), 7.59 (1H, d, J = 8.4 Hz), 6.74 (1H, d, J = 8.4 Hz), 4.88-4.77 (1H, m), 4.67-4.55 (2H, m), 4.02-3.95 (1H, m), 3.82-3.76 (1H, m), 3.66 (1H, dd, J = 2.6, 13.1 Hz), 3.43-3.34 (2H, m), 3.30-3.21 (1H, m), 2.48-2.42 (1H, m), 1.47-1.36 (2H, m), 1.28-1.14 (1H, m), 0.86 (3H, t, J = 7.4 Hz). |
| 43 | 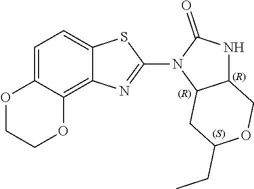 | LC-MS [M + H]⁺/Rt (min): 362.2/2.140 (Method C); ¹H-NMR (DMSO-d₆) δ: 7.85 (1H, s), 7.29 (1H, d, J = 8.6 Hz), 6.80 (1H, d, J = 8.6 Hz), 4.86-4.73 (1H, m), 4.43-4.24 (4H, m), 4.03-3.93 (1H, m), 3.83-3.75 (1H, m), 3.66 (1H, dd, J = 2.7, 13.1 Hz), 3.30-3.20 (1H, m), 2.47-2.36 (1H, m), 1.47-1.37 (2H, m), 1.26-1.13 (1H, m), 0.86 (3H, t, J = 7.4 Hz). |

### Example 44

The compound of Example 44 was obtained by using the corresponding raw material compounds according to the method described in Reference Example 14.

TABLE 63

| Example | Chemical structure | Physical property data |
|---|---|---|
| 44 | 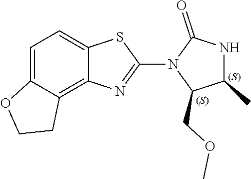 | LC-MS [M + H]⁺/Rt (min): 320.1/0.737 (Method B); ¹H-NMR (CDCl₃) δ: 7.46 (1H, d, J = 8.7 Hz), 6.76 (1H, d, J = 8.7 Hz), 4.89 (1H, br s), 4.78-4.74 (1H, m), 4.68-4.64 (2H, m), 4.25-4.18 (1H, m), 3.98 (1H, dd, J = 10.5, 6.4 Hz), 3.80 (1H, dd, J = 10.5, 2.1 Hz), 3.48-3.42 (2H, m), 3.32 (3H, s), 1.43 (3H, d, J = 6.4 Hz). |

### Examples 45 to 66

The compounds of Examples 45 to 66 were obtained by using the corresponding raw material compounds according to the method described in Example 1.

TABLE 64-1

| Example | Chemical structure | Physical property data |
|---|---|---|
| 45 | 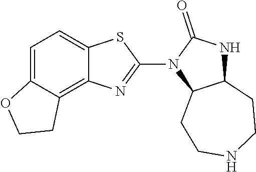 [SMILES: O=C1N[C@H]2CCNCC[C@H]2N1c1nc2c3c(ccc2s1)OCC3] | LC-MS [M + H]⁺/Rt (min): 331.2/0.610 (Method B); ¹H-NMR (CDCl₃, 50° C.) δ: 7.45 (1H, d, J = 7.9 Hz), 6.75 (1H, d, J = 7.9 Hz), 4.98- 4.92 (1H, m), 4.86 (1H, br s), 4.68-4.61 (2H, m), 4.30-4.24 (1H, m), 3.47-3.41 (2H, m), 3.13-3.06 (1H, m), 3.04-2.98 (1H, m), 2.88-2.75 (2H, m), 2.51-2.44 (1H, m), 2.39- 2.30 (1H, m), 2.11-2.04 (1H, m), 2.00-1.91 (1H, m). |
| 46 | 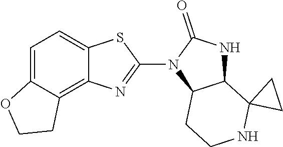 [SMILES: O=C1N[C@@H]2[C@@H](CCNC23CC3)N1c1nc2c3c(ccc2s1)OCC3] | LC-MS [M + H]⁺/Rt (min): 343.1/0.476 (Method B); ¹H-NMR (CDCl₃) δ: 7.46 (1H, d, J = 8.6 Hz), 6.77 (1H, d, J = 8.6 Hz), 4.99 (1H, s), 4.93-4.85 (1H, m), 4.72-4.61 (2H, m), 3.45 (2H, t, J = 8.6 Hz), 3.16 (1H, d, J = 6.7 Hz), 3.05-2.99 (1H, m), 2.80-2.73 (1H, m), 2.64-2.56 (1H, m), 1.67-1.47 (2H, m), 0.86-0.73 (2H, m), 0.59-0.50 (2H, m). |
| 47 | 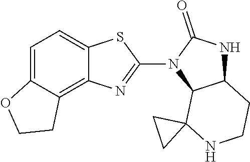 [SMILES: O=C1N[C@H]2CCNC3(CC3)[C@H]2N1c1nc2c3c(ccc2s1)OCC3] | LC-MS [M + H]⁺/Rt (min): 343.1/0.484 (Method B); ¹H-NMR (CDCl₃) δ: 7.45 (1H, d, J = 8.5 Hz), 6.76 (1H, d, J = 8.5 Hz), 5.07 (1H, s), 4.66 (2H, t, J = 8.8 Hz), 4.61 (1H, d, J = 8.3 Hz), 4.31-4.27 (1H, m), 3.42 (2H, t, J = 8.8 Hz), 3.16-3.08 (1H, m), 3.02- 2.96 (1H, m), 2.19-2.06 (1H, m), 1.81-1.74 (1H, m), 1.38-1.28 (1H, m), 0.82-0.77 (1H, m), 0.74-0.61 (2H, m). |
| 48 | 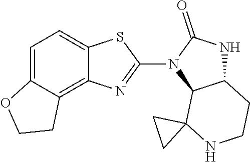 [SMILES: O=C1N[C@@H]2CCNC3(CC3)[C@H]2N1c1nc2c3c(ccc2s1)OCC3] | LC-MS [M + H]⁺/Rt (min): 343.2/0.481 (Method B); ¹H-NMR (CDCl₃) δ: 7.45 (1H, d, J = 8.3 Hz), 6.80 (1H, d, J = 8.3 Hz), 5.22 (1H, s), 4.72-4.58 (2H, m), 4.36 (1H, d, J = 11.0 Hz), 3.78-3.70 (1H, m), 3.56-3.40 (2H, m), 3.17-3.11 (1H, m), 2.91-2.78 (1H, m), 2.13- 2.06 (1H, m), 1.88-1.82 (1H, m), 1.80-1.68 (1H, m), 1.40-1.21 (1H, m), 0.76-0.66 (1H, m), 0.58-0.46 (2H, m). |
| 49 | 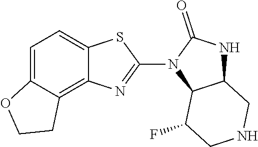 [SMILES: O=C1N[C@H]2CNC[C@H](F)[C@H]2N1c1nc2c3c(ccc2s1)OCC3] | LC-MS [M + H]⁺/Rt (min): 335.1/0.476 (Method B). ¹H-NMR (DMSO-d₆) δ: 7.68 (1H, s), 7.59 (1H, d, J = 7.9 Hz), 6.74 (1H, d, J = 7.9 Hz), 5.25-5.08 (1H, m), 4.85-4.74 (1H, m), 4.65-4.55 (2H, m), 3.83-3.78 (1H, m), 3.46- 3.33 (2H, m), 3.20-3.10 (1H, m), 3.04-2.98 (1H, m), 2.88-2.70 (2H, m), 2.05 (1H, br s). |

TABLE 64-2

| | | |
|---|---|---|
| 50 | 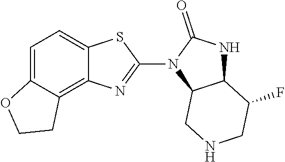 [SMILES: O=C1N[C@H]2[C@H](F)CNC[C@H]2N1c1nc2c3c(ccc2s1)OCC3] | LC-MS [M + H]⁺/Rt (min): 335.1/0.444 (Method B). ¹H-NMR (DMSO-d₆) δ: 8.02 (1H, s), 7.58 (1H, d, J = 7.9 Hz), 6.73 (1H, d, J = 7.9 Hz), 4.81-4.58 (4H, m), 4.25-4.19 (1H, m), 3.50-3.42 (1H, m), 3.36 (2H, t, J = 9.1 Hz), 3.03-2.95 (1H, m), 2.84-2.65 (2H, m). |
| 51 | 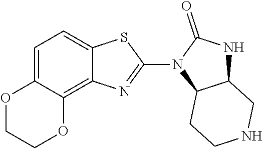 [SMILES: O=C1N[C@H]2CNCC[C@H]2N1c1nc2c3c(ccc2s1)OCCO3] | LC-MS [M +H]⁺/Rt (min): 333.1/0.443 (Method B); ¹H-NMR (DMSO-d₆) δ: 7.71 (1H, s), 7.27 (1H, d, J = 8.6 Hz), 6.78 (1H, d, J = 8.6 Hz), 4.64-4.55 (1H, m), 4.37-4.26 (4H, m), 3.75-3.68 (1H, m), 2.97-2.90 (1H, m), 2.84- 2.76 (2H, m), 2.46-2.40 (1H, m), 2.32-2.24 (1H, m), 1.59-1.50 (1H, m). |
| 52 | 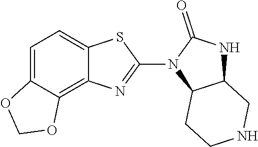 [SMILES: O=C1N[C@H]2CNCC[C@H]2N1c1nc2c3c(ccc2s1)OCO3] | LC-MS [M + H]⁺/Rt (min): 319.1/0.464 (Method B); ¹H-NMR (DMSO-d₆) δ: 7.79 (1H, s), 7.34 (1H, d, J = 8.2 Hz), 6.90 (1H, d, J = 8.2 Hz), 6.09-6.07 (2H, m), 4.66-4.58 (1H, m), 3.77-3.72 (1H, m), 2.97-2.92 (1H, m), 2.85- 2.76 (2H, m), 2.46-2.42 (1H, m), 2.31-2.06 (2H, m), 1.59-1.49 (1H, m). |
| 53 | 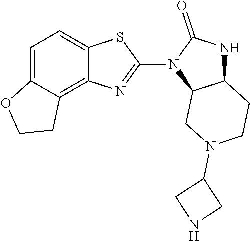 [SMILES: O=C1N[C@H]2CCN(C3CNC3)C[C@H]2N1c1nc2c3c(ccc2s1)OCC3] | LC-MS [M + H]⁺/Rt (min): 372.3/0.547 (Method A); ¹H-NMR (DMSO-d₆) δ: 7.76 (1H, s), 7.58 (1H, d, J = 8.5 Hz), 6.73 (1H, d, J = 8.5 Hz), 4.65-4.56 (3H, m), 4.11-4.04 (1H, m), 4.00-3.94 (1H, m), 3.40-3.26 (2H, m), 3.21- 3.08 (5H, m), 2.48-2.40 (1H, m), 2.17-2.04 (2H, m), 1.93-1.72 (2H, m). |
| 54 | 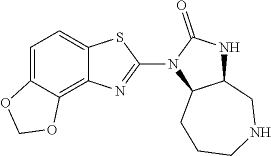 [SMILES: O=C1N[C@H]2CNCCC[C@H]2N1c1nc2c3c(ccc2s1)OCO3] | LC-MS [M + H]⁺/Rt (min): 333.2/0.576 (Method A); ¹H-NMR (DMSO-d₆) δ: 7.83 (1H, s), 7.32 (1H, d, J = 7.9 Hz), 6.89 (1H, d, J = 7.9 Hz), 6.08 (2H, s), 4.71-4.64 (1H, m), 4.05- 3.97 (1H, m), 3.01-2.95 (1H, m), 2.80-2.57 (4H, m), 2.32-2.06 (2H, m), 1.62-1.36 (2H, m). |
| 55 | 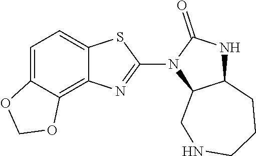 [SMILES: O=C1N[C@H]2CCCNC[C@H]2N1c1nc2c3c(ccc2s1)OCO3] | LC-MS [M + H]⁺/Rt (min): 333.2/0.507 (Method A); ¹H-NMR (DMSO-d₆) δ: 7.88 (1H, s), 7.32 (1H, d, J = 8.6 Hz), 6.89 (1H, d, J = 8.6 Hz), 6.08-6.06 (2H, m), 4.62-4.57 (1H, m), 4.19-4.13 (1H, m), 3.17-3.04 (1H, m), 2.79- 2.54 (3H, m), 1.89-1.65 (3H, m), 1.48-1.37 (1H, m). |

TABLE 64-3

| | | |
|---|---|---|
| 56 | 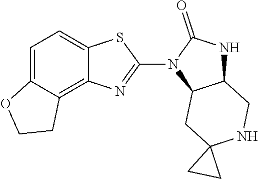 [SMILES: O=C1N[C@H]2CNC3(CC3)C[C@H]2N1c1nc2c3c(ccc2s1)OCC3] | LC-MS [M + H]⁺/Rt (min): 343.2/0.495 (Method B); ¹H-NMR (DMSO-d₆) δ: 7.80 (1H, brs), 7.56 (1H, d, J = 8.2 Hz), 6.71 (1H, d, J = 8.2 Hz), 4.73 (1H, q, J = 7.3 Hz), 4.63-4.53 (2H, m), 3.77-3.75 (1H, m), 3.40-3.31 (2H, m), 2.97-2.93 (2H, m), 1.93-1.84 (2H, m), 0.50-0.32 (4H, m). |
| 57 | 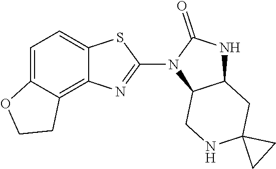 [SMILES: O=C1N[C@H]2CC3(CC3)NC[C@H]2N1c1nc2c3c(ccc2s1)OCC3] | LC-MS [M + H]⁺/Rt (min): 343.2/0.452 (Method B) |
| 58 | 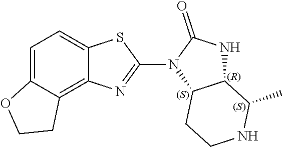 | LC-MS [M + H]⁺/Rt (min): 331.3/0.497 (Method B) |
| 59 | 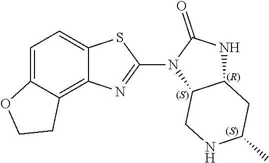 | LC-MS [M + H]⁺/Rt (min): 331.2/0.425 (MethodB) |
| 60 | 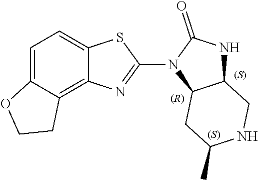 | LC-MS [M + H]⁺/Rt (min): 331.2/0.460 (Method B) |
| 61 | 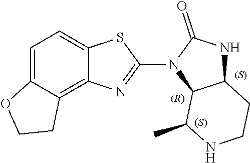 | LC-MS [M + H]⁺/Rt (min): 331.2/0.428 (Method B) |
| 62 | 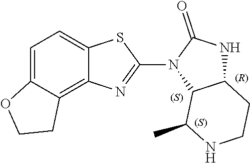 | LC-MS [M + H]⁺/Rt (min): 331.2/0.446 (Method B) |
| 63 | 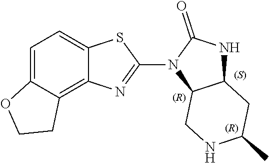 | LC-MS [M + H]⁺/Rt (min): 331.2/0.391 (Method B) |
| 64 | 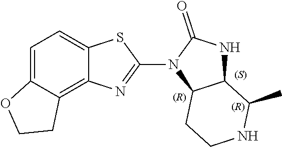 | LC-MS [M + H]⁺/Rt (min): 331.2/0.451 (Method B) |

TABLE 64-4

| | | |
|---|---|---|
| 65 | 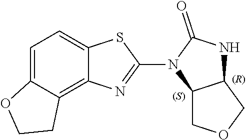 | LC-MS [M + H]⁺/Rt (min): 304.3/1.534 (Method D); ¹H-NMR (DMSO-d₆) δ: 8.14 (1H, s), 7.60 (1H, d, J = 8.5 Hz), 6.75 (1H, d, J = 8.5 Hz), 5.08 (1H, dd, J = 4.4, 8.3 Hz), 4.61 (2H, t, J = 9.0 Hz), 4.40 (1H, dd, J = 4.1, 8.3 Hz), 4.19 (1H, d, J = 10.4 Hz), 3.82 (1H, d, J = 9.8 Hz), 3.71 (1H, dd, J = 4.4, 10.4 Hz), 3.56 (1H, dd, J = 4.1, 9.8 Hz), 3.36 (2H, t, J = 9.0 Hz). |
| 66 | 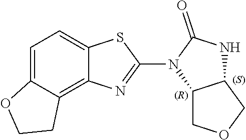 | LC-MS [M + H]⁺/Rt (min): 304.3/1.534 (Method D); ¹H-NMR (DMSO-d₆) δ: 8.14 (1H, s), 7.60 (1H, d, J = 8.5 Hz), 6.75 (1H, d, J = 8.5 Hz), 5.08 (1H, dd, J = 4.5, 8.3 Hz), 4.61 (2H, t, J = 9.0 Hz), 4.40 (1H, dd, J = 4.1, 8.3 Hz), 4.19 (1H, d, J = 10.4 Hz), 3.82 (1H, d, J = 9.8 Hz), 3.71 (1H, dd, J = 4.5, 10.4 Hz), 3.56 (1H, dd, J = 4.1, 9.8 Hz). 3.36 (2H, t, J = 9.0 Hz). |

### Examples 67 to 108

The compounds of Examples 67 to 108 were obtained by using the corresponding raw material compounds according to the method described in Example 7 or Example 8.

TABLE 65-1

| Example | Chemical structure | Physical property data |
|---|---|---|
| 67 | 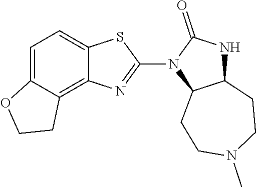 [SMILES: CN1CC[C@@H]2NC(=O)N(c3nc4c5c(ccc4s3)OCC5)[C@@H]2CC1] | LC-MS [M + H]⁺/Rt (min): 345.3/0.549 (Method A); ¹H-NMR (DMSO-d₆) δ: 7.90 (1H, s), 7.56 (1H, d, J = 8.5 Hz), 6.71 (1H, d, J = 8.5 Hz), 4.80-4.73 (1H, m), 4.64-4.54 (2H, m), 4.18-4.11 (1H, m), 3.34 (2H, t, J = 8.9 Hz), 2.58-2.44 (2H, m), 2.42-2.32 (3H, m), 2.32- 2.17 (1H, m), 2.20 (3H, s), 1.98-1.78 (2H, m). |
| 68 | 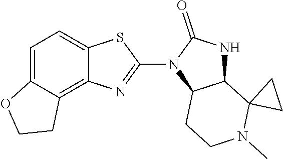 [SMILES: CN1CC[C@@H]2[C@@H](NC(=O)N2c2nc3c4c(ccc3s2)OCC4)C12CC2] | LC-MS [M + H]⁺/Rt (min): 357.1/0.489 (Method B); ¹H-NMR (CDCl₃) δ: 7.46 (1H, d, J = 8.5 Hz), 6.77 (1H, d, J = 8.5 Hz), 4.97 (1H, s), 4.93-4.85 (1H, m), 4.72-4.62 (2H, m), 3.45 (2H, t, J = 8.7 Hz), 3.16 (1H, d, J = 7.3 Hz), 3.04-2.87 (2H, m), 2.54 (3H, s), 2.43-2.35 (1H, m), 2.07-1.97 (1H, m), 0.96- 0.88 (1H, m), 0.76-0.71 (1H, m), 0.65-0.59 (1H, m), 0.52-0.47 (1H, m). |
| 69 | 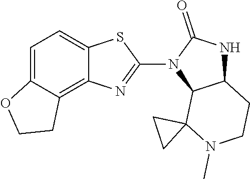 [SMILES: CN1CC[C@@H]2NC(=O)N(c3nc4c5c(ccc4s3)OCC5)[C@@H]2C12CC2] | LC-MS [M + H]⁺/Rt (min): 357.1/0.504 (Method B); ¹H-NMR (CDCl₃) δ: 7.45 (1H, d, J = 8.5 Hz), 6.76 (1H, d, J = 8.5 Hz), 4.85 (1H, s), 4.69-4.62 (3H, m), 4.28-4.24 (1H, m), 3.46 (2H, t, J = 8.9 Hz), 3.11-3.02 (1H, m), 2.98-2.91 (1H, m), 2.38 (3H, s), 2.37- 2.30 (1H, m), 1.73-1.66 (1H, m), 1.37-1.22 (1H, m), 0.88-0.72 (2H, m), 0.65-0.59 (1H, m). |
| 70 | 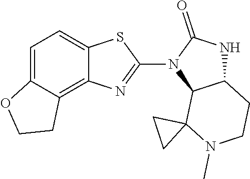 [SMILES: CN1CC[C@H]2NC(=O)N(c3nc4c5c(ccc4s3)OCC5)[C@@H]2C12CC2] | LC-MS [M + H]⁺/Rt (min): 357.2/0.522 (Method B); ¹H-NMR (CDCl₃) δ: 7.45 (1H, d, J = 8.5 Hz), 6.79 (1H, d, J = 8.5 Hz), 5.19 (1H, s), 4.67 (2H, t, J = 8.7 Hz), 4.56 (1H, d, J = 11.0 Hz), 3.76-3.65 (1H, m), 3.56-3.42 (2H, m), 3.12-2.90 (2H, m), 2.69 (3H, s), 2.16-2.05 (1H, m), 1.89-1.83 (1H, m), 1.77- 1.71 (1H, m), 0.78-0.69 (1H, m), 0.61-0.49 (2H, m). |
| 71 | 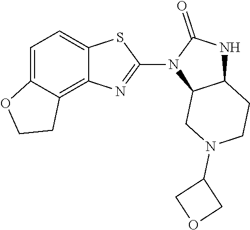 [SMILES: O=C1N[C@H]2CCN(C3COC3)C[C@H]2N1c1nc2c3c(ccc2s1)OCC3] | LC-MS [M + H]⁺/Rt (min): 373.2/0.516 (Method B); ¹H-NMR (CDCl₃) δ: 7.45 (1H, d, J = 8.2 Hz), 6.76 (1H, d, J = 8.2 Hz), 4.89-4.83 (2H, m), 4.71-4.55 (6H, m), 4.13-4.07 (1H, m), 3.54-3.36 (4H, m), 2.57-2.54 (1H, m), 2.27-2.20 (1H, m), 2.16-2.04 (2H, m), 1.97- 1.92 (1H, m). |

TABLE 65-2

| | | |
|---|---|---|
| 72 | 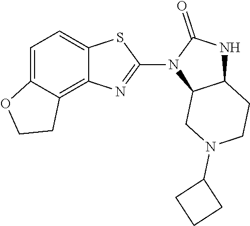 [SMILES: O=C1N[C@H]2CCN(C3CCC3)C[C@H]2N1c1nc2c3c(ccc2s1)OCC3] | LC-MS [M + H]⁺/Rt (min): 371.1/0.506 (Method B); ¹H-NMR (DMSO-d₆) δ: 7.74 (1H, s), 7.57 (1H, d, J = 8.2 Hz), 6.72 (1H, d, J = 8.2 Hz), 4.66-4.56 (3H, m), 3.97-3.94 (1H, m), 3.48-3.23 (3H, m), 2.78-2.68 (1H, m), 2.56- 2.45 (1H, m), 2.07-1.68 (8H, m), 1.65-1.50 (2H, m). |
| 73 | 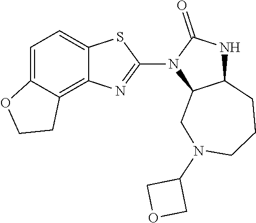 [SMILES: O=C1N[C@H]2CCCN(C3COC3)C[C@H]2N1c1nc2c3c(ccc2s1)OCC3] | LC-MS [M + H]⁺/Rt (min): 387.2/0.496 (Method B); ¹H-NMR (DMSO-d₆) δ: 7.93 (1H, s), 7.56 (1H, d, J = 8.5 Hz), 6.71 (1H, d, J = 8.5 Hz), 4.74-4.67 (1H, m), 4.65-4.46 (5H, m), 4.27-4.21 (1H, m), 4.18-4.11 (1H, m), 3.71- 3.64 (1H, m), 3.39-3.27 (2H, m), 3.26-3.17 (1H, m), 2.83-2.68 (1H, m), 2.53-2.40 (1H, m), 2.27-2.18 (1H, m), 2.00-1.89 (1H, m), 1.81-1.69 (2H, m), 1.53-1.45 (1H, m). |
| 74 | 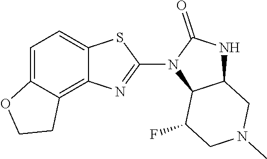 [SMILES: CN1C[C@@H]2NC(=O)N(c3nc4c5c(ccc4s3)OCC5)[C@@H]2[C@@H](F)C1] | LC-MS [M + H]⁺/Rt (min): 349.1/0.471 (Method B). ¹H-NMR (DMSO-d₆) δ: 7.83 (1H, s), 7.59 (1H, d, J = 8.2 Hz), 6.74 (1H, d, J = 8.2 Hz), 5.38-5.21 (1H, m), 4.76-4.65 (1H, m), 4.64-4.54 (2H, m), 4.04-3.99 (1H, m), 3.43- 3.32 (2H, m), 3.04-2.94 (1H, m), 2.91-2.86 (1H, m), 2.43-2.27 (2H, m), 2.22 (3H, s). |
| 75 | 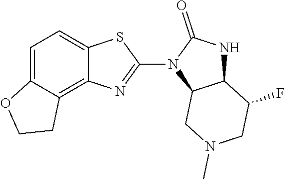 [SMILES: CN1C[C@@H](F)[C@@H]2NC(=O)N(c3nc4c5c(ccc4s3)OCC5)[C@@H]2C1] | LC-MS [M + H]⁺/Rt (min): 349.1/0.450 (Method B). ¹H-NMR (DMSO-d₆) δ: 8.07 (1H, s), 7.59 (1H, d, J = 8.2 Hz), 6.74 (1H, d, J = 8.2 Hz), 4.95-4.74 (2H, m), 4.66-4.55 (2H, m), 4.23-4.17 (1H, m), 3.45-3.33 (2H, m), 3.32- 3.26 (1H, m), 2.93-2.85 (1H, m), 2.43-2.34 (1H, m), 2.29-2.22 (1H, m), 2.24 (3H, s). |
| 76 | 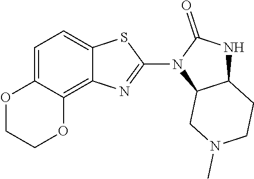 [SMILES: CN1CC[C@@H]2NC(=O)N(c3nc4c5c(ccc4s3)OCCO5)[C@@H]2C1] | LC-MS [M + H]⁺/Rt (min): 347.1/0.440 (Method B); ¹H-NMR (DMSO-d₆) δ: 7.71 (1H, s), 7.27 (1H, d, J = 8.5 Hz), 6.79 (1H, d, J = 8.5 Hz), 4.64-4.57 (1H, m), 4.39-4.27 (4H, m), 3.99-3.93 (1H, m), 3.33-3.24 (1H, m), 2.56- 2.50 (1H, m), 2.16 (3H, s), 2.13-2.04 (1H, m), 1.94-1.76 (3H, m). |
| 77 | 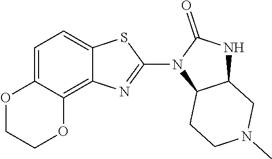 [SMILES: CN1CC[C@@H]2[C@H](C1)NC(=O)N2c1nc2c3c(ccc2s1)OCCO3] | LC-MS [M + H]⁺/Rt (min): 347.1/0.456 (Method B); ¹H-NMR (DMSO-d₆) δ: 7.82 (1H, s), 7.27 (1H, d, J = 8.5 Hz), 6.78 (1H, d, J = 8.5 Hz), 4.52-4.45 (1H, m), 4.38-4.23 (4H, m), 3.90-3.87 (1H, m), 2.76-2.71 (1H, m), 2.58- 2.51 (1H, m), 2.38-2.25 (2H, m), 2.17 (3H, s), 2.04-1.95 (1H, m), 1.86-1.75 (1H, m). |

TABLE 65-3

| | | |
|---|---|---|
| 78 | 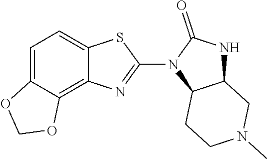 [SMILES: CN1CC[C@@H]2[C@H](C1)NC(=O)N2c1nc2c3c(ccc2s1)OCO3] | LC-MS [M + H]⁺/Rt (min): 333.1/0.428 (Method B); ¹H-NMR (DMSO-d₆) δ: 7.90 (1H, s), 7.34 (1H, d, J = 8.2 Hz), 6.91 (1H, d, J = 8.2 Hz), 6.10-6.07 (2H, m), 4.55-4.47 (1H, m), 3.93-3.88 (1H, m), 2.78-2.72 (1H, m), 2.59- 2.52 (1H, m), 2.37-2.25 (2H, m), 2.18 (3H, s), 2.03-1.96 (1H, m), 1.83-1.74 (1H, m). |
| 79 | 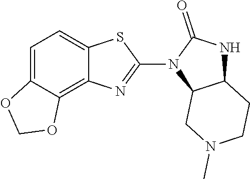 [SMILES: CN1CC[C@@H]2NC(=O)N(c3nc4c5c(ccc4s3)OCO5)[C@@H]2C1] | LC-MS [M + H]⁺/Rt (min): 333.1/0.440 (Method B); ¹H-NMR (DMSO-d₆) δ: 7.79 (1H, s), 7.35 (1H, d, J = 8.2 Hz), 6.91 (1H, d, J = 8.2 Hz), 6.11-6.09 (2H, m), 4.66-4.59 (1H, m), 4.01-3.94 (1H, m), 3.30-3.24 (1H, m), 2.57- 2.51 (1H, m), 2.17 (3H, s), 2.13-2.04 (1H, m), 1.97-1.77 (3H, m). |
| 80 | 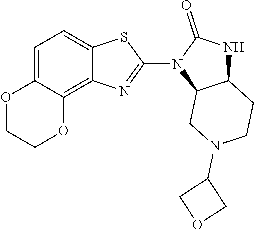 [SMILES: O=C1N[C@H]2CCN(C3COC3)C[C@H]2N1c1nc2c3c(ccc2s1)OCCO3] | LC-MS [M + H]⁺/Rt (min): 389.3/0.450 (Method B); ¹H-NMR (DMSO-d₆) δ: 7.76 (1H, s), 7.27 (1H, d, J = 8.6 Hz), 6.78 (1H, d, J = 8.6 Hz), 4.66-4.60 (1H, m), 4.53-4.47 (2H, m), 4.40-4.25 (6H, m), 4.01-3.98 (1H, m), 3.52- 3.46 (1H, m), 3.15-3.09 (1H, m), 2.50-2.48 (1H, m), 2.24-2.15 (2H, m), 1.96-1.76 (2H, m). |
| 81 | 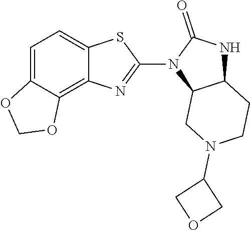 [SMILES: O=C1N[C@H]2CCN(C3COC3)C[C@H]2N1c1nc2c3c(ccc2s1)OCO3] | LC-MS [M + H]⁺/Rt (min): 375.2/0.458 (Method B); ¹H-NMR (DMSO-d₆) δ: 7.84 (1H, s), 7.34 (1H, d, J = 7.9 Hz), 6.91 (1H, d, J = 7.9 Hz), 6.11-6.08 (2H, m), 4.68-4.61 (1H, m), 4.54-4.48 (2H, m), 4.40-4.33 (2H, m), 4.03- 4.00 (1H, m), 3.51-3.44 (1H, m), 3.16-3.11 (1H, m), 2.52-2.45 (1H, m), 2.21-2.12 (2H, m), 1.97-1.76 (2H, m). |
| 82 | 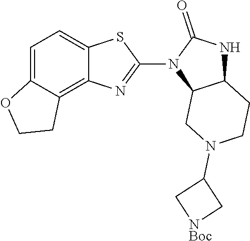 [SMILES: CC(C)(C)OC(=O)N1CC(N2CC[C@@H]3NC(=O)N(c4nc5c6c(ccc5s4)OCC6)[C@@H]3C2)C1] | LC-MS [M + H]⁺/Rt (min): 472.3/0.723 (Method B); ¹H-NMR (CDCl₃) δ: 7.45 (1H, d, J = 7.9 Hz), 6.77 (1H, d. J = 7.9 Hz), 4.99-4.80 (2H, m), 4.67 (2H, t, J = 9.2 Hz), 4.14-4.06 (1H, m), 3.96-3.74 (4H, m), 3.53-3.38 (1H, m), 3.44 (2H, t, J = 9.2 Hz), 3.16-3.05 (1H, m), 2.64-2.54 (1H, m), 2.29-1.90 (4H, m), 1.41 (9H, s). |
| 83 | 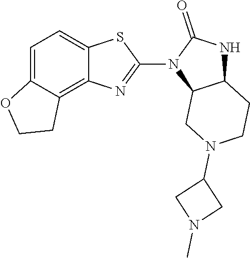 [SMILES: CN1CC(N2CC[C@@H]3NC(=O)N(c4nc5c6c(ccc5s4)OCC6)[C@@H]3C2)C1] | LC-MS [M + H]⁺/Rt (min): 386.3/0.594 (Method A); ¹H-NMR (CDCl₃) δ: 7.45 (1H, d, J = 7.9 Hz), 6.77 (1H, d, J = 7.9 Hz), 5.03 (1H, s), 4.83-4.76 (1H, m), 4.71-4.62 (2H, m), 4.11- 4.04 (1H, m), 3.95-3.84 (2H, m), 3.55-3.22 (6H, m), 2.60 (3H, s), 2.55-2.48 (1H, m), 2.36-2.25 (2H, m), 2.10-1.99 (1H, m), 1.97- 1.89 (1H, m). |

TABLE 65-4

| | | |
|---|---|---|
| 84 | 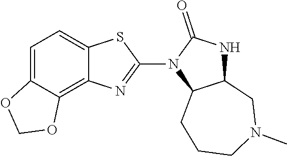 [SMILES: CN1CCC[C@@H]2[C@H](C1)NC(=O)N2c1nc2c3c(ccc2s1)OCO3] | LC-MS [M + H]⁺/Rt (min): 347.2/0.589 (Method A); ¹H-NMR (DMSO-d₆) δ: 7.86 (1H, s), 7.33 (1H, d, J = 8.2 Hz), 6.90 (1H, d, J = 8.2 Hz), 6.08 (2H, s), 4.68-4.61 (1H, m), 4.12- 4.05 (1H, m), 2.70-2.63 (1H, m), 2.60-2.51 (2H, m), 2.39-2.26 (2H, m), 2.30 (3H, s), 2.09-1.96 (1H, m), 1.69-1.44 (2H, m). |
| 85 | 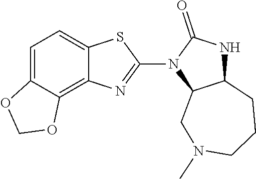 [SMILES: CN1CCC[C@@H]2NC(=O)N(c3nc4c5c(ccc4s3)OCO5)[C@@H]2C1] | LC-MS [M + H]⁺/Rt (min): 347.2/0.536 (Method A); ¹H-NMR (DMSO-d₆) δ: 7.90 (1H, s), 7.32 (1H, d, J = 7.9 Hz), 6.89 (1H, d, J = 7.9 Hz), 6.11-6.07 (2H, m), 4.68-4.63 (1H, m), 4.18-4.11 (1H, m), 3.24-3.15 (1H, m), 2.92- 2.84 (1H, m), 2.47-2.36 (2H, m), 2.18 (3H, s), 1.88-1.69 (3H, m), 1.51-1.43 (1H, m). |
| 86 | 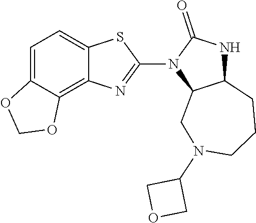 [SMILES: O=C1N[C@H]2CCCN(C3COC3)C[C@H]2N1c1nc2c3c(ccc2s1)OCO3] | LC-MS [M + H]⁺/Rt (min): 389.2/0.562 (Method A); ¹H-NMR (DMSO-d₆) δ: 7.95 (1H, s), 7.33 (1H, d, J = 7.9 Hz), 6.90 (1H, d, J = 7.9 Hz), 6.11-6.06 (2H, m), 4.71-4.66 (1H, m), 4.45-4.34 (3H, m), 4.21-4.16 (1H, m), 4.09- 4.03 (1H, m), 3.67-3.60 (1H, m), 2.99-2.89 (2H, m), 2.35-2.26 (2H, m), 1.92-1.73 (3H, m), 1.55-1.45 (1H, m). |
| 87 | 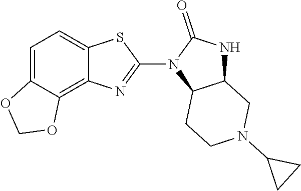 [SMILES: O=C1N[C@H]2CN(C3CC3)CC[C@H]2N1c1nc2c3c(ccc2s1)OCO3] | LC-MS [M + H]⁺/Rt (min): 359.2/0.582 (Method A); ¹H-NMR (DMSO-d₆) δ: 7.85 (1H, s), 7.34 (1H, d, J = 8.3 Hz), 6.90 (1H, d, J = 8.3 Hz), 6.10-6.07 (2H, m), 4.57-4.50 (1H, m), 3.91-3.87 (1H, m), 2.98-2.89 (1H, m), 2.80- 2.70 (1H, m), 2.61-2.53 (1H, m), 2.36-2.26 (2H, m), 1.75-1.65 (2H, m), 0.46-0.39 (2H, m), 0.35-0.24 (2H, m). |
| 88 | 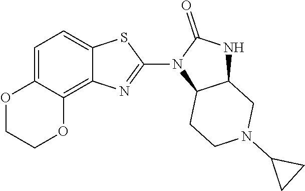 [SMILES: O=C1N[C@H]2CN(C3CC3)CC[C@H]2N1c1nc2c3c(ccc2s1)OCCO3] | LC-MS [M + H]⁺/Rt (min): 373.2/0.549 (Method A); ¹H-NMR (DMSO-d₆) δ: 7.77 (1H, s), 7.26 (1H, d, J = 8.6 Hz), 6.78 (1H, d, J = 8.6 Hz), 4.54-4.47 (1H, m), 4.37-4.26 (4H, m), 3.90-3.83 (1H, m), 2.96-2.89 (1H, m), 2.76- 2.70 (1H, m), 2.59-2.53 (1H, m), 2.35-2.25 (2H, m), 1.75-1.65 (2H, m), 0.45-0.39 (2H, m), 0.34-0.23 (2H, m). |
| 89 | 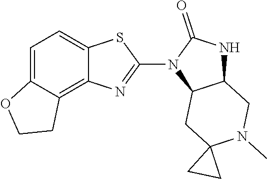 [SMILES: CN1C[C@@H]2NC(=O)N(c3nc4c5c(ccc4s3)OCC5)[C@@H]2CC12CC2] | LC-MS [M + H]⁺/Rt (min): 357.1/0.507 (Method B); ¹H-NMR (DMSO-d₆) δ: 7.94 (1H, brs), 7.57 (1H, d, J = 8.2 Hz), 6.72 (1H, d, J = 8.2 Hz), 4.74-4.67 (1H, m), 4.64-4.53 (2H, m), 3.83-3.80 (1H, m), 3.36-3.32 (2H, m), 3.01- 2.89 (2H, m), 2.40 (3H, s), 2.16 (1H, m), 1.90-1.76 (1H, m), 0.58-0.30 (4H, m). |

TABLE 65-5

| | | |
|---|---|---|
| 90 | 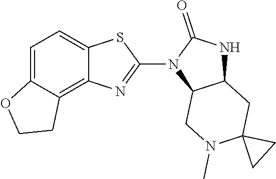 [SMILES: CN1C[C@@H]2[C@H](CC13CC3)NC(=O)N2c1nc2c3c(ccc2s1)OCC3] | LC-MS [M + H]⁺/Rt (min): 357.2/0.462 (Method B); ¹H-NMR (DMSO-d₆) δ: 7.83 (1H, brs), 7.57 (1H, d, J = 8.2 Hz), 6.72 (1H, d, J = 8.2 Hz), 4.81-4.72 (1H, m), 4.63-4.57 (2H, m), 4.08 (1H, m), 3.40-3.33 (2H, m), 2.87-2.81 (1H, m), 2.35-2.30 (1H, m), 2.34 (3H, s), 1.23-1.15 (1H, m), 0.58-0.28 (4H, m). |
| 91 | 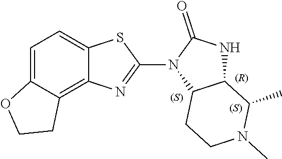 | LC-MS [M+H]+/Rt (min): 345.2/0.450 (Method B); ¹H-NMR (DMSO-d₆) δ: 7.86 (1H, brs), 7.57 (1H, d, J = 8.2 Hz), 6.73 (1H, d, J = 8.2 Hz), 4.62-4.55 (3H, m), 3.77 (1H, m), 3.36 (2H, t, J = 8.9 Hz), 2.67 (1H, d, J = 11.0 Hz), 2.41-2.36 (1H, m), 2.18-2.14 (1H, m), 2.06-2.04 (1H, m), 1.53-1.50 (1H, m), 1.13 (3H, d, J = 5.9 Hz). |
| 92 | 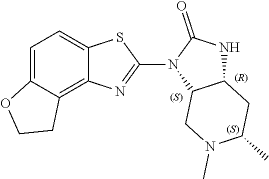 | LC-MS [M+H]+/Rt (min): 345.3/0.442 (Method B); ¹H-NMR (CDCl₃) δ: 7.45 (1H, d, J = 8.7 Hz), 6.76 (1H, d, J = 8.7 Hz), 4.95 (1H, brs), 4.70-4.58 (3H, m), 3.95 (1H, dd, J = 13.5, 7.1 Hz), 3.67 (1H, dd, J = 13.2, 5.0 Hz), 3.45 (2H, t, J = 8.7 Hz), 2.84 (1H, dd, J = 13.2, 4.6 Hz), 2.60-2.38 (1H, m), 2.30 (3H, s), 2.13-2.01 (1H, m), 1.68-1.62 (1H, m), 1.14 (3H, d, J = 6.6 Hz). |
| 93 | 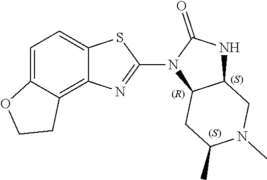 | LC-MS [M+H]+/Rt (min): 345.2/0.490 (Method B); ¹H-NMR (DMSO-d₆) δ: 7.82 (1H, brs), 7.58 (1H, d, J = 8.4 Hz), 6.72 (1H, d, J = 8.4 Hz), 4.69-4.55 (3H, m), 3.90 (1H, m), 3.45- 3.34 (2H, m), 2.90 (1H, m), 2.37-2.30 (2H, m), 2.17 (3H, s), 2.15 (1H, s), 2.03-1.98 (1H, m), 1.40-1.32 (1H, m), 0.96 (3H, d, J = 6.7 Hz). |
| 94 | 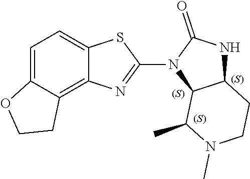 | LC-MS [M+H]+/Rt (min): 345.2/0.481 (Method B); ¹H-NMR (CDCl₃) δ: 7.46 (1H, d, J = 8.4 Hz), 6.76 (1H, d, J = 8.4 Hz), 4.91 (1H, t, J = 7.1 Hz), 4.72 (1H, brs), 4.68-4.61 (2H, m), 4.11 (1H, t, J = 6.2 Hz), 3.87-3.80 (1H, m), 3.49-3.42 (2H, m), 2.79-2.72 (1H, m), 2.42 (3H, s), 2.41-2.36 (1H, m), 2.15-2.05 (1H, m), 1.87-1.84 (1H, m), 0.89 (3H, d, J = 6.8 Hz). |

TABLE 65-6

| | | |
|---|---|---|
| 95 | 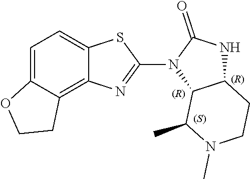 | LC-MS [M + H]⁺/Rt (min): 345.2/0.474 (Method B); ¹H-NMR (CDCl₃) δ: 7.47 (1H, d, J = 8.8 Hz), 6.77 (1H, d, J = 8.8 Hz), 5.30 (1H, brs), 4.84-4.77 (1H, m), 4.66 (2H, t, J = 8.7 Hz), 3.55-3.51 (1H, m), 3.47 (2H, t, J = 8.7 Hz), 3.38-3.33 (1H, m), 2.66-2.59 (1H, m), 2.47 (3H, s), 2.30-2.25 (1H, m), 2.18- 2.10 (1H, m), 1.76-1.66 (1H, m), 1.59 (3H, d, J = 6.0 Hz). |
| 96 | 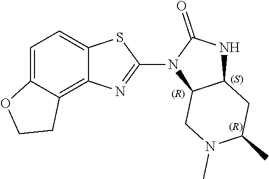 | LC-MS[M + H]⁺/Rt (min): 345.1/0.449 (Method B); ¹H-NMR (CDCl₃) δ: 7.47 (1H, d, J = 8.8 Hz), 6.78 (1H, d, J = 8.8 Hz), 5.13 (1H, brs), 4.84-4.47 (3H, m), 3.95 (1H, dd, J = 12.8, 7.3 Hz), 3.70-3.61 (1H, m), 3.47-3.42 (2H, m), 2.84 (1H, dd, J = 13.5, 4.3 Hz), 2.60-2.38 (1H, m), 2.31 (3H, s), 2.12-2.02 (1H, m), 1.68-1.61 (1H, m), 1.14 (3H, d, J = 6.4 Hz). |
| 97 | 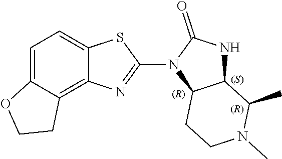 | LC-MS [M + H]⁺/Rt (min): 345.1/0.453 (Method B); ¹H-NMR (CDCl₃) δ: 7.45 (1H, d, J = 8.8 Hz), 6.76 (1H, d, J = 8.8 Hz), 5.01 (1H, brs), 4.74-4.61 (3H, m), 3.81-3.79 (1H, m), 3.44 (2H, t, J = 8.8 Hz), 2.87-2.82 (1H, m), 2.65-2.59 (1H, m), 2.31 (3H, s), 2.30-2.25 (1H, m), 2.19-2.12 (1H, m), 1.82-1.71 (1H, m), 1.26 (3H, d, J = 6.9 Hz). |
| 98 | 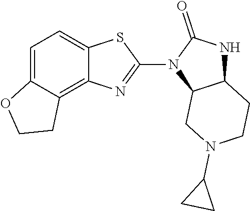 [SMILES: O=C1N[C@H]2CCN(C3CC3)C[C@H]2N1c1nc2c3c(ccc2s1)OCC3] | LC-MS [M + H]⁺/Rt (min): 357.1/0.489 (Method B); ¹H-NMR (CDCl₃) δ: 7.45 (1H, d, J = 8.8 Hz), 6.76 (1H, d, J = 8.8 Hz), 4.81-4.74 (1H, m), 4.69-4.64 (3H, m), 4.05-4.00 (1H, m), 3.73-3.69 (1H, m), 3.45 (2H, t, J = 8.7 Hz), 2.85-2.81 (1H, m), 2.53-2.46 (1H, m), 2.39-2.29 (1H, m), 2.01-1.83 (2H, m), 1.69- 1.61 (1H, m), 0.48-0.38 (4H, m). |
| 99 | 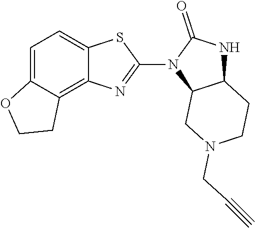 [SMILES: C#CCN1CC[C@@H]2NC(=O)N(c3nc4c5c(ccc4s3)OCC5)[C@@H]2C1] | LC-MS [M + H]⁺/Rt (min): 355.1/0.530 (Method B); ¹H-NMR (CDCl₃) δ: 7.47 (1H, d, J = 8.4 Hz), 6.76 (1H, d, J = 8.4 Hz), 4.91-4.85 (1H, m), 4.74 (1H, brs), 4.68-4.62 (2H, m), 4.09-4.05 (1H, m), 3.59-3.51 (1H, m), 3.49- 3.41 (3H, m), 3.37-3.35 (2H, m), 2.72-2.68 (1H, m), 2.57-2.51 (1H, m), 2.24-2.22 (1H, m), 2.13-2.03 (1H, m), 1.96-1.92 (1H, m). |

TABLE 65-7

| | | |
|---|---|---|
| 100 | 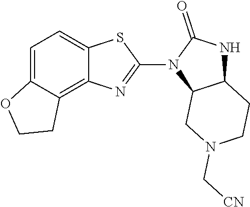 [SMILES: N#CCN1CC[C@@H]2NC(=O)N(c3nc4c5c(ccc4s3)OCC5)[C@@H]2C1] | LC-MS [M + H]⁺/Rt (min): 356.1/0.681 (Method B); ¹H-NMR (CDCl₃) δ: 7.46 (1H, d, J = 8.2 Hz), 6.78 (1H, d, J = 8.2 Hz), 4.90-4.82 (2H, m), 4.67 (2H, t, J = 8.2 Hz), 4.11-4.08 (1H, m), 3.59-3.43 (3H, m), 3.47-3.43 (2H, m), 2.71-2.66 (2H, m), 2.63-2.58 (1H, m), 2.15-2.03 (1H, m), 2.00-1.94 (1H, m). |
| 101 | 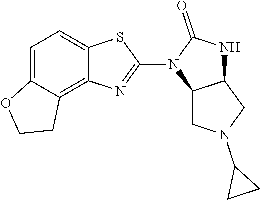 [SMILES: O=C1N[C@H]2CN(C3CC3)C[C@H]2N1c1nc2c3c(ccc2s1)OCC3] | LC-MS [M + H]⁺/Rt (min): 343.1/0.502 (Method B); ¹H-NMR (DMSO-d₆) δ: 8.00 (1H, brs), 7.56 (1H, d, J = 8.2 Hz), 6.72 (1H, d, J = 8.2 Hz), 4.95-4.89 (1H, m), 4.62-4.57 (2H, m), 4.26-4.22 (1H, m), 3.39-3.22 (4H, m), 2.86 (1H, d, J = 10.1 Hz), 2.68-2.64 (1H, m), 2.53-2.49 (1H, m), 1.65-1.60 (1H, m), 0.44- 0.23 (4H, m). |
| 102 | 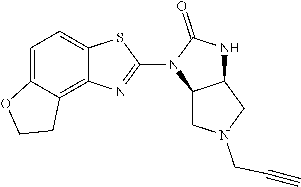 [SMILES: C#CCN1C[C@@H]2NC(=O)N(c3nc4c5c(ccc4s3)OCC5)[C@@H]2C1] | LC-MS [M + H]⁺/Rt (min): 341.1/0.544 (Method B); ¹H-NMR (DMSO-d₆) δ: 8.06 (1H, s), 7.57 (1H, d, J = 8.2 Hz), 6.72 (1H, d, J = 8.2 Hz), 4.97-4.92 (1H, m), 4.62-4.57 (2H, m), 4.29-4.25 (1H, m), 3.43-3.42 (2H, m), 3.36 (2H, t, J = 8.9 Hz), 3.26-3.21 (1H, m), 3.16 (1H, t, J = 2.3 Hz), 2.83-2.81 (1H, m), 2.58- 2.54 (1H, m), 2.46-2.41 (1H, m). |
| 103 | 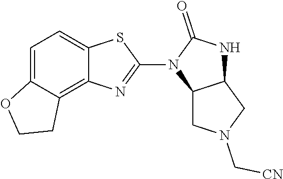 [SMILES: N#CCN1C[C@@H]2NC(=O)N(c3nc4c5c(ccc4s3)OCC5)[C@@H]2C1] | LC-MS [M + H]⁺/Rt (min): 342.1/0.656 (Method B); ¹H-NMR (DMSO-d₆) δ: 8.11 (1H, s), 7.58 (1H, d, J = 8.2 Hz), 6.73 (1H, d, J = 8.2 Hz), 5.01-4.97 (1H, m), 4.61-4.57 (2H, m), 4.34-4.31 (1H, m), 3.96-3.85 (2H, m), 3.39- 3.31 (3H, m), 2.88 (1H, d, J = 10.1 Hz), 2.60-2.55 (1H, m), 2.51-2.44 (1H, m). |
| 104 | 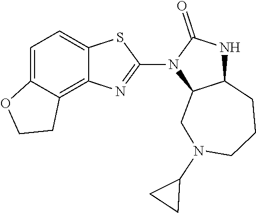 [SMILES: O=C1N[C@H]2CCCN(C3CC3)C[C@H]2N1c1nc2c3c(ccc2s1)OCC3] | LC-MS [M + H]⁺/Rt (min): 371.2/0.525 (Method B); ¹H-NMR (CDCl₃) δ: 7.46 (1H, d, J = 8.5 Hz), 6.76 (1H, d, J = 8.5 Hz), 4.82-4.77 (2H, m), 4.67 (2H, t, J = 8.8 Hz), 4.21-4.10 (1H, m), 3.69-3.64 (1H, m), 3.47 (2H, t, J = 8.5 Hz), 3.01-2.96 (2H, m), 2.60-2.56 (1H, m), 2.05-1.46 (5H, m), 0.65-0.32 (4H, m). |
| 105 | 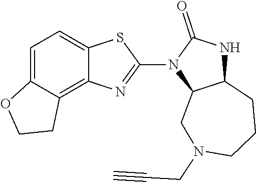 [SMILES: C#CCN1CCC[C@@H]2NC(=O)N(c3nc4c5c(ccc4s3)OCC5)[C@@H]2C1] | LC-MS [M + H]⁺/Rt (min): 369.3/0.528 (Method B); ¹H-NMR (CDCl₃) δ: 7.45 (1H, d, J = 8.5 Hz), 6.79 (1H, d, J = 8.5 Hz), 5.89-5.78 (1H, m), 5.09-5.00 (1H, m), 4.67 (2H, t, J = 10.4 Hz), 4.54-4.40 (2H, m), 4.03-3.92 (2H, m), 3.64-3.52 (1H, m), 3.50-3.37 (3H, m), 3.28-3.18 (1H, m), 2.47-1.82 (5H, m). |

TABLE 65-8

| | | |
|---|---|---|
| 106 | 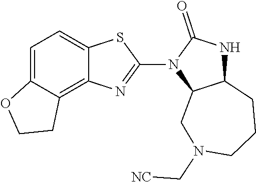 [SMILES: N#CCN1CCC[C@@H]2NC(=O)N(c3nc4c5c(ccc4s3)OCC5)[C@@H]2C1] | LC-MS [M + H]⁺/Rt (min): 370.2/0.731 (Method B); ¹H-NMR (CDCl₃) δ: 7.46 (1H, d, J = 8.5 Hz), 6.77 (1H, d, J = 8.5 Hz), 4.86-4.78 (2H, m), 4.67 (2H, t, J = 8.8 Hz), 4.28-4.24 (1H, m), 3.65-3.58 (1H, m), 3.53-3.36 (4H, m), 3.19-3.16 (1H, m), 2.72-2.71 (2H, m), 1.90-1.74 (4H, m). |
| 107 | 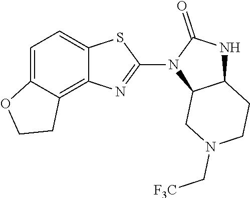 [SMILES: O=C1N[C@H]2CCN(CC(F)(F)F)C[C@H]2N1c1nc2c3c(ccc2s1)OCC3] | LC-MS [M + H]⁺/Rt (min): 399.2/0.881 (Method B); ¹H-NMR (CDCl₃) δ: 7.45 (1H, d, J = 7.3 Hz), 6.77 (1H, d, J = 8.5 Hz), 4.89-4.83 (1H, m), 4.70-4.63 (3H, m), 4.10-4.06 (1H, m), 3.70-3.67 (1H, m), 3.45 (2H, t, J = 8.8 Hz), 3.08-2.99 (2H, m), 2.86-2.59 (3H, m), 2.14-1.88 (2H, m). |
| 108 | 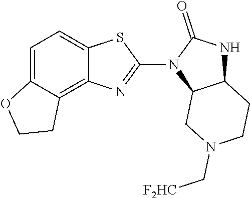 [SMILES: O=C1N[C@H]2CCN(CC(F)F)C[C@H]2N1c1nc2c3c(ccc2s1)OCC3] | LC-MS [M + H]⁺/Rt (min): 381.1/0.730 (Method B); ¹H-NMR (CDCl₃) δ: 7.45 (1H, d, J = 8.4 Hz), 6.77 (1H, d, J = 8.4 Hz), 6.05-5.77 (1H, m), 4.92-4.78 (2H, m), 4.67 (2H, t, J = 9.2 Hz), 4.09-4.05 (1H, m), 3.68-3.65 (1H, m), 3.45 (2H, t. J = 9.2 Hz), 2.84-2.77 (3H, m), 2.63-2.50 (2H, m), 2.21-2.04 (1H, m), 1.94-1.90 (1H, m). |

### Example 109

### (3aS,7aR)-5-Cyclopropyl-3-(7,8-dihydrofuro[3,2-e][1,3]benzothiazol-2-yl)octahydro-2H-imidazo[4,5-c]pyridin-2-one

### Example 110

### (3aR,7aS)-5-Cyclopropyl-3-(7,8-dihydrofuro[3,2-e][1,3]benzothiazol-2-yl)octahydro-2H-imidazo[4,5-c]pyridin-2-one

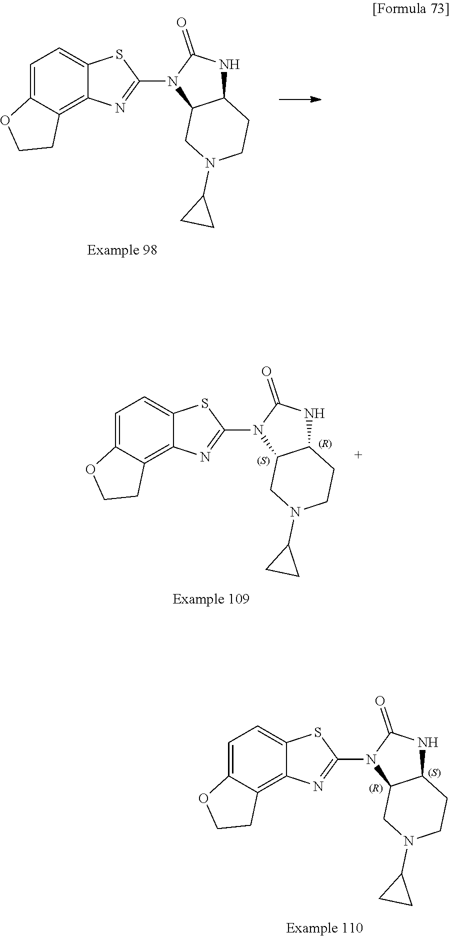

The compound of Example 98 (12.0 mg) was optically fractionated under the following conditions to obtain the title compounds (Example 109: 1.5 mg-first peak: 9.40 min, Example 110: 1.1 mg-second peak: 10.8 min).

Column: CHIRALPAK® IG; Solvents: liquid A: chloroform, liquid B: methanol, liquid C: diethylamine; Mobile phase condition: A/B/C=99/1/0.002; Flow rate: 7 mL/min; Detection UV: 280 nm; Column temperature: 40° C.

TABLE 66

| Example | Physical property data |
|---|---|
| 109 | LC-MS [M + H]⁺/Rt (min): 357.2/0.515 (Method B); ¹H-NMR (CDCl₃) δ: 7.45 |
| | (1H, d, J = 8.2 Hz), 6.76 (1H, d, J = 8.2 Hz), 4.82-4.62 (4H, m), 4.07- |
| | 4.01 (1H, m), 3.76-3.65 (1H, m), 3.56-3.42 (2H, m), 2.87-2.78 (1H, m), |
| | 2.55-2.33 (2H, m), 2.02-1.84 (2H, m), 1.67-1.60 (1H, m), 0.48-0.35 (4H, |
| | m) |
| 110 | LC-MS [M + H]⁺/Rt (min): 357.1/0.513 (Method B); ¹H-NMR (CDCl₃) δ: 7.45 |
| | (1H, d, J = 8.5 Hz), 6.76 (1H, d, J = 8.5 Hz), 4.83-4.62 (4H, m), 4.07- |
| | 4.00 (1H, m), 3.75-3.68 (1H, m), 3.57-3.42 (2H, m), 2.88-2.79 (1H, m), |
| | 2.56-2.32 (2H, m), 2.02-1.84 (2H, m), 1.68-1.61 (1H, m), 0.49-0.36 (4H, |
| | m). |

### Example 111

### [(3aS,7aR)-3-(7,8-Dihydrofuro[3,2-e][1,3]benzothiazol-2-yl)-2-oxooctahydro-5H-imidazo[4,5-c]pyridin-5-yl]acetonitrile

### Example 112

### [(3aR,7aS)-3-(7,8-Dihydrofuro[3,2-e][1,3]benzothiazol-2-yl)-2-oxooctahydro-5H-imidazo[4,5-c]pyridin-5-yl]acetonitrile

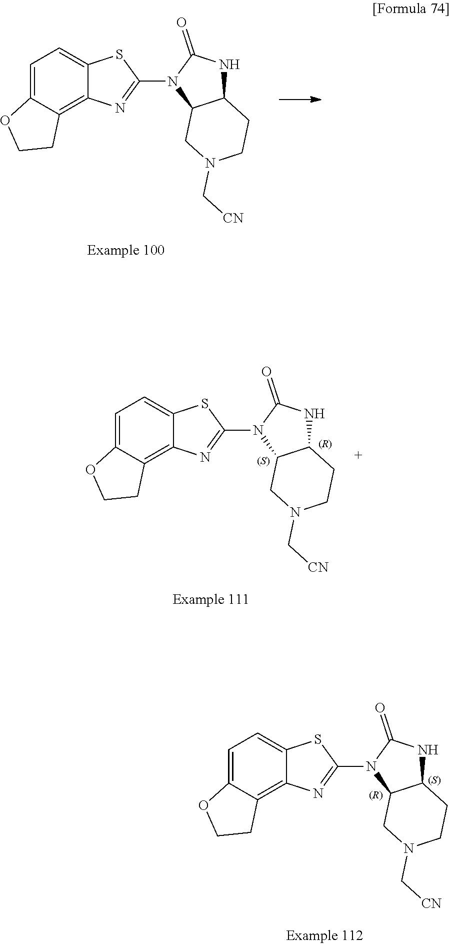

The compound of Example 100 (30.0 mg) was optically fractionated under the following conditions to obtain the title compounds (Example 111: 13.8 mg-First Peak: 13.0 min, Example 112: 12.2 mg-Second Peak: 14.5 min).

Column: CHIRALPAK® IC; Solvents: liquid A: chloroform, liquid B: methanol, liquid C: diethylamine; Mobile phase condition: A/B/C=98/1/0.001; Flow rate: 10 mL/min; Detection UV: 254 nm; Column temperature: 40° C.

TABLE 67

| Example | Physical property data |
|---|---|
| 111 | LC-MS [M + H]⁺/Rt (min): 356.2/0.755 (Method A); ¹H-NMR (DMSO-d₆) δ: 7.81 |
| | (1H, s), 7.59 (1H, d, J = 8.5 Hz), 6.74 (1H, d, J = 8.5 Hz), 4.75-4.68 |
| | (1H, m), 4.66-4.56 (2H, m), 4.03-3.98 (1H, m), 3.78 (2H, s), 3.43-3.34 |
| | (3H, m), 2.66-2.59 (1H, m), 2.42-2.35 (1H, m), 2.22 (1H, dd, J = 8.9, |
| | 11.3 Hz), 1.98-1.81 (2H, m). |
| 112 | LC-MS [M + H]⁺/Rt (min): 356.2/0.755 (Method A); ¹H-NMR (DMSO-d₆) δ: 7.81 |
| | (1H, s), 7.59 (1H, d, J = 8.5 Hz), 6.74 (1H, d, J = 8.5 Hz), 4.75-4.68 |
| | (1H, m), 4.66-4.56 (2H, m), 4.03-3.98 (1H, m), 3.78 (2H, s), 3.43-3.34 |
| | (3H, m), 2.66-2.59 (1H, m), 2.42-2.35 (1H, m), 2.22 (1H, dd, J = 9.2, |
| | 11.0 Hz), 1.98-1.81 (2H, m). |

### Example 113

### (3aS,7aR)-3-(7,8-Dihydrofuro[3,2-e][1,3]benzothiazol-2-yl)-5-(2-propyn-1-yl)octahydro-2H-imidazo[4,5-c]pyridin-2-one

### Example 114

### (3aR,7aS)-3-(7,8-Dihydrofuro[3,2-e][1,3]benzothiazol-2-yl)-5-(2-propyn-1-yl)octahydro-2H-imidazo[4,5-c]pyridin-2-one

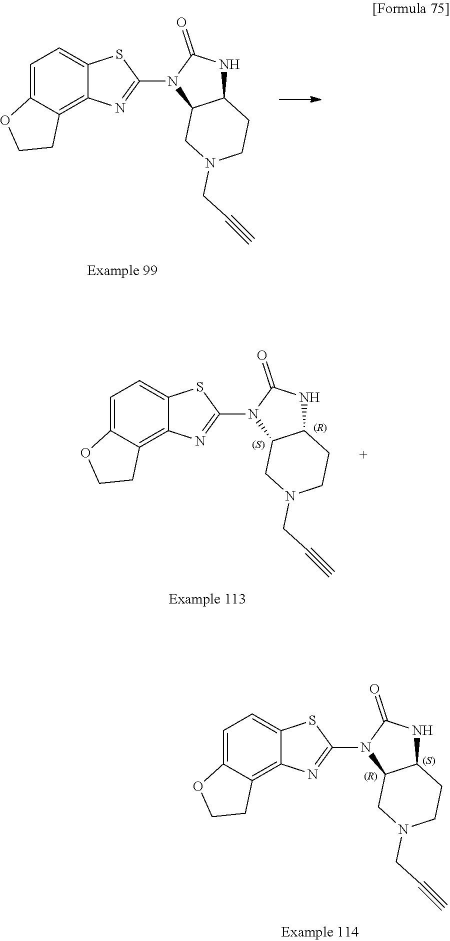

The compound of Example 99 (30.0 mg) was optically fractionated under the following conditions to obtain the title compounds (Example 113: 15.4 mg-First Peak: 10.2 min, Example 114: 13.7 mg-Second Peak: 11.3 min).

Column: CHIRALPAK® IC; Solvents: liquid A: chloroform, liquid B: methanol, liquid C: diethylamine; Mobile phase condition: A/B/C=99/1/0.001; Flow rate: 10 mL/min; Detection UV: 254 nm; Column temperature: 40° C.

TABLE 68

| Example | Physical property data |
|---|---|
| 113 | LC-MS [M + H]⁺/Rt (min): 355.2/0.608 (Method A); ¹H-NMR (DMSO-d₆) δ: 7.76 |
| | (1H, s), 7.58 (1H, d, J = 8.5 Hz), 6.73 (1H, d, J = 8.5 Hz), 4.70-4.56 |
| | (3H, m), 4.01-3.95 (1H, m), 3.41-3.35 (3H, m), 3.32-3.30 (2H, m), 3.14 |
| | (1H, t, J = 2.1 Hz), 2.62-2.55 (1H, m), 2.38-2.30 (1H, m), 2.17 (1H, dd, |
| | J = 9.2, 11.6 Hz), 1.94-1.79 (2H, m). |
| 114 | LC-MS [M + H]⁺/Rt (min): 355.2/0.610 (Method A); ¹H-NMR (DMSO-d₆) δ: 7.76 |
| | (1H, s), 7.58 (1H, d, J = 8.5 Hz), 6.73 (1H, d, J = 8.5 Hz), 4.70-4.56 |
| | (3H, m), 4.01-3.95 (1H, m), 3.41-3.35 (3H, m), 3.32 (2H, d J = 2.1 Hz). |
| | 3.14 (1H, t, J = 2.1 Hz), 2.62-2.55 (1H, m), 2.38-2.30 (1H, m), 2.17 |
| | (1H, dd, J = 9.2, 11.6 Hz), 1.94-1.79 (2H, m). |

### Examples 115 to 118

The compounds of Examples 115 to 118 were obtained by using the corresponding raw material compounds according to the method described in Example 19.

TABLE 69

| Example | Chemical structure | Physical property data |
|---|---|---|
| 115 | 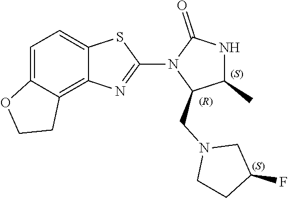 | LC-MS [M + H]⁺/Rt (min): 377.2/0.491 (Method B); ¹H-NMR (CDCl₃) δ: 7. 46 (1H, d, J = 8.4 Hz), 6.76 (1H, d, J = 8. 4 Hz), 5.21-5.07 (1H, m), 4.81-4.73 (2H, m), 4.67 (2H, t, J = 8.8 Hz), 4.28-4.19 (1H, m), 3.51-3.35 (2H, m), 3.15-3.04 (2H, m), 2.96-2.75 (3H, m), 2.61-2.54 (1H, m), 2.18-1.93 (2H, m), 1.46 (3H, d, J = 6. 7 Hz). |
| 116 | 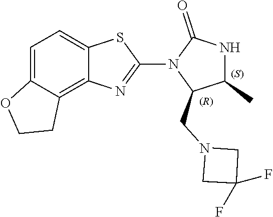 | LC-MS [M + H]⁺/Rt (min): 381.2/0.652 (Method B); ¹H-NMR (CDCl₃) δ: 7.48 (1H, d, J = 8.4 Hz), 6.78 (1H, d, J = 8.4 Hz), 4.85 (1H, brs), 4.69 (2H, t, J = 7.9 Hz), 4.59-4.50 (2H, m), 4.29-4.21 (1H, m), 4.03-3.63 (3H, m), 3.47-3.42 (2H, m), 3.33-3.05 (2H, m), 1.47 (3H, d, J = 6.7 Hz). |
| 117 | 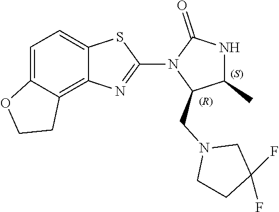 | LC-MS [M + H]⁺/Rt (min): 395.2/0.685 (Method B); ¹H-NMR (CDCl₃) δ: 7.46 (1H, d, J = 8.8 Hz), 6.78 (1H, d, J = 8.8 Hz), 4.87 (1H, br s), 4.79-4.65 (4H, m), 4.34-4.21 (1H, m), 3.46-3.40 (2H, m), 3.32-2.81 (5H, m), 2.38- 2.20 (2H, m), 1.47 (3H, d, J = 7.0 Hz). |
| 118 | 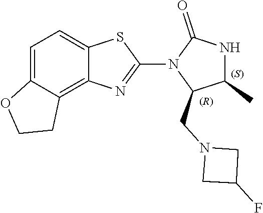 | LC-MS [M + H]⁺/Rt (min): 363.1/0.490 (Method B); ¹H-NMR (CDCl₃) δ: 7.46 (1H, d, J = 8.5 Hz), 6.77 (1H, d, J = 8.5 Hz), 5.13-4.98 (1H, m), 4.86 (1H, brs), 4.68 (2H, t, J = 8.8 Hz), 4.59-4.54 (1H, m), 4.22-4.16 (1H, m), 3.78-3.62 (2H, m), 3.55-3.34 (3H, m), 3.21-3.02 (2H, m), 2.92-2.88 (1H, m), 1.42 (3H, d, J = 6.7 Hz). |

### Examples 119 to 124

The compounds of Examples 119 to 124 were obtained by using the corresponding raw material compounds according to the method described in Example 21.

TABLE 70

| Example | Chemical structure | Physical property data |
|---|---|---|
| 119 | 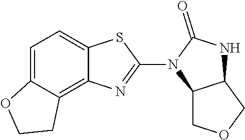 [SMILES: O=C1N[C@H]2COC[C@H]2N1c1nc2c3c(ccc2s1)OCC3] | LC-MS [M + H]⁺/Rt (min): 304.0/2.907 (Method C); ¹H-NMR (DMSO-d₆) δ: 8.15 (1H, s), 7.60 (1H, td, J = 0.8, 8.4 Hz), 6.75 (1H, d, J = 8.4 Hz), 5.08 (1H, dd, J = 4.4, 8.3 Hz), 4.65-4.54 (2H, m), 4.40 (1H, dd, J = 4.1, 8.3 Hz), 4.19 (1H, d, J = 10.4 Hz), 3.82 (1H, d, J = 10.0 Hz), 3.70 (1H, dd, J = 4.7, 10.4 Hz), 3.56 (1H, dd, J = 4.1, 10.0 Hz), 3.41-3.26 (2H, m). |
| 120 | 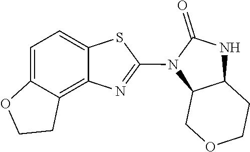 [SMILES: O=C1N[C@H]2CCOC[C@H]2N1c1nc2c3c(ccc2s1)OCC3] | LC-MS [M + H]⁺/Rt (min): 318.2/1.43 (Method E); ¹H-NMR (DMSO-d₆) δ: 7.90 (1H, s), 7.61- 7.57 (1H, m), 6.74 (1H, d, J = 8.4 Hz), 4.70-4.55 (3H, m), 4.21-4.10 (2H, m), 3.77- 3.67 (2H, m), 3.65-3.56 (1H, m), 3.41-3.34 (2H, m), 2.07-1.95 (1H, m), 1.76-1.68 (1H, m). |
| 121 | 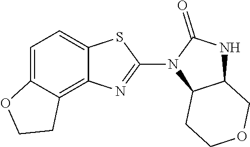 [SMILES: O=C1N[C@H]2COCC[C@H]2N1c1nc2c3c(ccc2s1)OCC3] | LC-MS [M + H]⁺/Rt (min): 318.2/1.38 (Method E); ¹H-NMR (DMSO-d₆) δ: 7.90 (1H, s), 7.59 (1H, dd, J = 0.9, 8.4 Hz), 6.74 (1H, d, J = 8.4 Hz), 4.84-4.74 (1H, m), 4.68-4.54 (2H, m), 3.87-3.72 (3H, m), 3.71-3.62 (1H, m), 3.50-3.27 (3H, m), 2.43-2.31 (1H, m), 1.85- 1.70 (1H, m). |
| 122 | 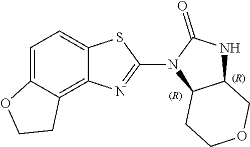 | LC-MS [M + H]⁺/Rt (min): 318.2/1.57 (Method E); ¹H-NMR (DMSO-d₆) δ: 7.91 (1H, s), 7.60 (1H, d, J = 8.4 Hz), 6.74 (1H, d, J = 8.4 Hz), 4.84-4.74 (1H, m), 4.61 (1H, td, J = 2.8, 8.8 Hz), 3.87-3.69 (3H, m), 3.66 (1H, dd, J = 3.1, 13.2 Hz), 3.53-3.29 (3H, m), 2.43-2.31 (1H, m), 1.83-1.72 (1H, m). |
| 123 | 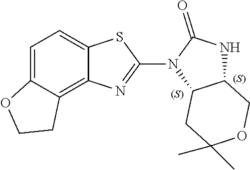 | LC-MS [M + H]⁺/Rt (min): 346.2/2.157 (Method C); ¹H-NMR (DMSO-d₆) δ: 7.91 (1H, s), 7.58 (1H, d, J = 8.4 Hz), 6.73 (1H, d, J = 8.4 Hz), 4.93-4.77 (1H, m), 4.68-4.52 (2H, m), 3.92-3.89 (1H, m), 3.75 (1H, dd, J = 3.1, 12.9 Hz), 3.55 (1H, dd, J = 2.6, 12.9 Hz), 3.38 (1H, t, J = 9.2 Hz), 2.16-2.04 (2H, m), 1.19 (3H, s), 1.04 (3H, s). |
| 124 | 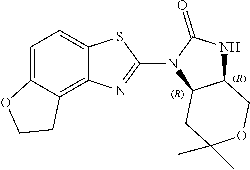 | LC-MS [M + H]⁺/Rt (min): 346.1/2.157 (Method C); ¹H-NMR (DMSO-d₆) δ: 7.92 (1H, s), 7.62- 7.55 (1H, m), 6.74 (1H, d, J = 8.4 Hz), 4.88-4.83 (1H, m), 4.69-4.50 (2H, m), 3.92- 3.89 (1H, m), 3.75 (1H, dd, J = 3.2, 12.9 Hz), 3.55 (1H, dd, J = 2.6, 12. 9 Hz), 3.38 (1H, t, J = 9.2 Hz), 2.16-2.04 (2H, m), 1.19 (3H, s), 1.04 (3H, s). |

### Examples 125 to 140

The compounds of Examples 125 to 140 were obtained by using the corresponding raw material compounds according to the method described in Reference Example 14.

TABLE 71-1

| Example | Chemical structure | Physical property data |
|---|---|---|
| 125 | 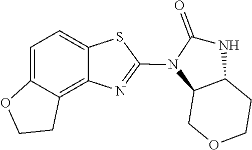 [SMILES: O=C1N[C@@H]2CCOC[C@H]2N1c1nc2c3c(ccc2s1)OCC3] | LC-MS [M + H]⁺/Rt (min): 318.2/1.69 (Method E); ¹H-NMR (DMSO-d₆) δ: 8.07 (1H, s), 7.65-7.59 (1H, m), 6.77 (1H, d, J = 8.4 Hz), 5.10-5.03 (1H, m), 4.66- 4.57 (2H, m), 4.04-3.97 (1H, m), 3.79- 3.65 (2H, m), 3.64-3.46 (2H, m), 3.38 (2H, t, J = 8.8 Hz), 2.00-1.92 (1H, m), 1.85-1.72 (1H, m). |
| 126 | 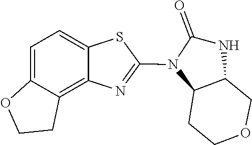 [SMILES: O=C1N[C@@H]2COCC[C@H]2N1c1nc2c3c(ccc2s1)OCC3] | LC-MS [M + H]⁺/Rt (min): 318.2/1.64 (Method E); ¹H-NMR (DMSO-d₆) δ: 7.87 (1H, s), 7.65-7.59 (1H, m), 6.77 (1H, d, J = 8.4 Hz), 4.64-4.57 (2H, m), 4.11- 4.00 (2H, m), 3.98-3.89 (1H, m), 3.53- 3.41 (2H, m), 3.36 (2H, t, J = 8.8 Hz), 3.07-2.96 (1H, m), 2.09-1.96 (1H, m). |
| 127 | 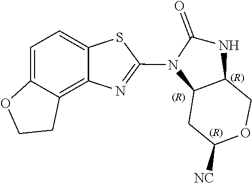 | LC-MS [M + H]⁺/Rt (min): 343.1/2.023 (Method C); ¹H-NMR (DMSO-d₆) δ: 8.02 (1H, s), 7.61 (1H, d, J = 8.4 Hz), 6.76 (1H, d, J = 8.4 Hz), 4.97-4.83 (2H, m), 4.62 (1H, ddd, J = 3.5, 8.0, 9.3 Hz), 3.96 (1H, dd, J = 2.5, 8.5 Hz), 3.82 (2H, d, J = 2.4 Hz), 3.46-3.33 (2H, m), 2.77-2.63 (1H, m), 2.32 (1H, td, J = 7.4, 14.5 Hz). |
| 128 | 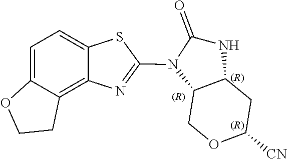 | LC-MS [M + H]⁺/Rt (min): 343.1/2.057 (Method C); ¹H-NMR (DMSO-d₆) δ: 8.15 (1H, s), 7.61 (1H, d, J = 8.4 Hz), 6.75 (1H, d, J = 8.4 Hz), 5.08 (1H, dd, J = 3.8, 6.8 Hz), 4.74 (1H, td, J = 4.8, 8.6 Hz), 4.67-4.55 (2H, m), 4.27-4.08 (3H, m), 3.44-3.31 (2H, m), 2.45-2.24 (1H, m), 2.21-1.99 (1H, m). |
| 129 | 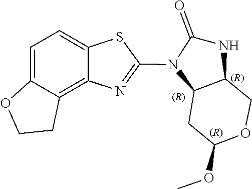 | LC-MS [M + H]⁺/Rt (min): 348.1/1.49 (Method E); ¹H-NMR (DMSO-d₆) δ: 7.94 (1H, s), 7.59 (1H, d, J = 8.4 Hz), 6.74 (1H, d, J = 8.4 Hz), 4.78 (1H, tdd, J = 2.7, 5.3, 8.8 Hz), 3.86-3.71 (2H, m), 3.38 (1H, dt, J = 4.9, 8.6 Hz), 3.30 (3H, s), 2.65 (1H, ddd, J = 3.7, 5.9, 13.7 Hz), 1.71 (1H, ddd, J = 8.0, 9.5, 13.7 Hz). |

TABLE 71-2

| | | |
|---|---|---|
| 130 | 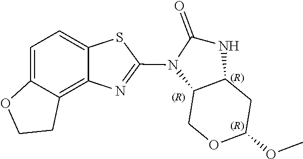 | LC-MS [M + H]⁺/Rt (min): 348.1/1.80 (Method E); ¹H-NMR (DMSO-d₆) δ: 7.94 (1H, s), 7.59 (1H, d, J = 8.4 Hz), 6.74 (1H, d, J = 8.4 Hz), 4.78 (1H, tdd, J = 2.7, 5.3, 8.8 Hz), 3.86-3.71 (2H, m), 3.38 (1H, dt, J = 4.9, 8.6 Hz), 3.30 (3H, s), 2.65 (1H, ddd, J = 3.7, 5.9, 13.7 Hz), 1.71 (1H, ddd, J = 8.0, 9.5, 13.7 Hz). |
| 131 | 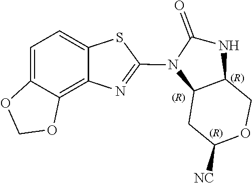 | LC-MS [M + H]⁺/Rt (min): 345.1/2.023 (Method C); ¹H-NMR (DMSO-d₆) δ: 8.08 (1H, s), 7.38 (1H, d, J = 8.3 Hz), 6.94 (1H, d, J = 8.3 Hz), 6.17-6.02 (2H, m), 4.99- 4.78 (2H, m), 3.99 (1H, dd, J = 2.7, 8.7 Hz), 3.84 (1H, dd, J = 2.7, 13.0 Hz), 3.78 (1H, dd, J = 2.3, 13.0 Hz), 2.72- 2.61 (1H, m), 2.44-2.35 (1H, m). |
| 132 | 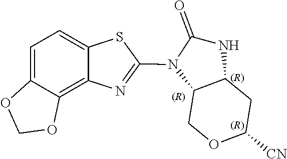 | LC-MS [M + H]⁺/Rt (min): 345.1/2.023 (Method C); ¹H-NMR (DMSO-d₆) δ: 8.20 (1H, s), 7.38 (1H, d, J = 8.3 Hz), 6.94 (1H, d, J = 8.3 Hz), 6.18-6.01 (2H, m), 5.08 (1H, dd, J = 3.6, 7.0 Hz), 4.73 (1H, td, J = 4.5, 8.9 Hz), 4.22 (1H, dd, J = 4.3, 9.0 Hz), 4.12 (1H, d, J = 4.5), 2.46-2.34 (1H, m), 2.05 (1H, td, J = 3.9, 15.2 Hz). |
| 133 | 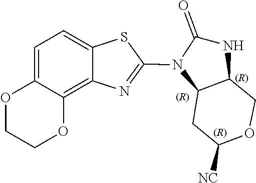 | LC-MS [M + H]⁺/Rt (min): 359.1/1.973 (Method C); ¹H-NMR (DMSO-d₆) δ: 8.00 (1H, s), 7.30 (1H, d, J = 8.6 Hz), 6.81 (1H, d, J = 8.6 Hz), 4.96-4.80 (2H, m), 4.43- 4.24 (4H, m), 4.02-3.92 (1H, m), 3.83 (1H, dd, J = 2.8, 13.0 Hz), 3.78 (1H, dd, J = 2.4, 13.0 Hz), 2.67 (1H, td, J = 5.1, 14.2 Hz), 2.40 (1H, td, J = 6.8, 14.2 Hz). |
| 134 | 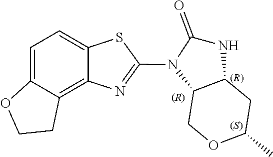 | LC-MS [M + H]⁺/Rt (min): 332.1/2.124 (Method C); ¹H-NMR (DMSO-d₆) δ: 8.09 (1H, s), 7.60 (1H, d, J = 8.4 Hz), 6.75 (1H, d, J = 8.4 Hz), 4.99 (1H, dd, J = 2.3, 13.1 Hz), 4.61 (2H, t, J = 8.8 Hz), 4.38 (1H, td, J = 2.7, 8.0 Hz), 3.98 (1H, dt, J = 6.0, 8.4 Hz), 3.81 (1H, dd, J = 3.2, 13.1 Hz), 3.59 (1H, ddd, J = 3.6, 6.4, 9.7 Hz), 3.37 (2H, dt, J = 3.0, 8.7 Hz), 2.04 (1H, ddd, J = 3.6, 5.9, 13.7 Hz). 1.35-1.21 (1H, m), 1.13 (3H, d, J = 6.2 Hz). |

TABLE 71-3

| | | |
|---|---|---|
| 135 | 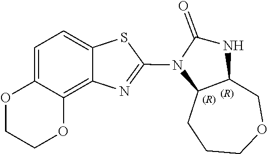 | LC-MS [M + H]⁺/Rt (min): 348.1/2.023 (Method C); ¹H-NMR (DMSO-d₆) δ: 7.89 (1H, s), 7.28 (1H, d, J = 8.5 Hz), 6.79 (1H, d, J = 8.5 Hz), 4.78 (1H, ddd, J = 2.7, 7.4, 9.9 Hz), 4.43-4.25 (4H, m), 4.12-4.02 (1H, m), 3.73-3.69 (2H, m), 3.69-3.64 (1H, m), 3.52 (1H, dt, J = 5.5, 11.6 Hz), 2.61-2.56 (1H, m), 2.27-2.19 (1H, m), 1.61-1.50 (2H, m). |
| 136 | 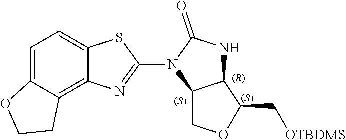 | LC-MS [M + H]⁺/Rt (min): 448.2/2.773 (Method C) |
| 137 | 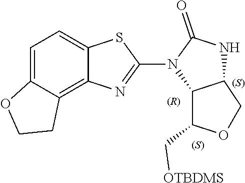 | LC-MS [M + H]⁺/Rt (min): 448.2/2.690 (Method C) |
| 138 | 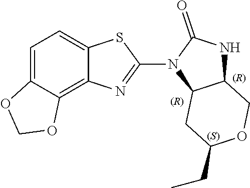 | LC-MS [M + H]⁺/Rt (min): 348.1/2.223 (Method C); ¹H-NMR (DMSO-d₆) δ: 7.92 (1H, s), 7.36 (1H, d, J = 8.3 Hz), 6.92 (1H, d, J = 8.3 Hz), 6.15-6.06 (2H, m), 4.86-4.76 (1H, m), 4.03-3.93 (1H, m), 3.84-3.76 (1H, m), 3.70-3.62 (1H, m), 3.29-3.21 (1H, m), 2.47-2.38 (1H, m), 1.48-1.37 (2H, m), 1.28-1.13 (1H, m), 0.86 (3H, t, J = 7.4 Hz) . |
| 139 | 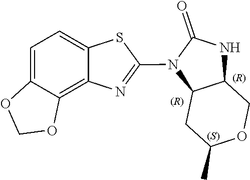 | LC-MS [M + H]⁺/Rt (min): 334.1/2.107 (Method C); ¹H-NMR (DMSO-d₆) δ: 7.92 (1H, s), 7.36 (1H, d, J = 8.3 Hz), 6.93 (1H, d, J = 8.3 Hz), 6.11 (1H, d, J = 1.1 Hz), 6.09 (1H, d, J = 1.1 Hz), 4.83- 4.77 (1H, m), 3.99-3.89 (1H, m), 3.79 (1H, d, J = 7.2 Hz), 3.67 (1H, dd, J = 2.7, 13.2 Hz), 3.49-3.42 (1H, m), 2.47- 2.42 (1H, m), 1.23-1.14 (1H, m), 1.10 (3H, t. J = 6.1 Hz). |
| 140 | 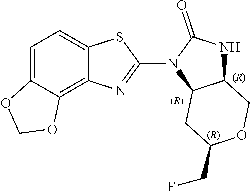 | LC-MS [M + H]⁺/Rt (min): 352.1/2.073 (Method C); ¹H-NMR (DMSO-d₆) δ: 7.97 (s, 1H), 7.37 (d, J = 8.3 Hz, 1H), 6.93 (d, J = 8.3 Hz, 1H), 6.11 (d, J = 1.1 Hz, 1H), 6.09 (d, J = 1.1 Hz, 1H), 4.88 (dt, J = 9.8, 6.9 Hz, 1H), 4.54-4.16 (m, 2H), 4.06-3.92 (m, 1H), 3.85 (d, J = 7.4 Hz, 1H), 3.79-3.60 (m, 2H), 2.43 (ddd, J = 13.3, 6.7, 2.3 Hz, 1H), 1.40- 1.19 (m, 1H). |

### Examples 141 to 143

The compounds of Examples 141 to 143 were obtained by using the corresponding raw material compounds according to the method described in Reference Example 70.

TABLE 72

| Example | Chemical structure | Physical property data |
|---|---|---|
| 141 | 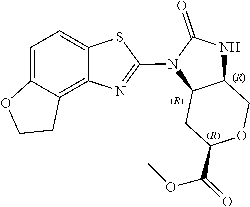 | LC-MS [M + H]⁺/Rt (min): 376.1/2.006 (Method C); ¹H-NMR (DMSO-d₆) δ: 7.96 (1H, s), 7.60 (1H, d, J = 8.4 Hz), 6.75 (1H, d, J = 8.4 Hz), 4.92 (1H, q, J = 7.7 Hz), 4.66-4.53 (2H, m), 4.28 (1H, dd, J = 3.5, 10.2 Hz), 3.95 (1H, dd, J = 1.6, 13.0 Hz), 3.91-3.84 (1H, m), 3.78 (1H, dd, J = 2.7, 13.0 Hz), 3.55 (3H, s), 3.43-3.34 (2H, m), 2.69 (1H, ddd, J = 3.6, 6.0, 13.6 Hz), 1.87-1.67 (1H, m) . |
| 142 | 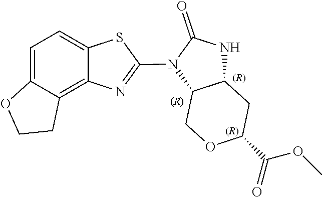 | LC-MS [M + H]⁺/Rt (min): 376.1/2.007 (Method C) |
| 143 | 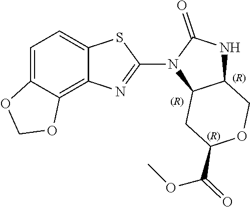 | LC-MS [M + H]⁺/Rt (min): 378.1/2.006 (Method C) |

### Examples 144 to 147

The compounds of Examples 144 to 147 were obtained by using the corresponding raw material compounds according to the method described in Reference Example 72.

TABLE 73

| Example | Chemical structure | Physical property data |
|---|---|---|
| 144 | 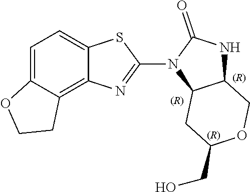 | LC-MS [M + H]⁺/Rt (min): 348.2/1.856 (Method C); ¹H-NMR (DMSO-d₆) δ: 7.90 (1H, s), 7.60 (1H, d, J = 8.4 Hz), 6.74 (1H, d, J = 8.4 Hz), 4.85 (1H, td, J = 6.8, 9.7 Hz), 4.70 (1H, t, J = 5.6 Hz), 4.66-4.53 (2H, m), 3.98 (1H, d, J = 13.1 Hz), 3.80 (1H, d, J = 7.2 Hz), 3.68 (1H, dd, J = 2.7, 13.0 Hz), 3.45-3.33 (5H, m), 263-2.40 (1H, m), 1.33- 1.15 (1H, m). |
| 145 | 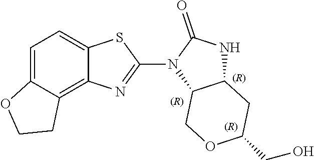 | LC-MS [M + H]⁺/Rt (min): 348.1/1.873 (Method C); ¹H-NMR (DMSO-d₆) δ: 8.08 (1H, s), 7.60 (1H, d, J = 8.4 Hz), 6.75 (1H, d, J = 8.4 Hz), 4.70 (1H, t, J = 5.8 Hz), 4.61 (2H, t, J = 8.8 Hz), 4.46 (1H, td, J = 3.2, 8.4 Hz), 4.04 (2H, dt, J = 5.7, 8.2 Hz), 3.88 (1H, dd, J = 3.5, 12.9 Hz), 3.54 (1H, td, J = 4.8, 9.6 Hz), 3.49-3.26 (4H, m), 1.99 (1H, ddd, J = 4.3, 5.8, 13.9 Hz), 1.43 (1H, td, J = 8.8, 13.9 Hz). |
| 146 | 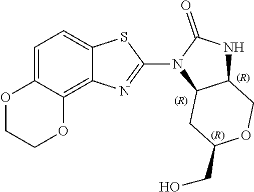 | LC-MS [M + H]⁺/Rt (min): 364.1/1.840 (Method C); ¹H-NMR (DMSO-d₆) δ: 7.87 (1H, s), 7.29 (1H, d, J = 8.6 Hz), 6.80 (1H, d, J = 8.6 Hz), 4.81 (1H, td, J = 6.8, 9.9 Hz), 4.76- 4.66 (1H, m), 4.42-4.23 (4H, m), 4.02-3.90 (1H, m), 3.80 (1H, d, J = 7.4 Hz), 3.68 (1H, dd, J = 2.8, 13.0 Hz), 3.46-3.24 (3H, m), 2.50-2.40 (1H, m), 1.30-1.15 (1H, m). |
| 147 | 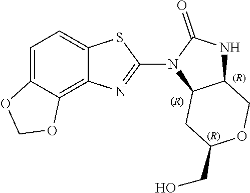 | LC-MS [M + H]⁺/Rt (min): 350.1/1.890 (Method C); ¹H-NMR (DMSO-d₆) δ: 7.95 (1H, s), 7.37 (1H, dd, J = 1.9, 8.3 Hz), 6.93 (1H, dd, J = 1.9, 8.3 Hz), 6.11-6.09 (2H, m), 4.87- 4.79 (1H, m), 4.72-4.67 (1H, m), 3.98 (1H, d, J = 12.7 Hz), 3.87-3.76 (1H, m), 3.68 (1H, d, J = 13.0 Hz), 3.47-3.31 (3H, m), 2.61-2.42 (1H, m), 1.35-1.14 (1H, m). |

### Example 148

The compound of Example 148 was obtained by using the corresponding raw material compounds according to the method described in Reference Example 75.

TABLE 74

| Example | Chemical structure | Physical property data |
|---|---|---|
| 148 | 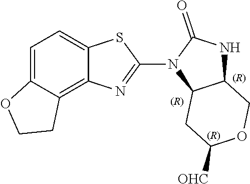 | LC-MS [M + H]⁺/Rt (min): 346.1/1.907 (Method C) |

### Example 149

The compound of Example 149 was obtained by using the corresponding raw material compounds according to the method described in Reference Example 76.

TABLE 75

| Example | Chemical structure | Physical property data |
|---|---|---|
| 149 | 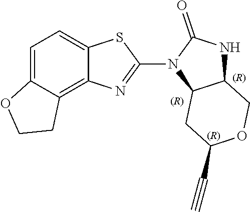 | LC-MS [M + H]⁺/Rt (min): 342.1/2.123 (Method C); ¹H-NMR (DMSO-d₆) δ: 7.93 (1H, s), 7.60 (1H, d, J = 8. 4 Hz), 6.75 (1H, d, J = 8.4 Hz), 4.93-4.80 (1H, m), 4.66-4.50 (2H, m), 4.34 (1H, td, J = 2.6, 10.5 Hz), 3.95-3.86 (1H, m), 3.83 (1H, d, J = 7.6 Hz), 3.72 (1H, dd, J = 2.8, 13.1 Hz), 3.45 (1H, d, J = 2.1 Hz), 3.41-3.33 (2H, m), 2.72-2.57 (1H, m), 1.76-1.60 (1H, m). |

### Example 150

### (3aR,7aR)-1-(7,8-Dihydrofuro[3,2-e][1,3]benzothiazol-2-yl)-6-hydroxyhexahydropyrano[3,4-d]imidazol-2(3H)-one

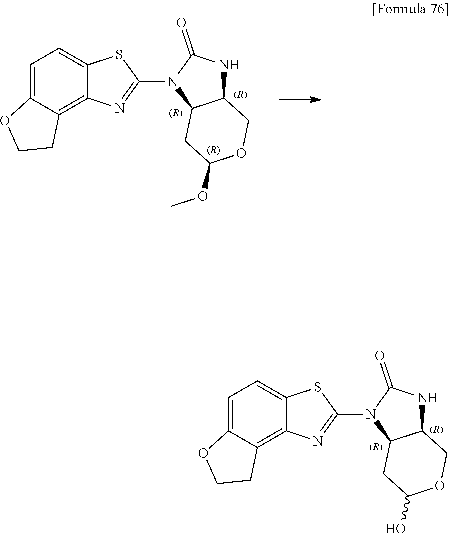

1 N Hydrochloric acid (4 mL) was added to a 1,4-dioxane solution (3 mL) of the compound of Example 129 (53.0 mg) at room temperature, and the resulting mixture was stirred at room temperature for 1 hour and at 60° C. for 2 hours. The reaction mixture was cooled to room temperature, then saturated aqueous sodium bicarbonate was added, and the resulting mixture was extracted with ethyl acetate. The organic layer was dried over sodium sulfate, filtered, and then concentrated under reduced pressure. The residue was washed with ethyl acetate to obtain the title compound (8.0 mg).

LC-MS [M+H]⁺/Rt (min): 334.1/2.761 (Method C); 1H-NMR (DMSO-d₆) δ: 7.95 (1H, s), 7.59 (1H, d, J=8.4 Hz), 6.74 (1H, d, J=8.4 Hz), 6.40 (1H, d, J=4.9 Hz), 5.00-4.85 (1H, m), 4.81 (1H, td, J=4.4, 9.1 Hz), 4.67-4.52 (2H, m), 3.98-3.84 (2H, m), 3.49-3.43 (1H, m), 3.42-3.34 (1H, m), 2.77-2.68 (1H, m), 2.00-1.90 (2H, m).

### Example 151

### (3aR,7aR)-1-(7,8-Dihydrofuro[3,2-e][1,3]benzothiazol-2-yl)-6-fluorohexahydropyrano[3,4-d]imidazol-2(3H)-one

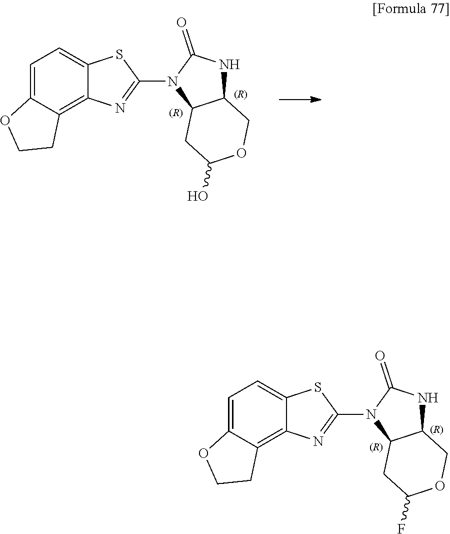

Diethylaminosulfur trifluoride (0.041 mL) was added to a dichloromethane/1,4-dioxane solution (6 mL/9 mL) of Example 150 (69.0 mg) under ice cooling, and the resulting mixture was stirred under ice cooling for 15 minutes. Saturated aqueous sodium bicarbonate was added to the reaction mixture under ice cooling, and the resulting mixture was extracted with chloroform. The organic layer was dried over sodium sulfate, filtered, and then concentrated under reduced pressure. The residue was purified by silica gel column chromatography (chloroform/methanol) to obtain the title compound (20.0 mg).

LC-MS [M+H]⁺/Rt (min): 336.1/2.123 (Method C); ¹H-NMR (DMSO-d₆) δ: 8.08 (1H, s), 7.60 (1H, d, J=8.4 Hz), 6.75 (1H, d, J=8.4 Hz), 5.67 (1H, ddd, J=5.0, 6.9, 60.9 Hz), 4.94-4.83 (1H, m), 4.67-4.54 (2H, m), 4.13-3.96 (2H, m), 3.68 (1H, d, J=12.6 Hz), 3.45-3.33 (2H, m), 3.22-3.02 (1H, m), 2.32-2.14 (1H, m).

### Example 152

The compound of Example 152 was obtained by using the corresponding raw material compounds according to the method described in Reference Example 75.

TABLE 76

| Example | Chemical structure | Physical property data |
|---|---|---|
| 152 | 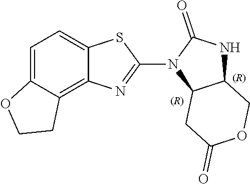 | LC-MS [M + H]⁺/Rt (min): 332.1/1.956 (Method C); ¹H-NMR (DMSO-d₆) δ: 8.24 (1H, s), 7.61 (1H, d, J = 8.4 Hz), 6.77 (1H, d, J = 8.4 Hz), 5.17 (1H, ddd, J = 2.2, 5.2, 10.1 Hz), 4.68-4.55 (2H, m), 4.48 (1H, dd, J = 2.1, 12.5 Hz), 4.36 (1H, d, J = 10.2 Hz), 4.22 (1H, dd, J = 1.7, 12.5 Hz), 3.53-3.32 (3H, m), 3.18 (1H, dd, J = 5.2, 15.8 Hz). |

### Examples 153 and 154

The compounds of Examples 153 and 154 were obtained by using the corresponding raw material compounds according to the method described in Reference Example 147.

TABLE 77

| Example | Chemical structure | Physical property data |
|---|---|---|
| 153 | 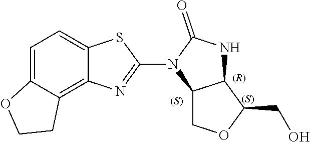 | LC-MS [M + H]⁺/Rt (min): 334.1/1.890 (Method C); ¹H-NMR (DMSO-d₆) δ: 8.01 (1H, s), 7.60 (1H, d, J = 8.4 Hz), 6.75 (1H, d, J = 8.4 Hz), 5.14-5.06 (1H, m), 4.79 (1H, t, J = 5.5 Hz), 4.67-4.53 (2H, m), 4.40-4.31 (1H, m), 4.23-4.16 (1H, m), 3.82-3.74 (1H, m), 3.70-3.51 (3H, m), 3.39-3.33 (3H, m). |
| 154 | 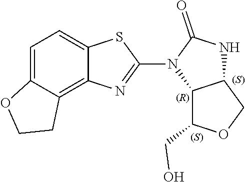 | LC-MS [M + H]⁺/Rt (min): 334.1/1.890 (Method C); ¹H-NMR (DMSO-d₆) δ: 8.21 (1H, s), 7.61 (1H, d, J = 8.4 Hz), 6.77 (1H, d, J = 8.4 Hz), 5.17-5.10 (1H, m), 4.97-4.90 (1H, m), 4.63 (1H, t, J = 8.8 Hz), 4.49-4.42 (1H, m), 3.87-3.80 (2H, m), 3.75-3.66 (1H, m), 3.62-3.55 (1H, m), 3.43-3.28 (3H, m). |

### Examples 155 and 156

The compounds of Examples 155 and 156 were obtained by using the corresponding raw material compounds according to the method described in Reference Example 14.

TABLE 78

| Example | Chemical structure | Physical property data |
|---|---|---|
| 155 | 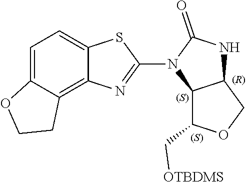 | LC-MS [M + H]⁺/Rt (min): 448.0/2.840 (Method C) |
| 156 | 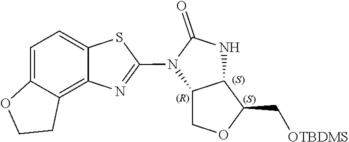 | LC-MS [M + H]⁺/Rt (min): 448.2/2.823 (Method C) |

### Examples 157 and 158

The compounds of Examples 157 and 158 were obtained by using the corresponding raw material compounds according to the method described in Reference Example 147.

TABLE 79

| Example | Chemical structure | Physical property data |
|---|---|---|
| 157 | 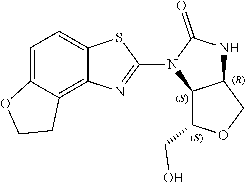 | LC-MS [M + H]⁺/Rt (min): 334.1/1.907 (Method C); ¹H-NMR (DMSO-d₆) δ: 8.19 (1H, s), 7.60 (1H, d, J = 8.4 Hz), 6.75 (1H, d, J = 8.4 Hz), 4.96 (1H, dd, J = 5.4, 5.4 Hz), 4.92 (1H, dd, J = 2.3, 8.5 Hz), 4.68-4.56 (2H, m), 4.44-4.37 (1H, m), 4.18-4.12 (1H, m), 4.10-4.05 (1H, m), 3.84-3.69 (3H, m), 3.39- 3.33 (2H, m). |
| 158 | 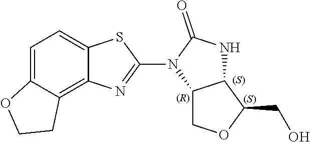 | LC-MS [M + H]⁺/Rt (min): 334.1/1.890 (Method C); ¹H-NMR (DMSO-d₆) δ: 8.26 (1H, s), 7.60 (1H, d, J = 8.4 Hz), 6.75 (1H, d, J = 8.4 Hz), 5.11-5.03 (1H, m), 4.95 (1H, dd, J = 5.4, 5.4 Hz), 4.66-4.55 (2H, m), 4.28-4.17 (2H, m), 4.01-3.95 (1H, m), 3.88-3.81 (1H, m), 3.55-3.47 (2H, m), 3.39-3.33 (2H, m). |

### Example 159

The compound of Example 159 was obtained by using the corresponding raw material compounds according to the method described in Reference Example 74.

TABLE 80

| Example | Chemical structure | Physical property data |
|---|---|---|
| 159 | 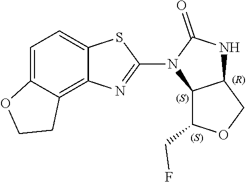 | LC-MS [M + H]⁺/Rt (min): 336.1/2.157 (Method C); ¹H-NMR (DMSO-d₆) δ: 8.26 (1H, s), 7.61 (1H, d, J = 8.4 Hz), 6.76 (1H, d, J = 8.4 Hz), 4.94 (1H, dd, J = 3.0, 8.7 Hz), 4.92- 4.72 (2H, m), 4.67-4.56 (2H, m), 4.46-4.40 (1H, m), 4.40-4.30 (1H, m), 4.11-4.03 (1H, m), 3.79-3.73 (1H, m), 3.41-3.34 (2H, m). |

### Example 160

The compound of Example 160 was obtained by using the corresponding raw material compounds according to the method described in Reference Example 14.

TABLE 81

| Example | Chemical structure | Physical property data |
|---|---|---|
| 160 | 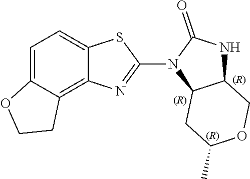 | LC-MS [M + H]⁺/Rt (min): 332.1/2.123 (Method C); ¹H-NMR (DMSO-d₆) δ: 7.87 (1H, s), 7.59 (1H, d, J = 8.4 Hz), 6.74 (1H, d, J = 8.4 Hz), 4.70-4.53 (3H, m), 4.30 (1H, dd, J = 5.6, 11.8 Hz), 4.19-4.08 (1H, m), 3.78- 3.63 (1H, m), 3.45 (1H, dd, J = 8.2, 11.8 Hz), 3.40-3.33 (2H, m), 1.87-1.82 (1H, m), 1.64 (1H, ddd, J = 4.2, 11.3, 15.1 Hz), 1.13 (3H, d, J = 6.2 Hz). |

### Example 161

(3aR,6R,6aS)-1-(7,8-Dihydrofuro[3,2-e][1,3]benzothiazol-2-yl)-6-methyltetrahydro-1H-furo[3,4-d]imidazol-2(3H)-one

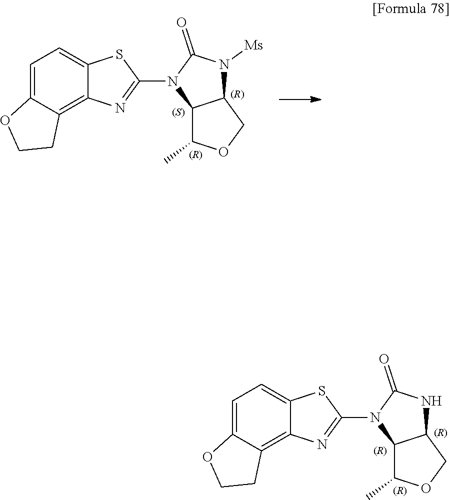

1.0 M Tetrahydrofuran solution (0.024 mL) of tetrabutylammonium fluoride was added to a tetrahydrofuran solution (3 mL) of Reference Example 290 (1.0 mg) at room temperature, and the resulting mixture was stirred at room temperature for 2 days. The reaction mixture was concentrated under reduced pressure, and then the residue was purified by silica gel column chromatography (hexane/ethyl acetate) to obtain the title compound (0.6 mg).

LC-MS [M+H]⁺/Rt (min): 318.1/2.123 (Method C); 1H-NMR (DMSO-d₆) δ: 8.17 (1H, s), 7.60 (1H, d, J=8.4 Hz), 6.75 (1H, d, J=8.4 Hz), 4.68 (1H, dd, J=2.4, 8.7 Hz), 4.65-4.58 (2H, m), 4.46-4.38 (1H, m), 4.29-4.21 (1H, m), 4.02-3.94 (1H, m), 3.65 (1H, dd, J=2.6, 9.9 Hz), 3.50-3.38 (2H, m), 1.38 (3H, d, J=6.6 Hz).

### Example 162

### [(3aS,4R,6aR)-3-(7,8-Dihydrofuro[3,2-e][1,3]benzothiazol-2-yl)-2-oxohexahydro-1H-furo[3,4-d]imidazol-4-yl]acetonitrile

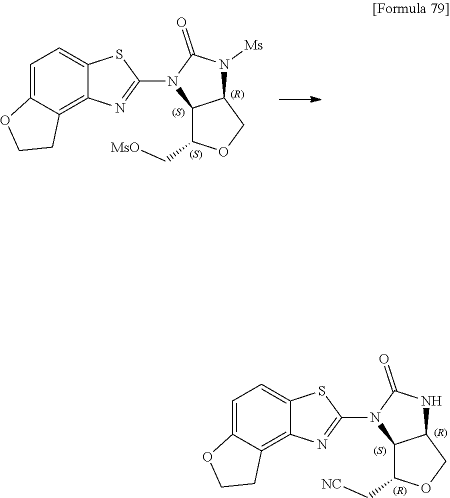

Potassium cyanide (4.0 mg) was added to a dimethyl sulfoxide solution (2 mL) of Reference Example 285 (20.0 mg) at room temperature, and the resulting mixture was stirred at 80° C. for 1 hour. Potassium cyanide (16.5 mg) was further added, and the resulting mixture was stirred at 80° C. for 35 minutes, then 1.0 M tetrahydrofuran solution (0.204 mL) of tetrabutylammonium fluoride was added at room temperature, and the resulting mixture was stirred at 70° C. for 5 hours. The reaction mixture was concentrated under reduced pressure, and then the residue was purified by silica gel column chromatography (hexane/ethyl acetate) to obtain the title compound (2.4 mg).

LC-MS [M+H]⁺/Rt (min): 343.1/2.090 (Method C); 1H-NMR (DMSO-d₆) δ: 8.27 (1H, s), 7.61 (1H, d, J=8.4 Hz), 6.76 (1H, d, J=8.4 Hz), 4.82 (1H, dd, J=3.1, 8.9 Hz), 4.69-4.56 (2H, m), 4.49-4.56 (2H, m), 4.49-4.41 (1H, m), 4.37 (1H, dt, J=3.1, 6.2 Hz), 4.13 (1H, dd, J=5.2, 9.9 Hz), 3.72 (1H, dd, J=3.3, 10.0 Hz 3.45-3.36 (2H, m), 3.19-3.17 (2H, m).

### Test Example 1

[Inhibition Test on Activity of DYRK Family (DYRK1A, DYRK1B, DYRK2, and DYRK3)]

(Method for Measuring Kinase Activity)

The kinase activity was measured by mobility shift assay (MSA) method using QuickScout Screening Assist™ MSA (commercially available kit manufactured by Carna Biosciences, Inc.). The substrate of the kinase reaction used was an FITC-labeled DYRKtide peptide included in the kit. An assay buffer [20 mM HEPES, 0.01% Triton X-100™, 2 mM dithiothreitol, pH 7.5]was used to create a substrate mixture solution with a substrate (4 μM), MgCl₂(20 mM), and ATP (DYRK1A: 100 μM; DYRK1B: 200 μM; DYRK2: 40 μM; and DYRK3: 20 μM). In addition, kinases (DYRK1A: manufactured by Carna Biosciences, Inc., Cat. No. 04-130; DYRK1B: manufactured by Carna Biosciences, Inc., Cat. No. 04-131; DYRK2; manufactured by Carna Biosciences, Inc., Cat. No. 04-132; and DYRK3; manufactured by Carna Biosciences, Inc., Cat. No. 04-133) were diluted with the assay buffer to prepare enzyme solutions (DYRK1A: 0.2 ng/μL; DYRK1B: 0.08 ng/μL; DYRK2: 0.04 ng/μL; and DYRK3: 0.25 ng/μL). The 10 mM solution of the test compound in DMSO was further diluted with DMSO to 10 levels of the concentration (0.00003 mM, 0.0001 mM, 0.0003 mM, 0.001 mM, 0.003 mM, 0.01 mM, 0.03 mM, 0.1 mM, 0.3 mM, and 1 mM), each of which was subjected to 25-fold dilution with the assay buffer to obtain a drug solution (4% DMSO solution). 5 μL of the drug solution or a control solution (4% DMSO-assay buffer), 5 μL of the substrate mixture solution, and 10 μL of the enzyme solution were mixed in the wells of a polypropylene 384-well plate and allowed to react at room temperature for 1 hour, and then the reaction was quenched by adding 60 μL of the termination buffer included in the kit. Subsequently, the quantities of the substrates (S) and the phosphorylated substrate (P) in the reaction solution were measured using LabChip EZ Reader II system (manufactured by Caliper Life Sciences) according to the protocol of the assay kit.

(Method for Evaluating Inhibitory Activity)

The heights of the peaks of the “substrate” and the “phosphorylated substrate” were expressed as S and P, respectively, and a blank containing the assay buffer instead of the enzyme solution was also measured.

The inhibition rate (%) of the test compound was calculated according to the following equation:

Inhibition rate (%)=(1−(C−A)/(B−A))×100
wherein, A, B, and C represent P/(P+S) of the blank well, P/(P+S) of the control solution well, and P/(P+S) of the compound-containing well, respectively.

The IC₅₀ value was calculated via a regression analysis of the inhibition rate and the test compound concentration (logarithmic value).

(Evaluation Result)

The inhibiting activities of representative compounds of the present invention are shown against DYRK1A, DYRK1B, DYRK2, and DYRK3 in Tables. The kinase activity inhibitory effect was indicated with the mark *** at an IC₅₀ value of less than 0.01 μM; the mark ** at 0.01 μM or more and less than 0.1 μM; the mark * at 0.1 μM or more and less than 1 μM; and the mark—at 1 μM or more.

TABLE 82

| | Test Compound | | | |
|---|---|---|---|---|
| Example | Inhibitory activity | | | |
| No. | DYRK 1A | DYRK 1B | DYRK 2 | DYRK 3 |
| 1 | ** | NT | — | * |
| 2 | ** | NT | * | ** |
| 3 | * | * | — | * |
| 4 | * | NT | — | * |
| 5 | ** | NT | * | * |
| 7 | ** | ** | — | — |
| 8 | ** | ** | — | — |
| 9 | ** | ** | * | * |
| 10 | * | NT | — | — |
| 11 | ** | ** | — | — |
| 12 | ** | NT | * | * |
| 13 | ** | NT | — | — |
| 14 | ** | ** | * | ** |
| 15 | ** | NT | — | — |
| 16 | * | NT | — | — |
| 17 | ** | NT | — | — |
| 18 | ** | NT | * | * |
| 19 | ** | NT | * | * |
| 20 | * | NT | - | - |
| 21 | *** | NT | * | * |
| 22 | * | NT | — | — |
| 23 | *** | *** | * | * |
| 24 | ** | ** | — | — |
| 25 | ** | NT | — | — |
| 26 | — | NT | — | — |
| 27 | — | — | — | — |
| 28 | ** | ** | — | — |

TABLE 83

| | Test Compound | | | |
|---|---|---|---|---|
| Example | Inhibitory activity | | | |
| No. | DYRK 1A | DYRK 1B | DYRK 2 | DYRK 3 |
| 29 | ** | NT | — | — |
| 30 | ** | NT | — | — |
| 31 | *** | NT | — | — |
| 32 | *** | NT | — | — |
| 33 | * | NT | — | — |
| 34 | *** | ** | — | — |
| 35 | — | NT | — | — |
| 36 | *** | *** | — | — |
| 37 | *** | NT | * | * |
| 38 | *** | *** | * | * |
| 39 | *** | *** | * | * |
| 40 | *** | *** | * | ** |
| 41 | *** | *** | * | ** |
| 42 | *** | *** | * | * |
| 43 | *** | *** | * | * |

TABLE 84

| | Test Compound | | | |
|---|---|---|---|---|
| Example | Inhibitory activity | | | |
| No. | DYRK 1A | DYRK 1B | DYRK 2 | DYRK 3 |
| 44 | *** | NT | * | * |
| 45 | ** | NT | * | ** |
| 46 | ** | NT | * | * |
| 47 | * | NT | — | * |
| 48 | ** | NT | * | ** |
| 49 | ** | NT | * | * |
| 50 | * | NT | — | * |
| 51 | ** | NT | * | * |
| 52 | * * | NT | * | * |
| 53 | * | NT | — | * |
| 54 | ** | NT | * | * |
| 55 | * | NT | — | * |
| 56 | * | NT | — | * |
| 57 | * | NT | — | * |
| 65 | *** | NT | ** | ** |
| 66 | * | NT | — | — |
| 67 | * | NT | — | * |
| 68 | * | NT | — | * |
| 69 | ** | NT | — | — |
| 70 | ** | NT | — | * |
| 71 | ** | NT | — | — |
| 72 | ** | NT | — | — |
| 73 | *** | NT | * | — |
| 74 | * | NT | — | * |
| 75 | ** | NT | — | — |
| 76 | ** | NT | — | — |
| 77 | ** | NT | — | * |
| 78 | ** | NT | — | * |
| 79 | ** | NT | — | — |
| 80 | ** | NT | — | — |
| 81 | ** | NT | — | — |
| 82 | ** | NT | — | — |
| 83 | * | NT | — | * |
| 84 | ** | NT | * | * |
| 85 | ** | NT | — | — |
| 86 | ** | NT | — | — |
| 87 | * | NT | * | — |
| 88 | * | NT | — | — |
| 89 | * | NT | — | — |
| 90 | ** | NT | * | * |
| 91 | ** | NT | * | ** |

TABLE 85

| | Test Compound | | | |
|---|---|---|---|---|
| Example | Inhibitory activity | | | |
| No. | DYRK 1A | DYRK 1B | DYRK 2 | DYRK 3 |
| 92 | * | NT | — | — |
| 93 | ** | NT | — | * |
| 94 | * | NT | — | — |
| 95 | * | NT | * | * |
| 96 | * | NT | — | — |
| 97 | * | NT | — | — |
| 98 | *** | NT | — | — |
| 99 | *** | NT | * | * |
| 100 | *** | ** | — | — |
| 101 | ** | NT | — | — |
| 102 | ** | NT | — | * |
| 103 | ** | NT | — | * |
| 104 | *** | NT | * | * |
| 105 | ** | NT | — | — |
| 106 | ** | NT | — | * |
| 107 | ** | NT | — | — |
| 108 | ** | NT | — | — |
| 109 | — | NT | — | — |
| 110 | *** | NT | — | — |
| 111 | * | NT | — | — |
| 112 | *** | NT | — | * |
| 113 | * | NT | — | |
| 114 | *** | NT | * | * |
| 115 | ** | NT | * | — |
| 116 | ** | NT | — | — |
| 117 | ** | NT | — | — |
| 118 | ** | NT | — | — |
| 119 | ** | ** | ** | ** |
| 120 | ** | NT | * | * |
| 121 | ** | NT | * | * |
| 122 | *** | *** | * | * |
| 123 | — | NT | — | — |
| 124 | *** | NT | * | * |
| 125 | ** | NT | * | * |
| 126 | * | NT | * | * |
| 127 | *** | NT | * | * |
| 128 | — | NT | — | — |
| 129 | ** | NT | * | * |
| 130 | * | NT | — | — |
| 131 | *** | NT | * | * |
| 132 | — | NT | — | — |

TABLE 86

| | Test Compound | | | |
|---|---|---|---|---|
| Example | Inhibitory activity | | | |
| No. | DYRK 1A | DYRK 1B | DYRK 2 | DYRK 3 |
| 133 | *** | NT | * | * |
| 134 | * | NT | — | — |
| 135 | ** | NT | * | * |
| 138 | *** | *** | * | * |
| 139 | *** | *** | * | ** |
| 140 | *** | *** | ** | ** |
| 141 | ** | NT | — | — |
| 144 | *** | NT | — | * |
| 145 | * | NT | — | — |
| 146 | *** | NT | — | * |
| 147 | *** | NT | — | * |
| 149 | *** | NT | — | * |
| 150 | ** | NT | * | * |
| 151 | *** | NT | * | ** |
| 152 | *** | NT | ** | ** |
| 153 | ** | NT | * | * |
| 154 | — | NT | — | — |
| 157 | * * | ** | * | * |
| 158 | * | * | — | — |
| 159 | *** | ** | ** | ** |
| 160 | * | * | — | — |
| 161 | ** | ** | ** | * |
| 162 | * * | ** | * | * |

These results have shown that the test compounds (Compounds (1) of the present invention) have potent DYRK-inhibitory activities.

### Test Example 2

[Hematological Test by Oral Administration Using Mice]

In the present test, mice aged 8 to 12 weeks (B6C₃F1, female, Charles River Laboratories Japan, Inc.) were used. A 0.5% methylcellulose solution or an Example compound suspended in a 0.5% methylcellulose solution was orally administered as a single dose or as repeated doses of 10 mL/kg to the mice, and on or after the day after the final administration, the blood collected from the posterior vena cava under isoflurane anesthesia was subjected to anticoagulation treatment with a blood collection tube containing EDTA-2K, and the reticulocyte count was quantified by using a multi-item automated hematology analyzer (manufactured by Sysmex Corporation).

### INDUSTRIAL APPLICABILITY

The compound provided by the present invention is useful as a prophylactic or therapeutic agent for disease which is known to be involved in abnormal cell response through DYRK1A, for example, Alzheimer's disease, Parkinson's disease, Down's syndrome, mental retardation, memory impairment, memory loss, neuropsychiatric disorder such as depression, and cancers such as brain tumors. The compound is a DYRK1B inhibitor also useful as a prophylactic or therapeutic pharmaceutical (pharmaceutical composition) for cancers such as pancreatic cancer. Since DYRK2 controls p53 to induce apoptosis in response to DNA damages, the compound provided by the present invention is further useful as a prophylactic or therapeutic pharmaceutical (pharmaceutical composition) for bone resorption disease and osteoporosis. The compound provided by the present invention is a DYRK3 inhibitor also useful as a prophylactic or therapeutic pharmaceutical (pharmaceutical composition) for sickle-cell anemia, bone resorption disease in chronic kidney disease, and osteoporosis. The compound is also useful, as a compound inhibiting DYRK, for reagents to be used in pathological imaging and for reagents for basic experiments and research regarding the above diseases.

1. A compound represented by the following formula (1): 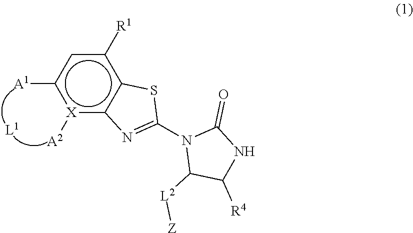
wherein
A¹ represents an oxygen atom or a nitrogen atom (═N—),
A² represents CR^{B}, CR^{C}R^{D}, an oxygen atom, or NR^{A1}
L¹ represents optionally substituted methylene, optionally substituted ethylene, optionally substituted methine, optionally substituted ethanediylidene, ═N—, or NR^{A2},
R^{A1}, R^{A2}, R^{B}, R^{C}, and R^{D} each independently represent a hydrogen atom or optionally substituted C₁₋₆ alkyl,
R¹ represents a hydrogen atom, a halogen atom, or optionally substituted C₁₋₆ alkyl,
X represents a carbon atom or a nitrogen atom,
L² represents optionally substituted C₁₋₄ alkylene,
R^{E} represents optionally substituted C₁-6 alkyl,
Z represents —NR²R³ or —OR⁷,
R⁷ represents optionally substituted C₁₋₆ alkyl, or optionally substituted C₁₋₇ alkylene formed together with R⁴, wherein R⁴ and R⁷, together with the carbon atom and the oxygen atom to which they, respectively, are attached, form an optionally substituted 5- to 11-membered saturated heterocycle,
R² represents a hydrogen atom, optionally substituted C₁₋₆ alkyl, C(O)—R^{E}, C₃₋₁₀ cycloalkyl, C₂₋₆alkynyl, or a cyclic group of a 4- to 11-membered saturated heterocycle,
R³ represents a hydrogen atom, optionally substituted C₁₋₆ alkyl, or C(O)—R^{E}, and
R⁴ represents optionally substituted C₁₋₆ alkyl,
wherein R² and R³, together with the nitrogen atom to which they are attached, may form an optionally substituted 4- to 11-membered saturated heterocycle, or R³ and R⁴, together with the nitrogen atom and the carbon atom to which they, respectively, are attached, may form an optionally substituted 4- to 11-membered saturated heterocycle, or any carbon atom on the saturated heterocycle constituted by R², R³, and the nitrogen atom, and R⁴ together may form a 4- to 11-membered saturated heterocycle,
or a pharmaceutically acceptable salt thereof.

2. The compound according to claim 1 or a pharmaceutically acceptable salt thereof, wherein Z represents —NR²R³, and R² and R³ each independently represent a hydrogen atom, optionally substituted C₁₋₆ alkyl, or C(O)—R^{E}, wherein R² and R³, together with the nitrogen atom to which they are attached, may form an optionally substituted 4- to 8-membered saturated heterocycle.

3. The compound according to claim 1 or a pharmaceutically acceptable salt thereof, wherein Z represents —NR²R³, and R³ and R⁴, together with the nitrogen atom and the carbon atom to which they, respectively, are attached, form an optionally substituted 4- to 8-membered saturated heterocycle.

4. The compound according to claim 1 or a pharmaceutically acceptable salt thereof, wherein R¹ is a hydrogen atom.

5. The compound according to claim 1 or a pharmaceutically acceptable salt thereof, wherein X is a carbon atom.

6. The compound according to claim 5 or a pharmaceutically acceptable salt thereof, wherein A¹ is an oxygen atom, A² is methylene, and L¹ is methylene.

7. The compound according to claim 1 or a pharmaceutically acceptable salt thereof, wherein
formula (1) is represented by the following formula (1a): 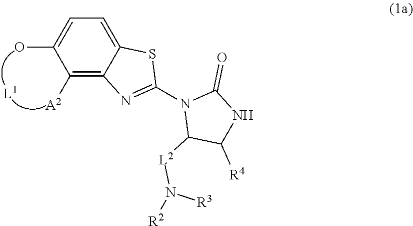
wherein
A² represents optionally substituted methylene or an oxygen atom,
L¹ represents optionally substituted methylene or optionally substituted ethylene,
L² represents optionally substituted C₁₋₄ alkylene,
R² and R³ each independently represent a hydrogen atom, optionally substituted C₁₋₆ alkyl, or C(O)—R^{E}, wherein R² and R³, together with the nitrogen atom to which they are attached, may form an optionally substituted 4- to 8-membered saturated heterocycle,
R^{E} represents optionally substituted C₁₋₆ alkyl, and
R⁴ is optionally substituted C₁₋₆ alkyl.

8. The compound according to claim 7, wherein R² and R³ each independently are optionally substituted C₁₋₆ alkyl, or a pharmaceutically acceptable salt thereof.

9. The compound according to claim 7 or a pharmaceutically acceptable salt thereof, wherein R² and R³, together with the nitrogen atom to which they are attached, form an optionally substituted 4- to 8-membered saturated heterocycle.

10. The compound according to claim 7 or a pharmaceutically acceptable salt thereof, wherein L² is C₁₋₃ alkylene.

11. The compound according to claim 1 or a pharmaceutically acceptable salt thereof, wherein
formula (1) is represented by the following formula (1b): 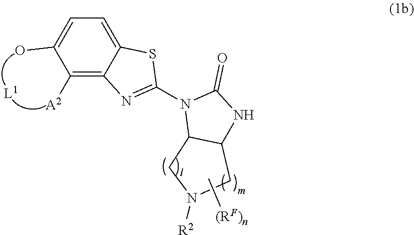
wherein
A² represents optionally substituted methylene or an oxygen atom,
L¹ represents optionally substituted methylene or optionally substituted ethylene,
l and m each independently represent 1, 2, or 3, wherein a sum of 1 and m is 5 or less,
n represents 1, 2, 3, or 4,
R² represents a hydrogen atom, optionally substituted C₁₋₆ alkyl, C₃₋₁₀ cycloalkyl, or C(O)—R^{E},
R^{E} represents optionally substituted C₁₋₆ alkyl,
R^{F} represents a hydrogen atom, a halogen atom, or optionally substituted C₁₋₆ alkyl,
when n is 2, 3, or 4, each R^{F} may be the same or different, and two R^{F} on the same carbon atom, together with the carbon atom to which they are each attached, may form a spiro ring consisting of a 4- to 8-membered saturated heterocycle or a 3- to 8-membered saturated carbocycle, or two
R^{F} on different carbon atoms may bond together to form a crosslink.

12. The compound according to claim 11 or a pharmaceutically acceptable salt thereof, wherein R² is a hydrogen atom, optionally substituted C₁₋₆ alkyl, or C₃₋₁₀ cycloalkyl.

13. The compound according to claim 11 or a pharmaceutically acceptable salt thereof, wherein R² is optionally substituted C₁₋₆ alkyl.

14. The compound according to claim 11 or a pharmaceutically acceptable salt thereof, wherein 1 and m are each independently 1 or 2.

15. The compound according to claim 1, wherein Z is —OR⁷, and R⁴ and R⁷, together with the carbon atom and the oxygen atom to which they, respectively, are attached, form an optionally substituted 5- to 8-membered saturated heterocycle, or a pharmaceutically acceptable salt thereof.

16. The compound according to claim 1 or a pharmaceutically acceptable salt thereof, wherein
formula (1) is represented by the following formula (1c): 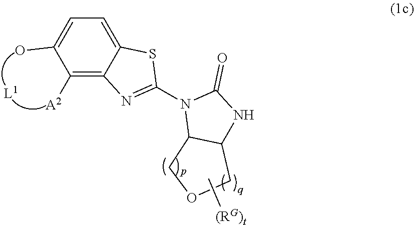
wherein
A² represents optionally substituted methylene or an oxygen atom,
L¹ represents optionally substituted methylene or optionally substituted ethylene,
p and q each independently represent 1, 2, or 3, wherein a sum of p and q is 5 or less,
t represents 1, 2, 3, or 4,
R^{G} represents a hydrogen atom, a halogen atom, optionally substituted C₁₋₆ alkyl, optionally substituted C₁₋₆ alkoxy, or a CN group,
when t is 2, 3, or 4, each R^{G} may be the same or different, and two R^{G} on the same carbon atom, together with the carbon atom to which they are each attached, may form a spiro ring consisting of a 4- to 8-membered saturated heterocycle or a 3- to 8-membered saturated carbocycle, or two R^{G} on different carbon atoms may bond together to form a cross-link.

17. The compound according to claim 1 or a pharmaceutically acceptable salt thereof, wherein the compound is selected from the group of the following compounds:
cis-3-(7,8-dihydrofuro[3,2-e][1,3]benzothiazol-2-yl)-5-methyloctahydro-2H-imidazo[4,5-c]pyridin-2-one (Example 7);
cis-3-(7,8-dihydrofuro[3,2-e][1,3]benzothiazol-2-yl)-5-(2-methoxyethyl)octahydro-2H-imidazo[4,5-c]pyridin-2-one (Example 8);
cis-1-(7,8-dihydrofuro[3,2-e][1,3]benzothiazol-2-yl)-5-methyloctahydro-2H-imidazo[4,5-c]pyridin-2-one (Example 9);
cis-3-(7,8-dihydrofuro[3,2-e][1,3]benzothiazol-2-yl)-5-ethyloctahydro-2H-imidazo[4,5-c]pyridin-2-one (Example 11);
cis-1-(7,8-dihydrofuro[3,2-e][1,3]benzothiazol-2-yl)-5-methylhexahydropyrrolo[3,4-d]imidazol-2(1H)-one (Example 14);
cis-3-(7,8-dihydrofuro[3,2-e][1,3]benzothiazol-2-yl)-5-(propan-2-yl)octahydro-2H-imidazo[4,5-c]pyridin-2-one (Example 15);
cis-3-(7,8-dihydrofuro[3,2-e][1,3]benzothiazol-2-yl)-5-methyloctahydroimidazo[4,5-c]azepin-2(1H)-one (Example 17);
(3aS,7aS)-3-(7,8-dihydrofuro[3,2-e][1,3]benzothiazol-2-yl)-5-(oxetan-3-yl)octahydro-2H-imidazo[4,5-c]pyridin-2-one (Example 19);
(3aR,7aR)-1-(2H-[1,3]dioxolo[4,5-e][1,3]benzothiazol-7-yl)hexahydropyrano[3,4-d]imidazol-2(3H)-one (Example 21);
(4S,5R)-1-(7,8-dihydrofuro[3,2-e][1,3]benzothiazol-2-yl)-4-methyl-5-[(morpholin-4-yl)methyl]imidazolidin-2-one (Example 23);
(4R,5R)-1-(7,8-dihydrofuro[3,2-e][1,3]benzothiazol-2-yl)-4-methyl-5-[(morpholin-4-yl)methyl]imidazolidin-2-one (Example 24);
(3aR,7aS)-3-(7,8-dihydrofuro[3,2-e][1,3]benzothiazol-2-yl)-5-methyloctahydro-2H-imidazo[4,5-c]pyridin-2-one (Example 28);
(3aR,7aS)-5-cyclopropyl-3-(7,8-dihydro[1,4]dioxino[2,3-e][1,3]benzothiazol-2-yl)octahydro-2H-imidazo[4,5-c]pyridin-2-one (Example 34);
(3aR,7aS)-5-cyclopropyl-3-(2H-[1,3]dioxolo[4,5-e][1,3]benzothiazol-7-yl)octahydro-2H-imidazo[4,5-c]pyridin-2-one (Example 36);
(3aR,7aR)-1-(7,8-dihydro[1,4]dioxino[2,3-e][1,3]benzothiazol-2-yl)hexahydropyrano[3,4-d]imidazol-2(3H)-one (Example 37);
(3aR,6S,7aR)-1-(7,8-dihydrofuro[3,2-e][1,3]benzothiazol-2-yl)-6-methylhexahydropyrano[3,4-d]imidazol-2(3H)-one (Example 38);
(3aR,6S,7aR)-1-(7,8-dihydro[1,4]dioxino[2,3-e][1,3]benzothiazol-2-yl)-6-methylhexahydropyrano[3,4-d]imidazol-2(3H)-one (Example 39);
(3aR,6R,7aR)-1-(7,8-dihydrofuro[3,2-e][1,3]benzothiazol-2-yl)-6-(fluoromethyl)hexahydropyrano[3,4-d]imidazol-2(3H)-one (Example 40);
(3aR,6R,7aR)-1-(7,8-dihydro[1,4]dioxino[2,3-e][1,3]benzothiazol-2-yl)-6-(fluoromethyl)hexahydropyrano[3,4-d]imidazol-2(3H)-one (Example 41);
(3aR,6S,7aR)-1-(7,8-dihydrofuro[3,2-e][1,3]benzothiazol-2-yl)-6-ethylhexahydropyrano[3,4-d]imidazol-2(3H)-one (Example 42);
(3aR,6S,7aR)-1-(7,8-dihydro[1,4]dioxino[2,3-e][1,3]benzothiazol-2-yl)-6-ethylhexahydropyrano[3,4-d]imidazol-2(3H)-one (Example 43);
(4S,5S)-1-(7,8-dihydrofuro[3,2-e][1,3]benzothiazol-2-yl)-5-(methoxymethyl)-4-methylimidazolidin-2-one (Example 44);
(3aR,6aS)-1-(7,8-dihydrofuro[3,2-e][1,3]benzothiazol-2-yl)tetrahydro-1H-furo[3,4-d]imidazol-2(3H)-one (Example 65);
rac-(3aR,7aS)-3-(7,8-dihydrofuro[3,2-e][1,3]benzothiazol-2-yl)-5-(oxetan-3-yl)octahydro-2H-imidazo[4,5-c]pyridin-2-one (Example 71);
rac-(3aR,7aS)-5-cyclobutyl-3-(7,8-dihydrofuro[3,2-e][1,3]benzothiazol-2-yl)octahydro-2H-imidazo[4,5-c]pyridin-2-one (Example 72);
rac-(3aR,8aS)-3-(7,8-dihydrofuro[3,2-e][1,3]benzothiazol-2-yl)-5-(oxetan-3-yl)octahydroimidazo[4,5-c]azepin-2(1H)-one (Example 73);
rac-(3aR,7aS)-3-(7,8-dihydro[1,4]dioxino[2,3-e][1,3]benzothiazol-2-yl)-5-methyloctahydro-2H-imidazo[4,5-c]pyridin-2-one (Example 76);
rac-(3aR,7aS)-1-(7,8-dihydro[1,4]dioxino[2,3-e][1,3]benzothiazol-2-yl)-5-methyloctahydro-2H-imidazo[4,5-c]pyridin-2-one (Example 77);
rac-(3aR,7aS)-1-(2H-[1,3]dioxolo[4,5-e][1,3]benzothiazol-7-yl)-5-methyloctahydro-2H-imidazo[4,5-c]pyridin-2-one (Example 78);
rac-(3aR,7aS)-3-(2H-[1,3]dioxolo[4,5-e][1,3]benzothiazol-7-yl)-5-methyloctahydro-2H-imidazo[4,5-c]pyridin-2-one (Example 79);
rac-(3aR,7aS)-3-(7,8-dihydro[1,4]dioxino[2,3-e][1,3]benzothiazol-2-yl)-5-(oxetan-3-yl)octahydro-2H-imidazo[4,5-c]pyridin-2-one (Example 80);
rac-(3aR,7aS)-3-(2H-[1,3]dioxolo[4,5-e][1,3]benzothiazol-7-yl)-5-(oxetan-3-yl)octahydro-2H-imidazo[4,5-c]pyridin-2-one (Example 81);
rac-(3aR,8aS)-3-(2H-[1,3]dioxolo[4,5-e][1,3]benzothiazol-7-yl)-5-methyloctahydroimidazo[4,5-c]azepin-2(1H)-one (Example 85);
rac-(3aR,8aS)-3-(2H-[1,3]dioxolo[4,5-e][1,3]benzothiazol-7-yl)-5-(oxetan-3-yl)octahydroimidazo[4,5-c]azepin-2(1H)-one (Example 86);
(3aS,6S,7aR)-1-(7,8-dihydrofuro[3,2-e][1,3]benzothiazol-2-yl)-5,6-dimethyloctahydro-2H-imidazo[4,5-c]pyridin-2-one (Example 93);
rac-(3aR,7aS)-5-cyclopropyl-3-(7,8-dihydrofuro[3,2-e][1,3]benzothiazol-2-yl)octahydro-2H-imidazo[4,5-c]pyridin-2-one (Example 98);
rac-(3aR,7aS)-3-(7,8-dihydrofuro[3,2-e][1,3]benzothiazol-2-yl)-5-(2-propyn-1-yl)octahydro-2H-imidazo[4,5-c]pyridin-2-one (Example 99);
rac-[(3aR,7aS)-3-(7,8-dihydrofuro[3,2-e][1,3]benzothiazol-2-yl)-2-oxooctahydro-5H-imidazo[4,5-c]pyridin-5-yl]acetonitrile (Example 100);
rac-(3aR,6aS)-1-(7,8-dihydrofuro[3,2-e][1,3]benzothiazol-2-yl)-5-(2-propyn-1-yl)hexahydropyrrolo[3,4-d]imidazol-2(1H)-one (Example 102);
rac-[(3aR,6aS)-1-(7,8-dihydrofuro[3,2-e][1,3]benzothiazol-2-yl)-2-oxohexahydropyrrolo[3,4-d]imidazol-5(1H)-yl]acetonitrile (Example 103);
rac-(3aR,8aS)-5-cyclopropyl-3-(7,8-dihydrofuro[3,2-e][1,3]benzothiazol-2-yl)octahydroimidazo[4,5-c]azepin-2(1H)-one (Example 104);
rac-(3aR,8aS)-3-(7,8-dihydrofuro[3,2-e][1,3]benzothiazol-2-yl)-5-(2-propyn-1-yl)octahydroimidazo[4,5-c]azepin-2(1H)-one (Example 105);
rac-[(3aR,8aS)-3-(7,8-dihydrofuro[3,2-e][1,3]benzothiazol-2-yl)-2-oxooctahydroimidazo[4,5-c]azepin-5(1H)-yl]acetonitrile (Example 106);
rac-(3aR,7aS)-3-(7,8-dihydrofuro[3,2-e][1,3]benzothiazol-2-yl)-5-(2,2,2-trifluoroethyl)octahydro-2H-imidazo[4,5-c]pyridin-2-one (Example 107);
rac-(3aR,7aS)-5-(2,2-difluoroethyl)-3-(7,8-dihydrofuro[3,2-e][1,3]benzothiazol-2-yl)octahydro-2H-imidazo[4,5-c]pyridin-2-one (Example 108);
(3aR,7aS)-5-cyclopropyl-3-(7,8-dihydrofuro[3,2-e][1,3]benzothiazol-2-yl)octahydro-2H-imidazo[4,5-c]pyridin-2-one (Example 110);
[(3aR,7aS)-3-(7,8-dihydrofuro[3,2-e][1,3]benzothiazol-2-yl)-2-oxooctahydro-5H-imidazo[4,5-c]pyridin-5-yl]acetonitrile (Example 112);
(3aR,7aS)-3-(7,8-dihydrofuro[3,2-e][1,3]benzothiazol-2-yl)-5-(2-propyn-1-yl)octahydro-2H-imidazo[4,5-c]pyridin-2-one (Example 114);
(4S,5R)-1-(7,8-dihydrofuro[3,2-e][1,3]benzothiazol-2-yl)-5-{[(3S)-3-fluoropyrrolidin-1-yl]methyl}-4-methylimidazolidin-2-one (Example 115);
(4S,5R)-5-[(3,3-difluoroazetidin-1-yl)methyl]-1-(7,8-dihydrofuro[3,2-e][1,3]benzothiazol-2-yl)-4-methylimidazolidin-2-one (Example 116);
(4S,5R)-5-[(3,3-difluoropyrrolidin-1-yl)methyl]-1-(7,8-dihydrofuro[3,2-e][1,3]benzothiazol-2-yl)-4-methylimidazolidin-2-one (Example 117);
(4S,5R)-1-(7,8-dihydrofuro[3,2-e][1,3]benzothiazol-2-yl)-5-[(3-fluoroazetidin-1-yl)methyl]-4-methylimidazolidin-2-one (Example 118);
rac-(3aR,7aR)-3-(7,8-dihydrofuro[3,2-e][1,3]benzothiazol-2-yl)hexahydropyrano[3,4-d]imidazol-2(3H)-one (Example 120);
rac-(3aR,7aR)-1-(7,8-dihydrofuro[3,2-e][1,3]benzothiazol-2-yl)hexahydropyrano[3,4-d]imidazol-2(3H)-one (Example 121);
(3aR,7aR)-1-(7,8-dihydrofuro[3,2-e][1,3]benzothiazol-2-yl)hexahydropyrano[3,4-d]imidazol-2(3H)-one (Example 122);
(3aR,7aR)-1-(7,8-dihydrofuro[3,2-e][1,3]benzothiazol-2-yl)-6,6-dimethylhexahydropyrano[3,4-d]imidazol-2(3H)-one (Example 124);
(3aR,6R,7aR)-1-(7,8-dihydrofuro[3,2-e][1,3]benzothiazol-2-yl)-2-oxooctahydropyrano[3,4-d]imidazole-6-carbonitrile (Example 127);
(3aR,6R,7aR)-1-(7,8-dihydrofuro[3,2-e][1,3]benzothiazol-2-yl)-6-methoxyhexahydropyrano[3,4-d]imidazol-2(3H)-one (Example 129);
(3aR,6R,7aR)-1-(2H-[1,3]dioxolo[4,5-e][1,3]benzothiazol-7-yl)-2-oxooctahydropyrano[3,4-d]imidazole-6-carbonitrile (Example 131);
(3aR,6R,7aR)-1-(7,8-dihydro[1,4]dioxino[2,3-e][1,3]benzothiazol-2-yl)-2-oxooctahydropyrano[3,4-d]imidazole-6-carbonitrile (Example 133);
(3aR,8aR)-1-(7,8-dihydro[1,4]dioxino[2,3-e][1,3]benzothiazol-2-yl)hexahydro-1H-oxepino[3,4-d]imidazol-2(3H)-one (Example 135);
(3aR,6S,7aR)-1-(2H-[1,3]dioxolo[4,5-e][1,3]benzothiazol-7-yl)-6-ethylhexahydropyrano[3,4-d]imidazol-2(3H)-one (Example 138);
(3aR,6S,7aR)-1-(2H-[1,3]dioxolo[4,5-e][1,3]benzothiazol-7-yl]-6-methylhexahydropyrano[3,4-d]imidazol-2(3H)-one (Example 139);
(3aR,6R,7aR)-1-(2H-[1,3]dioxolo[4,5-e][1,3]benzothiazol-7-yl]-6-(fluoromethyl)hexahydropyrano[3,4-d]imidazol-2(3H)-one (Example 140);
(3aR,6R,7aR)-1-(7,8-dihydrofuro[3,2-e][1,3]benzothiazol-2-yl)-6-(hydroxymethyl)hexahydropyrano[3,4-d]imidazol-2(3H)-one (Example 144);
(3aR,6R,7aR)-1-(7,8-dihydro[1,4]dioxino[2,3-e][1,3]benzothiazol-2-yl)-6-(hydroxymethyl)hexahydropyrano[3,4-d]imidazol-2(3H)-one (Example 146);
(3aR,6R,7aR)-1-(2H-[1,3]dioxolo[4,5-e][1,3]benzothiazol-7-yl)-6-(hydroxymethyl)hexahydropyrano[3,4-d]imidazol-2(3H)-one (Example 147);
(3aR,6R,7aR)-1-(7,8-dihydrofuro[3,2-e][1,3]benzothiazol-2-yl)-6-ethynylhexahydropyrano[3,4-d]imidazol-2(3H)-one (Example 149);
(3aR,7aR)-1-(7,8-dihydrofuro[3,2-e][1,3]benzothiazol-2-yl)-6-fluorohexahydropyrano[3,4-d]imidazol-2(3H)-one (Example 151);
(3aR,6S,6aS)-1-(7,8-dihydrofuro[3,2-e][1,3]benzothiazol-2-yl)-6-(hydroxymethyl)tetrahydro-1H-furo[3,4-d]imidazol-2(3H)-one (Example 157);
(3aR,6S,6aS)-1-(7,8-dihydrofuro[3,2-e][1,3]benzothiazol-2-yl)-6-(fluoromethyl)tetrahydro-1H-furo[3,4-d]imidazol-2(3H)-one (Example 159);
(3aR,6R,6aS)-1-(7,8-dihydrofuro[3,2-e][1,3]benzothiazol-2-yl)-6-methyltetrahydro-1H-furo[3,4-d]imidazol-2(3H)-one (Example 161); and
[(3aS,4R,6aR)-3-(7,8-dihydrofuro[3,2-e][1,3]benzothiazol-2-yl)-2-oxohexahydro-1H-furo[3,4-d]imidazol-4-yl]acetonitrile (Example 162).

18. A medicament comprising the compound according to claim 1 or a pharmaceutically acceptable salt thereof as an active ingredient.

19. A pharmaceutical composition comprising the compound according to claim 1 or a pharmaceutically acceptable salt thereof as an active ingredient.

20. A therapeutic agent and/or a prophylactic agent for a disease involving DYRK, comprising the compound according to claim 1 or a pharmaceutically acceptable salt thereof as an active ingredient.

21. The therapeutic agent and/or the prophylactic agent according to claim 20, wherein the disease involving DYRK is frontotemporal dementia, progressive supranuclear palsy, corticobasal degeneration, Lewy body dementia, vascular dementia, traumatic brain injury, chronic traumatic encephalopathy, stroke, Alzheimer's disease, Parkinson's disease, Down's disease, or depression, and mental retardation, memory impairment, memory loss, learning disability, intellectual disability, cognitive dysfunction, mild cognitive impairment, or dementia symptom associated therewith, or brain tumor, pancreatic cancer, ovarian cancer, osteosarcoma, large intestine cancer, lung cancer, bone resorption disease, osteoporosis, sickle cell anemia, chronic renal disease, or bone resorption disease.
